(19) United States
(12) Patent Application Publication
(10) Pub. No.: US 2025/0144078 A1
(43) Pub. Date: May 8, 2025

(54) THIOSTREPTON-INSPIRED COMPOUNDS FOR TREATMENT OF CANCER AND PREPARATION THEREOF

(71) Applicant: RS Oncology, llc, Cambridge, MA (US)

(72) Inventors: George N. Naumov, Brookline, MA (US); Jarrett B. Duncan, Dunbarton, NH (US); Paul R. Werkhoven, Nijmegen (NL); Bart DeCorte, Southampton, PA (US); Brian Cunniff, Jericho, VT (US); Johannes W.G. Meissner, Nijmegen (NL)

(21) Appl. No.: 18/837,725

(22) PCT Filed: Feb. 15, 2023
(86) PCT No.: PCT/US23/13118

§ 371 Date: Aug. 12, 2024

Related U.S. Application Data
(60) Provisional application No. 63/341,238, filed on May 12, 2022.
(60) Provisional application No. 63/310,400, filed on Feb. 15, 2022.

(51) Int. Cl. A61K 31/4192; A61K 31/165; A61K 31/343; A61K 31/40; A61K 31/4172; A61K 31/421; A61K 31/426; A61K 31/427; A61K 31/428; A61K 31/429; A61K 31/437; A61K 31/4439; A61K 31/497; A61K 31/501; A61P 35/00; C07C 237/24; C07C 237/34; C07D 207/16; C07D 233/90; C07D 249/10; C07D 263/34; C07D 277/56; C07D 277/68; C07D 307/94; C07D 333/24; C07D 417/04; C07D 417/12; C07D 471/04; C07D 487/04; C07D 513/04

(52) U.S. Cl. CPC A61K 31/4192; A61K 31/165; A61K 31/343; A61K 31/40; A61K 31/4172; A61K 31/421; A61K 31/426; A61K 31/427; A61K 31/428; A61K 31/429; A61K 31/437; A61K 31/4439; A61K 31/497; A61K 31/501; A61P 35/00; C07C 237/24; C07C 237/34; C07D 207/16; C07D 233/90; C07D 249/10; C07D 263/34; C07D 277/56; C07D 277/68; C07D 307/94; C07D 333/24; C07D 417/04; C07D 417/12; C07D 471/04; C07D 487/04; C07D 513/04

(57) ABSTRACT

Disclosed herein are structures, compositions and syntheses of pharmaceutically relevant compounds inspired by thiostrepton, as well as methods of treating cancer with such compounds.

### CROSS REFERENCE TO RELATED APPLICATIONS

This application claims the benefit of priority to U.S. Provisional Patent Application No. 63/310,400, filed Feb. 15, 2022, and U.S. Provisional Patent Application No. 63/341,238, filed May 12, 2022, each of which is hereby incorporated by reference in its entirety.

### BACKGROUND

Thiostrepton is a cyclic oligopeptide antibiotic that is also known by other names such as Bryamycin, Thiactin, alaninamide, HR4S203Y18, etc. Recent studies have shown that thiostrepton also has promising anticancer activity. There remains a need for thiostrepton derivatives having beneficial pharmacological properties.

### SUMMARY

In certain embodiments, the present invention provides a series of compounds having the structure of Formula (IA):

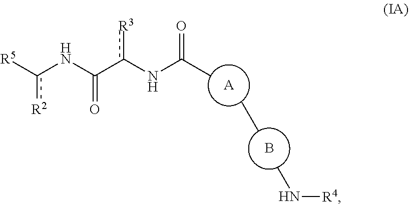

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety;
each [symbol] is independently a single bond or a double bond; and
any hydrogen atom is optionally replaced with a deuterium.

In certain embodiments, the present invention also provides a series of compounds having the structure of Formula (IB):

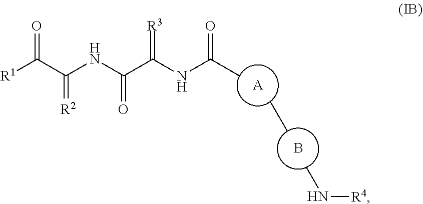

or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety; and
any hydrogen atom is optionally replaced with a deuterium.

Also provided herein are methods of treating cancer, comprising administering to a subject in need thereof any of the pharmaceutical compositions described herein.

### BRIEF DESCRIPTION OF THE FIGURES

FIG. 1: Cell viability assay data in Malignant Mesothelioma (MM) cells (HMESO cell line) treated with compound (1)-HCl or (5)-HCl. N=2 technical and 2 biological replicates conducted by 2 different lab technicians. All compounds went into solution in 100% DMSO. (5)-HCl precipitated at concentrations of ˜20-80 μM when added to complete tissue culture media.

FIG. 2A: View (40× magnification) of HMESO mesothelioma cells treated with (5)-HCl.

FIG. 2B: View (20× magnification) of HMESO mesothelioma cells treated with (5)-HCl.

FIG. 3: Western blots of PRX3 from HMESO mesothelioma cells treated with (1) or (5).

FIG. 4: Bar graph of PRX3 (PRX3-X-PRX3): Monomer ratio of HMESO mesothelioma cells treated with (1) or (5).

FIG. 5 is a table summarizing the results of Examples 42-44 for various compounds described herein. NT indicates “not tested.” For EC₅₀ values, “A” represents an EC₅₀ less than or equal to 5.0 μM, “B” represents an EC₅₀ from 5.1 μM to 10.0 μM, “C” represents an EC₅₀ from 10.1 μM to 50.0 μM, and “D” represents an EC₅₀ greater than 50.0 μM.

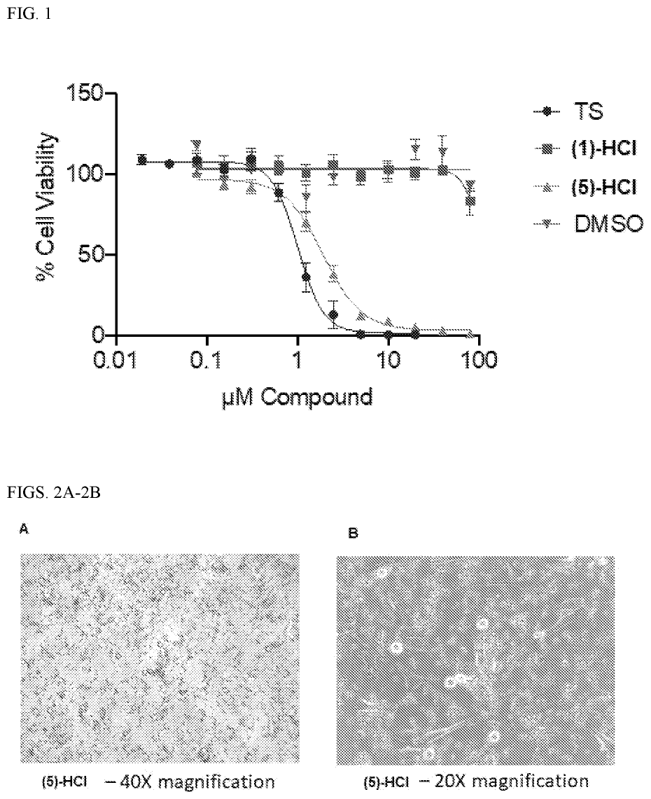

### DETAILED DESCRIPTION

### Pharmaceutical Compositions

The compositions and methods described herein may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound described herein and a pharmaceutically acceptable carrier. Pharmaceutically acceptable carriers are well known in the art and include, as a non-limiting example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound described herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound described herein, with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

Formulations described herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods described herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors that influence the effective amount may include, but are not limited to, the severity of the patient's condition, the disorder being treated, the stability of the compound, and, if desired, another type of therapeutic agent being administered with the compound described herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods described herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

The present disclosure includes the use of pharmaceutically acceptable salts of compounds described herein in the compositions and methods described herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, l-pyroglutamic acid, salicylic acid, sebacic acid, stearic acid, succinic acid, sulfuric acid, 1 tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., when at least 5% of drug product is detectable systemically with industry acceptable methodology, or when the two agents are simultaneously effective in the patient, which may include synergistic effects of the two agents). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic agents.

A “therapeutically effective amount” or a “therapeutically effective dose” of a compound or other agent described herein is an amount of a drug or an agent that, when administered to a subject will have the intended therapeutic effect. The full therapeutic effect does not necessarily occur by administration of one dose of such a drug or agent, and may occur only after administration of a series of doses (multiple consecutive doses). Thus, a therapeutically effective amount may be administered in one or more administrations. The precise effective amount needed for a subject will depend upon, for example, the subject's size, health and age, and the nature and extent of the condition being treated, such as cancer.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds for laboratory use, or for subsequent conversion to a pharmaceutically acceptable acid addition salt.

The term “pharmaceutically acceptable basic addition salt” as used herein means any non-toxic organic or inorganic base addition salt of any acid compounds or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in an R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce IPA or a salt thereof. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “linker” as used herein means any chemical functionality that “links,” or connects with chemical bonds, any two or more other chemical functionalities in a pharmaceutically relevant molecule. As a non-limiting example of the use of linkers in pharmaceutical settings, antibody-drug conjugations (ADCs) comprise a pharmaceutically active small-molecule, drug, or toxin connected to a large-molecule antibody by a linker.

Examples of common linker types include both cleavable and non-cleavable linkers. Cleavable linkers include chemical functionalities that can be cleaved in response to physiological stimuli such as chemical gradients, pH changes, or enzymatic activity. Non-limiting examples include acid- or base-labile functional groups, pyrophosphate diester, disulfide bonds, peptides, β-glucuronides, etc. Non-cleavable linkers comprise chemical functionalities that are generally less labile to the aforementioned physiological stimuli, and non-limiting examples include certain alkyl groups and organic polymeric functionalities.

The term “reactive linker moiety” as used herein refers to a chemical structure having a terminal moiety that can react and form a covalent bond with another moiety (such as the mitochondrial targeting moiety).

The term “mitochondrial targeting peptide,” “mitochondrial targeting sequence,” “mitochondrial targeting moiety,” as used herein are art-recognized terms referring to a chemical functionality (the peptide, sequence, or moiety), which “target,”—i.e., are readily transported to and absorbed by—mitochondrial membranes (J. Zielonka, B. Kalyanaraman, et al., 2017). As used herein, mitochondrial targeting moieties may include but are not limited to the following species: berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenylethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (L-cyclohexyl alanine-D-arginine)3, a mitochondrial-targeted nanocarrier, a DDDK peptide, glycyrrhetinic acid, α-tocopheryl succinate (α-TOS), a graphene oxide nano carrier, PEG-proapoptotic peptide (KLAKLAK)2, a Dmt-D-Arg-Phe-Lys-NH2 peptide, pyruvaldehyde, N-Nonyl acridine orange, quinoline, styrl-azinium fluorophores, or 15d-PGJ2. Exemplary mitochondrial targeting moieties are listed in See. J Zielonka et al, Chem Rev 2017, 117, p 10043-10120; K L Horton et al, Chemistry & Biology 2008, 15, pp 375-382; G Battogtokh et al, Front Pharmacol 2018, 9:922; U.S. Pat. Nos. 9,173,952 and 9,132,198, the contents of each of which are incorporated by reference herein.

It is understood that substituents and substitution patterns on the compounds described herein can be selected by one of ordinary skilled person in the art to result in chemically stable compounds that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

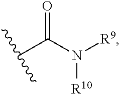

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

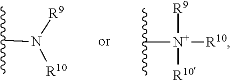

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” used alone or as part of a larger moiety as in “aralkyl”, “aralkoxy”, or “aryloxyalkyl”, refers to monocyclic and bicyclic ring systems having a total of five to fourteen ring members, wherein each ring atom is carbon, at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic carbocyclic rings.

The term “carbamate” is art-recognized and refers to a group

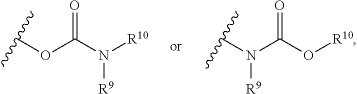

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridinyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl or heteroaryl rings such that the resulting bi- or multicyclic ring system as a whole is fully aromatic. Nonlimiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, and phenoxazinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

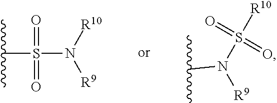

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “protecting group” is an art-recognized term referring to chemical functionalities that can modify (usually covalently) an existing labile functionality on a target molecule. This modification “protects” the labile functionality during subsequent reaction steps, and the protecting group can be removed as needed, termed “deprotection.” As a non-limiting example, the t-butyloxycarbonyl (Boc or boc) group is commonly used to covalently modify and “protect” terminal amine groups in synthetic chemistry.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds.

In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-7-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(RT)S(O)₂R^{†}; wherein each R is independently hydrogen, C₁₋₆ aliphatic which may be substituted as defined below, unsubstituted —OPh, or a substituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group and the substituted 5-6-membered saturated, partially unsaturated, or aryl ring having 0-4 heteroatoms independently selected from nitrogen, oxygen, or sulfur of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

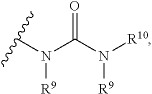

wherein R⁹ and R¹⁰ independently represent hydrogen or a hydrocarbyl.

The term “tautomer” refers to each of two or more isomers of a compound that exist together in equilibrium, and are interchanged by migration of an atom or group within the molecule, such as a hydrogen atom. Exemplary tautomers of the present disclosure include, but are not limited to

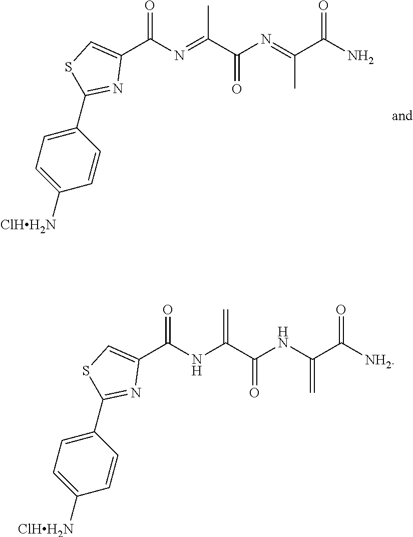

Depiction in this disclosure of one tautomer within a genus or compound species is intended to encompass the compound as drawn and all of its tautomers. Specifically, for the example above, either of those structures also discloses

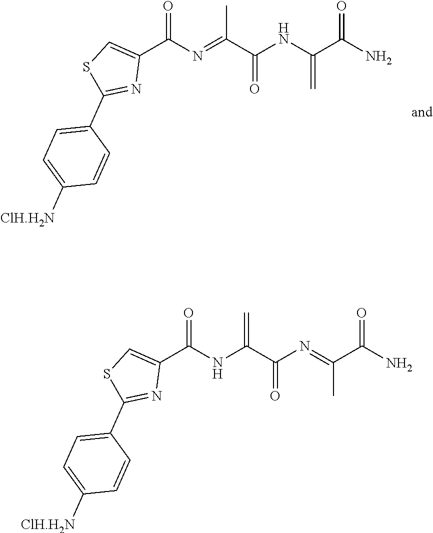

for a series of 2n distinct species where n is the number of tautomeric sites on the molecule.

For the purposes of this disclosure, any of the embodiments described herein applies to any of the generic structural formulas described herein, if properly dependent.

In certain embodiments, the present invention provides a series of compounds compound having the structure of Formula (IA):

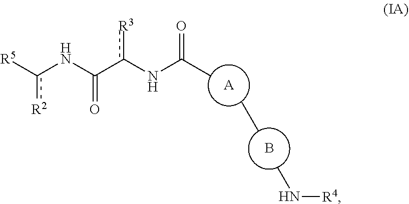

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety;
each [symbol] is independently a single bond or a double bond; and
any hydrogen atom is optionally replaced with a deuterium.

In certain embodiments, the present invention provides a series of compounds having the structure of Formula (2-1):

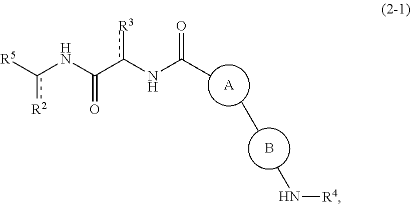

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, -L′, or -L-Y;
L′, when present, is a reactive linker moiety comprising an alkyne group;
L, when present, is a linker moiety;
Y, when present, is a mitochondrial targeting moiety;
each [symbol] is independently a single bond or a double bond;
any hydrogen atom is optionally replaced with a deuterium;
provided that
if Ring A is selected from unsubstituted thiazolyl, oxazolyl, cyclopentyl, and bicyclo[2.1.1]hexane and Ring B—NHR⁴ is

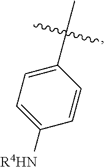

then
a) R⁴ is -L′-Y or L; or
b) R² and R³ are not both ═CH₂ or both ═CH(alkyl).

In certain embodiments, L is

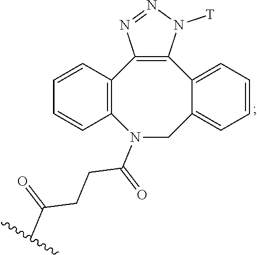

T is selected from alkyl and (—CH₂CH₂—O—)ᵥ—; v is an integer selected from 3-9; and T is bonded to Y.

In certain embodiments, the present invention also provides a series of compounds having the structure of Formula (2-2):

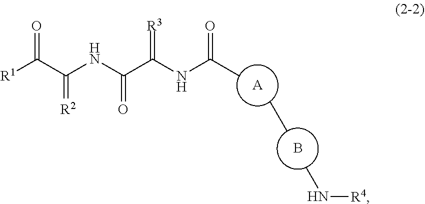

or a pharmaceutically acceptable salt thereof, wherein:
R¹ is —NH₂ or —O—CH₃;
R² is ═CH₂ or CH(alkyl);
R³ is ═CH₂ or CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, -L′, or -L-Y;
L′, when present, is a reactive linker moiety comprising an alkyne group;
L, when present, is a linker moiety;
Y, when present, is a mitochondrial targeting moiety; and
any hydrogen atom is optionally replaced with a deuterium;
provided that
if Ring A is selected from unsubstituted thiazolyl, oxazolyl, cyclopentyl, and bicyclo[2.1.1]hexane and Ring B—NIR⁴ is

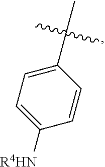

then
a) R⁴ is -L′-Y or L; or
b) R² and R³ are not both ═CH₂ or both ═CH(alkyl).

In certain embodiments, L is

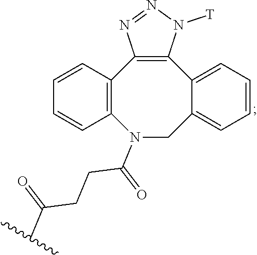

T is selected from alkyl and (—CH₂CH₂—O—)ᵥ—; v is an integer selected from 3-9; and T is bonded to Y.

In certain embodiments, the present invention also provides a series of compounds having the structure of Formula (2-3):

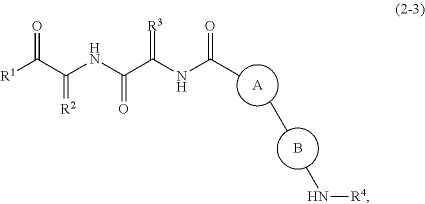

or a pharmaceutically acceptable salt thereof, wherein:
R¹ is —NH₂ or —O—CH₃;
R² is ═CH₂ or ═CH(alkyl);
R³ is ═CH₂ or ═CH(alkyl);
Ring A is heteroaryl, cycloalkyl or heterocyclyl;
Ring B is aryl, heteroaryl, cycloalkyl or heterocyclyl;
R⁴ is hydrogen, a protecting group, or —C(O)—CH₃; and
any hydrogen atom is optionally replaced with a deuterium;
provided that
if Ring A is selected from unsubstituted thiazolyl, oxazolyl, cyclopentyl, and bicyclo[2.1.1]hexane and Ring B—NIR⁴ is

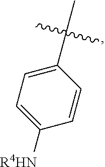

then
R² and R³ are not both ═CH₂ or both ═CH(alkyl).

In certain embodiments, the present invention provides a series of compounds having the structure of Formula (2-4):

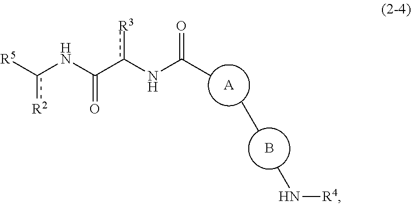

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is selected from

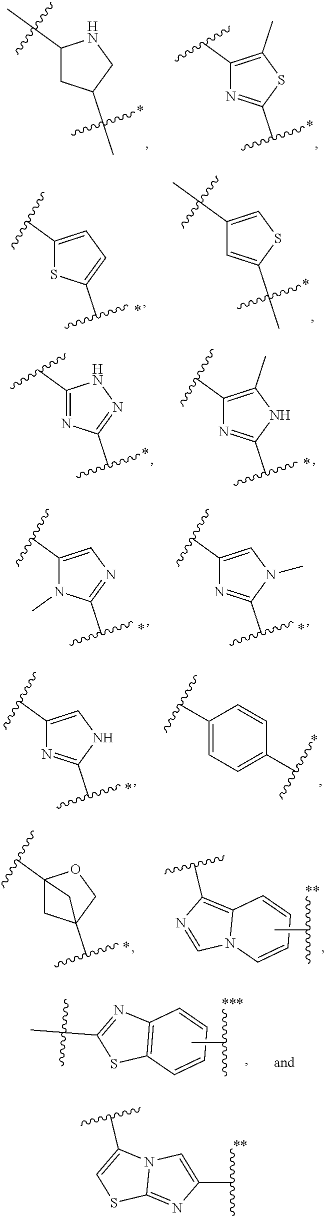

wherein * denotes a bond to Ring B and ** and *** denote —NH—R⁴;
Ring B is absent or present and, when present, is aryl, heteroaryl, cycloalkyl, or heterocyclyl;
R⁴ is hydrogen, a protecting group, —C(O)—CH₃, -L′, or -L-Y;
L′, when present, is a reactive linker moiety comprising an alkyne group;
L, when present, is a linker moiety;
Y, when present is a mitochondrial targeting moiety;
each [symbol] is independently a single bond or a double bond; and
any hydrogen atom is optionally replaced with a deuterium.

In certain embodiments, L is

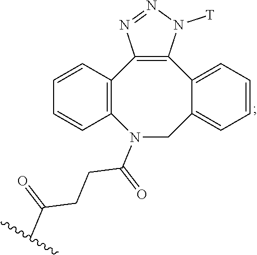

T is selected from alkyl and (—CH₂CH₂—O—)ᵥ—; v is an integer selected from 3-9; and T is bonded to Y.

In certain embodiments, the present invention provides a series of compounds having the structure of Formula (2-5):

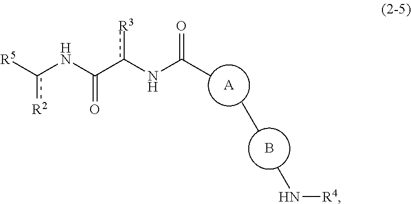

or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B—NHR⁴ is selected from

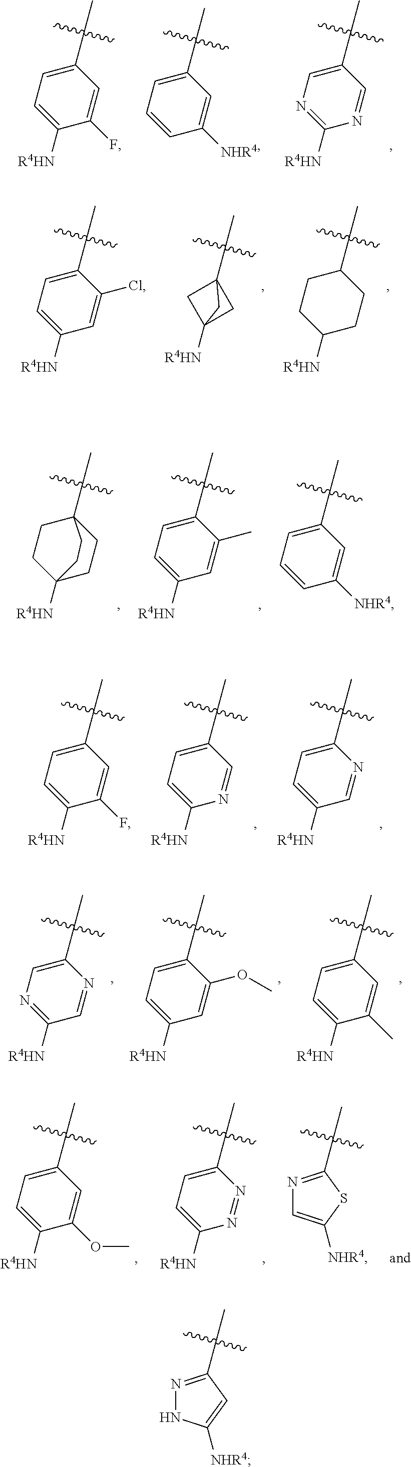

R⁴ is hydrogen, a protecting group, —C(O)—CH₃, -L′, or -L-Y;
L′, when present, is a reactive linker moiety comprising an alkyne group;
L, when present, is a linker moiety;
Y, when present, is a mitochondrial targeting moiety;
each [symbol] is independently a single bond or a double bond; and
any hydrogen atom is optionally replaced with a deuterium.

In certain embodiments, L is

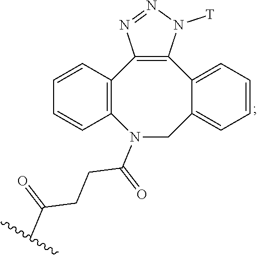

T is selected from alkyl and (—CH₂CH₂—O—)ᵥ—; v is an integer selected from 3-9; and T is bonded to Y.

In certain embodiments, the present invention provides a series of compounds having the structure of Formula (2-6):

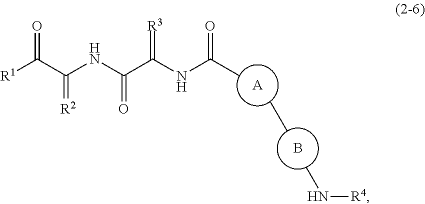

or a pharmaceutically acceptable salt thereof, wherein:
R¹ is —NH₂ or —O—CH₃;
R² is ═CH₂ or ═CH(alkyl);
R³ is ═CH₂ or ═CH(alkyl);
Ring A is thiazolyl substituted with alkyl or thiophenyl;
Ring B—NHR⁴ is

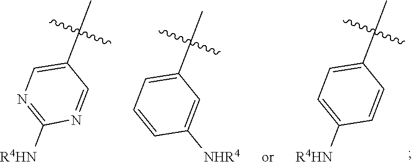

R⁴ is a protecting group or -L′;
L′, when present, is a reactive linker moiety comprising an alkyne group;
any hydrogen atom is optionally replaced with a deuterium.

In certain embodiments, R⁵ is —C(O)—R¹; and R¹ is —OCH₃. In other embodiments, R⁵ is —C(O)—R¹; and R¹ is —NH₂.

In certain embodiments, when Ring A is polycyclic, then Ring B is absent.

In certain embodiments, Ring A is a 5-membered ring or a 5-membered ring fused to a second ring.

In certain embodiments, Ring A is a 5-membered heteroaryl. In some embodiments, Ring A is a bicyclic heteroaryl.

In certain embodiments, Ring A is thiazolyl, thiophenyl, oxazolyl, or imidazolyl. In other embodiments, Ring A is thiazolyl, thiophenyl, or oxazolyl. In some embodiments, Ring A is pyrrolidinyl. In other embodiments, Ring A is thiazolyl.

In certain embodiments, Ring A is:

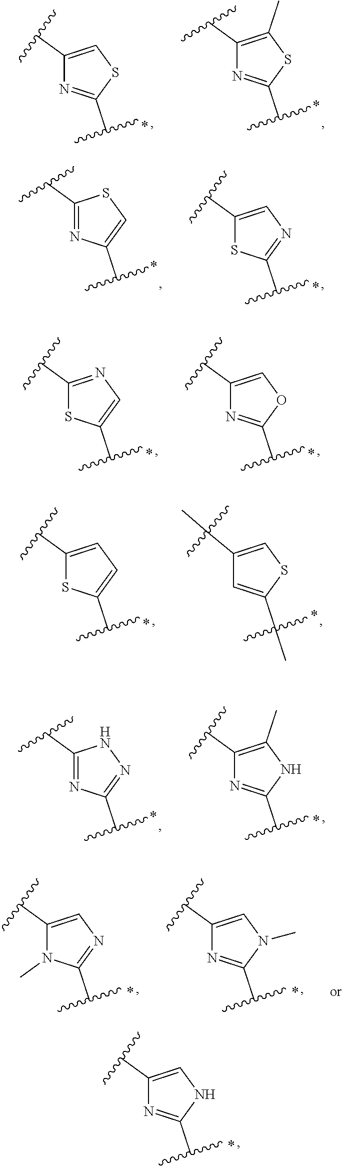

wherein * denotes a bond to Ring B.

In certain embodiments, Ring A is:

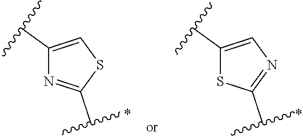 [SMILES: *c1csc(*)n1 ; *c1cnc(*)s1]

wherein * denotes a bond to Ring B.

In certain embodiments, Ring A is:

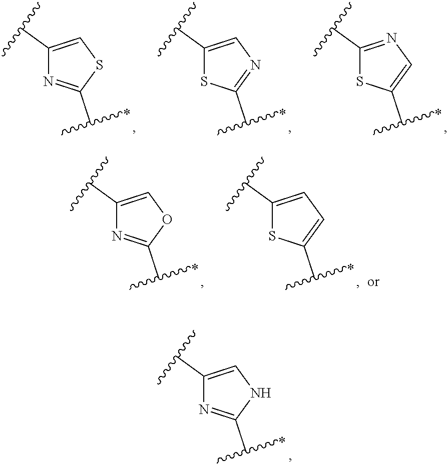 [SMILES: *c1csc(*)n1 ; *c1cnc(*)s1 ; *c1cnc(*)s1 ; *c1coc(*)n1 ; *c1ccc(*)s1 ; *c1c[nH]c(*)n1]

wherein * denotes a bond to Ring B.

In certain embodiments, Ring A is:

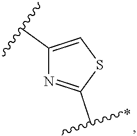 [SMILES: *c1csc(*)n1]

wherein * denotes a bond to Ring B.

In certain embodiments, Ring A is a 5-membered cycloalkyl or heterocyclyl.

In certain embodiments, Ring A is cyclopentyl or tetrahydrofuranyl.

In certain embodiments, Ring A is

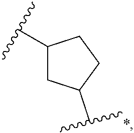 [SMILES: *C1CCC(*)C1]

wherein * denotes a bond to Ring B.

In certain embodiments, Ring A is a bridged bicyclic cycloalkyl or heterocyclyl.

In certain embodiments, Ring A is bicyclo[2.1.1]hexyl or oxabicyclo[2.1.1]hexyl.

In certain embodiments, Ring A is

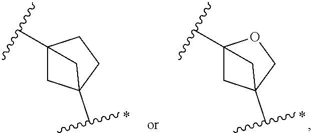 [SMILES: *C12CCC(*)(C1)C2 ; *C12COC(*)(C1)C2]

wherein * denotes a bond to Ring B.

In certain embodiments, wherein Ring A is phenyl.

In certain embodiments, Ring A is

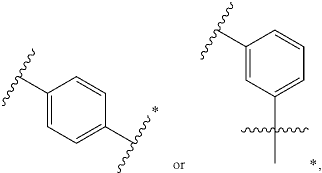 [SMILES: *c1ccc(*)cc1 ; *c1cccc(*)c1]

wherein * denotes a bond to Ring B.

In certain embodiments, Ring A is a polycyclic aryl, heteroaryl, cycloalkyl, or heterocyclyl.

In certain embodiments, Ring A is a bicyclic heteroaryl.

In certain embodiments, Ring A is:

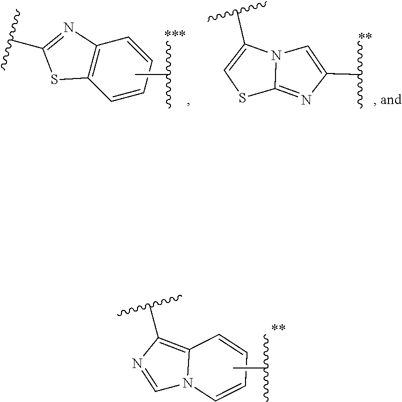

wherein *** and ** denote Ring B or —NH—R⁴.

In certain embodiments, Ring B is a 6-membered ring.

In certain embodiments, B is phenyl.

In certain embodiments, Ring B is unsubstituted phenyl. In other embodiments, Ring B is a halogen-substituted phenyl.

In certain embodiments, Ring B is

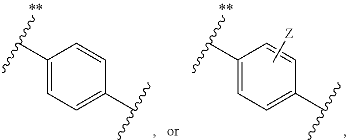

wherein Z is halo and ** denotes a bond to Ring A. In some embodiments, Z is selected from fluoro or chloro.

In certain embodiments, Ring B is:

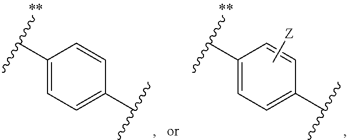

wherein Z is alkoxy, such as methoxy, or alkyl, such as methyl.

In other embodiments, Ring B is

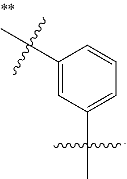 [SMILES: *c1cccc(*)c1]

In certain embodiments, Ring B is a 6-membered heteroaryl.

In certain embodiments, Ring B is pyridinyl, pyrimidinyl pyrazinyl, or pyridazinyl.

In certain embodiments, Ring B is pyridinyl, pyrimidinyl or pyrazinyl.

In certain embodiments, Ring B is:

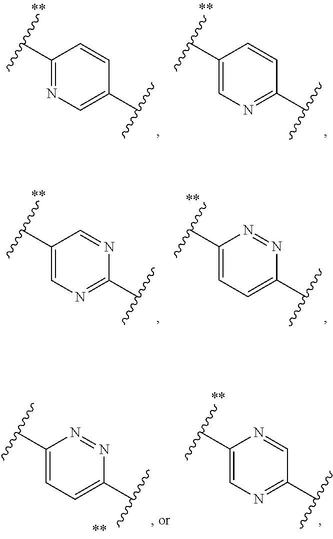 [SMILES: *c1ccc(*)nc1 ; *c1ccc(*)nc1 ; *c1cnc(*)nc1 ; *c1ccc(*)nn1 ; *c1ccc(*)nn1 ; *c1cnc(*)cn1]

wherein ** denotes a bond to Ring A.

In certain embodiments, Ring B is a bridged bicyclic cycloalkyl.

In certain embodiments, Ring A is bicyclo[2.2.2]octanyl or bicyclo[1.1.1]pentanyl.

In certain embodiments, Ring B is:

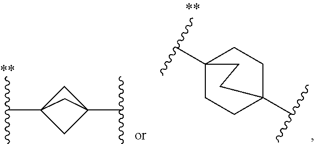

wherein Z is halo and ** denotes a bond to Ring A.

In certain embodiments, Ring B is selected from

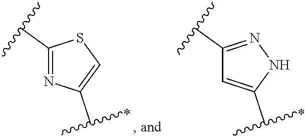 [SMILES: *c1csc(*)n1 ; *c1cc(*)[nH]n1]

where * denotes a bond to —NHR⁴.

In certain embodiments, R⁵ is —CN.

In certain embodiments, the compound of Formula (IA) has the structure of Formula (IA-1):

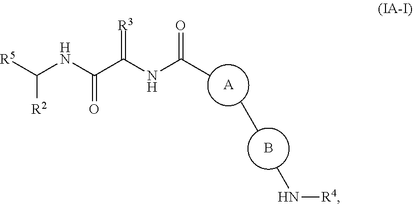

or a pharmaceutically acceptable salt thereof,
wherein
R² is —H or —CH₃; and
R³ is ═CH₂ or ═CH(alkyl); and
each of Ring A, Ring B, R¹, R⁴ and R⁵ are any of the embodiments set forth above for Formula (IA).

In certain embodiments of Formula (IA-1), R² is —H and R³ is ═CH₂, or R² is —CH₃ and R³ is ═CH₂.

In certain embodiments, the compound of Formula (IA) has the structure of Formula (IA-2):

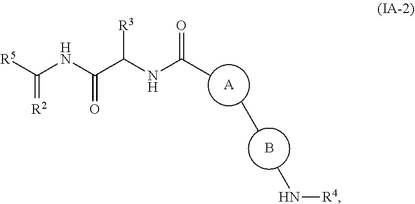

or a pharmaceutically acceptable salt thereof,
wherein
R² is ═CH₂ or ═CH(alkyl); and
R³ is —H or —CH₃; and
each of Ring A, Ring B, R¹, R⁴ and R⁵ are any of the embodiments set forth above for Formula (IA).

In certain embodiments of Formula (IA-2), R² is ═CH₂ and R³ is —H, or R² is ═CH₂ and R³ is —CH₃.

In certain embodiments, the compound of Formula (IA) has the structure of Formula (IA-3):

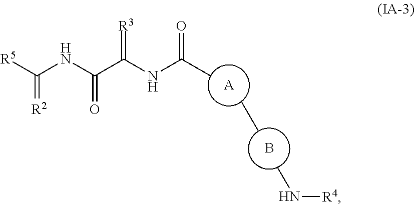

or a pharmaceutically acceptable salt thereof,
wherein
R² is ═CH₂ or ═CH(alkyl); and
R³ is ═CH₂ or ═CH(alkyl); and
each of Ring A, Ring B, R¹, R⁴ and R⁵ are any of the embodiments set forth above for Formula (IA).

In certain embodiments of Formula (IA-3), R² is ═CH₂ and R³ is ═CH₂, R² is ═CH₂ and R³ is ═CH(CH₃), or R² is ═CH(CH₃) and R³ is ═CH₂.

In certain embodiments, the compound of Formula (IA) has the structure of Formula (IB):

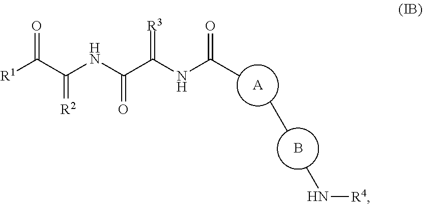

or a pharmaceutically acceptable salt thereof, wherein
each of Ring A, Ring B, R¹, R², R³ and R⁴ are any of the embodiments set forth above for Formula (IA).

In certain embodiments, the compound of Formula (IA) has the structure of Formula (IB):

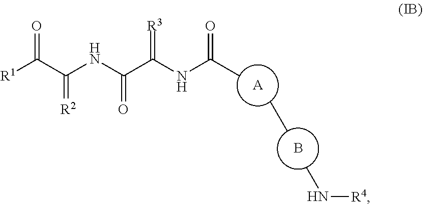

or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl or heterocyclyl;
Ring B is aryl, heteroaryl, cycloalkyl or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety; and
any hydrogen atom is optionally replaced with a deuterium.

In certain embodiments of Formula (IB), Ring A is a 5-membered ring or a 5-membered ring fused or bridged to a second ring. In certain embodiments, Ring A is thiazolyl, thiophenyl, oxazolyl, cyclopentyl, or bicyclo[2.1.1]hexyl.

In certain embodiments of Formula (IB), Ring A is thiazolyl.

In certain embodiments of Formula (IB), Ring A is:

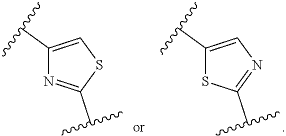 [SMILES: *c1csc(*)n1 ; *c1cnc(*)s1]

In certain embodiments of Formula (IB), Ring A is:

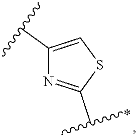 [SMILES: *c1csc(*)n1]

wherein * denotes a bond to Ring B.

In certain embodiments of Formula (IB), Ring B is a six-membered ring. In certain embodiments, Ring B is phenyl or pyridyl. In certain embodiments, Ring B is unsubstituted phenyl.

In certain embodiments of Formula (IB), Ring B is a halogen-substituted phenyl.

In certain embodiments of Formula (IB), R² and R³ are different.

In certain embodiments of Formula (IB), R² and R³ are the same. In certain embodiments, R² is CH(Me) or —CH₂.

In certain embodiments of Formula (IB), R³ is CH(Me) or —CH₂.

In certain embodiments of Formula (IB), R² and R³ are each CH₂.

In certain embodiments of Formula (IB), R⁴ is hydrogen, a protecting group, or —C(O)—CH₃.

In certain embodiments, the compound is selected from:

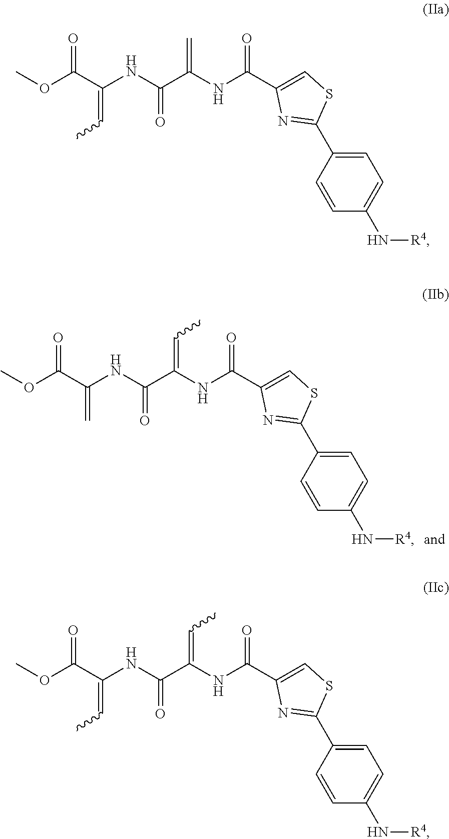

wherein R⁴ is hydrogen, a protecting group, or —C(O)—CH₃; and
wherein a bond drawn as “ [symbol] ” denotes either possible stereochemistry of the attached alkene, E or Z.

In certain embodiments, the compound is selected from:

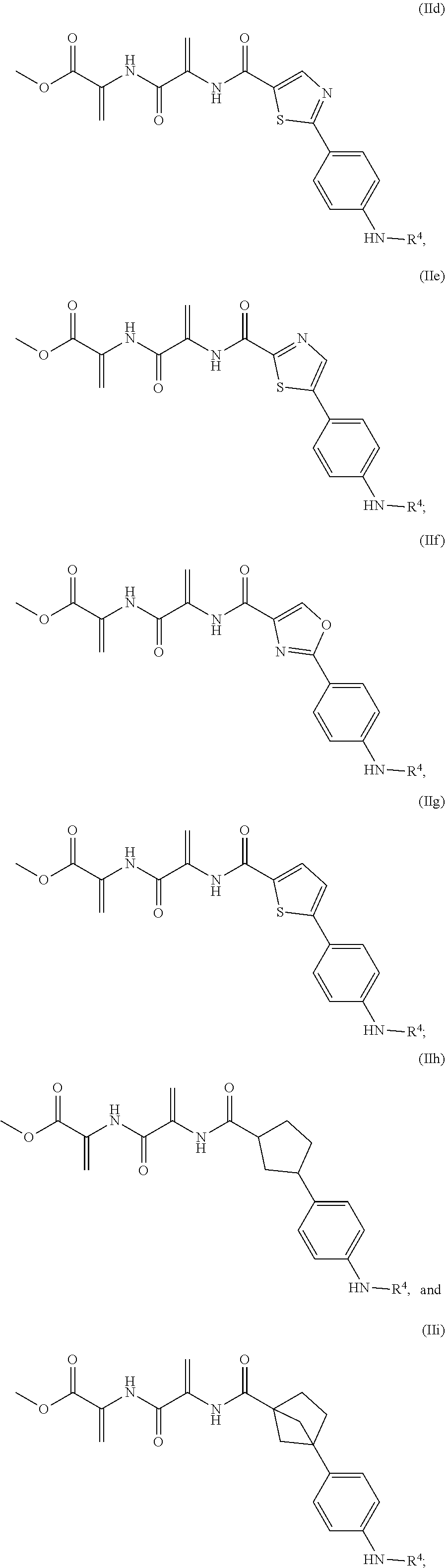

wherein R⁴ is hydrogen, a protecting group, or —C(O)—CH₃.

In certain embodiments, the compound is selected from:

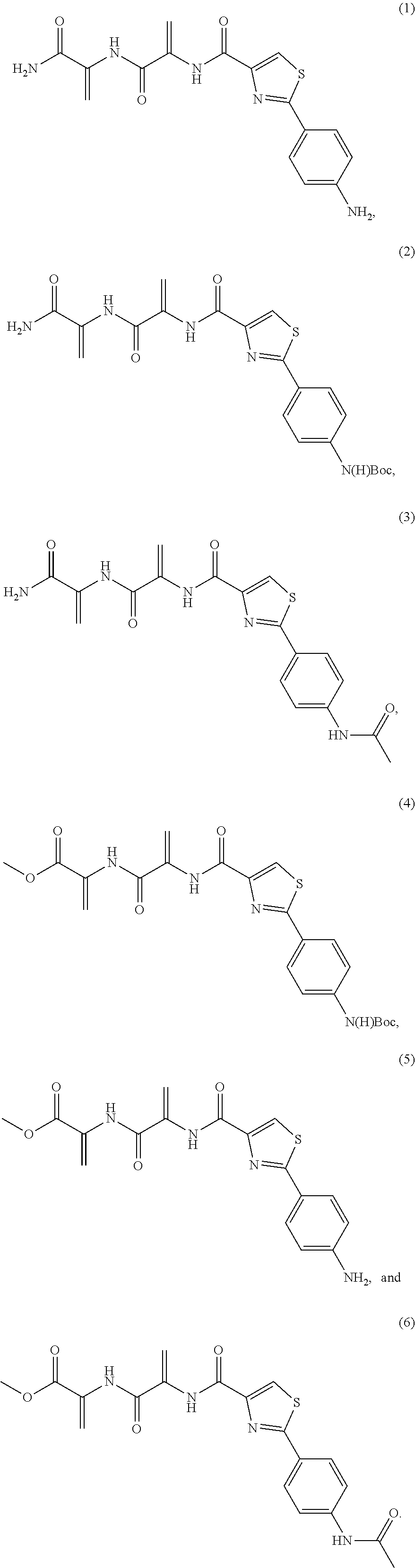

In certain embodiments, the compound is selected from:

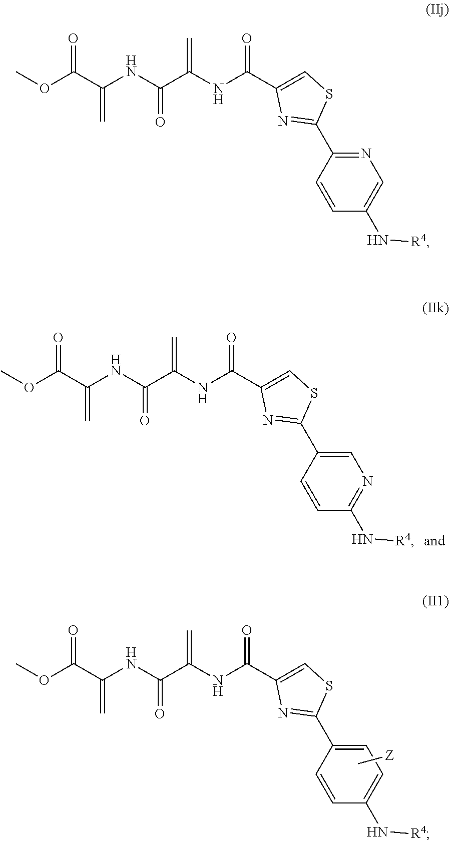

wherein R⁴ is hydrogen, a protecting group, or —C(O)—CH₃;
and wherein Z is selected from fluorine, chlorine, bromine, and iodine.

In certain embodiments, the compound is selected from:

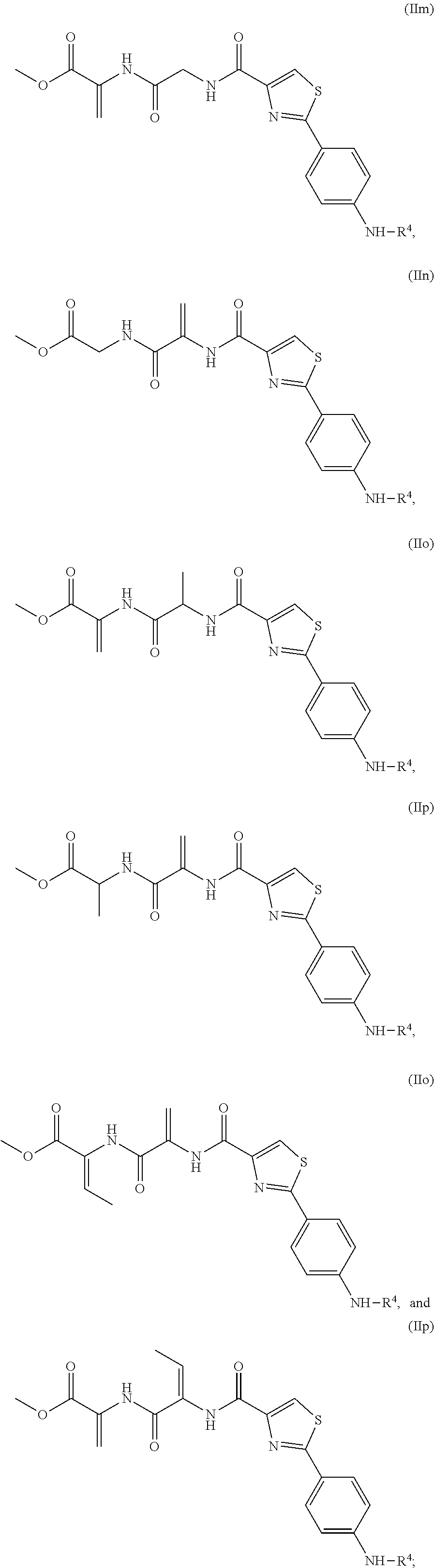

wherein R⁴ is hydrogen, a protecting group, or —C(O)—CH₃.

In certain embodiments, the compound is selected from:

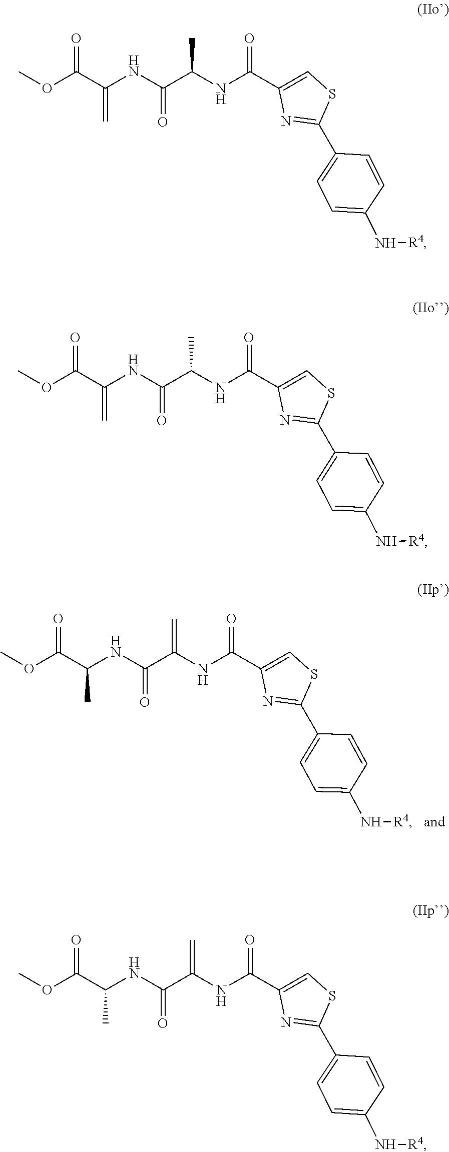

wherein R⁴ is hydrogen, a protecting group, or —C(O)—CH₃.

In certain embodiments, the protecting group is Boc.

In certain embodiments, R⁴ is -L′. In certain embodiments, L′ is —C(O)—X—C(O)OH or —C(O)—X—C(O)NH₂; X is —(CH₂)ₙ—, and n is 2, 3, 4 or 5.

In certain embodiments, the compound is selected from:

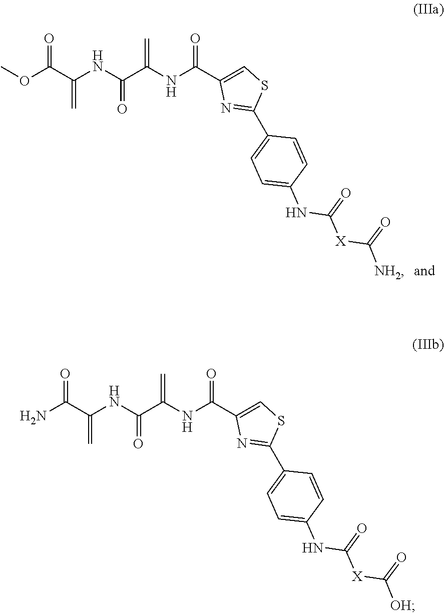

wherein X is —(CH₂)ₙ—; and n is 2, 3, 4 or 5.

In certain embodiments, the compound is selected from:

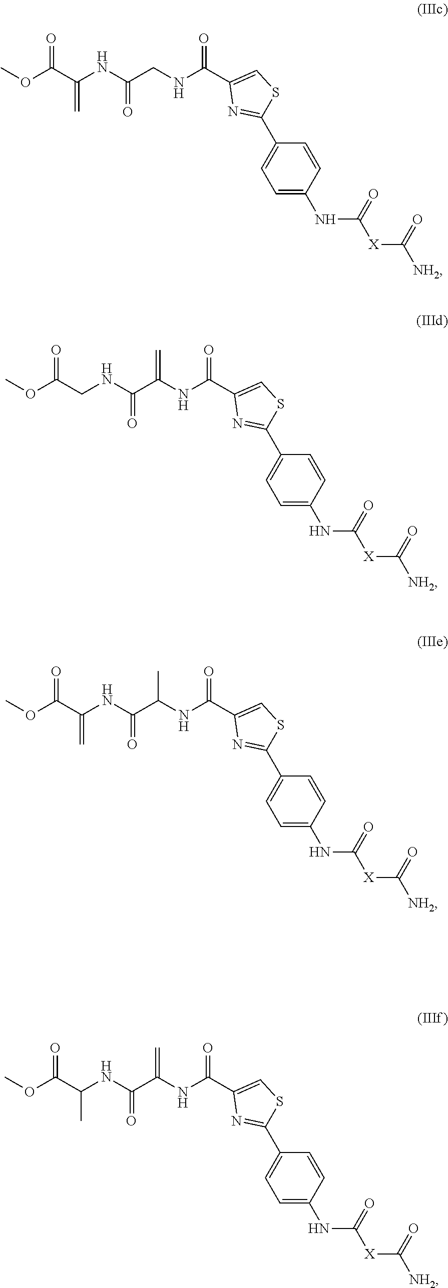

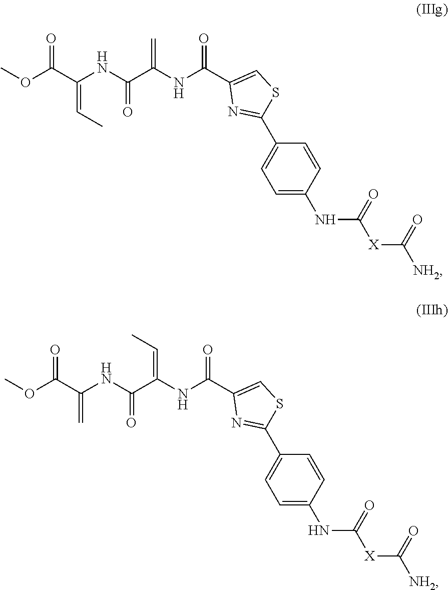

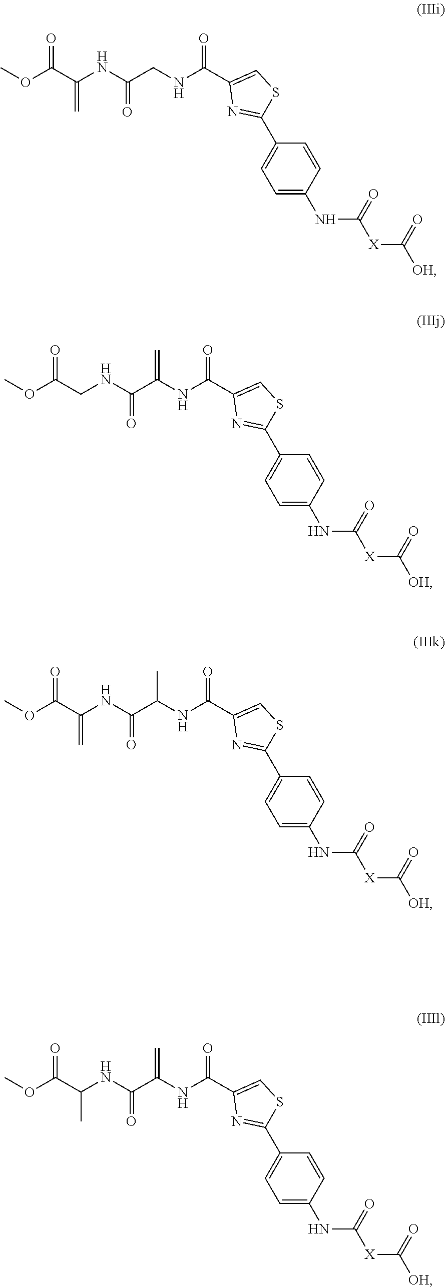

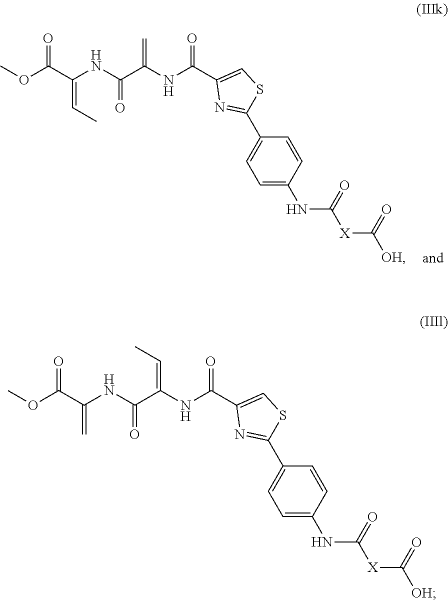

wherein X is —(CH₂)ₙ—; and
n is 2, 3, 4 or 5. In certain embodiments, R⁴ is -L-Y. In certain embodiments, L is a cleavable linker. In certain embodiments, L is a non-cleavable linker. In certain embodiments, L has a chain length of about 2 to about 30 atoms. In certain embodiments, L has a chain length of about 5 to about 20 atoms.

In certain embodiments, L is —C(O)—X—C(O)—; X is —(CH₂)ₙ—; and n is 2, 3, 4 or 5.

In certain embodiments, L is —C(O)—X—C(O)—; X is —(CH₂CH₂—O—)ₘ—(CH₂CH₂)—; and m is 2, 3, 4, 5, or 6.

In certain embodiments, R⁴ is L′ wherein the alkyne group is

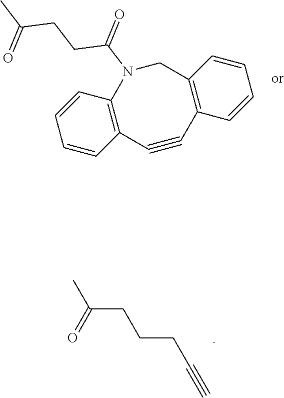

In certain embodiments, the compound is selected from:

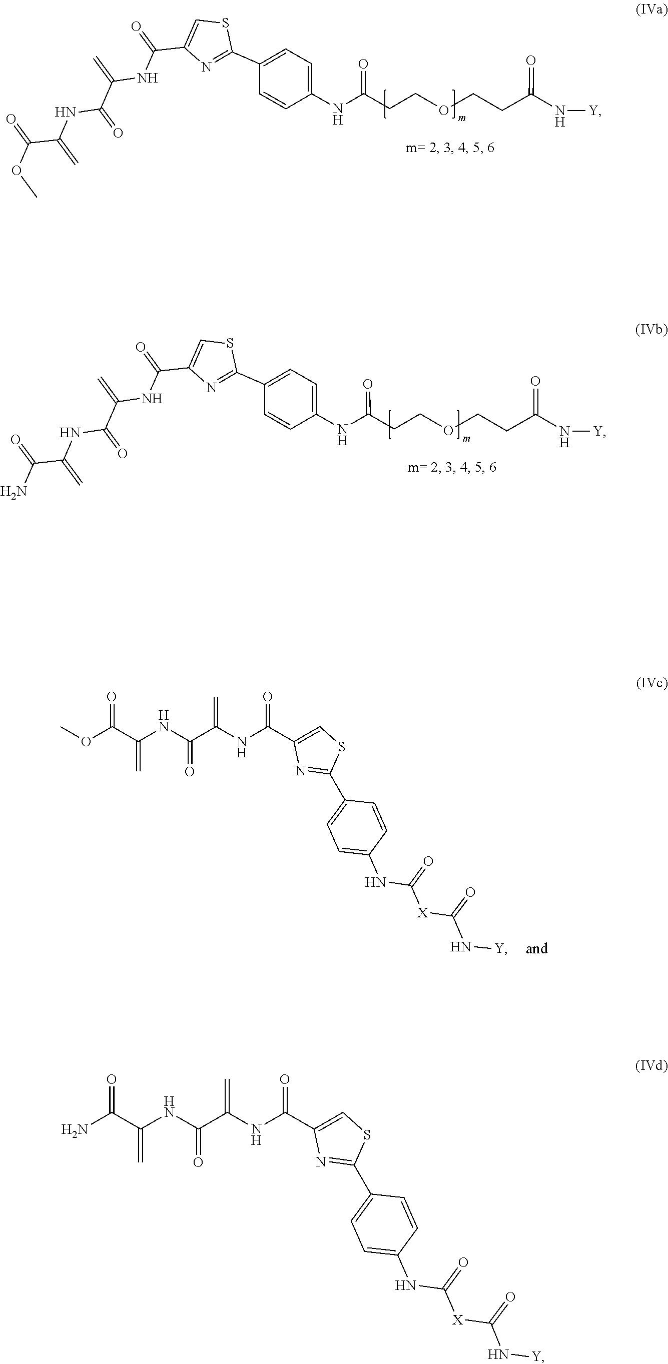

wherein X is —(CH₂)ₙ—; and n is 2, 3, 4 or 5.

In certain embodiments, the compound is selected from:

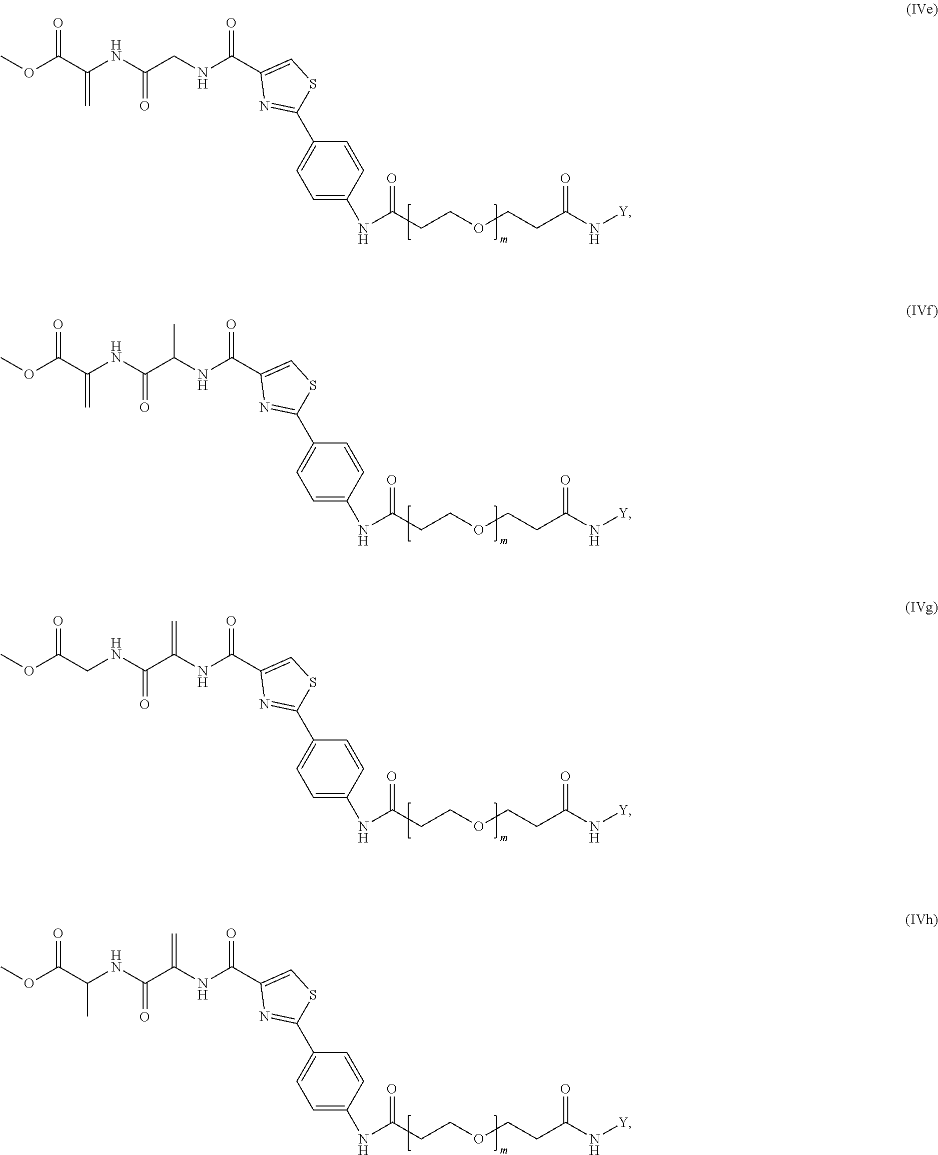

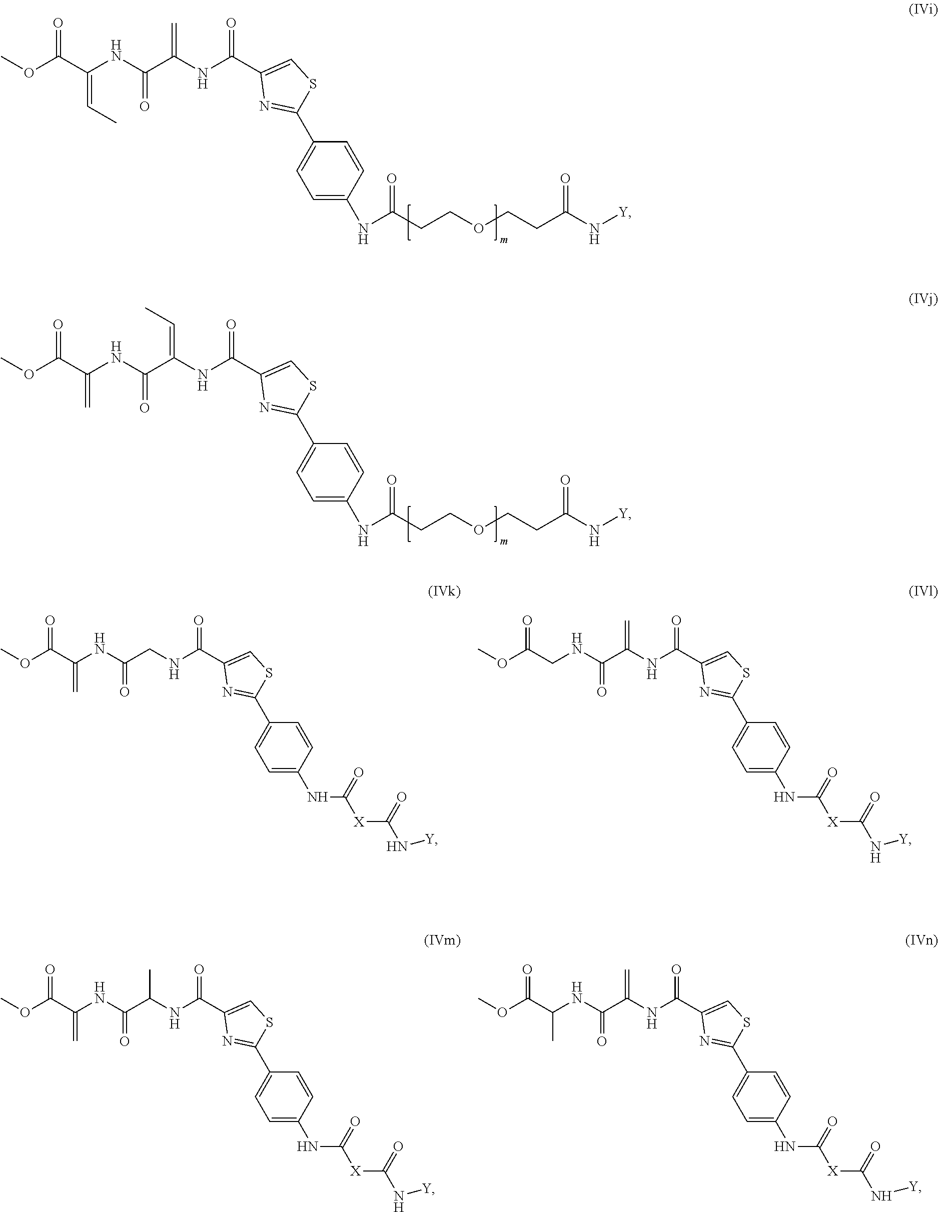

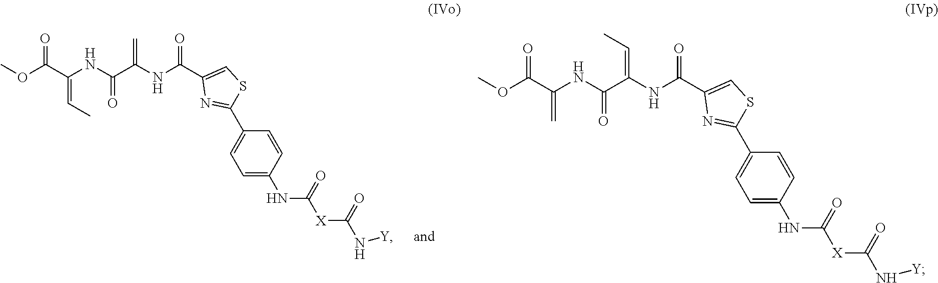

wherein X is —(CH₂)ₙ—;
m is 2, 3, 4, 5, or 6; and
n is 2, 3, 4 or 5.

In certain embodiments, L′ comprises an alkynyl or azido.

In certain embodiments, wherein R⁴ is —C(O)—X′—C≡CH or —C(O)—X′—N₃; X′ is —(CH₂)ₙ—; and n is 2, 3, 4 or 5.

In certain embodiments, L comprises a heteroaryl. In other embodiments, L comprises a triazolyl.

In certain embodiments, R⁴ is

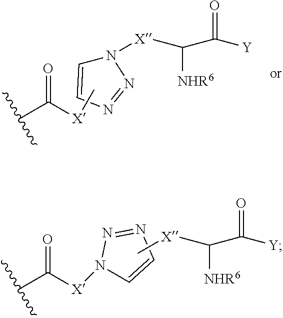

R⁶ is —H or —C(O)CH₃;
X′ is —(CH₂)ₙ—;
X″ is —(CH₂)ₒ—;
n is 2, 3, 4 or 5; and
o is 2, 3, 4 or 5.

In certain embodiments, R⁴ is

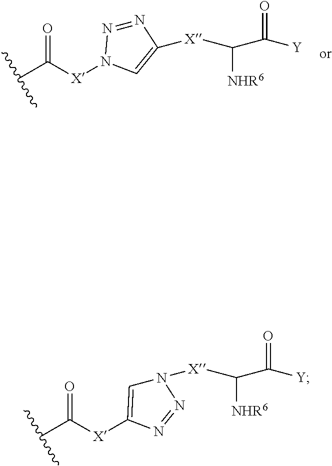

R⁶ is —H or —C(O)CH₃;
X′ is —(CH₂)ₙ—;
X″ is —(CH₂)ₒ—;
n is 2, 3, 4 or 5; and
o is 2, 3, 4 or 5.

In certain embodiments, R⁴ is

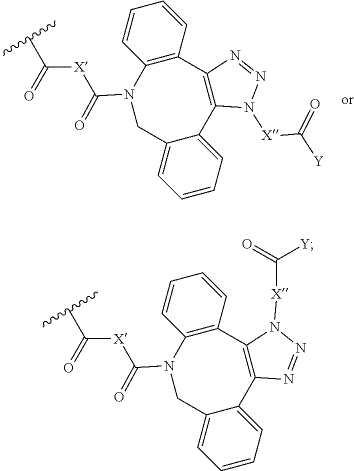

X′ is —(CH₂)ₙ—;
X″ is —(CH₂)ₒ—;
n is 2, 3, 4 or 5; and
o is 2, 3, 4 or 5.

In certain embodiments, Y is a berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenylethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (L-cyclohexyl alanine-D-arginine)3, a mitochondrial-targeted nanocarrier, a DDDK peptide, glycyrrhetinic acid, α-tocopheryl succinate (α-TOS), a graphene oxide nano carrier, PEG-proapoptotic peptide (KLAKLAK)2, a Dmt-D-Arg-Phe-Lys-NH₂ peptide, pyruvaldehyde, N-Nonyl acridine orange, quinoline, styryl fluorophores, or 15d-PGJ2.

In certain embodiments, Y is a mitochondrial penetrating peptide.

In certain embodiments, Y has the structural formula (V):

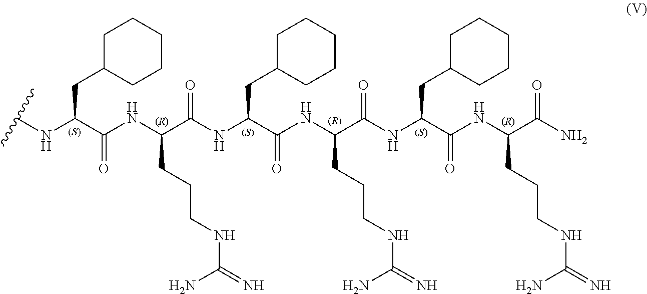

In certain embodiments, the compound is selected from:

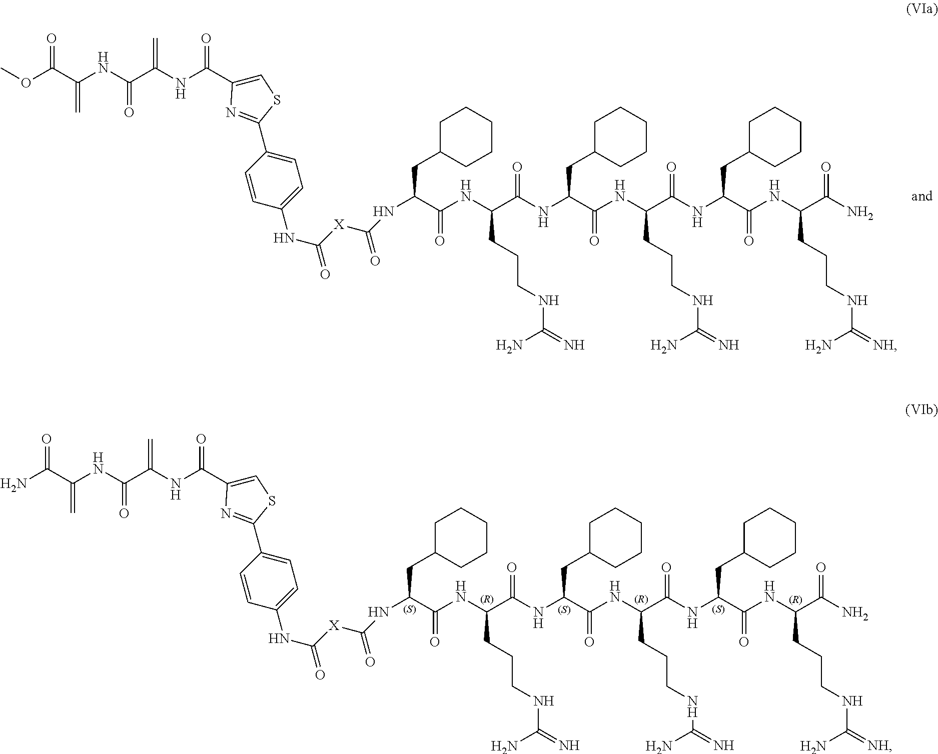

or a pharmaceutically acceptable salt thereof; wherein X is [—(CH₂)ₙ-] or [—(CH₂CH₂—O—)ₘ—(CH₂CH₂)—]; n is 3, 4 or 5; and m is 2, 3, 4, 5, or 6.

In certain embodiments, the present invention provides a pharmaceutically acceptable composition comprising any of the compounds described herein; and a pharmaceutically acceptable carrier.

In certain embodiments, the composition is formulated for oral or parenteral delivery.

In certain embodiments, the compound is contained within a nanoparticle, liposome or micelle, wherein the nanoparticle, liposome, or micelle is conjugated to a mitochondrial targeting moiety.

In certain embodiments, the present invention discloses a composition comprising a compound of Formula (IA) or any subformula thereof, wherein R⁴ is hydrogen, a protecting group, or —C(O)—CH₃, or any of the more specific embodiments thereof described herein contained within a nanoparticle, liposome or micelle, wherein the nanoparticle, liposome, or micelle is conjugated to a mitochondrial targeting moiety.

In certain embodiments, the composition is formulated for oral or parenteral delivery.

In certain embodiments, the present invention discloses a composition comprising a compound of Formula (IB), wherein R⁴ is hydrogen, a protecting group, or —C(O)—CH₃, or any of the more specific embodiments thereof described herein contained within a nanoparticle, liposome or micelle, wherein the nanoparticle, liposome, or micelle is conjugated to a mitochondrial targeting moiety.

In certain embodiments, the nanoparticle, liposome or micelle is selected from poly(ethylene glycol), poly(ε-caprolactone), polysaccharides, poly[(2-hydroxypropyl)-methacrylic acid], poly(lactic-co-glycolic acid), and any combinations of the foregoing.

In certain embodiments, the present invention discloses a method of treating a cancer (e.g., solid tumor or hematological cancer) comprising administering to a subject in need thereof a therapeutically effective amount of any of the compounds described herein, or a composition of that compound. In certain embodiments, the cancer (solid tumor or hematological) is selected from lung, breast, prostate, melanoma, esophageal, leukemia, cervical, liver, colon, gastric, colorectal, glioblastoma, head and neck, pancreatic, mesothelioma, and ovarian. In certain embodiments, the cancer is selected from mesothelioma, lung, ovarian, and breast.

In certain embodiments, the present invention discloses a compound selected from:

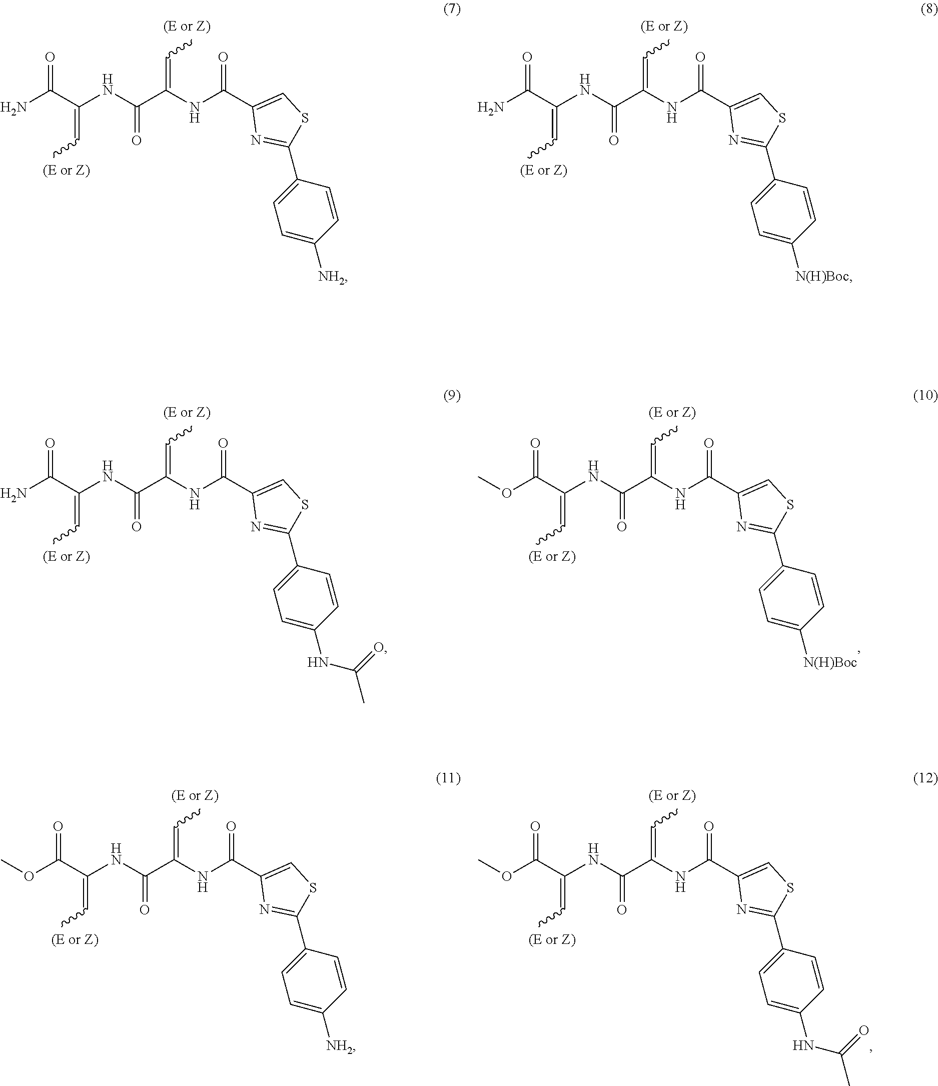

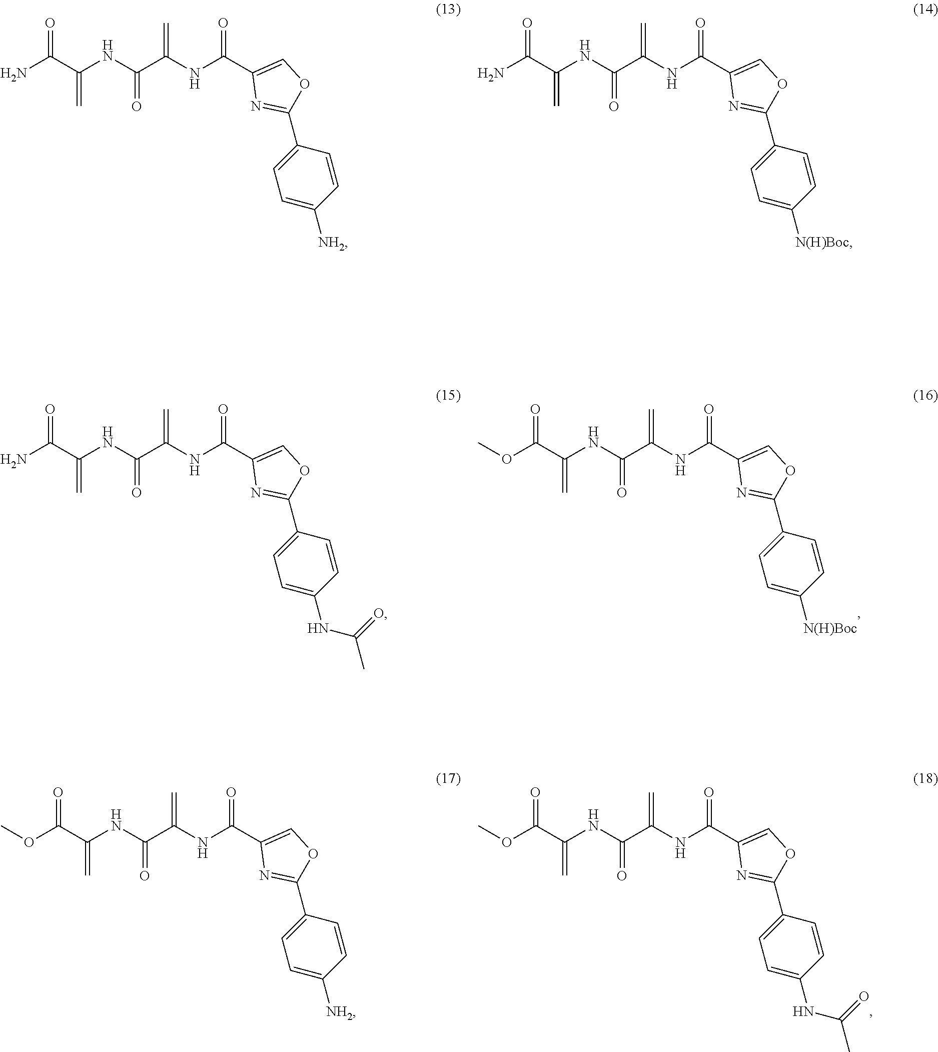

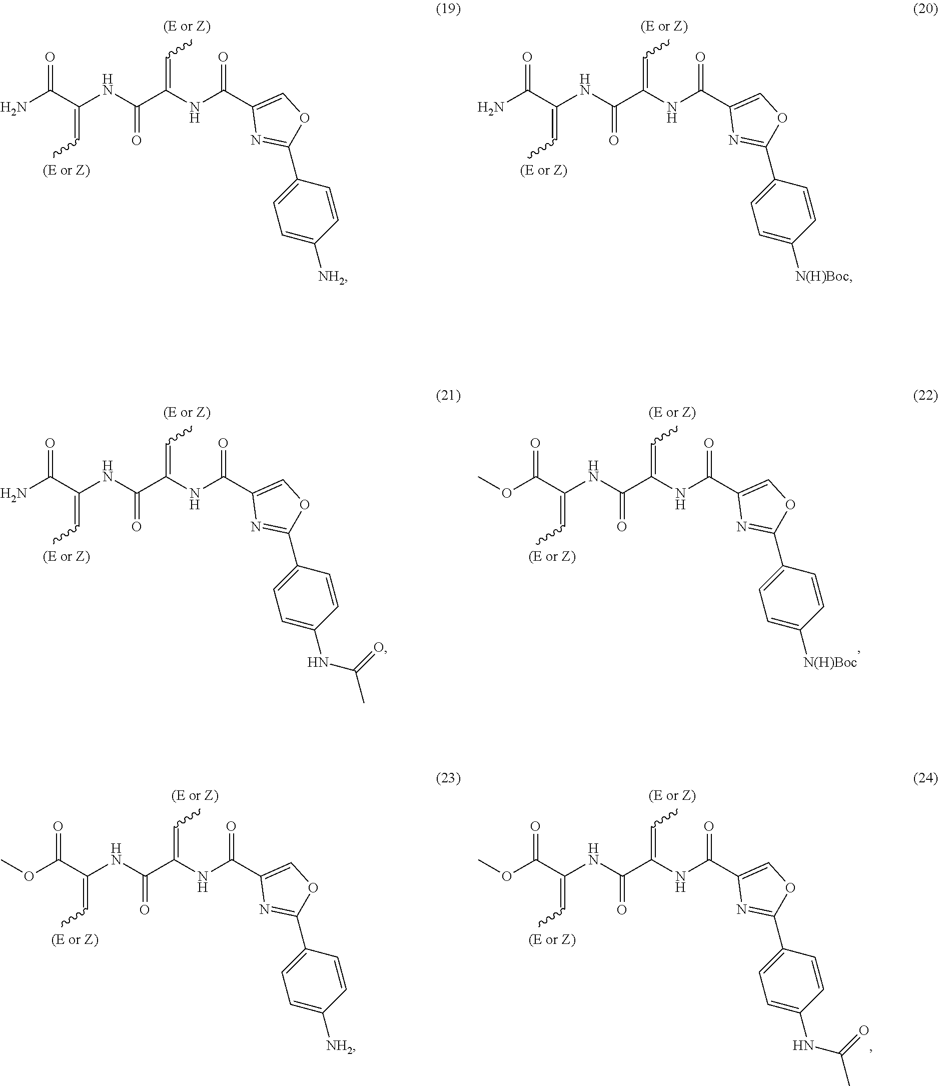

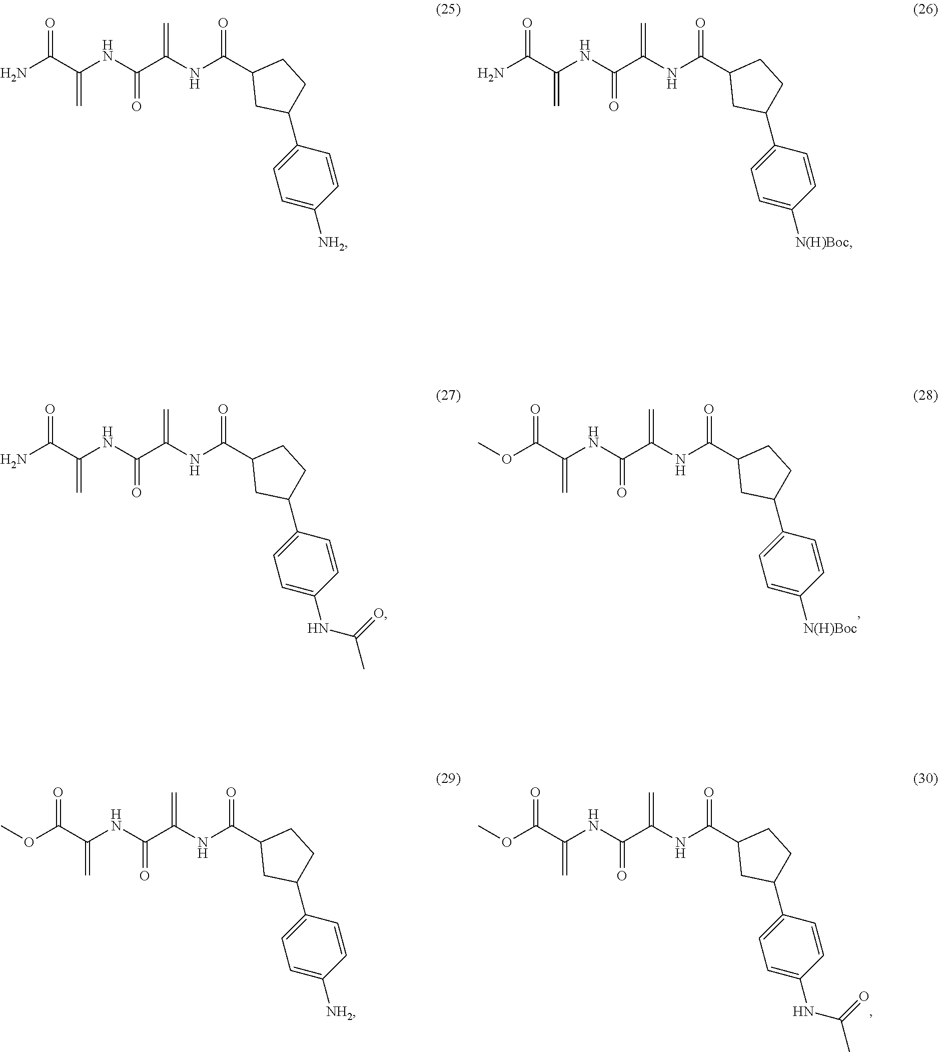

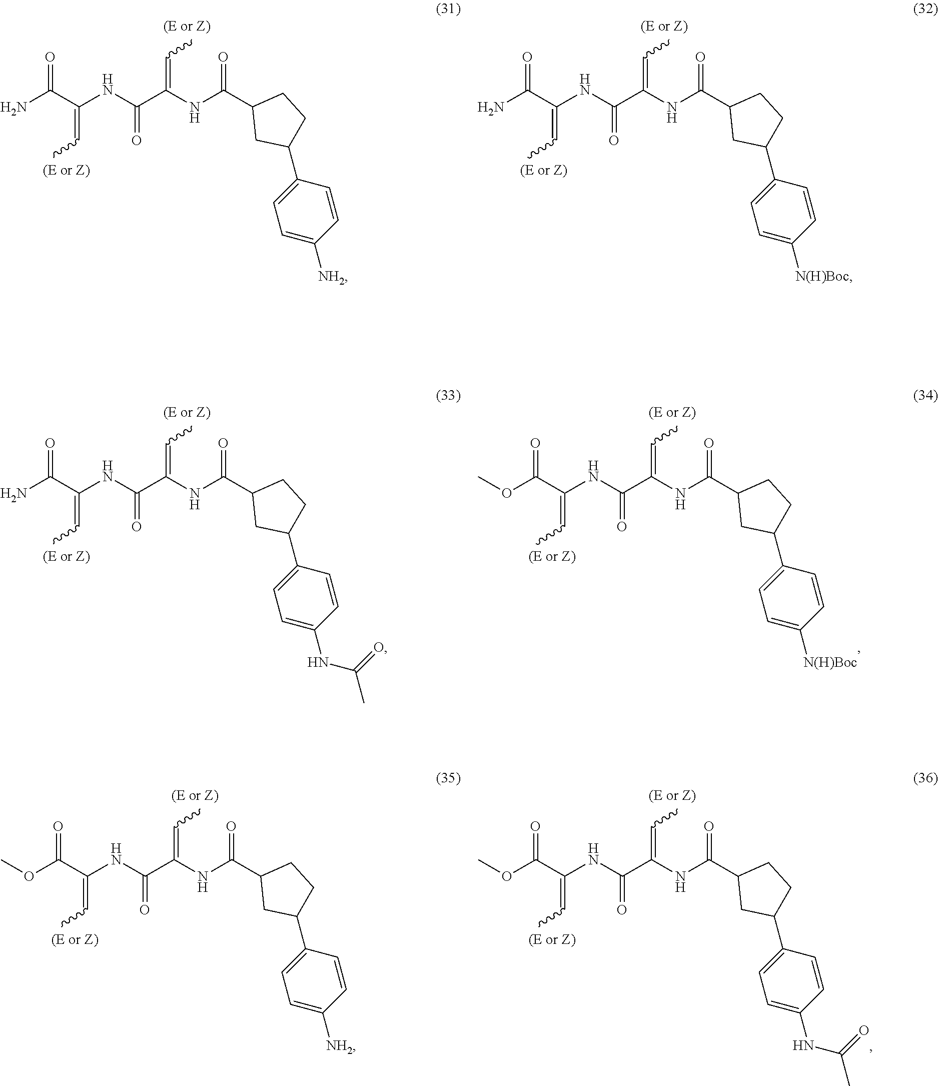

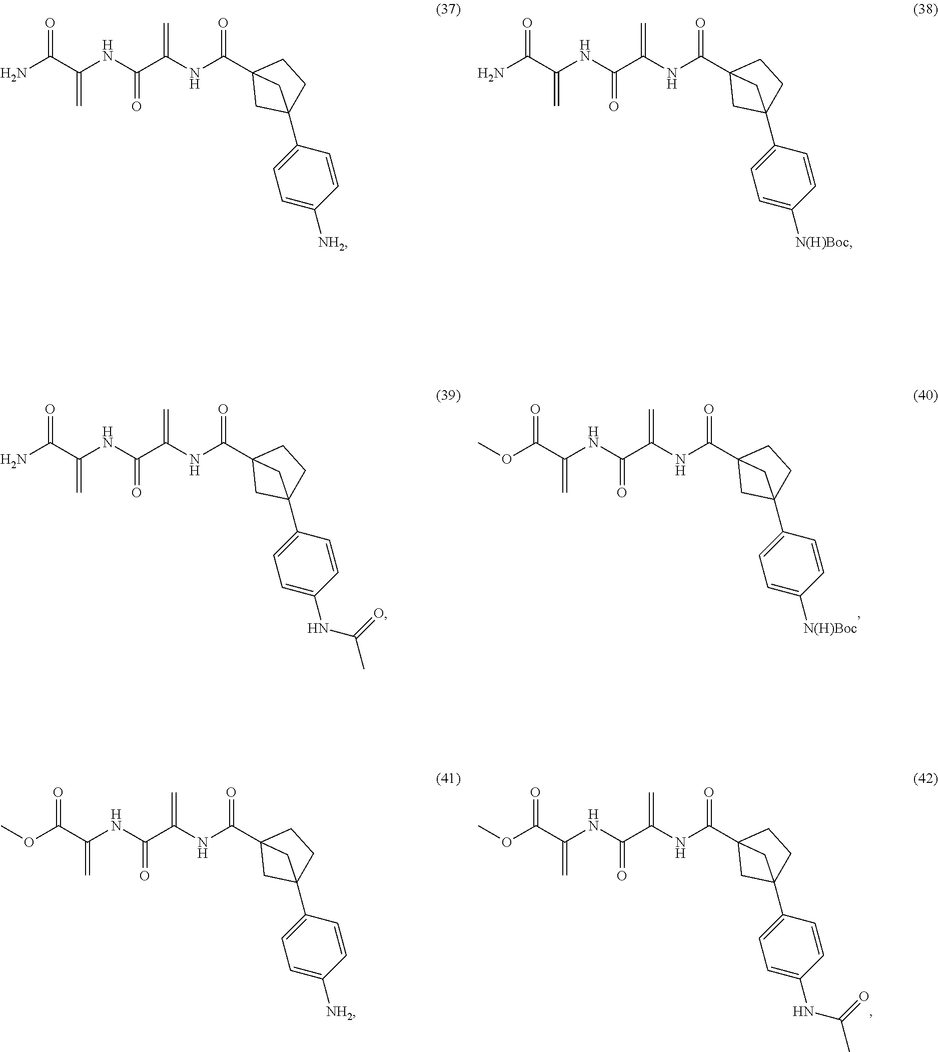

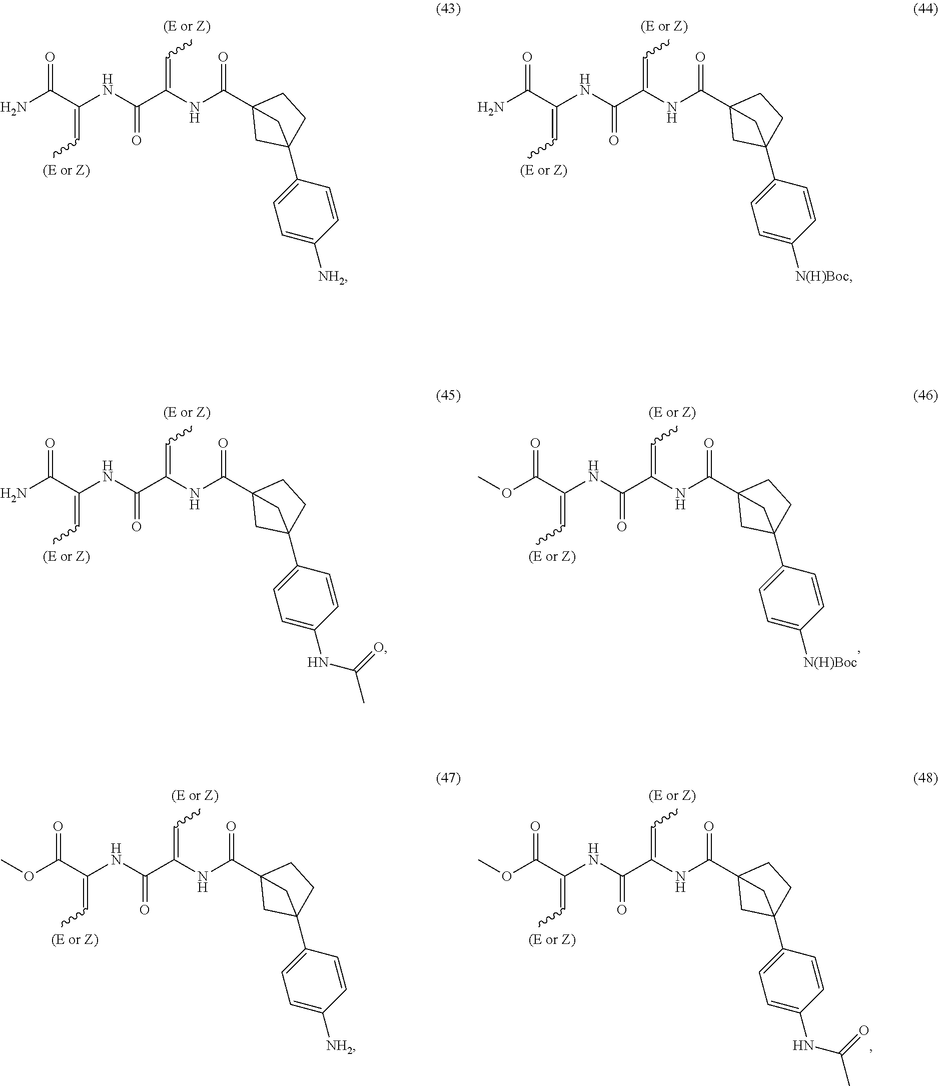

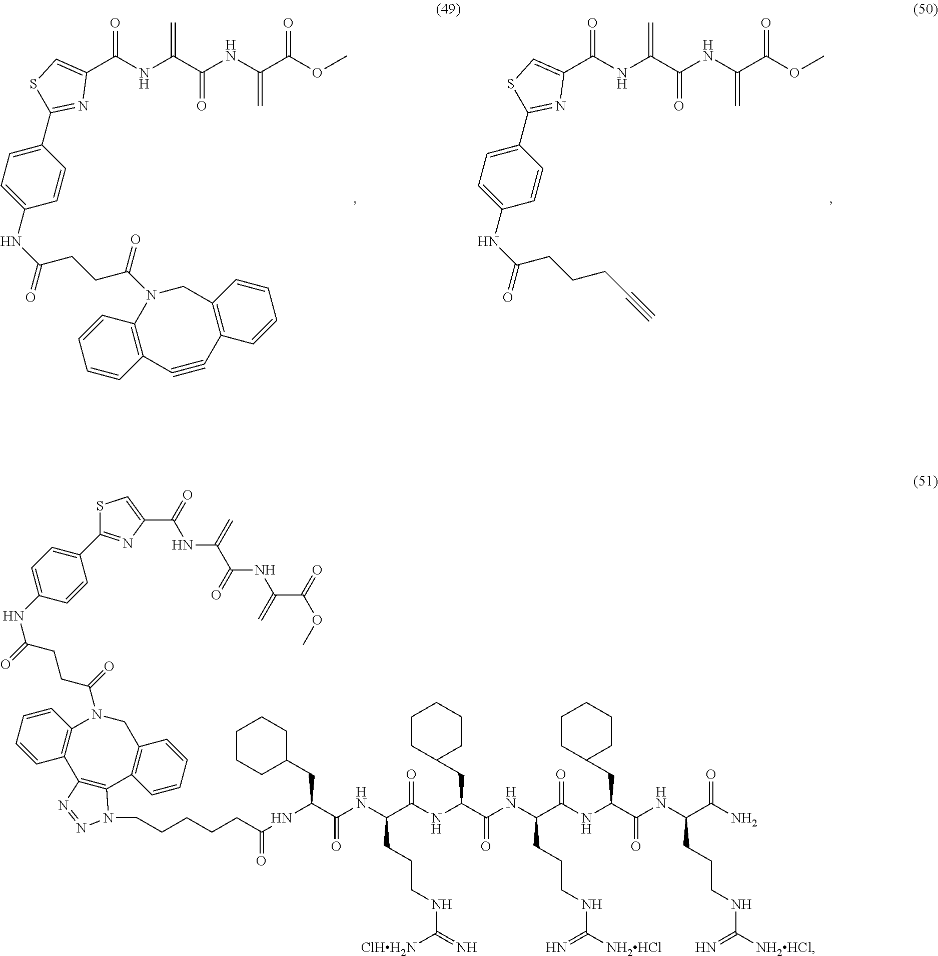

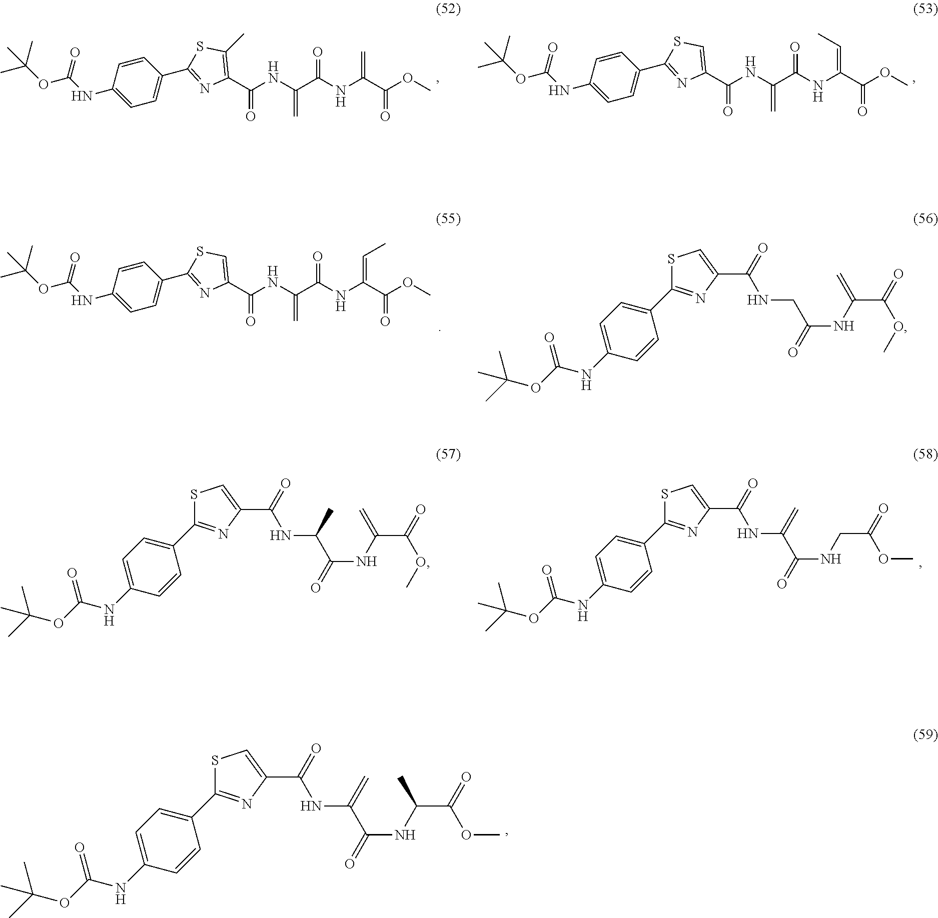

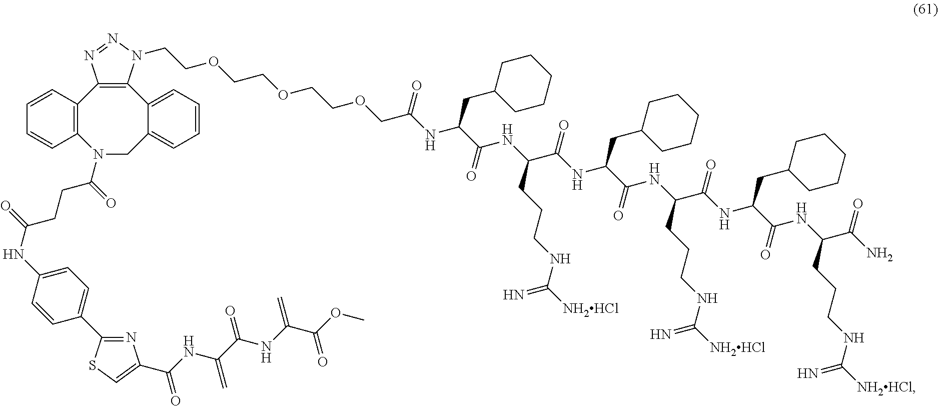

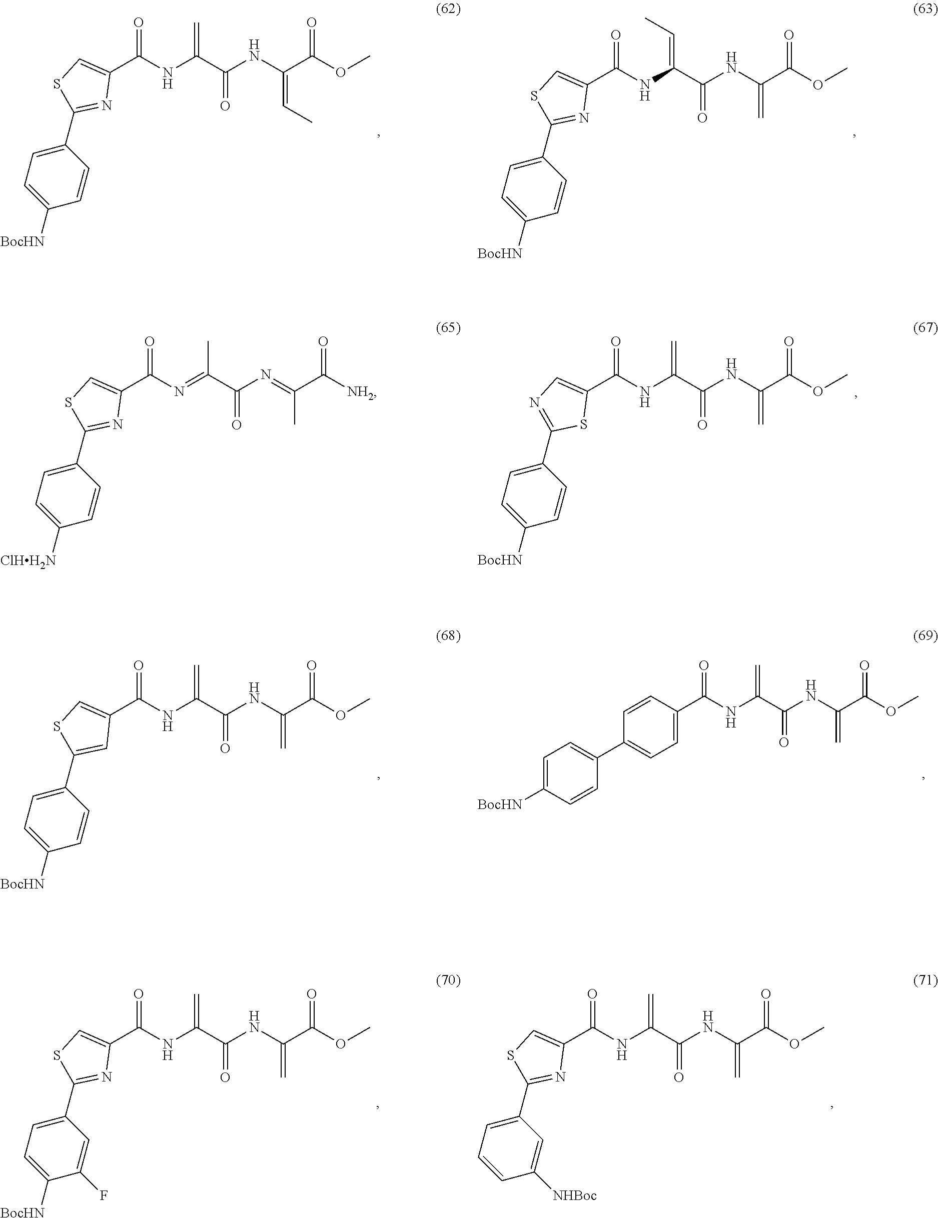

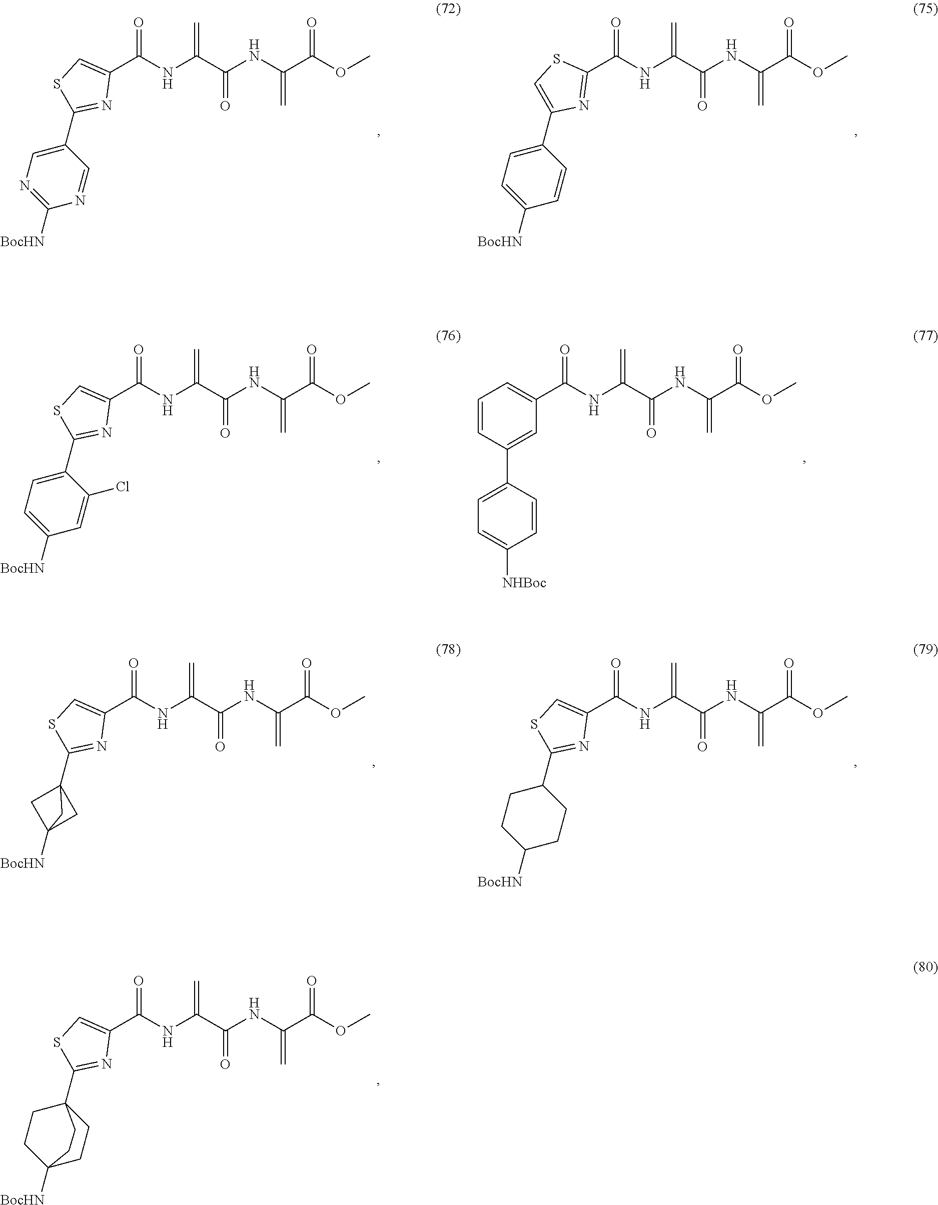

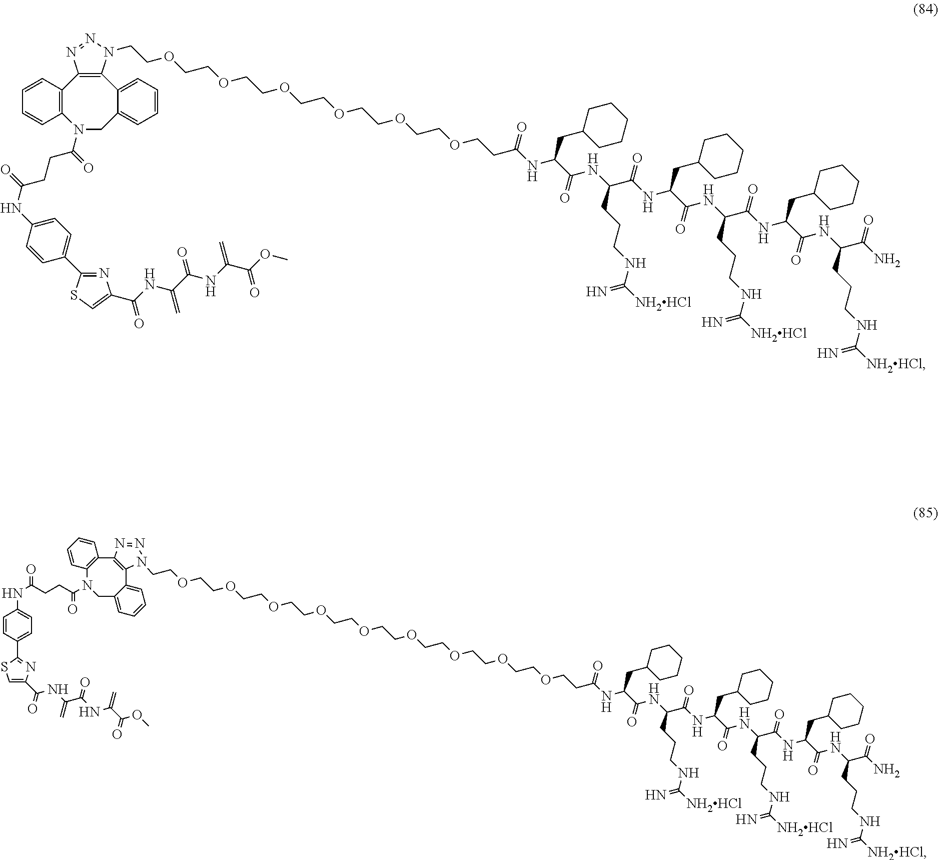

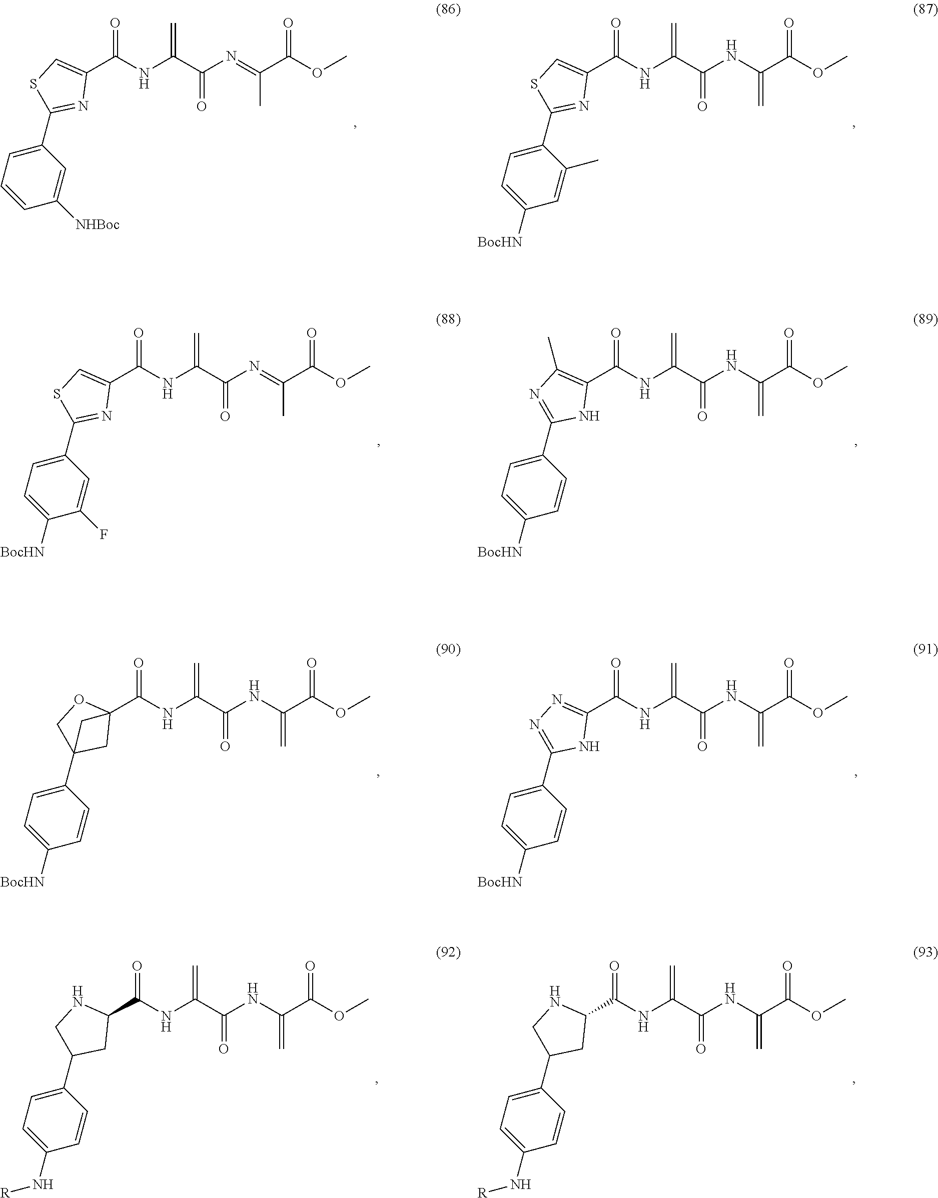

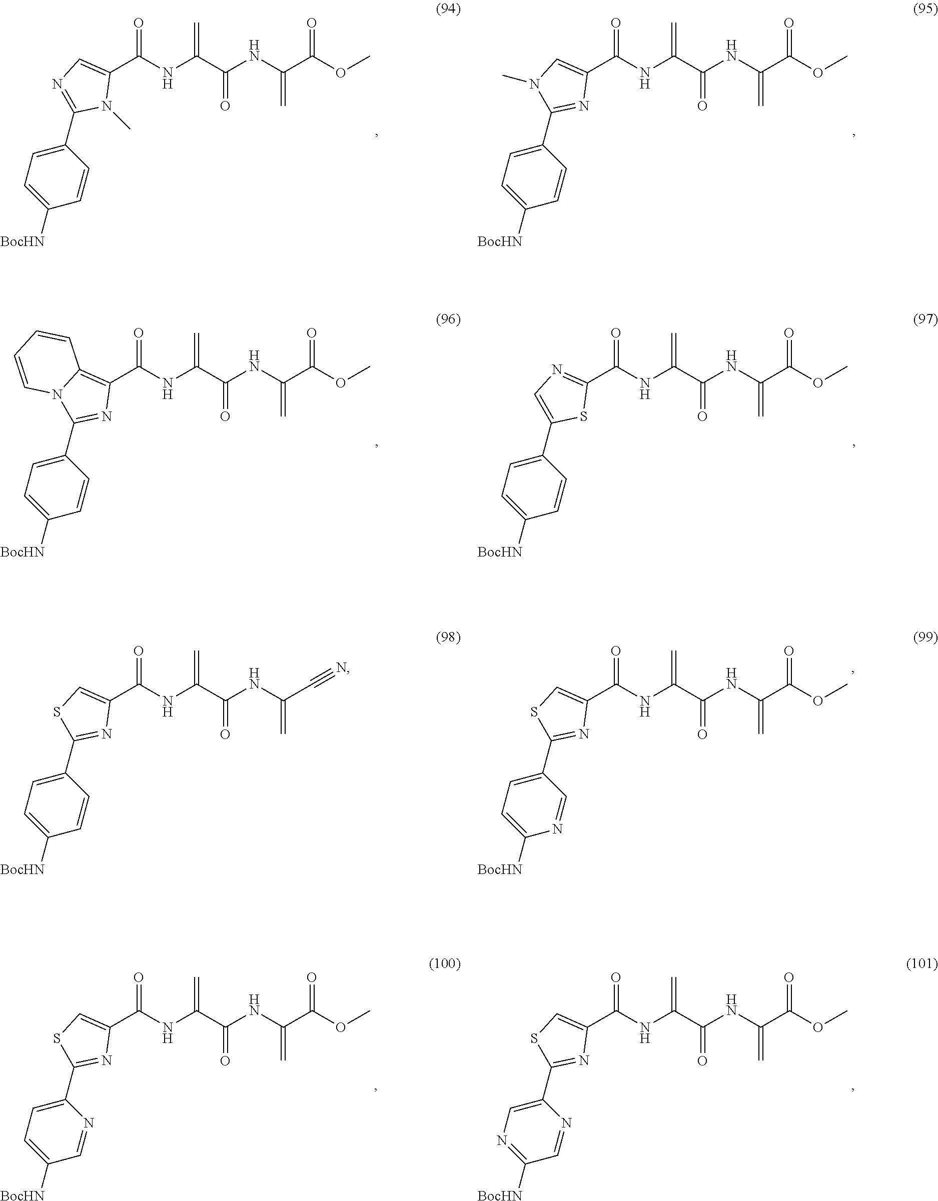

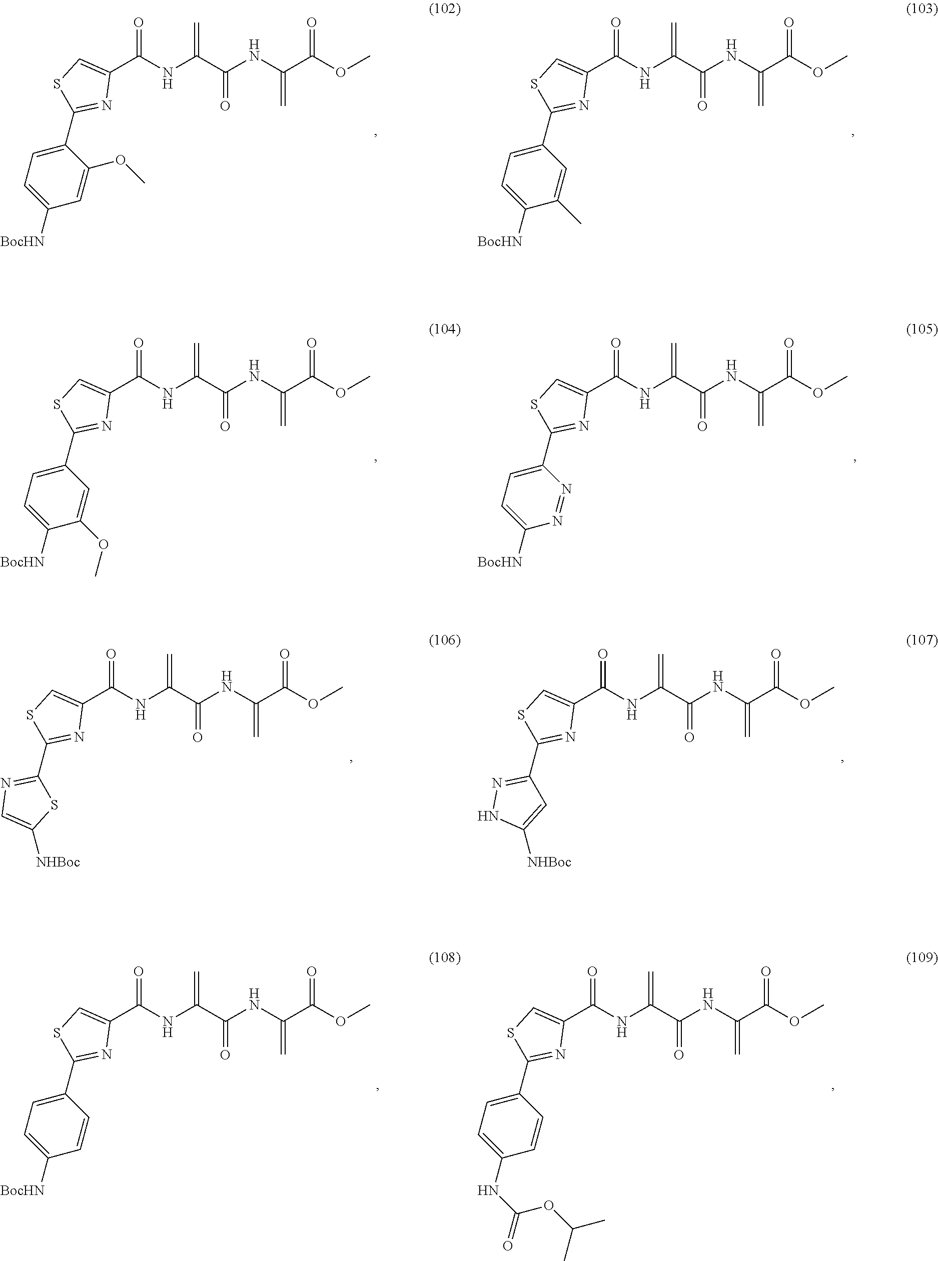

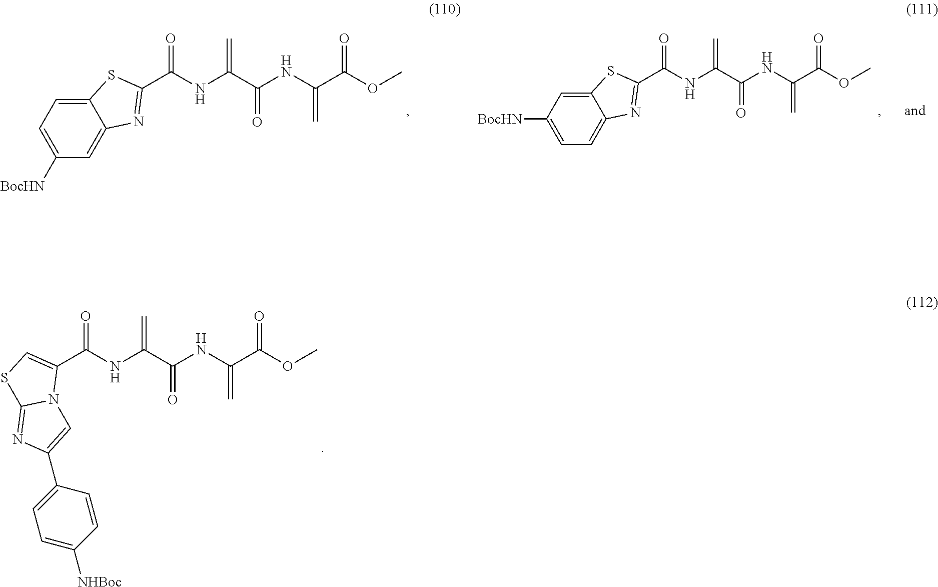 [SMILES: C=C(NC(=O)c1nc2cc(NC(=O)OC(C)(C)C)ccc2s1)C(=O)NC(=C)C(=O)OC ; C=C(NC(=O)c1nc2ccc(NC(=O)OC(C)(C)C)cc2s1)C(=O)NC(=C)C(=O)OC ; C=C(NC(=O)c1csc2nc(-c3ccc(NC(=O)OC(C)(C)C)cc3)cn12)C(=O)NC(=C)C(=O)OC]

### EXAMPLES

### General Experimental Procedure 1

### Suzuki Coupling

Starting boronic acid or boronic ester (1 eq.) was introduced into a flask under N₂. 1,2-Dimethoxyethane (0.1 M), bromide (1.05 Eq), sodium carbonate 2 M aqueous solution (5 eq.) were added. The mixture was then degassed with N₂ for a couple of minutes. Then palladiumtetrakis (0.05 eq.) was added and the mixture was stirred at 90° C. (ext) until full conversion (overnight). The reaction was then stopped. Water was added, and the mixture was extracted with EtOAc. Brine was added to improve separation. The organic layer was dried over Na₂SO₄, filtered, and concentrated. The crude material was purified by automated FCC. Desired products were obtained with 40-70% yield. Note: in some reactions the hydrolyzed product was also observed. This was isolated by acidifying the aqueous phase and extracted with EtOAc twice. Combined organics were washed with brine, dried over Na₂SO₄, filtered, and concentrated to provide the hydrolyzed product.

### General Experimental Procedure 2

### Ester Hydrolysis

To a solution of ester (1 eq.) in THF (0.05 M) was added Lithium hydroxide monohydrate (4 eq.) as solution in water (2M) and the mixture was stirred for 2-16 hours at room temperature. The mixture was diluted EtOAc and washed with 1 M HCl and brine (30 mL each), dried over Na₂SO₄, filtered, and concentrated to provide the desired product.

### General Experimental Procedure 3

### EDCI Mediated Amide Coupling

To a solution of acid (1 eq.) and amine (1 eq.) in DCM (0.05 M) were added DiPEA (3 eq.), cyanic (E)-2-(hydroxyimino)butanoic anhydride (1.3 eq.) and EDCI (1.3 eq.) and the resulting mixture was stirred at room temperature for 16 hours. The mixture was washed with 1M HCl, water, NaHCO₃ and brine, dried over Na₂SO₄, filtered, and concentrated. The crude material was purified by automated FCC to afford the desired product.

### General Experimental Procedure 4

### HATU Mediated Amide Coupling

To a solution of acid (1 eq.) and amine (1 eq.) in DCM (0.05 M) were added DiPEA (3 eq.) and HATU (1.2 eq.) and the resulting mixture was stirred at room temperature for 2-16 hours. The mixture was washed with 1M HCl, water, NaHCO₃ and brine, dried over Na₂SO₄, filtered, and concentrated. The crude material was purified by automated FCC to afford the desired product.

### General Experimental Procedure 5

### Acetylation of Serine Sidechains

To a solution of the free alcohol (1 eq.) in MeCN (0.05 M) were added triethylamine (2 eq./alcohol), DMAP (0.2 eq.) and acetic anhydride (1.1 eq/alcohol) and the resulting mixture was stirred for 2 hours. The reaction mixture was diluted with water and the MeCN was removed in vacuo. The mixture was extracted with EtOAc and the organic phase was washed with brine, dried over Na₂SO₄, filtered, and concentrated. The crude material was purified by automated FCC to afford the desired product.

### General Experimental Procedure 6

### Stepwise Elimination of Mono-TBDPS Protected Bisserine

To a solution of mono-TBDPS protected bisserine derivative (1 eq.) in DCM (0.05 M) was added triethylamine (10 eq.) and methane sulfonyl chloride (1.2 eq.) and the resulting mixture was stirred at room temperature for 1 hour. The reaction was followed by LCMS. Optionally, additional triethylamine was added. Upon completion, a solution of tetrabutylammonium fluoride in THF (1 M, 2 eq.) was added and the mixture was stirred for 1 hour. The mixture was washed with 1M HCl and brine, dried over Na₂SO₄, filtered, and concentrated. The residue was taken up in CH₂Cl₂ and triethylamine (10 eq.) and methane sulfonyl chloride (1.2 eq.) were added. The resulting mixture was stirred for 1 hour at room temperature. The reaction was followed by LCMS. Optionally, additional triethylamine was added. The mixture was washed with 1M HCl and brine, dried over Na₂SO₄, filtered, and concentrated. The crude material was purified by automated reverse phase FCC.

### General Experimental Procedure 7

### Stepwise Elimination of Mono-TBDPS, Mono-Acetate

To a solution of mono-TBDPS mono-acetyl bisserine derivative (1 eq.) in CH₂Cl₂ (0.05 M) was added DBU (2 eq.) and the mixture was stirred at room temperature for 1-16 h. Then a solution of TBAF in THF (1 M, 2 eq.) was added and the mixture stirred for 2 hours. The mixture was concentrated and purified by automated FCC. To a solution of the mono-eliminated intermediate in CH₂Cl₂ (0.05 M) were added triethylamine (6 eq.) and methanesulfonyl chloride (1.5 eq.). Optionally, additional triethylamine was added. The mixture was washed with 1M HCl and brine, dried over Na₂SO₄, filtered, and concentrated. The crude material was purified by automated reverse phase FCC.

### General Experimental Procedure 8

### Elimination of Bisacetate

A solution of the bisacetate in THF/CH₂Cl₂ (1/1, v/v, 0.05 M) was cooled to 0° C. and DBU (4 eq.) was added. The mixture was stirred at 0° C. for 2 hours. The mixture was diluted with CH₂Cl₂ and water and HCl (aq. 1M) were added to acidify the mixture to pH=4. The mixture was extracted with CH₂Cl₂ (3×) and the combined organic layers were washed with brine, dried over Na₂SO₄, filtered, and concentrated. The crude material was purified by automated reverse phase FCC.

### General Experimental Procedure 9

### Preparation of Primary Amide

To a solution of carboxylic acid (1 eq.) in DMF (0.1 M) or CH₂Cl₂ (0.15 M) were added NH₄Cl (3 eq.), DiPEA (3.5 eq.) and HATU (1.1 eq.). The mixture was stirred at room temperature overnight. The reaction was quenched with water. In case of clear solution, then the mixture was extracted with EtOAc. The organic layer was washed with brine, dried over Na₂SO₄, filtered and concentrated. The crude material was purified by automated FCC. In case of precipitate, then the mixture was filtered and washed with water and the solvent used in the reaction. The solid was recovered, concentrated to remove the traces of solvent, and afforded the desired primary amide.

### General Experimental Procedure 10

### Thiation Primary Amide

To a solution of primary amide (1 eq.) in THF (0.2 M) was added Lawesson's reagent (0.7 eq.) and the mixture was stirred at 80° C. (ext.) until full conversion. Then the reaction was quenched with NaHCO₃ sat. solution and the mixture was extracted with EtOAc. The organic layer was washed with brine, dried over Na₂SO₄, filtered and concentrated. The crude material was purified by automated FCC.

### General Experimental Procedure 11

Thiazole Formation from Thioamide

To a solution of primary thioamide (1 eq.) in EtOH (0.2 M) was added ethyl bromopyruvate (1.1 eq.) and the resulting mixture was stirred at room temperature or 50° C. (ext.) until full conversion. The reaction was quenched with NaHCO₃ sat. solution and the mixture was extracted with EtOAc. The organic layer was washed with brine, dried over Na₂SO₄, filtered and concentrated. The crude material was purified by automated FCC.

### Analysis Methods

### LCMS Method “21020335B TFA LCMS-5 C3”

System: Agilent 1290 series with UV detector and HP 6130 MSD mass detector
Column: Waters XBridge BEH C18 XP (2.1×50 mm; 2.5 μm)
Mobile phase A: 0.05% TFA in Milli-Q
Mobile phase B: Acetonitrile
Pump Flow: 0.6 ml/min
UV Detection: 215.8 nm
Injection Volume: 0.1 L
Run Time: 3.5 min
Column Temperature: 35° C.
Pump Program: Gradient

| Time (min) | % A | % B |
|---|---|---|
| 0.0 | 95 | 5 |
| 0.5 | 95 | 5 |
| 2.5 | 10 | 90 |
| 3.5 | 10 | 90 |

### LCMS Method “21020335C TFA LCMS-5 C4”

System: Agilent 1290 series with UV detector and HP 6130 MSD mass detector
Column: Waters XBridge BEH C18 XP (2.1×50 mm; 2.5 μm)
Mobile phase A: 0.05% TFA in Milli-Q
Mobile phase B: Acetonitrile
Pump Flow: 0.6 ml/min
UV Detection: 215.8 nm
Injection Volume: 0.1 L
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

### LCMS Method “22010199 LCMS-5 C3”

System: Agilent 1290 series with UV detector and HP 6130 MSD mass detector
Column: Waters XBridge BEH C18 XP (2.1×50 mm; 2.5 μm)
Mobile phase A: 10 mM NH₄OAc (Water/Methanol/Acetonitrile 900/60/40)
Mobile phase B: 10 mM NH₄OAc (Water/Methanol/Acetonitrile 100/540/360)
Pump Flow: 0.6 ml/min
UV Detection: 215.8 nm
Injection Volume: 0.3 L
Run Time: 3.5 min
Column Temperature: 35° C.
Pump Program: Gradient

| Time (min) | % A | % B |
|---|---|---|
| 0.0 | 80 | 20 |
| 1.5 | 0 | 100 |
| 3.0 | 0 | 100 |

### LCMS Method “22010199A TFA LCMS-5 C1”

System: Agilent 1290 series with UV detector and HP 6130 MSD mass detector
Column: Waters Acquity HSS T3 (2.1×75 mm; 1.8 μm)
Mobile phase A: 0.05% TFA in Milli-Q
Mobile phase B: Acetonitrile
Pump Flow: 0.6 ml/min
UV Detection: 215.8 nm
Injection Volume: 0.8 L
Run Time: 3.0 min
Column Temperature: 35° C.
Pump Program: Gradient

| Time (min) | % A | % B |
|---|---|---|
| 0.0 | 50 | 50 |
| 1.5 | 10 | 90 |
| 3.0 | 10 | 90 |

LCMS Method “22010199C TFA LCMS-5 C3”
System: Agilent 1290 series with UV detector and HP 6130 MSD mass detector
Column: Waters XBridge BEH C18 XP (2.1×50 mm; 2.5 μm)
Mobile phase A: 0.05% TFA in Milli-Q
Mobile phase B: Acetonitrile
Pump Flow: 0.6 ml/min
UV Detection: 215.8 nm
Injection Volume: 0.8 L
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

### LCMS Method “22010199D TFA LCMS-5 C8”

System: Agilent 1290 series with UV detector and HP 6130 MSD mass detector
Column: Zorbax SB-C8 (2.1×50 mm; 1.8 μm)
Mobile phase A: 0.05% TFA in Milli-Q
Mobile phase B: Acetonitrile
Pump Flow: 0.6 ml/min
UV Detection: 215.8 nm
Injection Volume: 0.1 L
Run Time: 3.0 min
Column Temperature: 35° C.
Pump Program: Gradient

| Time (min) | % A | % B |
|---|---|---|
| 0.0 | 80 | 20 |
| 1.5 | 10 | 90 |
| 3.0 | 10 | 90 |

### LCMS Method “30833 LCMS-6”

System: Agilent 1260 series with UV detector, ELSD 1260 detector and Agilent 6120 mass detector
Column: Waters XBridge BEH C18 XP (2.1×50 mm, 2.5 um)
Mobile phase A: 0.1% TFA in Milli-Q
Mobile phase B: 0.1% TFA in Acetonitrile
Pump Flow: 0.6 ml/min
UV Detection: 215.8 nm
Injection Volume: 0.6 L
Run Time: 6.0 min
Column Temperature: 35° C.
Pump Program: Gradient

| Time (min) | % A | % B |
|---|---|---|
| 0.0 | 80 | 20 |
| 0.5 | 80 | 20 |
| 3.5 | 10 | 90 |
| 6.0 | 10 | 90 |

LCMS Method “General 3 basic”
System: Waters Acquity UPLC with PDA detector and QDA mass detector
Column: Waters XBridge BEH C18 (2.1×50 mm; 2.5 m)
Mobile phase A: 10 mM ammonium bicarbonate in water (pH 9.5)
Mobile phase B: Acetonitrile
Pump Flow: 0.6 ml/min
UV Detection: λmax
Injection Volume: 0.4 l
Run Time: 2.5 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |

LCMS Method “General 3 acidic”
System: Waters Acquity UPLC with PDA detector and QDA mass detector
Column: Waters XBridge BEH C18 (2.1×50 mm; 2.5 m)
Mobile phase A: 0.1% (v/v) Trifluoroacetic acid in water
Mobile phase B: Acetonitrile
Pump Flow: 0.6 ml/min
UV Detection: λmax
Injection Volume: 0.4 l
Run Time: 2.5 min
Column Temperature: 30° C.
Pump Program: Gradient

| Time (min) | % A | % B |
|---|---|---|
| 0.0 | 80 | 20 |
| 1.5 | 5 | 95 |
| 2.5 | 5 | 95 |
| 2.6 | 85 | 15 |

### Example 1A: Exemplary Syntheses of Thiostrepton-Inspired Structure A1

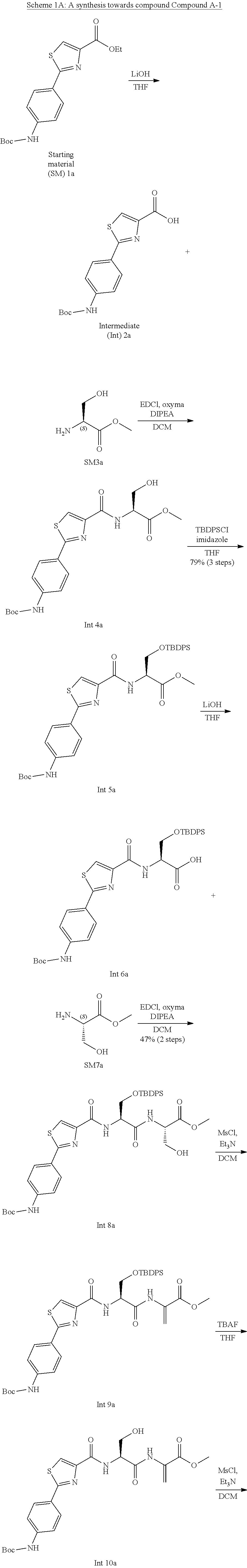

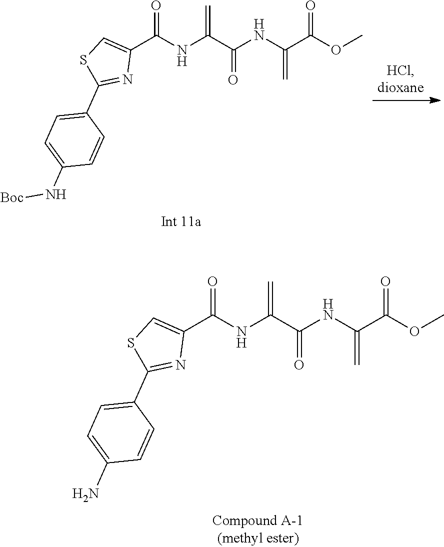 [SMILES: C=C(NC(=O)c1csc(-c2ccc(NC(=O)OC(C)(C)C)cc2)n1)C(=O)NC(=C)C(=O)OC ; C=C(NC(=O)c1csc(-c2ccc(N)cc2)n1)C(=O)NC(=C)C(=O)OC]

Commercially available thiazole ethyl ester SM1a was hydrolyzed using LiOH to provide carboxylic acid Int 2a. Subsequent amide coupling with serine methyl ester (SM3) provided amide Int 4a. The free hydroxyl group was protected by treatment with TBDPS-Cl to provide compound Int 5a after column chromatography (79% over 3 steps). Hydrolysis of the methyl ester using LiOH provided carboxylic acid derivative Int 6a, which was subsequently coupled with serine methyl ester SM7a to provide dipeptide Int 8a, purified by column chromatography (47% over 2 steps). Elimination of the hydroxyl moiety was achieved by treatment with mesyl chloride and NEt₃ and provided dehydroalanine derivative Int 9a. TBAF mediated TBDPS-removal followed by a second elimination reaction provided bisdehydroalanine derivative Int 11a.

### Example 1B: Exemplary Syntheses of Thiostrepton-Inspired Structure A1 ((5)-HCl)

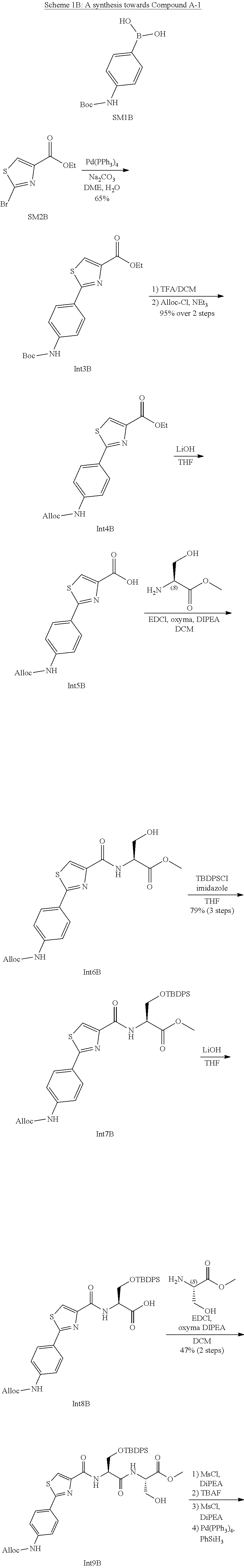

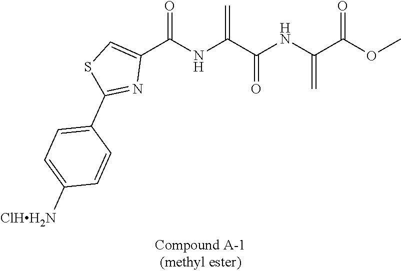

Suzuki coupling between boronic ester SM1B and bromide SM2B provided compound Int3B in moderate yield (65%). The Boc-protecting group was swapped to Alloc in 95% yield to provide Alloc protected compound Int4B. Hydrolysis of the methyl ester and subsequent EDCI mediated coupling of serine methyl ester provided compound Int6B. The free alcohol was protected with TBDPS and after purification compound Int7B was isolated in 79% over 3 steps. Hydrolysis of the methyl ester and subsequent EDCI mediated coupling with serine methyl ester provided compound Int9B (45% yield). Elimination of the hydroxyl was achieved by treatment with mesyl chloride and NEt₃. TBAF mediated TBDPS-removal followed by a second elimination reaction provided bisdehydroalanine motive. Finally, the Alloc group was removed by treatment with tetrakis in presence of a scavenger to provide Compound A-1.

### Alternate Synthesis:

### 2-(4-((tert-butoxycarbonyl)amino)phenyl)thiazole-4-carboxylic acid Intermediate 2

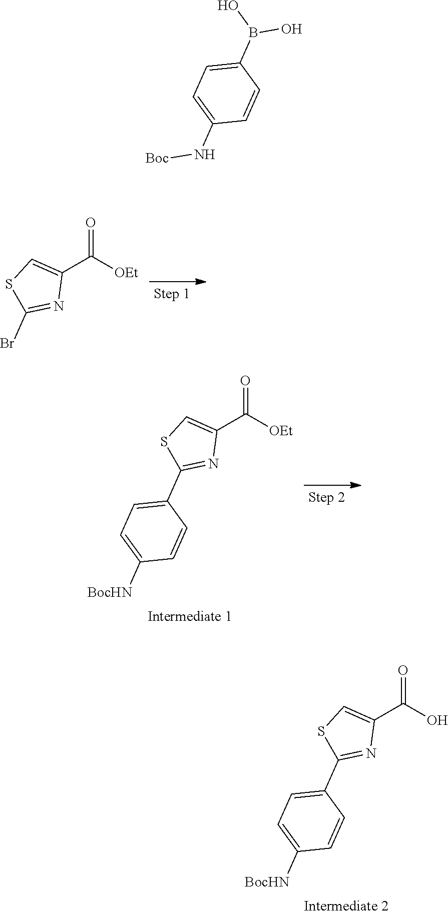 [SMILES: CC(C)(C)OC(=O)Nc1ccc(B(O)O)cc1 ; CCOC(=O)c1csc(Br)n1 ; "Step 1" ; CCOC(=O)c1csc(-c2ccc(NC(=O)OC(C)(C)C)cc2)n1 ; "Step 2" ; CC(C)(C)OC(=O)Nc1ccc(-c2nc(C(=O)O)cs2)cc1]

Step 1. Ethyl 2-(4-((tert-butoxycarbonyl)amino)phenyl)thiazole-4-carboxylate (Intermediate 1) was prepared following General experimental procedure 1. 4-(tert-butoxycarbonylamino)phenylboronic acid (2.5 g, 11 mmol) and ethyl 2-bromothiazole-4-carboxylate (2.5 g, 11 mmol) gave ethyl 2-(4-((tert-butoxycarbonyl)amino)phenyl)thiazole-4-carboxylate (Intermediate 1) (2.4 g, 6.9 mmol, 65%) as an off-white solid. ¹H NMR (400 MHz, CDCl₃) δ 8.08 (s, 1H), 7.96-7.88 (m, 2H), 7.47-7.40 (m, 2H), 6.59 (s, 1H), 4.42 (q, J=7.1 Hz, 2H), 1.51 (s, 9H), 1.41 (t, J=7.1 Hz, 3H).

Step 2. 2-(4-((tert-butoxycarbonyl)amino)phenyl)thiazole-4-carboxylic acid (Intermediate 2) was prepared following General experimental procedure 2. 2-(4-((tert-butoxycarbonyl)amino)phenyl)thiazole-4-carboxylate (Intermediate 1) (0.93 g, 2.7 mmol) gave 2-(4-((tert-butoxycarbonyl)amino)phenyl)thiazole-4-carboxylic acid (Intermediate 2) (0.82 g, 2.6 mmol, 97%) as an off-white solid. ¹H NMR (400 MHz, CDCl₃) δ 8.09 (s, 1H), 7.89-7.82 (m, 2H), 7.45-7.34 (m, 2H), 1.47 (s, 9H), 1.24-1.13 (m, 1H).

### Methyl 2-(2-(2-(4-aminophenyl)thiazole-4-carboxamido)acrylamido)acrylate hydrochloride (Compound A1)

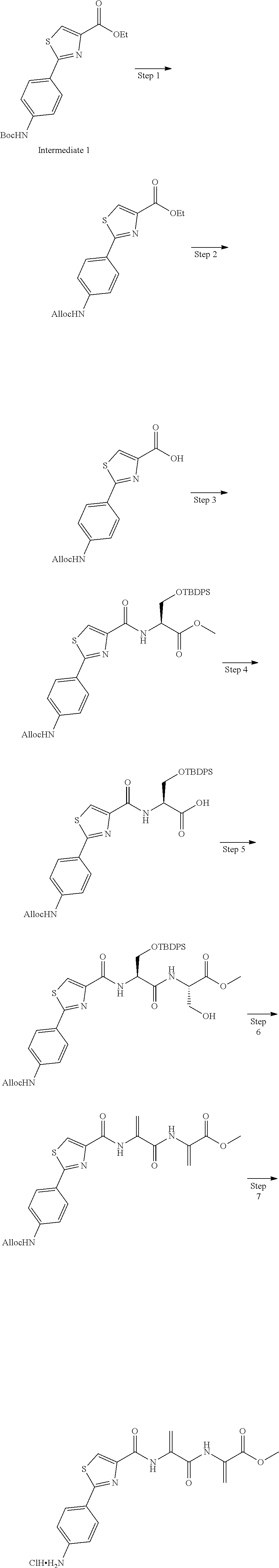

Step 1. Ethyl 2-(4-(((allyloxy)carbonyl)amino)phenyl)thiazole-4-carboxylate was prepared with the following procedure. To a solution of ethyl 2-(4-((tert-butoxycarbonyl)amino)phenyl)thiazole-4-carboxylate (Intermediate 1) (2.4 g, 1 eq., 6.9 mmol) in CH₂Cl₂ (10 mL) was added TFA (10 mL) and the resulting mixture was stirred at room temperature for 2 hours. The mixture was concentrated in vacuo and the residue was taken up in THF (50 mL). To the mixture was added pyridine (2.8 mL, 5 eq., 34 mmol) followed by allyl chloroformate (0.96 mL, 1.3 eq., 9.0 mmol) and the resulting mixture was stirred for 1 hour. The mixture was diluted with EtOAc, washed with 1 M HCl and brine, dried over Na₂SO₄, and concentrated to provide crude ethyl 2-(4-(((allyloxy)carbonyl)amino)phenyl)thiazole-4-carboxylate as a yellow solid. The crude material was used as such in the next step.

Step 2. 2-(4-(((allyloxy)carbonyl)amino)phenyl)thiazole-4-carboxylic acid was prepared following General experimental procedure 2. Crude ethyl 2-(4-(((allyloxy)carbonyl)amino)phenyl)thiazole-4-carboxylate gave 2-(4-(((allyloxy)carbonyl)amino)phenyl)thiazole-4-carboxylic acid (2.0 g, 6.6 mmol, 95%) as orange solid. ¹H NMR (400 MHz, CDCl₃) δ 8.20 (s, 1H), 7.95-7.87 (m, 2H), 7.50 (d, J=8.5 Hz, 2H), 6.83 (s, 1H), 5.96 (ddt, J=17.2, 10.4, 5.8 Hz, 1H), 5.37 (dq, J=17.2, 1.5 Hz, 1H), 5.32-5.24 (m, 1H), 4.68 (dt, J=5.8, 1.4 Hz, 2H)

Step 3. Methyl N-(2-(4-(((allyloxy)carbonyl)amino)phenyl)thiazole-4-carbonyl)-O-(tert-butyldiphenylsilyl)-L-serinate was prepared following General experimental procedure 3. 2-(4-(((allyloxy)carbonyl)amino)phenyl)thiazole-4-carboxylic acid 2-(4-(((allyloxy)carbonyl)amino)phenyl)thiazole-4-carboxylic acid (1.1 g, 1 eq., 3.6 mmol) and methyl O-(tert-butyldiphenylsilyl)-L-serinate hydrochloride (1.7 g, 1.2 eq., 4.3 mmol) gave methyl N-(2-(4-(((allyloxy)carbonyl)amino)phenyl)thiazole-4-carbonyl)-O-(tert-butyldiphenylsilyl)-L-serinate (1.9 g, 3.0 mmol, 82%).

Step 4. N-(2-(4-(((allyloxy)carbonyl)amino)phenyl)thiazole-4-carbonyl)-O-(tert-butyldiphenylsilyl)-L-serine was prepared following General experimental procedure 2. Methyl N-(2-(4-(((allyloxy)carbonyl)amino)phenyl)thiazole-4-carbonyl)-O-(tert-butyldiphenylsilyl)-L-serinate (1.9 g, 1 eq., 3.0 mmol) gave N-(2-(4-(((allyloxy)carbonyl)amino)phenyl)thiazole-4-carbonyl)-O-(tert-butyldiphenylsilyl)-L-serine (1.8 g, 2.8 mmol, 93%) as a clear oil. The crude material was used as such in the next step.

Step 5. Methyl N-(2-(4-(((allyloxy)carbonyl)amino)phenyl)thiazole-4-carbonyl)-O-(tert-butyldiphenylsilyl)-L-seryl-L-serinate was prepared following General experimental procedure 3. N-(2-(4-(((allyloxy)carbonyl)amino)phenyl)thiazole-4-carbonyl)-O-(tert-butyldiphenylsilyl)-L-serine (1.8 g, 2.8 mmol) gave methyl N-(2-(4-(((allyloxy)carbonyl)amino)phenyl)thiazole-4-carbonyl)-O-(tert-butyldiphenylsilyl)-L-seryl-L-serinate (0.75 g, 1.0 mmol, 35%) as a yellow oil.

Step 6. Methyl 2-(2-(2-(4-(((allyloxy)carbonyl)amino)phenyl)thiazole-4-carboxamido)acrylamido)acrylate was prepared following General experimental procedure 6. Methyl N-(2-(4-(((allyloxy)carbonyl)amino)phenyl)thiazole-4-carbonyl)-O-(tert-butyldiphenylsilyl)-L-seryl-L-serinate (0.75 g, 1 eq., 1.0 mmol) gave crude methyl 2-(2-(2-(4-(((allyloxy)carbonyl)amino)phenyl)thiazole-4-carboxamido)acrylamido)acrylate (0.9 g) which was used without purification in the next step.

Step 7. Methyl 2-(2-(2-(4-aminophenyl)thiazole-4-carboxamido)acrylamido)acrylate was prepared with the following procedure. To a solution of crude methyl 2-(2-(2-(4-(((allyloxy)carbonyl)amino)phenyl)thiazole-4-carboxamido)acrylamido)acrylate (0.90 g) in DCM (20 mL) was added phenylsilane (1.1 g, 1.2 mL, 5 eq., 9.9 mmol) followed by tetrakis(triphenylphosphine)-palladium(0) (0.23 g, 0.1 eq., 0.20 mmol) and the resulting mixture was stirred for 1 hour. MeCN (4 mL) was added and the CH₂Cl₂ was removed in vacuo. The mixture was directly submitted to automated reverse phase FCC to provide methyl 2-(2-(2-(4-aminophenyl)thiazole-4-carboxamido)acrylamido)acrylate (8.5 mg, 23 μmol, 1.2%) as an offwhite solid. LCMS (21020335C TFA LCMS-5 C4) RT: 1.274 min; area % (215 nm): 79.1%; m/z=373.2 [M+H]⁺. ¹H NMR (400 MHz, MeOD) δ 8.38 (s, 1H), 8.33-8.17 (m, 2H), 7.57-7.52 (m, 2H), 6.60 (d, J=1.8 Hz, 1H), 6.29 (s, 1H), 6.00 (s, 1H), 5.76 (d, J=1.9 Hz, 1H), 3.86 (s, 3H).

### Example 2A: Exemplary Syntheses of Thiostrepton-Inspired Structure A2 ((1))

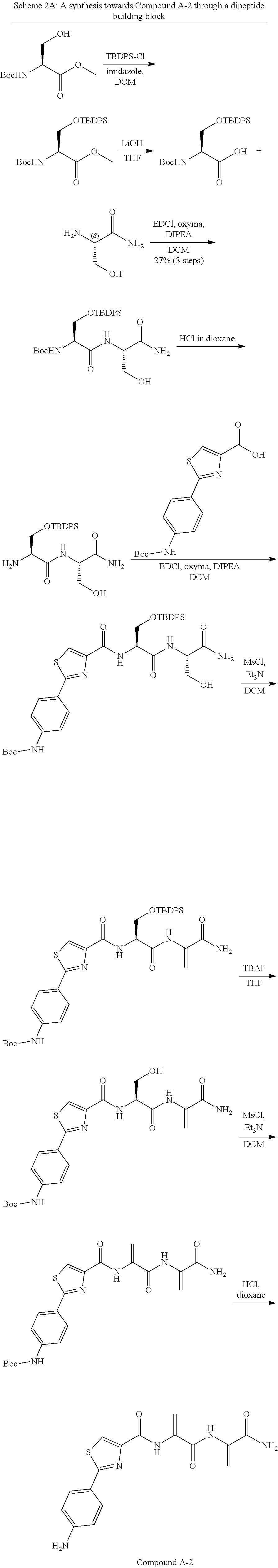

Compound A-2 was accessed by coupling between a thiazole derivative and a dipeptide. The dipeptide was synthesized starting from Boc-Ser-OMe. First the hydroxyl was protected by treatment with TBDPS-Cl to provide a serine derivative. The methyl ester was hydrolyzed by treatment with LiOH to provide a carboxylic acid, which was then coupled with serinamide to provide a dipeptide after column chromatography (27% over 3 steps). The Boc protecting group was removed by treatment with HCl and provided the dipeptide. EDCI-mediated coupling of the dipeptide and carboxylic acid provided a thiazole coupled dipeptide after column chromatography (51%). Elimination of the hydroxyl, mediated by mesyl chloride and NEt₃, followed by TBDPS removal by TBAF and a second elimination reaction provided Boc-protected compound after preparative HPLC purification. Treatment with HCl removed the Boc protecting group and provided Compound A-2.

### Example 2B: Exemplary Syntheses of Thiostrepton-Inspired Structure A2

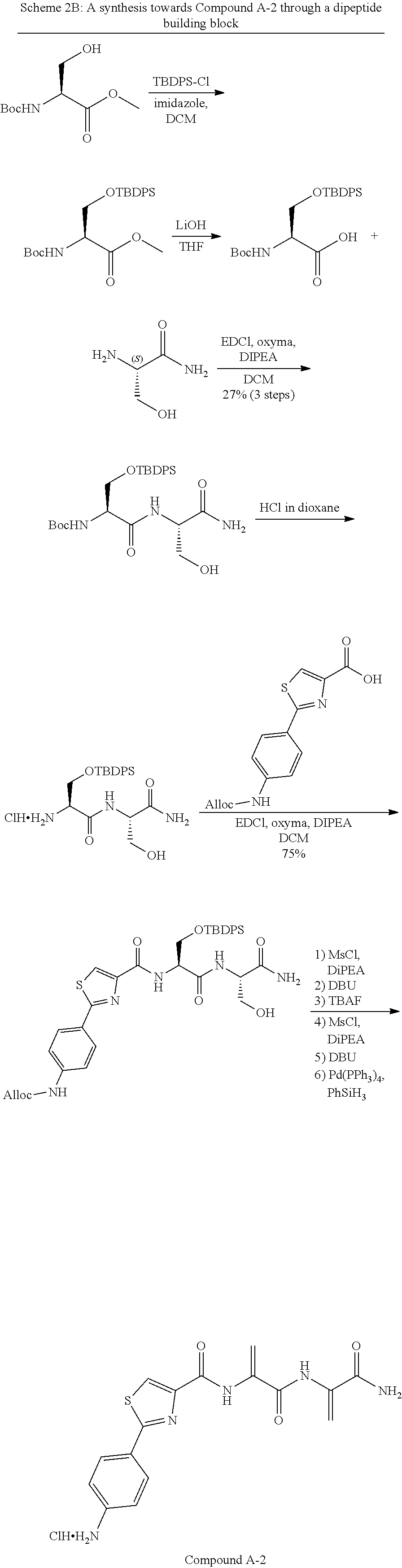

The hydroxyl-moiety of Boc-Ser-OMe was protected with TBDPS to provide Boc-Ser(TBDPS)—OMe. The methyl ester was subsequently removed by treatment with LiOH to provide an acid. EDCI-mediated peptide coupling with serinamide provided a dipeptide in 27% over 3 steps. Treatment with HCl removed the Boc-group to provide an amine. EDCI-mediated coupling between the amine and the acid, synthesis of which is shown above, provided the product in 75% over 2 steps. Elimination of the hydroxyl was achieved by treatment with mesyl chloride and NEt₃. TBAF mediated TBDPS-removal followed by a second elimination reaction provided bisdehydroalanine motive. Finally, the Alloc group was removed by treatment with palladium in presence of a scavenger to provide Compound A-2.

### Alternate Synthesis of Tautomer of Compound A-2/Tautomer ((1)/Tautomer):

### Methyl 2-(2-(2-(4-aminophenyl)thiazole-4-carboxamido)acrylamido)acrylate hydrochloride

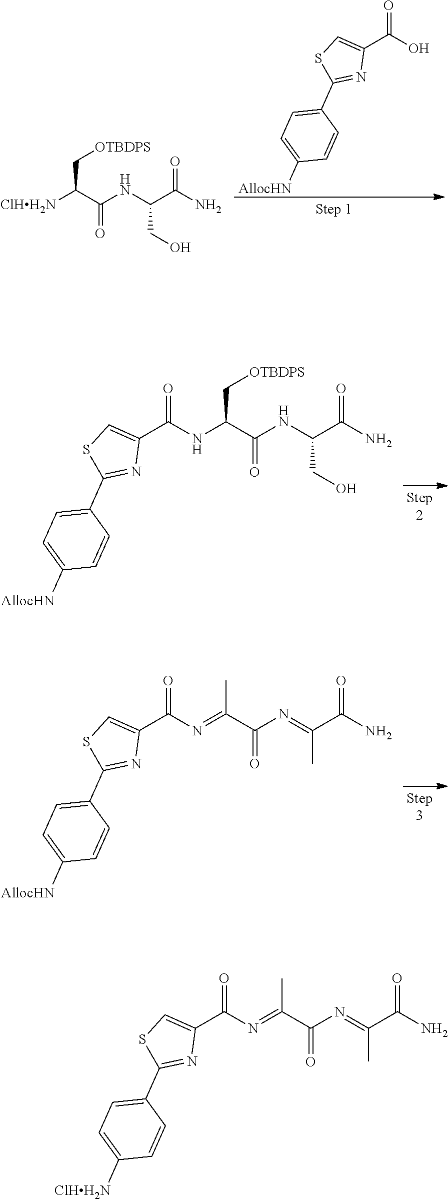

Step 1. allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate was prepared following General experimental procedure 3. 2-(4-(((allyloxy)carbonyl)amino)phenyl)thiazole-4-carboxylic acid (1.2 g, 1 eq., 3.8 mmol) (as prepared in step 2) and (S)-2-amino-N—((S)-1-amino-3-hydroxy-1-oxopropan-2-yl)-3-((tert-butyldiphenylsilyl)oxy)propanamide hydrochloride (as prepared in 7) (2.2 g, 1.2 eq., 4.6 mmol) gave allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (1.5 g, 2.1 mmol, 55%) as a white foam. LCMS (General 3 acidic) RT: 1.44 min; area % (254 nm): 92%; m/z=716.2 [M+H]⁺. ¹H NMR (400 MHz, CDCl₃) δ 8.35 (d, J=6.6 Hz, 1H), 8.06 (s, 1H), 7.89-7.81 (m, 2H), 7.65 (tt, J=6.2, 1.5 Hz, 4H), 7.38 (dddd, J=22.2, 20.7, 8.4, 6.7 Hz, 8H), 6.85 (s, 1H), 6.79 (s, 1H), 6.03-5.89 (m, 1H), 5.42-5.32 (m, 2H), 5.31-5.23 (m, 1H), 4.71-4.59 (m, 3H), 4.56 (dt, J=7.7, 4.1 Hz, 1H), 4.22 (ddd, J=17.4, 10.8, 3.3 Hz, 2H), 3.95 (dd, J=10.2, 4.9 Hz, 1H), 3.63 (dd, J=11.4, 4.5 Hz, 1H), 3.47 (s, 1H), 1.09 (s, 9H).

Step 2. allyl (4-(4-((3-((3-amino-3-oxoprop-1-en-2-yl)amino)-3-oxoprop-1-en-2-yl)carbamoyl)thiazol-2-yl)phenyl)carbamate was prepared following General experimental procedure 6. allyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (0.35 g, 0.49 mmol) gave crude allyl (4-(4-((3-((3-amino-3-oxoprop-1-en-2-yl)amino)-3-oxoprop-1-en-2-yl)carbamoyl)thiazol-2-yl)phenyl)carbamate (0.35 g), which was used as such in the next step.

Step 3. N-(3-((3-amino-3-oxoprop-1-en-2-yl)amino)-3-oxoprop-1-en-2-yl)-2-(4-aminophenyl)thiazole-4-carboxamide hydrochloride was prepared using the following procedure. To a solution of crude allyl (4-(4-((3-((3-amino-3-oxoprop-1-en-2-yl)amino)-3-oxoprop-1-en-2-yl)carbamoyl)thiazol-2-yl)phenyl)carbamate (0.35 g, 0.79 mmol) in DCM (10 mL) were added Silylbenzene (0.49 mL, 5 eq., 4.0 mmol) and tetrakis (92 mg, 0.1 eq., 79 μmol) and the resulting mixture was stirred for 1 hour. MeCN (4 mL) was added and the DCM was removed in vacuo. The mixture was directly submitted to automated reverse phase FCC to provide methyl 2-(2-(2-(4-aminophenyl)thiazole-4-carboxamido)acrylamido)acrylate hydrochloride as a white solid. LCMS (21020335B TFA LCMS-5 C3) RT: 1.138 min; area: 63.7% (215 nm); m/z=458.2 [M+H]⁺.

### Example 3: Exemplary Syntheses of Thiostrepton-Inspired Structure B

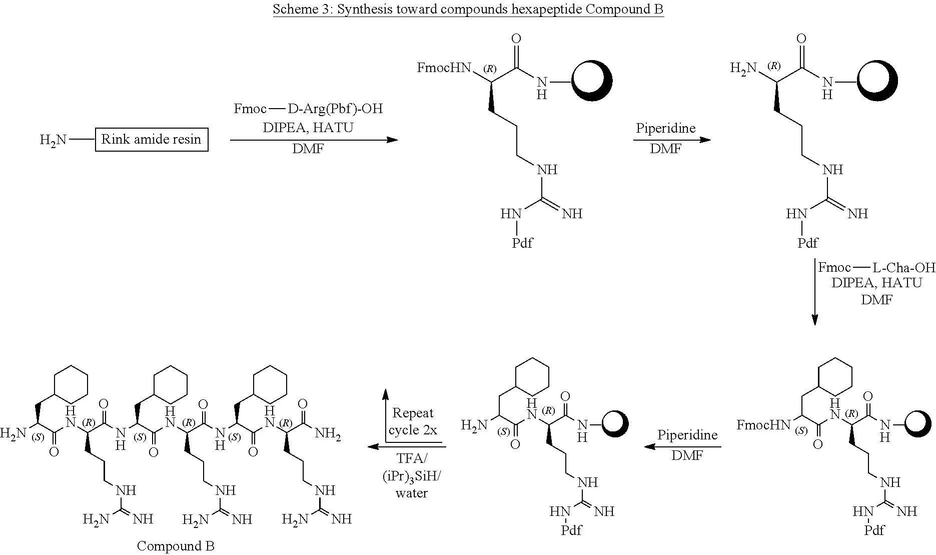

Sequential build-up SPPS of hexapeptide compound B was started using 5 gram Rink Amide resin (0.7 mmol/g) and using standard SPPS protocols. After full deprotection and simultaneous cleavage from the resin using TFA/TIPS/water (95/2.5/2.5), the crude peptide was obtained by precipitation from MTBE:heptane (1:1) and lyophilization. The crude peptide was purified using preparative HPLC to provide >95% purity of Compound B as the TFA salt. To convert to the HCl salt, the purified peptide was basified with NaOH and reacidified with HCl and subsequent preparative HPLC using HCl buffers provided the pure peptide as HCl salt.

### Alternate Synthesis:

### H-Cha-D-Arg-Cha-D-Arg-Cha-D-Arg-NH₂ HCl Salt

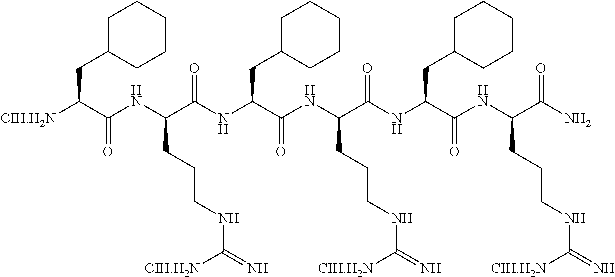

H-Cha-D-Arg-Cha-D-Arg-Cha-D-Arg-NH₂ HCl salt was prepared following General procedure for solid phase peptide synthesis on 2.8 mmol scale. Purification using HCl buffers provided the desired HCl salt form. H-Cha-D-Arg-Cha-D-Arg-Cha-D-Arg-NH₂ HCl salt (0.89 g, 0.82 mmol, 29%) was isolated as a white solid. LCMS (30833 LCMS-6): RT: 1.819 min; Area: 92.5% (215 nm), 99.7% (ELSD); m/z=945.9 [M+H]⁺

### Example 4: Compounds 1 and 2

### tert-butyl (4-(4-((3-((3-amino-3-oxoprop-1-en-2-yl)amino)-3-oxoprop-1-en-2-yl)carbamoyl)thiazol-2-yl)phenyl)carbamate & tert-butyl (4-(4-(((E)-1-(((E)-1-amino-1-oxopropan-2-ylidene)amino)-1-oxopropan-2-ylidene)carbamoyl)thiazol-2-yl)phenyl)carbamate

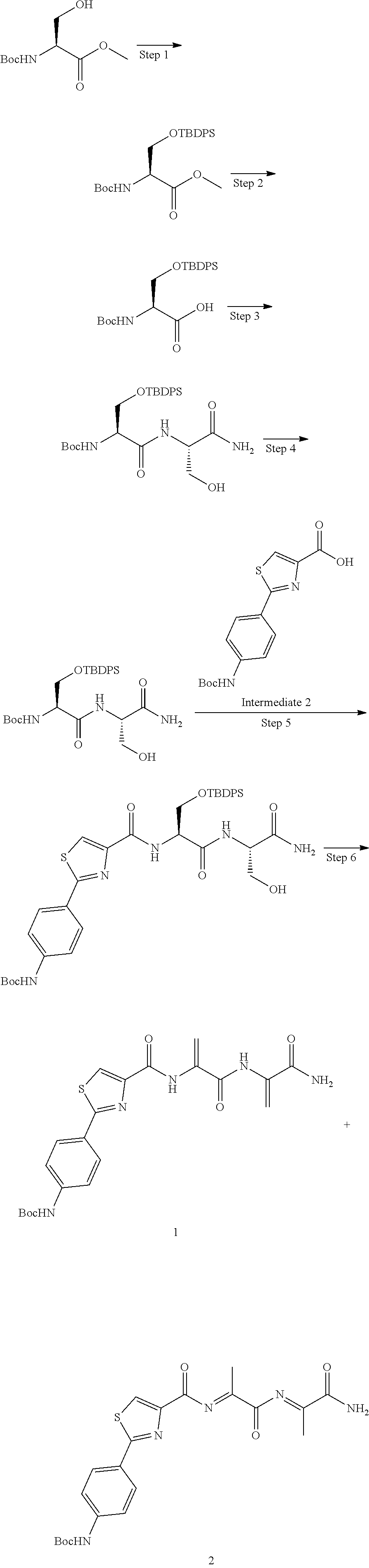

Step 1. Methyl N-(tert-butoxycarbonyl)-O-(tert-butyldiphenylsilyl)-L-serinate was prepared with the following procedure. To a solution of methyl (tert-butoxycarbonyl)-L-serinate (12 g, 1 eq., 55 mmol) in DCM (250 mL) and imidazole (8.2 g, 2.2 eq., 0.12 mol) was added TBDPS-Cl (17 g, 15 mL, 1.1 eq., 60 mmol) and the resulting mixture was stirred at room temperature overnight. The mixture was washed with 1 M HCl (200 mL) and brine (200 mL), dried over Na₂SO₄, filtered, and concentrated to give methyl N-(tert-butoxycarbonyl)-O-(tert-butyldiphenylsilyl)-L-serinate (24 g, 52 mmol, 96%) as cloudy oil, which solidified on standing. ¹H NMR (400 MHz, CDCl₃) δ 7.59 (m, 4H), 7.46-7.32 (m, 6H), 5.39 (d, J=8.8 Hz, 1H), 5.28 (d, J=1.0 Hz, 1H), 4.38 (dt, J=9.2, 3.1 Hz, 1H), 4.05 (dd, J=10.0, 3.0 Hz, 1H), 3.87 (dd, J=10.1, 3.1 Hz, 1H), 3.72 (s, 3H), 1.44 (s, 9H), 1.01 (s, 9H).

Step 2. N-(tert-butoxycarbonyl)-O-(tert-butyldiphenylsilyl)-L-serine was prepared following General experimental procedure 2. Methyl N-(tert-butoxycarbonyl)-O-(tert-butyldiphenylsilyl)-L-serinate (12 g, 26 mmol) gave N-(tert-butoxycarbonyl)-O-(tert-butyldiphenylsilyl)-L-serine (11.9 g, 26.8 mmol, quant.). The material was used in the next step without purification.

Step 3. tert-butyl ((S)-1-(((S)-1-amino-3-hydroxy-1-oxopropan-2-yl)amino)-3-((tert-butyldiphenylsilyl)oxy)-1-oxopropan-2-yl)carbamate was prepared following General experimental procedure 3. N-(tert-butoxycarbonyl)-O-(tert-butyldiphenylsilyl)-L-serine (10 g, 1 eq., 23 mmol) and L-serinamide hydrochloride (6.5 g, 2 eq., 46 mmol) gave tert-butyl ((S)-1-(((S)-1-amino-3-hydroxy-1-oxopropan-2-yl)amino)-3-((tert-butyldiphenylsilyl)oxy)-1-oxopropan-2-yl)carbamate (2.5 g, 4.7 mmol, 20%) as a slightly pink solid. ¹H NMR (400 MHz, CDCl₃) δ 7.59 (m, 4H), 7.48-7.33 (m, 6H), 7.21 (m, 1H), 6.83 (s, 1H), 5.33 (s, 1H), 5.23 (s, 1H), 4.50 (s, 1H), 4.22 (dd, J=11.3, 2.7 Hz, 1H), 4.16 (d, J=5.8 Hz, 1H), 4.09 (dd, J=10.3, 4.1 Hz, 1H), 3.83 (dd, J=10.4, 4.4 Hz, 1H), 3.60 (dd, J=11.4, 4.3 Hz, 1H), 1.43 (s, 9H), 1.04 (s, 9H).

Step 4. (S)-2-amino-N—((S)-1-amino-3-hydroxy-1-oxopropan-2-yl)-3-((tert-butyldiphenylsilyl)oxy)propanamide hydrochloride was prepared with the following procedure. Tert-butyl ((S)-1-(((S)-1-amino-3-hydroxy-1-oxopropan-2-yl)amino)-3-((tert-butyldiphenylsilyl)oxy)-1-oxopropan-2-yl)carbamate (2.5 g, 1 Eq, 4.7 mmol) was dissolved in 15 mL 4 M HCl in dioxane and the resulting mixture was stirred at room temperature for 90 minutes. The mixture was concentrated to provide (S)-2-amino-N—((S)-1-amino-3-hydroxy-1-oxopropan-2-yl)-3-((tert-butyldiphenylsilyl)oxy)propanamide hydrochloride (2.2 g, 4.6 mmol, 98%) as white solid. ¹H NMR (400 MHz, MeOD) δ 7.73-7.62 (m, 4H), 7.52-7.39 (m, 6H), 4.54 (t, J=5.4 Hz, 1H), 4.11 (t, J=4.5 Hz, 1H), 4.03 (d, J=4.5 Hz, 2H), 3.86-3.74 (m, 2H), 1.07 (s, 9H).

Step 5. tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate was prepared following General experimental procedure 3. 2-(4-((tert-butoxycarbonyl)amino)phenyl)thiazole-4-carboxylic acid (Intermediate 2) (0.70 g, 2.2 mmol) and (S)-2-amino-N—((S)-1-amino-3-hydroxy-1-oxopropan-2-yl)-3-((tert-butyldiphenylsilyl)oxy)propanamide hydrochloride (2.0 g, 2 eq., 4.4 mmol) gave tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (1.3 g, 1.8 mmol, 81%). %). LCMS (General 3 acidic) RT: 1.59 min; area % (214 nm): 75%; m/z=732.2 [M+H]⁺.

Step 6. tert-butyl (4-(4-((3-((3-amino-3-oxoprop-1-en-2-yl)amino)-3-oxoprop-1-en-2-yl)carbamoyl)thiazol-2-yl)phenyl)carbamate & tert-butyl (4-(4-(((E)-1-(((E)-1-amino-1-oxopropan-2-ylidene)amino)-1-oxopropan-2-ylidene)carbamoyl)thiazol-2-yl)phenyl)carbamate were prepared using the following procedure. To a solution of tert-butyl (4-(4-(((S)-1-(((S)-1-amino-3-hydroxy-1-oxopropan-2-yl)amino)-3-((tert-butyldiphenylsilyl)oxy)-1-oxopropan-2-yl)carbamoyl)thiazol-2-yl)phenyl)carbamate (0.13 g, 0.18 mmol) in CH₂Cl₂ (2 mL) were added triethylamine (0.12 mL, 5 eq., 0.89 mmol) and methansulfonyl chloride (21 μL, 1.5 eq., 0.27 mmol) and the resulting mixture was stirred at room temperature for 3 hours. The mixture was diluted with CH₂Cl₂ (25 mL) and washed with 1M HCl and brine, dried over Na₂SO₄, filtered and concentrated partially to reach a volume of roughly 10 mL. Then, DBU (54 μL, 2 eq., 0.36 mmol) was added and the mixture was stirred for 1 hour. A solution of TBAF in THF (0.36 mL, 1.0 molar, 2 eq., 0.36 mmol) was added and the mixture was stirred for 30 minutes. The mixture was diluted with CH₂Cl₂ (25 mL) and washed with 1M HCl and brine, dried over Na₂SO₄, filtered and concentrated partially to reach a volume of roughly 10 mL. To the resulting mixture were added triethylamine (0.12 mL, 5 eq., 0.89 mmol) and methanesulfonyl chloride (21 μL, 1.5 eq., 0.27 mmol) and the resulting mixture was stirred for 1 hour at room temperature. The mixture was diluted with CH₂Cl₂ (25 mL) and washed with 1M HCl and brine, dried over Na₂SO₄, filtered and concentrated partially to reach a volume of roughly 10 mL. DBU (54 μL, 2 eq., 0.36 mmol) was added and the resulting mixture was stirred for 1 hour. The mixture was diluted with CH₂Cl₂ (25 mL) and washed with 1M HCl and brine, dried over Na₂SO₄, filtered, and concentrated. The crude material was purified by automated reverse phase FCC to provide 1 (2.0 mg, 4.4 μmol, 2.4%) and 2 (8.0 mg, 17 μmol, 9.7%). 1: LCMS (22010199A TFA LCMS-5 C1) RT: 1.138 min; area: 63.7% (215 nm); m/z 458.2 [M+H]⁺. ¹H NMR (400 MHz, CDCl₃) δ 9.99 (s, 1H), 8.92 (s, 1H), 8.07 (d, J=5.2 Hz, 1H), 7.96-7.86 (m, 2H), 7.45 (m, 2H), 6.76 (d, J=2.2 Hz, 1H), 6.65 (d, J=2.1 Hz, 1H), 6.62 (s, 1H), 5.53 (t, J=1.9 Hz, 1H), 5.43-5.36 (m, 1H), 1.65 (s, 13H), 1.52 (s, 9H). 2: LCMS (22010199 LCMS-5 C3) RT: 1.138 min; area: 63.7% (215 nm); m/z 458.2 [M+H]. ¹H NMR (400 MHz, CDCl₃) δ 10.84 (s, 1H), 8.22 (s, 1H), 7.93-7.86 (m, 2H), 7.53-7.47 (m, 2H), 7.36 (s, 1H), 6.66 (s, 1H), 2.33 (s, 3H), 1.78 (s, 3H), 1.53 (s, 9H).

### Example 5: Compound 4

### Methyl 2-(2-(2-(4-((tert-butoxycarbonyl)amino)phenyl)thiazole-4-carboxamido)acrylamido)acrylate

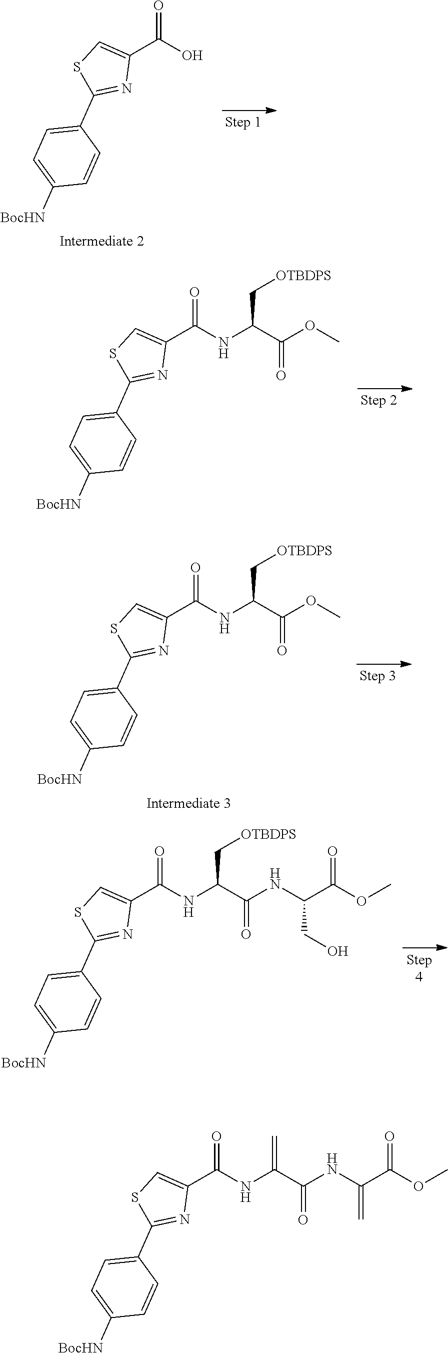

Step 1. Methyl N-(2-(4-((tert-butoxycarbonyl)amino)phenyl)thiazole-4-carbonyl)-O-(tert-butyldiphenylsilyl)-L-serinate was prepared following General experimental procedure 3. 2-(4-((tert-butoxycarbonyl)amino)phenyl)thiazole-4-carboxylic acid (Intermediate 2) (2.0 g, 6.2 mmol) and methyl O-(tert-butyldiphenylsilyl)-L-serinate hydrochloride (2.5 g, 1 eq., 6.2 mmol) gave methyl N-(2-(4-((tert-butoxycarbonyl)amino)phenyl)thiazole-4-carbonyl)-O-(tert-butyldiphenylsilyl)-L-serinate (3.5 g, 5.3 mmol, 85%)

Step 2. N-(2-(4-((tert-butoxycarbonyl)amino)phenyl)thiazole-4-carbonyl)-O-(tert-butyldiphenylsilyl)-L-serine (Intermediate 3) was prepared following General experimental procedure 2. Methyl N-(2-(4-((tert-butoxycarbonyl)amino)phenyl)thiazole-4-carbonyl)-O-(tert-butyldiphenylsilyl)-L-serinate (3.5 g, 5.3 mmol) gave crude N-(2-(4-((tert-butoxycarbonyl)amino)phenyl)thiazole-4-carbonyl)-O-(tert-butyldiphenylsilyl)-L-serine (Intermediate 3) as clear oil, which was used as such in the next step.

Step 3. Methyl N-(2-(4-((tert-butoxycarbonyl)amino)phenyl)thiazole-4-carbonyl)-O-(tert-butyldiphenylsilyl)-L-seryl-L-serinate was prepared following General experimental procedure 3. Crude N-(2-(4-((tert-butoxycarbonyl)amino)phenyl)thiazole-4-carbonyl)-O-(tert-butyldiphenylsilyl)-L-serine (Intermediate 3) (as obtained from step 2) and methyl L-serinate hydrochloride (1.2 g, 1.5 eq., 8.0 mmol) gave methyl N-(2-(4-((tert-butoxycarbonyl)amino)phenyl)thiazole-4-carbonyl)-O-(tert-butyldiphenylsilyl)-L-seryl-L-serinate (1.0 g, 1.3 mmol, 25%) as a yellow oil. LCMS (General 3 acidic) RT: 1.74 min; area % (254 nm): 87%; m/z=747.2 [M+H]⁺.

Step 4. Methyl 2-(2-(2-(4-((tert-butoxycarbonyl)amino)phenyl)thiazole-4-carboxamido)acrylamido)acrylate was prepared following General experimental procedure 6. Methyl N-(2-(4-((tert-butoxycarbonyl)amino)phenyl)thiazole-4-carbonyl)-O-(tert-butyldiphenylsilyl)-L-seryl-L-serinate (0.10 g, 1 eq., 0.13 mmol) gave methyl 2-(2-(2-(4-((tert-butoxycarbonyl)amino)phenyl)thiazole-4-carboxamido)acrylamido)acrylate (22 mg, 47 μmol, 35%) as a white solid. LCMS (22010199A TFA LCMS-5 C1) RT: 1.6884 min; area % (215 nm): 97.1%; m/z=473.2 [M+H]⁺. ¹H NMR (400 MHz, CDCl₃) δ 9.98 (s, 1H), 8.52 (s, 1H), 8.07 (s, 1H), 7.96-7.88 (m, 2H), 7.45 (d, J=8.7 Hz, 2H), 6.75 (d, J=2.2 Hz, 1H), 6.69 (s, 1H), 6.61 (s, 1H), 6.01 (d, J=1.3 Hz, 1H), 5.48 (t, J=1.9 Hz, 1H), 3.88 (s, 3H), 1.52 (s, 9H).

### Alternate Synthesis:

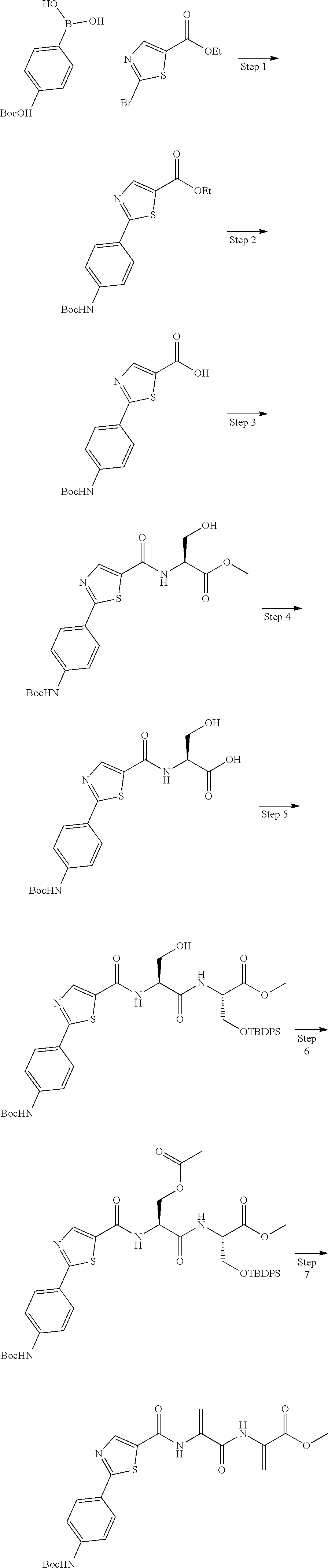

Step 1. ethyl 2-(4-((tert-butoxycarbonyl)amino)phenyl)thiazole-5-carboxylate was prepared following General experimental procedure 1. (4-((tert-butoxycarbonyl)amino)phenyl)boronic acid (2.0 g, 8.4 mmol) and ethyl 2-bromothiazole-5-carboxylate (2.0 g, 8.4 mmol) gave ethyl 2-(4-((tert-butoxycarbonyl)amino)phenyl)thiazole-5-carboxylate (1.26 g, 3.62 mmol, 43%) as a yellow solid. ¹H NMR (299 MHz, CDCl₃) δ 8.39 (s, 1H), 7.94 (d, J=8.8 Hz, 2H), 7.49 (d, J=8.6 Hz, 2H), 6.66 (s, 1H), 4.40 (q, J=7.1 Hz, 2H), 1.55 (d, J=1.0 Hz, 10H), 1.42 (t, J=7.1 Hz, 3H).

Step 2. 2-(4-((tert-butoxycarbonyl)amino)phenyl)thiazole-5-carboxylic acid was prepared following General experimental procedure 2. Ethyl 2-(4-((tert-butoxycarbonyl)amino)phenyl)thiazole-5-carboxylate (1.2 g, 3.4 mmol) gave 2-(4-((tert-butoxycarbonyl)amino)phenyl)thiazole-5-carboxylic acid (1.0 g, 3.1 mmol, 91%) as a white solid.

Step 3. methyl (2-(4-((tert-butoxycarbonyl)amino)phenyl)thiazole-5-carbonyl)-L-serinate was prepared following General experimental procedure 4. 2-(4-((tert-butoxycarbonyl)amino)phenyl)thiazole-5-carboxylic acid (1.6 g, 5.0 mmol) and H-Ser-OMe·HCl (0.93 g, 1.2 eq., 6.0 mmol) gave methyl (2-(4-((tert-butoxycarbonyl)amino)phenyl)thiazole-5-carbonyl)-L-serinate (1.4 g, 3.3 mmol, 67%) as an orange oil. LCMS (General 3 acidic) RT: 0.99 min; area % (254 nm): 88%; m/z=422.3 [M+H]⁺.

Step 4. (2-(4-((tert-butoxycarbonyl)amino)phenyl)thiazole-5-carbonyl)-L-serine was prepared following General experimental procedure 2. Methyl (2-(4-((tert-butoxycarbonyl)amino)phenyl)thiazole-5-carbonyl)-L-serinate (1.4 g, 3.3 mmol) gave (2-(4-((tert-butoxycarbonyl)amino)phenyl)thiazole-5-carbonyl)-L-serine (0.98 g, 2.4 mmol, 72%).

Step 5. Methyl N-((2-(4-((tert-butoxycarbonyl)amino)phenyl)thiazole-5-carbonyl)-L-seryl)-O-(tert-butyldiphenylsilyl)-L-serinate was prepared following General experimental procedure 4. (2-(4-((tert-butoxycarbonyl)amino)phenyl)thiazole-5-carbonyl)-L-serine (0.98 g, 2.4 mmol) and methyl O-(tert-butyldiphenylsilyl)-L-serinate hydrochloride (1.1 g, 1.2 eq. 2.9 mmol) gave crude methyl N-((2-(4-((tert-butoxycarbonyl)amino)phenyl)thiazole-5-carbonyl)-L-seryl)-O-(tert-butyldiphenylsilyl)-L-serinate (2.25 g) which was used as such in the next step

Step 6. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate was prepared following General experimental procedure 5. Crude methyl N-((2-(4-((tert-butoxycarbonyl)amino)phenyl)thiazole-5-carbonyl)-L-seryl)-O-(tert-butyldiphenylsilyl)-L-serinate (2.25 g) and acetic anhydride (0.34 mL, 3.6 mmol) gave methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.40 g, 0.51 mmol, 21% over 2 steps). LCMS (General 3 acidic) RT: 1.78 min; area % (214 nm): 100%; m/z=789.6 [M+H]⁺.

Step 7. Methyl 2-(2-(2-(4-((tert-butoxycarbonyl)amino)phenyl)thiazole-5-carboxamido)acrylamido)acrylate was prepared following General experimental procedure 7. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.19 g, 0.24 mmol) gave methyl 2-(2-(2-(4-((tert-butoxycarbonyl)amino)phenyl)thiazole-5-carboxamido)acrylamido)acrylate (7.8 mg, 16 μmol, 27%) as a white solid LC-MS (22010199A TFA LCMS-5 C1): RT: 1.353 min; Area 97.7% (215 nm), 96.7% (304 nm); m/z=473.2 [M+H]⁺. ¹H NMR (400 MHz, CDCl₃) δ (ppm) 8.66 (s, 1H), 8.57 (s, 1H), 8.24 (s, 1H), 7.92 (d, J=8.8 Hz, 2H), 7.48 (d, J=8.8 Hz, 2H), 6.72 (d, J=2.5 Hz, 1H), 6.67 (s, 1H), 6.63 (s, 1H), 6.04 (d, J=1.0 Hz, 1H), 5.49 (dd, J=2.3 Hz, J=1.3 Hz, 1H), 3.91 (s, 1H), 1.54 (s, 9H).

### Example 6: Compound 6

### Methyl 2-(2-(2-(4-acetamidophenyl)thiazole-4-carboxamido)acrylamido)acrylate

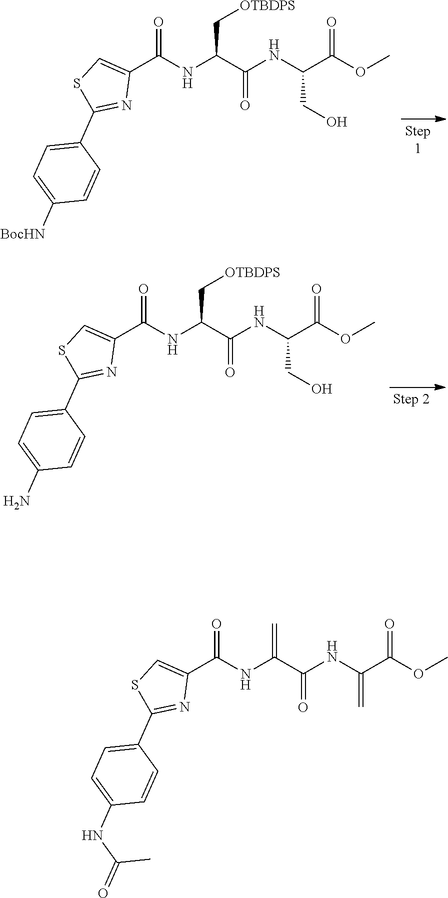

Step 1. Methyl N-(2-(4-aminophenyl)thiazole-4-carbonyl)-O-(tert-butyldiphenylsilyl)-L-seryl-L-serinate was prepared using the following procedure. Methyl N-(2-(4-((tert-butoxycarbonyl)amino)phenyl)thiazole-4-carbonyl)-O-(tert-butyldiphenylsilyl)-L-seryl-L-serinate (as prepared in step 3) (0.80 g, 1 eq., 1.1 mmol) was dissolved in CH₂Cl₂ (5 mL) and TFA (5 mL) was added. The resulting mixture was stirred at room temperature for 1 hour. The mixture was diluted with CH₂Cl₂ (50 mL) and poured into 1M NaOH (aq. 150 mL). The layers were separated, and the aqueous phase was extracted with CH₂Cl₂ (50 mL). The combined organic layers were washed with brine, dried over Na₂SO₄, filtered, and concentrated to provide methyl N-(2-(4-aminophenyl)thiazole-4-carbonyl)-O-(tert-butyldiphenylsilyl)-L-seryl-L-serinate (0.65 g, 1.0 mmol, 94%) as yellow oil.

Step 2. Methyl (S)-2-(2-(2-(4-acetamidophenyl)thiazole-4-carboxamido)-3-((methylsulfonyl)oxy)propanamido)acrylate was prepared using the following procedure. Methyl N-(2-(4-aminophenyl)thiazole-4-carbonyl)-O-(tert-butyldiphenylsilyl)-L-seryl-L-serinate (0.22 g, 1 eq., 0.34 mmol) dissolved in CH₂Cl₂ (5 mL) and DIPEA (0.18 mL, 3 eq., 1.0 mmol) and acetic acid (41 mg, 39 μL, 2 Eq, 0.68 mmol) were added. Then, COMU (0.17 g, 1.2 eq., 0.41 mmol) was added and the resulting mixture was stirred for 16 hours at room temperature. LCMS monitoring showed partial conversion to the bis acetylated product. Additional DiPEA (0.18 mL, 3 Eq, 1.0 mmol), acetic acid (39 μL, 2 eq., 0.68 mmol) and COMU (0.17 g, 1.2 eq., 0.41 mmol) were added and the mixture was stirred for 24 hours. The mixture was diluted with CH₂Cl₂ (20 mL) and washed with HCl (1M, 20 mL) NaHCO₃ (aq. sat. 20 mL) and brine, dried over Na₂SO₄, filtered, and concentrated. The residue was dissolved in CH₂Cl₂ (5 mL) and DBU (0.15 mL, 3 eq., 1.0 mmol) was added.

The resulting mixture was stirred for 1 hour. A solution of tetrabutylammonium fluoride in THF (0.51 mL, 1.0 M, 1.5 eq., 0.51 mmol) was added and the resulting mixture was stirred for 1 hour. The mixture was diluted with CH₂Cl₂ (20 mL) and washed with HCl (1M, 20 mL) and brine, dried over Na₂SO₄, filtered, and concentrated. The residue was dissolved in CH₂Cl₂ (5 mL) and methane sulfonyl chloride (40 μL, 1.5 eq., 0.51 mmol) and triethylamine (0.24 mL, 5 eq., 1.7 mmol) were added. The resulting mixture was stirred for 90 minutes at room temperature after which full mesylation and partial elimination were observed by LCMS monitoring. The mixture was diluted with CH₂Cl₂ (20 mL) and washed with HCl (1M, 20 mL) and brine, dried over Na₂SO₄, filtered, and concentrated. The residue was dissolved in CH₂Cl₂ (5 mL) and DBU (0.16 g, 0.15 mL, 3 Eq, 1.0 mmol) was added. The resulting mixture was stirred for 90 minutes at room temperature. The mixture was diluted with CH₂Cl₂ (20 mL) and washed with HCl (1M, 20 mL) and brine, dried over Na₂SO₄, filtered, and concentrated. The crude material was purified by automated reverse phase FCC to provide methyl 2-(2-(2-(4-acetamidophenyl)thiazole-4-carboxamido)acrylamido)acrylate (5.2 mg, 0.34 mmol, 3.7%) as white solid. LCMS (22010199C TFA LCMS-5 C3) RT: 1.062 min; area % (215 nm): 91.0%; m/z=415.0 [M+H]⁺. ¹H NMR (400 MHz, CDCl₃) δ 9.99 (s, 1H), 8.53 (s, 1H), 8.10 (s, 1H), 8.00-7.92 (m, 2H), 7.61 (d, J=8.3 Hz, 2H), 7.29 (s, 1H), 6.76 (d, J=2.2 Hz, 1H), 6.69 (s, 1H), 6.01 (d, J=1.4 Hz, 1H), 5.48 (t, J=1.9 Hz, 1H), 3.88 (s, 3H), 2.21 (s, 3H).

### Example 7: Compound 16

### Methyl 2-(2-(2-phenylthiazole-4-carboxamido)acrylamido)acrylate

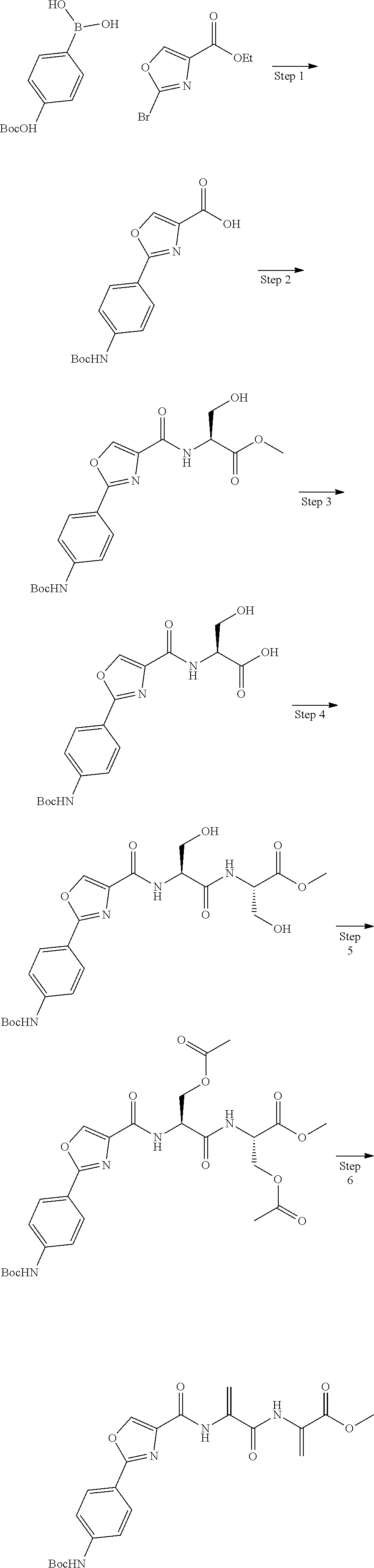

Step 1. 2-(4-((tert-butoxycarbonyl)amino)phenyl)oxazole-4-carboxylic acid was prepared using the following procedure. To a solution of (4-((tert-butoxycarbonyl)amino)phenyl)boronic acid (1.0 g, 4.2 mmol) and ethyl 2-bromooxazole-4-carboxylate (0.93 g, 4.2 mmol) in 1,2-Dimethoxyethane (25 mL) were added sodium carbonate (aq., 2 M, 21 mL, 10 eq., 42 mmol) and palladium tetrakis (0.24 g, 0.05 eq., 0.21 mmol). The resulting mixture was stirred at 80° C. for 16 hours. The mixture was cooled to room temperature and diluted with EtOAc. The mixture was washed with water. The aqueous layer was acidified by addition of 1 M HCl. The formed precipitate was isolated by filtration and dried under reduced pressure to provide 2-(4-((tert-butoxycarbonyl)amino)phenyl)oxazole-4-carboxylic acid (0.66 g, 2.2 mmol, 51%) as a white solid. ¹H NMR (299 MHz, CD₃OD) δ 8.52 (s, 1H), 8.00 (m, 2H), 7.60 (m, 2H), 1.55 (s, 9H).

Step 2. Methyl (2-(4-((tert-butoxycarbonyl)amino)phenyl)oxazole-4-carbonyl)-L-serinate was prepared following General experimental procedure 4. 2-(4-((tert-butoxycarbonyl)amino)phenyl)oxazole-4-carboxylic acid (0.66 g, 2.2 mmol) and methyl L-serinate hydrochloride (0.41 g, 1.2 eq., 2.6 mmol) gave methyl (2-(4-((tert-butoxycarbonyl)amino)phenyl)oxazole-4-carbonyl)-L-serinate (0.98 g, 2.4 mmol, quant.) as an orange oil.

Step 3. (2-(4-((tert-butoxycarbonyl)amino)phenyl)oxazole-4-carbonyl)-L-serine was prepared following General experimental procedure 2. Methyl (2-(4-((tert-butoxycarbonyl)amino)phenyl)oxazole-4-carbonyl)-L-serinate (0.98 g, 2.4 mmol) gave (2-(4-((tert-butoxycarbonyl)amino)phenyl)oxazole-4-carbonyl)-L-serine (0.64 g, 1.6 mmol, 68%) as an off-white solid. ¹H NMR (299 MHz, cd₃od) δ 8.43 (s, 1H), 8.05-7.96 (m, 2H), 7.60 (m, 2H), 4.71 m, 1H), 4.14-3.94 (m, 2H), 3.79-3.69 (m, 1H), 1.55 (s, 9H).

Step 4. Methyl (2-(4-((tert-butoxycarbonyl)amino)phenyl)oxazole-4-carbonyl)-L-seryl-L-serinate was prepared following General experimental procedure 4. (2-(4-((tert-butoxycarbonyl)amino)phenyl)oxazole-4-carbonyl)-L-serine (0.20 g, 0.51 mmol) and methyl L-serinate hydrochloride (95 mg, 1.2 eq., 0.61 mmol) gave methyl (2-(4-((tert-butoxycarbonyl)amino)phenyl)oxazole-4-carbonyl)-L-seryl-L-serinate (0.18 g, 0.37 mmol, 72%) as a colorless oil.

Step 5. Methyl O-acetyl-N—(O-acetyl-N-(2-(4-((tert-butoxycarbonyl)amino)phenyl)oxazole-4-carbonyl)-L-seryl)-L-serinate was prepared following General experimental procedure 5. Methyl (2-(4-((tert-butoxycarbonyl)amino)phenyl)oxazole-4-carbonyl)-L-seryl-L-serinate (0.18 g, 0.37 mmol) and acetic anhydride (90 μL, 2.6 eq., 0.95 mmol) gave methyl O-acetyl-N-(0-acetyl-N-(2-(4-((tert-butoxycarbonyl)amino)phenyl)oxazole-4-carbonyl)-L-seryl)-L-serinate (0.12 g, 0.20 mmol, 56%). ¹H NMR (299 MHz, CDCl₃) δ 8.26 (s, 1H), 7.98 (d, J=8.6 Hz, 2H), 7.79 (d, J=8.0 Hz, 1H), 7.51 (d, J=8.6 Hz, 2H), 7.32 (d, J=7.8 Hz, 1H)), 6.88 (s, 1H), 5.04 (m, 1H), 4.87 (m, 1H), 4.58-4.36 (m, 4H), 3.80 (m, 3H), 2.15-1.98 (m, 6H), 1.54 (s, 9H).

Step 6. Methyl (2-(2-(4-((tert-butoxycarbonyl)amino)phenyl)oxazole-4-carboxamido)acryloyl)-L-serinate was prepared following General experimental procedure 8. Methyl O-acetyl-N-(0-acetyl-N-(2-(4-((tert-butoxycarbonyl)amino)phenyl)oxazole-4-carbonyl)-L-seryl)-L-serinate (0.12 g, 0.20 mmol) gave methyl (2-(2-(4-((tert-butoxycarbonyl)amino)phenyl)oxazole-4-carboxamido)acryloyl)-L-serinate (29 mg, 64 μmol, 32%) as a white solid. LC-MS (22010199A TFA LCMS-5 C1): rt 1.560 min; Area 99.7% (215 nm), 99.2% (304 nm); m/z=[M+H]⁺=457.2 ¹H NMR (400 MHz, CDCl₃) δ (ppm) 9.57 (s, 1H), 8.54 (s, 1H), 8.25 (s, 1H), 8.02 (d, J=8.7 Hz, 2H), 7.49 (d, J=8.7 Hz, 2H), 6.75 (d, J=2.2 Hz), 6.71 (s, 1H), 6.64 (s, 1H), 6.03 (d, J=1.0 Hz, 1H), 5.49 (t, J=1.9 Hz, 1H), 3.90 (s, 3H), (s, 9H).

### Example 8: Compound 49

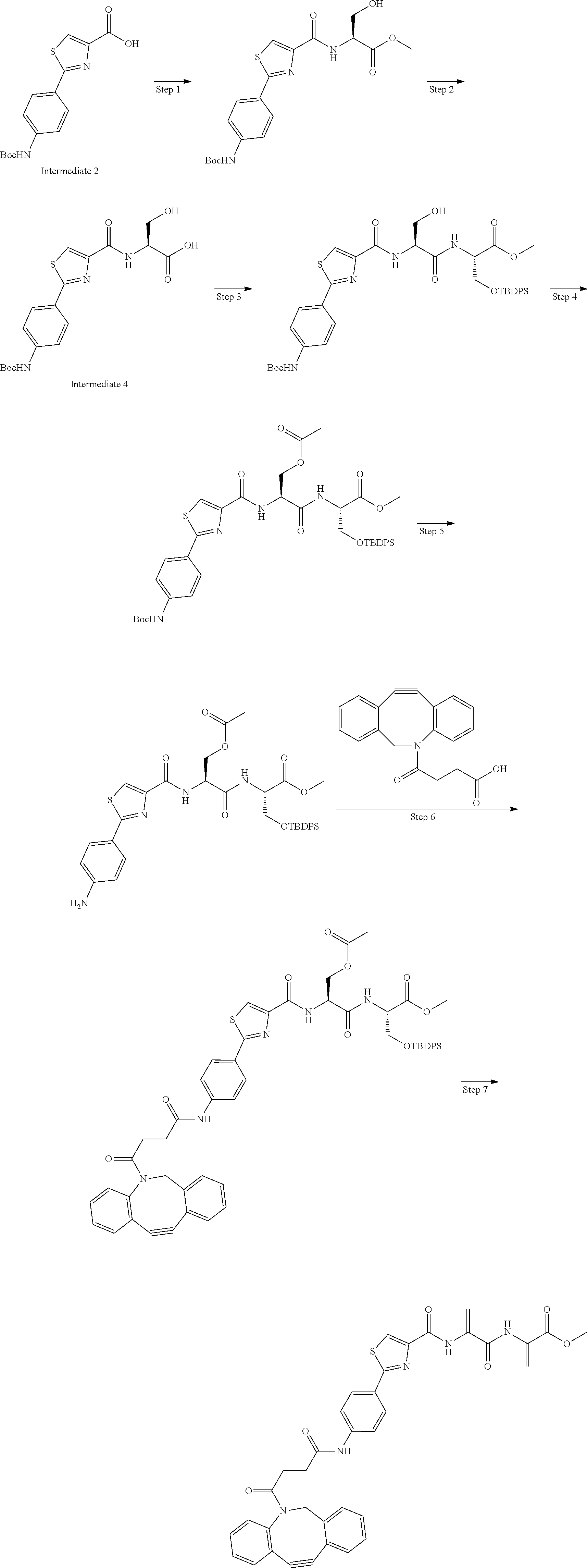

Step. 1 Methyl (2-(4-((tert-butoxycarbonyl)amino)phenyl)thiazole-4-carbonyl)-L-serinate was prepared following General experimental procedure 4. 2-(4-((tert-butoxycarbonyl)amino)phenyl)thiazole-4-carboxylic acid (Intermediate 2) (0.68 g, 2.1 mmol) and methyl L-serinate hydrochloride (0.40 g, 1.2 eq., 2.6 mmol) gave methyl (2-(4-((tert-butoxycarbonyl)amino)phenyl)thiazole-4-carbonyl)serinate (0.87 g, 2.1 mmol, 97%) as an off-white solid. LCMS (General 3 basic) RT: 1.44 min; area % (254 nm): 98%; m/z=420.3 [M−H]⁻.

Step 2. (2-(4-((tert-butoxycarbonyl)amino)phenyl)thiazole-4-carbonyl)-L-serine (Intermediate 4) was prepared following General experimental procedure 2. Methyl (2-(4-((tert-butoxycarbonyl)amino)phenyl)thiazole-4-carbonyl)-L-serinate (0.88 g, 2.1 mmol) gave (2-(4-((tert-butoxycarbonyl)amino)phenyl)thiazole-4-carbonyl)-L-serine (Intermediate 4) (0.84 g, 2.1 mmol, quant) as a white solid.

Step 3. Methyl N-((2-(4-((tert-butoxycarbonyl)amino)phenyl)thiazole-4-carbonyl)-L-seryl)-O-(tert-butyldiphenylsilyl)-L-serinate was prepared following General experimental procedure 4. (2-(4-((tert-butoxycarbonyl)amino)phenyl)thiazole-4-carbonyl)-L-serine (Intermediate 4) (0.87 g, 2.1 mmol) and L-Ser(OTBDPS)—OMe·HCl (1.0 g, 1.2 eq., 2.6 mmol) gave methyl N-((2-(4-((tert-butoxycarbonyl)amino)phenyl)thiazole-4-carbonyl)-L-seryl)-O-(tert-butyldiphenylsilyl)-L-serinate (1.5 g, 1.9 mmol, 91%) LCMS (General 3 acidic) RT: 1.75 min; area % (254 nm): 98%; m/z=747.6 [M+H]⁺.

Step 4. Methyl N—(O-acetyl-N-(2-(4-((tert-butoxycarbonyl)amino)phenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)phenyl)thiazole-4-carbonyl)-L-seryl)-O-(tert-butyldiphenylsilyl)-L-serinate (1.5 g, 1.9 mmol) gave methyl N-(0-acetyl-N-(2-(4-((tert-butoxycarbonyl)amino)phenyl)thiazole-4-carbonyl)-L-seryl)-O-(tert-butyldiphenylsilyl)-L-serinate (1.6 g, 2.0 mmol, quant) as a yellow foam. LCMS (General 3 acidic) RT: 1.84 min; area % (254 nm): 98%; m/z=789.6 [M+H]⁺.

Step 5. Methyl N—(O-acetyl-N-(2-(4-aminophenyl)thiazole-4-carbonyl)-L-seryl)-O-(tert-butyldiphenylsilyl)-L-serinate was prepared using the following procedure. To a solution of methyl N—(O-acetyl-N-(2-(4-((tert-butoxycarbonyl)amino)phenyl)thiazole-4-carbonyl)-L-seryl)-O-(tert-butyldiphenylsilyl)-L-serinate (1.6 g, 2.0 mmol) in CH₂Cl₂ (30 mL) was added TFA (4.6 mL, 30 eq. 60 mmol) and the resulting mixture was stirred for 1 hour. the reaction mixture was quenched with saturated aqueous NaHCO₃ until gas formation stopped and pH paper showed the mixture pH ˜7-8. The mixture was extracted with CH₂Cl₂ (3×100 ml) and the combined organics were dried over Na₂SO₄, filtered and concentrated to provide methyl N—(O-acetyl-N-(2-(4-aminophenyl)thiazole-4-carbonyl)-L-seryl)-O-(tert-butyldiphenylsilyl)-L-serinate (1.3 g, 1.8 mmol, 91%) LCMS (General 3 basic) RT: 2.01 min; area % (254 nm): 72%; m/z=687.4 [M−H]⁻.

Step 6. The DBCO coupled intermediate was prepared using the following procedure. To a solution of DBCO acid (0.37 g, 1.2 mmol) and triethylamine (0.19 mL, 1.1 eq., 1.3 mmol) in CH₂Cl₂ (6 mL) was added at 0° C. propyl chloroformate (0.15 mL, 1.1 Eq, 1.3 mmol) and the resulting mixture was stirred at 0° C. for 1 hour. A solution of methyl N—(O-acetyl-N-(2-(4-aminophenyl)thiazole-4-carbonyl)-L-seryl)-O-(tert-butyldiphenylsilyl)-L-serinate (0.61 g, 0.73 eq., 0.88 mmol) in CH₂Cl₂ (3 mL) was added and the resulting mixture was stirred for 2 hours at room temperature. The mixture was washed with NaHCO₃ (sat., 3×10 mL) and HCl (1M, 3×10 mL), dried over Na₂SO₄, filtered and concentrated. The crude material was purified by automated FCC to provide the desired product (0.52 g, 0.54 mmol, 45%) of a slightly yellow foam. LCMS (General 3 acidic) RT: 1.86 min; area % (254 nm): 86%; m/z=976.5 [M+H]⁺.

Step 7. DBCO coupled bisdehydroalanine compound was prepared following General experimental procedure 5. DBCO coupled intermediate (0.52 g, 0.54 mmol) gave DBCO coupled bisdehydroalanine compound (24 mg, 37 μmol, 6.9%) as a white solid. LCMS (22010199D TFA LCMS-5 C8) RT: 1.642 min; area % (215 nm): 79.2%; m/z=660.2 [M+H]⁺. ¹H NMR (400 MHz, CDCl₃) δ 10.05 (s, 1H), 8.56 (s, 1H), 8.42 (s, 1H), 8.09 (s, 1H), 7.84 (d, J=8.6 Hz, 2H), 7.71 (d, J=7.4 Hz, 1H), 7.50-7.28 (m, 8H), 7.19 (dd, J=7.4, 1.5 Hz, 1H), 6.78 (d, J=2.2 Hz, 1H), 6.73 (s, 1H), 6.03 (d, J=1.3 Hz, 1H), 5.50 (t, J=1.9 Hz, 1H), 5.19 (d, J=13.9 Hz, 1H), 3.91 (s, 3H), 3.73 (d, J=13.8 Hz, 1H), 2.86 (m, 1H), 2.45 (m, 2H), 2.07 (m, 1H).

### Example 9: Compound 50

### Methyl 2-(2-(2-(4-(hex-5-ynamido)phenyl)thiazole-4-carboxamido)acrylamido)acrylate

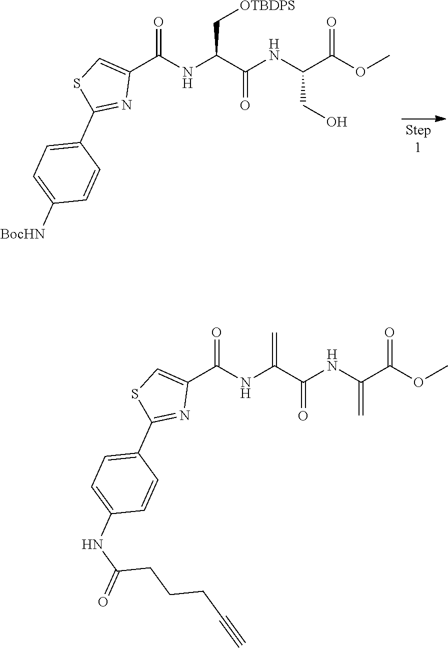

Step 1. Methyl 2-(2-(2-(4-(hex-5-ynamido)phenyl)thiazole-4-carboxamido)acrylamido)acrylate was prepared with the following procedure. Methyl N-(2-(4-aminophenyl)thiazole-4-carbonyl)-O-(tert-butyldiphenylsilyl)-L-seryl-L-serinate (as prepared in step 1) (0.22 g, 0.334 mmol) dissolved in CH₂Cl₂ (5 mL) and DiPEA (0.18 mL, 3 eq., 1.0 mmol) and hex-5-ynoic acid (56 μL, 1.5 eq., 0.51 mmol) were added. Then COMU (0.18 g, 1.2 eq., 0.41 mmol) was added and the resulting mixture was stirred for 16 hours at room temperature. LCMS monitoring showed partial conversion to the bis acylated product. Additional DiPEA (0.18 mL, 3 eq., 1.0 mmol), hex-5-ynoic acid (57 mg, 56 μL, 1.5 eq., 0.510 mmol) and COMU (0.18 mg, 1.2 eq., 0.41 mmol) were added and the resulting mixture was stirred for stirred for 24 hours. The mixture was diluted with CH₂Cl₂ (20 mL) and washed with HCl (1M, 20 mL) NaHCO₃ (aq. sat. 20 mL) and brine, dried over Na₂SO₄, filtered, and concentrated. The residue was dissolved in CH₂Cl₂ (5 mL) and added DBU (0.15 mL, 3 eq., 1.0 mmol) was added. The resulting mixture was stirred for 1 hour. A solution of tetrabutylammonium fluoride in THF (0.51 mL, 1.0 M, 1.5 eq., 0.51 mmol) was added and the resulting mixture was stirred for 1 hour. The mixture was diluted with CH₂Cl₂ (20 mL) and washed with HCl (1M, 20 mL) and brine, dried over Na₂SO₄, filtered, and concentrated. The residue was dissolved in CH₂Cl₂ (5 mL) and methane sulfonyl chloride (40 μL, 1.5 eq., 0.51 mmol) and triethylamine (0.17 g, 0.24 mL, 5 eq., 1.7 mmol) were added. The resulting mixture was stirred for 90 minutes at room temperature after which full mesylation and partial elimination were observed by LCMS monitoring. The mixture was diluted with CH₂Cl₂ (20 mL) and washed with HCl (1M, 20 mL) and brine, dried over Na₂SO₄, filtered, and concentrated. The residue was dissolved in CH₂Cl₂ (5 mL) and DBU (0.16 g, 0.15 mL, 3 eq., 1.0 mmol) was added. The resulting mixture was stirred for 90 minutes at room temperature. The mixture was diluted with CH₂Cl₂ (20 mL) and washed with HCl (1M, 20 mL) and brine, dried over Na₂SO₄, filtered, and concentrated. The crude material was purified by automated reverse phase FCC to provide methyl 2-(2-(2-(4-(hex-5-ynamido)phenyl)thiazole-4-carboxamido)acrylamido)acrylate (4.5 mg, 9.6 μmol, 2.8%) as a white solid. LCMS (22010199C TFA LCMS-5 C3) RT: 1.287 min; area % (215 nm): 93.0%; m/z=467.2 [M+H]⁺. ¹H NMR (400 MHz, CDCl₃) δ 9.98 (s, 1H), 8.53 (s, 1H), 8.00-7.92 (m, 2H), 7.62 (d, J=8.3 Hz, 2H), 7.31 (s, 1H), 6.76 (d, J=2.2 Hz, 1H), 6.69 (s, 1H), 6.01 (d, J=1.2 Hz, 1H), 5.48 (t, J=1.9 Hz, 1H), 3.88 (s, 3H), 2.55 (t, J=7.3 Hz, 2H), 2.34 (td, J=6.7, 2.6 Hz, 2H), 2.04-1.92 (m, 3H).

### Example 10: Compound 51

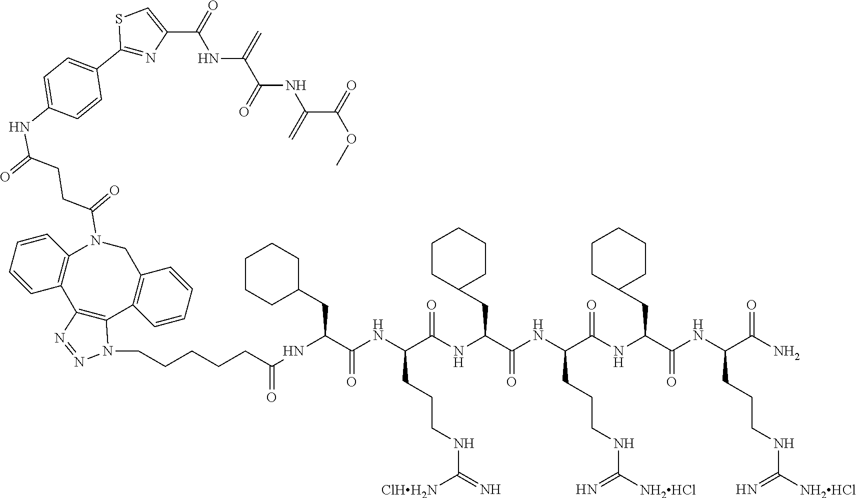

Compound 51 was prepared following General procedure for conjugation chemistry between azido-peptide and 49 (3.0 mg, 4.5 μmol) and 81 (5.4 mg, 4.5 μmol) gave 51 (3.5 mg, 1.9 μmol, 41%) as a white solid. LCMS (30833 LCMS-6): RT: 2.788 min & 2.822 min (regioisomers); Area: 95.3% (215 nm), 99.6% (ELSD); m/z=1744.1 [M+H]⁺

### Example 11: Compound 52

### Methyl 2-(2-(2-(4-((tert-butoxycarbonyl)amino)phenyl)-5-methylthiazole-4-carboxamido)acrylamido)acrylate

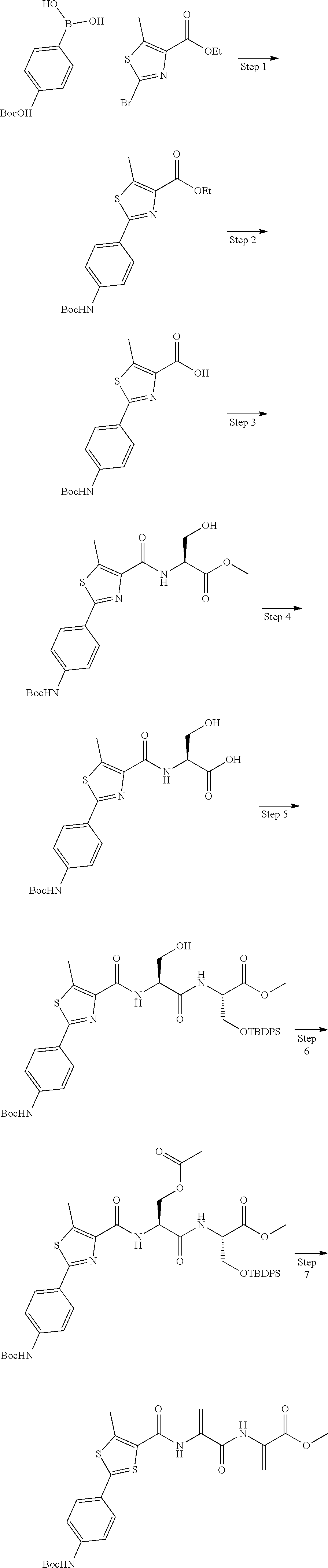

Step 1. Methyl 2-(4-((tert-butoxycarbonyl)amino)phenyl)-5-methylthiazole-4-carboxylate was prepared following General experimental procedure 1. Methyl-2-Bromo-5-methyl-thiazole-4-carboxylate (0.91 g, 3.8 mmol) and (4-((tert-butoxycarbonyl)amino)phenyl)boronic acid (0.86 g, 3.6 mmol) gave methyl 2-(4-((tert-butoxycarbonyl)amino)phenyl)-5-methylthiazole-4-carboxylate (1.1 g, 3.1 mmol 88%). LCMS (General 3 basic) RT: 1.80 min; area % (214 nm): 87%; m/z=347.2 [M−H]⁻.

Step 2. 2-(4-((tert-butoxycarbonyl)amino)phenyl)-5-methylthiazole-4-carboxylic acid was prepared following General experimental procedure 2. Methyl 2-(4-((tert-butoxycarbonyl)amino)phenyl)-5-methylthiazole-4-carboxylate (1.1 g, 3.1 mmol) gave methyl 2-(4-((tert-butoxycarbonyl)amino)phenyl)-5-methylthiazole-4-carboxylate (1.0 g, 3.1 mmol, quant). %). LCMS (General 3 basic) RT: 1.17 min; area % (214 nm): 81%; m/z=335.4 [M+H]⁺.

Step 3. Methyl (2-(4-((tert-butoxycarbonyl)amino)phenyl)-5-methylthiazole-4-carbonyl)serinate was prepared following General experimental procedure 3. 2-(4-((tert-butoxycarbonyl)amino)phenyl)-5-methylthiazole-4-carboxylic acid (1.1 g, 3.2 mmol) and L-Ser-OMe·HCl (0.60 g, 1.2 eq. 3.9 mmol) gave crude methyl (2-(4-((tert-butoxycarbonyl)amino)phenyl)-5-methylthiazole-4-carbonyl)serinate (2.6 g), which was used without purification in the next step. LCMS (General 3 basic) RT: 1.61 min; area % (214 nm): 69%; m/z=436.2 [M+H]⁺.

Step 4. (2-(4-((tert-butoxycarbonyl)amino)phenyl)-5-methylthiazole-4-carbonyl)serine was prepared following General experimental procedure 2. Crude methyl (2-(4-((tert-butoxycarbonyl)amino)phenyl)-5-methylthiazole-4-carbonyl)serinate (2.6 g crude) gave crude (2-(4-((tert-butoxycarbonyl)amino)phenyl)-5-methylthiazole-4-carbonyl)serine (1.8 g), which was used without purification in the next step. LCMS (General 3 basic) RT: 1.06 min; area % (214 nm): 69%; m/z=422.4 [M+H]⁺.

Step 5. Methyl N-((2-(4-((tert-butoxycarbonyl)amino)phenyl)-5-methylthiazole-4-carbonyl)seryl)-O-(tert-butyldiphenylsilyl)-L-serinate was prepared following General experimental procedure 4. (2-(4-((tert-butoxycarbonyl)amino)phenyl)-5-methylthiazole-4-carbonyl)serine (1.4 g, crude) and methyl O-(tert-butyldiphenylsilyl)-L-serinate hydrochloride (1.6 g, 4.0 mmol) gave methyl N-((2-(4-((tert-butoxycarbonyl)amino)phenyl)-5-methylthiazole-4-carbonyl)seryl)-O-(tert-butyldiphenylsilyl)-L-serinate (1.2 g, 1.5 mmol) as a clear yellow oil. LCMS (General 3 acidic) RT: 1.85 min; area % (214 nm): 73%; m/z=761.6 [M+H]⁺.

Step 6. Methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate was prepared following General experimental procedure 5. Methyl N-((2-(4-((tert-butoxycarbonyl)amino)phenyl)-5-methylthiazole-4-carbonyl)seryl)-O-(tert-butyldiphenylsilyl)-L-serinate (1.2 g, 1.5 mmol) and acetic anhydride (0.16 mL, 1.1 eq., 1.7 mmol) gave methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate (1.1 g, 1.3 mmol, 87%) as a yellow viscous oil. LCMS (General 3 basic) RT: 2.31 min; area % (214 nm): 86%; m/z=801.5 [M+H]⁺.

Step 7. Methyl 2-(2-(2-(4-((tert-butoxycarbonyl)amino)phenyl)-5-methylthiazole-4-carboxamido)acrylamido)acrylate was prepared following General experimental procedure 7. Methyl N—(O-acetyl-N-(2-(4-((tert-butoxycarbonyl)amino)phenyl)-5-methylthiazole-4-carbonyl)seryl)-O-(tert-butyldiphenylsilyl)-L-serinate (0.21 g, 0.26 mmol) gave methyl 2-(2-(2-(4-((tert-butoxycarbonyl)amino)phenyl)-5-methylthiazole-4-carboxamido)acrylamido)acrylate (11 mg, 23 μmol, 13%) as a white solid. LCMS (22010199C TFA LCMS-5 C3) RT: 1.702 min; area: 91.1% (215 nm); m/z 487.2 [M+H]⁺. ¹H NMR (400 MHz, CDCl₃) δ 10.08 (s, 1H), 8.51 (s, 1H), 7.88-7.80 (m, 2H), 7.42 (d, J=8.6 Hz, 2H), 6.72-6.66 (m, 2H), 6.57 (s, 1H), 6.00 (d, J=1.2 Hz, 1H), 5.42 (t, J=1.8 Hz, 1H), 3.88 (s, 3H), 2.85 (s, 3H), 1.52 (s, 9H).

### Example 12: Compound 53

### methyl (Z)-2-(2-(2-(4-((tert-butoxycarbonyl)amino)phenyl)thiazole-4-carboxamido)acrylamido)but-2-enoate

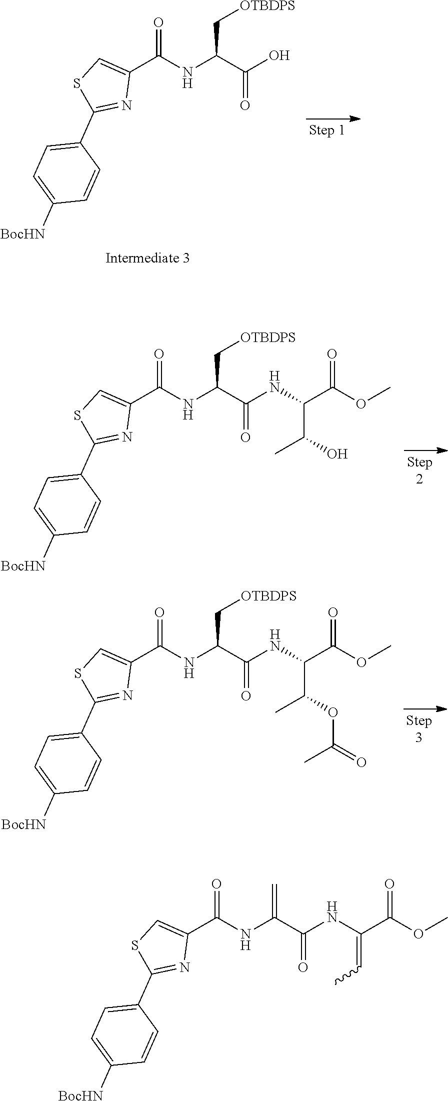

Step 1. Methyl N-(2-(4-((tert-butoxycarbonyl)amino)phenyl)thiazole-4-carbonyl)-O-(tert-butyldiphenylsilyl)seryl-L-threoninate was prepared following General experimental procedure 3. N-(2-(4-((tert-butoxycarbonyl)amino)phenyl)thiazole-4-carbonyl)-O-(tert-butyldiphenylsilyl)serine (Intermediate 3) (0.90 g, 1.4 mmol) and H-Thr-OMe·HCl (0.29 g, 1.2 eq., 1.7 mmol) gave methyl N-(2-(4-((tert-butoxycarbonyl)amino)phenyl)thiazole-4-carbonyl)-O-(tert-butyldiphenylsilyl)seryl-L-threoninate (0.29 g, 0.38 mmol, 27%) as a light yellow foam. LCMS (General 3 basic) RT: 2.20 min; area % (254 nm): 81%; m/z=761.3 [M+H]⁺.

Step 2. Methyl O-acetyl-N—(N-(2-(4-((tert-butoxycarbonyl)amino)phenyl)thiazole-4-carbonyl)-O-(tert-butyldiphenylsilyl)seryl)-L-threoninate was prepared following General experimental procedure 5. Methyl N-(2-(4-((tert-butoxycarbonyl)amino)phenyl)thiazole-4-carbonyl)-O-(tert-butyldiphenylsilyl)seryl-L-threoninate (0.29 g, 0.38 mmol) and acetic anhydride (37 μL, 1.05 eq., 0.39 mmol) gave methyl O-acetyl-N—(N-(2-(4-((tert-butoxycarbonyl)amino)phenyl)thiazole-4-carbonyl)-O-(tert-butyldiphenylsilyl)seryl)-L-threoninate (0.30 g, 0.37 mmol, 98%) as a light yellow foam.

Step 3. Methyl (Z)-2-(2-(2-(4-((tert-butoxycarbonyl)amino)phenyl)thiazole-4-carboxamido)acrylamido)but-2-enoate was prepared following General experimental procedure 7. Methyl O-acetyl-N—(N-(2-(4-((tert-butoxycarbonyl)amino)phenyl)thiazole-4-carbonyl)-O-(tert-butyldiphenylsilyl)seryl)-L-threoninate (0.10 g, 0.13 mmol) gave methyl (Z)-2-(2-(2-(4-((tert-butoxycarbonyl)amino)phenyl)thiazole-4-carboxamido)acrylamido)but-2-enoate (22 mg, 45 μmol, 36%). LCMS (22010199C TFA LCMS-5 C3) RT: 1.424 min; area % (215 nm): 92.9%; m/z=487.2 [M+H]⁺. ¹H NMR (400 MHz, CDCl₃) δ 9.97 (s, 1H), 8.07 (s, 1H), 7.94-7.86 (m, 2H), 7.48 (s, 1H), 7.42 (d, J=8.6 Hz, 2H), 6.93 (q, J=7.2 Hz, 1H), 6.76 (d, J=1.8 Hz, 1H), 6.59 (s, 1H), 5.53 (t, J=1.7 Hz, 1H), 3.79 (s, 3H), 1.82 (dd, J=7.2, 0.7 Hz, 3H), 1.51 (s, 9H).

### Example 13: Compound 55

### Methyl (E)-2-(2-(2-(4-((tert-butoxycarbonyl)amino)phenyl)thiazole-4-carboxamido)acrylamido)but-2-enoate

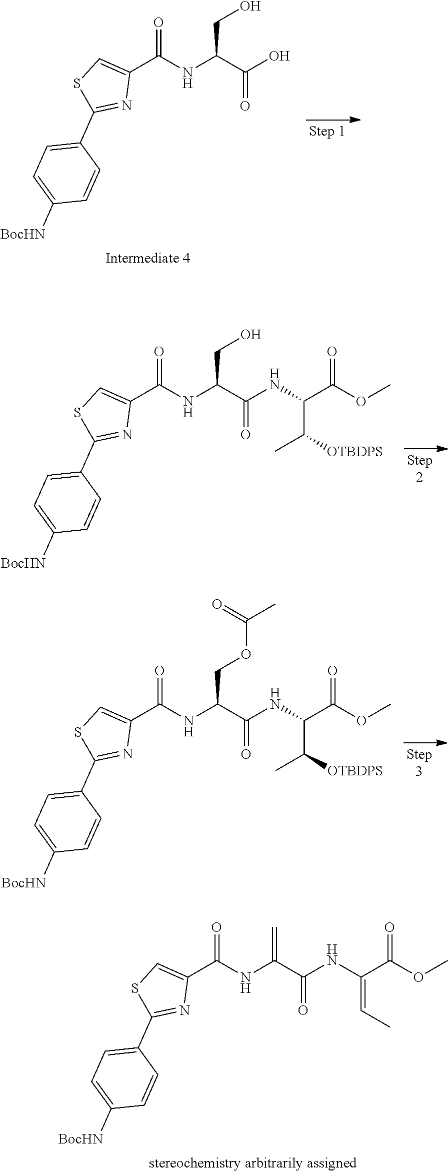

Step 1. Methyl N-((2-(4-((tert-butoxycarbonyl)amino)phenyl)thiazole-4-carbonyl)seryl)-O-(tert-butyldiphenylsilyl)-L-allothreoninate was prepared following General experimental procedure 4. (2-(4-((tert-butoxycarbonyl)amino)phenyl)thiazole-4-carbonyl)serine (Intermediate 4) (785 mg, 1.93 mmol) and methyl O-(tert-butyldiphenylsilyl)-L-allothreoninate (859 mg, 1.2 eq., 2.31 mmol) gave methyl N-((2-(4-((tert-butoxycarbonyl)amino)phenyl)thiazole-4-carbonyl)seryl)-O-(tert-butyldiphenylsilyl)-L-allothreoninate (1.54 g, 2.02 mmol, quant.) as a viscous oil. LCMS (General 3 acidic) RT: 1.81 min; area % (254 nm): 96%; m/z=761.3 [M+H]⁺.

Step 2. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate was prepared following General experimental procedure 5. Methyl N-((2-(4-((tert-butoxycarbonyl)amino)phenyl)thiazole-4-carbonyl)seryl)-O-(tert-butyldiphenylsilyl)-L-allothreoninate (1.6 g, 2.1 mmol) and acetic anhydride (0.22 mL, 1.1 eq., 2.3 mmol) gave methyl N—(O-acetyl-N-(2-(4-((tert-butoxycarbonyl)amino)phenyl)thiazole-4-carbonyl)seryl)-O-(tert-butyldiphenylsilyl)-L-allothreoninate (1.37 g, 1.71 mmol, 81%) as a yellow solid. LCMS (General 3 acidic) RT: 1.88 min; area % (254 nm): 75%; m/z=803.5 [M+H]⁺.

Step 3. Methyl (E)-2-(2-(2-(4-((tert-butoxycarbonyl)amino)phenyl)thiazole-4-carboxamido)acrylamido)but-2-enoate was prepared following General experimental procedure 7. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate (306 mg, 0.38 mmol) gave methyl methyl (E)-2-(2-(2-(4-((tert-butoxycarbonyl)amino)phenyl)thiazole-4-carboxamido)acrylamido)but-2-enoate (4.6 mg, 9.5 μmol, 2.5%). LCMS (22010199C TFA LCMS-5 C3) RT: 1.470 min; area: 93.8% (215 nm); m/z 487.2 [M+H]⁺. ¹H NMR (400 MHz, CDCl₃) δ 9.98 (s, 1H), 8.17 (s, 1H), 8.06 (s, 1H), 7.95-7.88 (m, 2H), 7.44 (d, J=8.6 Hz, 2H), 7.33 (q, J=7.7 Hz, 2H), 6.70 (d, J=2.0 Hz, 1H), 6.59 (s, 1H), 5.42 (s, 1H), 3.88 (s, 3H), 2.15 (d, J=7.7 Hz, 3H), 1.52 (s, 9H).

### Example 14: Compound 56

### Methyl 2-(2-(2-(4-((tert-butoxycarbonyl)amino)phenyl)thiazole-4-carboxamido)acetamido)acrylate

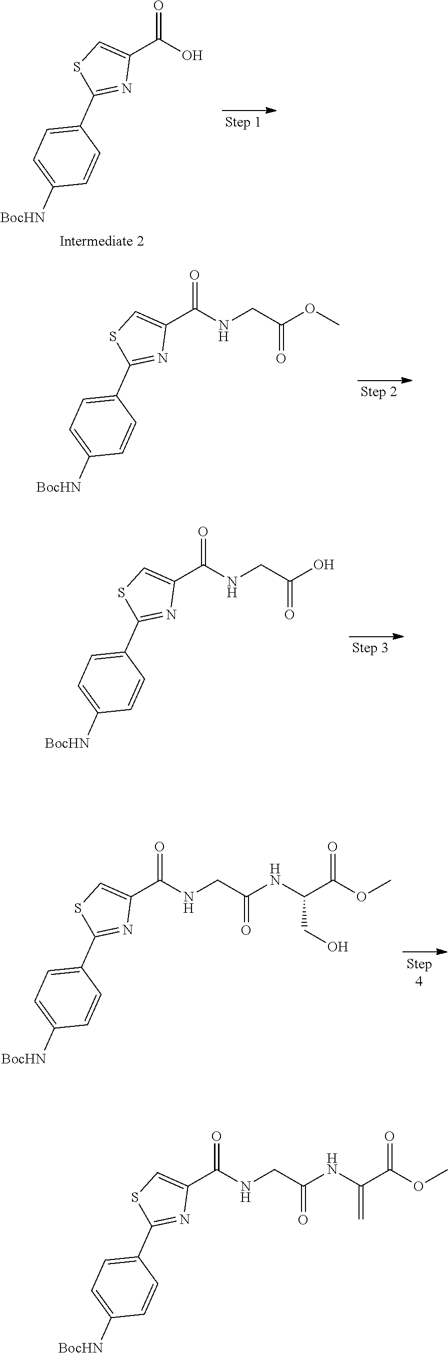 [SMILES: CC(C)(C)OC(=O)Nc1ccc(-c2nc(C(=O)O)cs2)cc1 ; COC(=O)CNC(=O)c1csc(-c2ccc(NC(=O)OC(C)(C)C)cc2)n1 ; CC(C)(C)OC(=O)Nc1ccc(-c2nc(C(=O)NCC(=O)O)cs2)cc1 ; COC(=O)[C@H](CO)NC(=O)CNC(=O)c1csc(-c2ccc(NC(=O)OC(C)(C)C)cc2)n1 ; C=C(NC(=O)CNC(=O)c1csc(-c2ccc(NC(=O)OC(C)(C)C)cc2)n1)C(=O)OC]

Step 1. Methyl (2-(4-((tert-butoxycarbonyl)amino)phenyl)thiazole-4-carbonyl)glycinate was prepared following General General experimental procedure 3. 2-(4-((tert-butoxycarbonyl)amino)phenyl)thiazole-4-carboxylic acid (Intermediate 2) (0.41 g, 1 eq., 1.3 mmol) and methyl glycinate hydrochloride (0.17 g, 1 eq., 1.3 mmol) gave methyl (2-(4-((tert-butoxycarbonyl)amino)phenyl)thiazole-4-carbonyl)glycinate (0.37 g, 0.94 mmol, 74%) as a white foam. LCMS (General 3 basic) RT: 1.63 min; area % (254 nm): 92%; m/z=392.2 [M+H]⁺. ¹H NMR (400 MHz, CDCl₃) δ 8.03 (s, 1H), 7.91-7.82 (m, 3H), 7.49-7.41 (m, 2H), 6.71 (s, 1H), 4.26 (d, J=5.7 Hz, 2H), 3.78 (s, 3H), 1.51 (s, 9H).

Step 2. (2-(4-((tert-butoxycarbonyl)amino)phenyl)thiazole-4-carbonyl)glycine was prepared following General experimental procedure 2. Methyl (2-(4-((tert-butoxycarbonyl)amino)phenyl)thiazole-4-carbonyl)glycinate (0.37 mg, 1 eq., 0.94 mmol) gave (2-(4-((tert-butoxycarbonyl)amino)phenyl)thiazole-4-carbonyl)glycine (0.32 g, 0.85 mmol, 90%) as a white solid. LCMS (General 3 basic) RT: 1.12 min; area % (254 nm): 96%; m/z=378.2 [M+H]⁺. ¹H NMR (400 MHz, DMSO) δ 9.69 (s, 1H), 8.71 (t, J=6.1 Hz, 1H), 8.24 (s, 1H), 7.98-7.90 (m, 2H), 7.65-7.57 (m, 2H), 3.96 (d, J=6.1 Hz, 2H), 1.49 (s, 9H).

Step 3. Methyl (2-(4-((tert-butoxycarbonyl)amino)phenyl)thiazole-4-carbonyl)glycyl-L-serinate was prepared following General experimental procedure 3. (2-(4-((tert-butoxycarbonyl)amino)phenyl)thiazole-4-carbonyl)glycine (0.31 g, 1 eq, 0.83 mol) and methyl L-serinate hydrochloride (0.16 g, 1.2 eq. 1.0 mmol) gave methyl (2-(4-((tert-butoxycarbonyl)amino)phenyl)thiazole-4-carbonyl)glycylserinate (0.29 mg, 0.60 mmol, 72%). LCMS (General 3 basic) RT: 1.47 min; area % (254 nm): 93%; m/z=479.3 [M+H]⁺. ¹H NMR (400 MHz, MeOD) δ 8.07 (s, 1H), 7.90-7.82 (m, 2H), 7.52-7.44 (m, 2H), 4.56 (t, J=4.3 Hz, 1H), 4.15 (d, J=2.1 Hz, 2H), 3.92-3.76 (m, 2H), 3.71 (s, 3H), 1.49 (s, 9H).

Step 4. Methyl 2-(2-(2-(4-((tert-butoxycarbonyl)amino)phenyl)thiazole-4-carboxamido)acetamido)acrylate was prepared using the following procedure. To a solution of methyl (2-(4-((tert-butoxycarbonyl)amino)phenyl)thiazole-4-carbonyl)glycylserinate (0.25 g, 1 eq., 0.51 mmol) in MeCN (30 mL) was added triethylamine (0.14 mL, 2 eq., 1.0 mmol) and the resulting mixture was stirred for 6 days at room temperature. The mixture was concentrated and purified by automated FCC and automated reverse phase FCC to provide methyl 2-(2-(2-(4-((tert-butoxycarbonyl)amino)phenyl)thiazole-4-carboxamido)acetamido)acrylate (28 mg, 61 μmol, 12%) as white solid. LCMS (22010199D TFA LCMS-5 C8) RT: 1.382 min; area % (215 nm): 98.0%; m/z=461.2 [M+H]⁺. ¹H NMR (400 MHz, CDCl₃) δ 8.36 (s, 1H), 8.09 (d, J=2.4 Hz, 1H), 8.00 (d, J=6.5 Hz, 1H), 7.91-7.80 (m, 2H), 7.45 (d, J=8.3 Hz, 2H), 6.61 (d, J=2.5 Hz, 2H), 5.92 (d, J=1.5 Hz, 1H), 4.24 (d, J=5.9 Hz, 2H), 3.82 (s, 3H), 1.52 (s, 9H).

### Example 15: Compound 57

### Methyl (S)-2-(2-(2-(4-((tert-butoxycarbonyl)amino)phenyl)thiazole-4-carboxamido)propanamido)acrylate

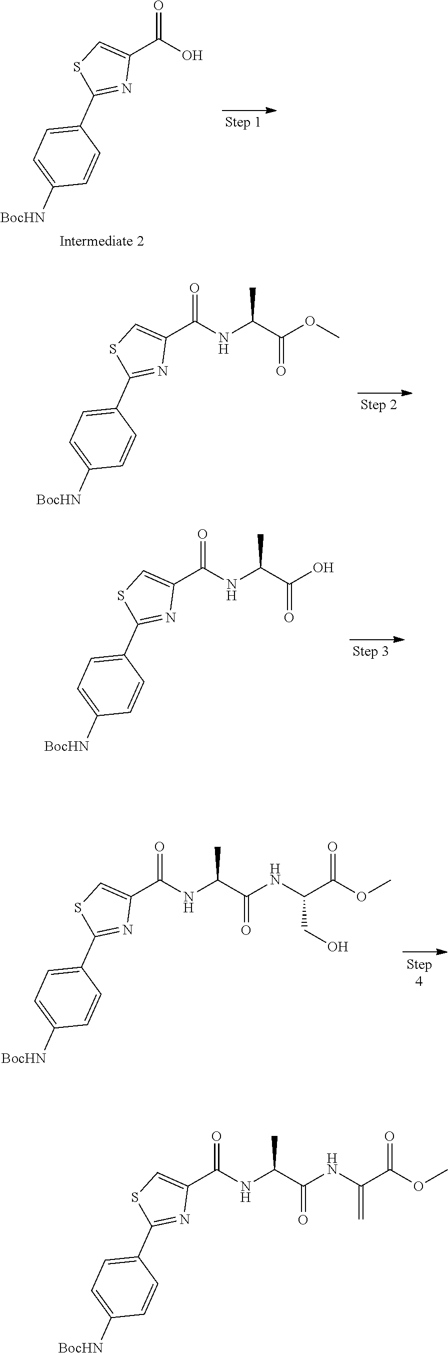 [SMILES: CC(C)(C)OC(=O)Nc1ccc(-c2nc(C(=O)O)cs2)cc1 ; COC(=O)[C@H](C)NC(=O)c1csc(-c2ccc(NC(=O)OC(C)(C)C)cc2)n1 ; C[C@H](NC(=O)c1csc(-c2ccc(NC(=O)OC(C)(C)C)cc2)n1)C(=O)O ; COC(=O)[C@H](CO)NC(=O)[C@H](C)NC(=O)c1csc(-c2ccc(NC(=O)OC(C)(C)C)cc2)n1 ; C=C(NC(=O)[C@H](C)NC(=O)c1csc(-c2ccc(NC(=O)OC(C)(C)C)cc2)n1)C(=O)OC]

Step 1. Methyl (2-(4-((tert-butoxycarbonyl)amino)phenyl)thiazole-4-carbonyl)-L-alaninate was prepared following General experimental procedure 3. 2-(4-((tert-butoxycarbonyl)amino)phenyl)thiazole-4-carboxylic acid (Intermediate 2) (0.53 g, 1.7 mmol) and H-Ala-OMe·HCl (0.27 g, 1.2 eq., 1.9 mmol) gave methyl (2-(4-((tert-butoxycarbonyl)amino)phenyl)thiazole-4-carbonyl)-L-alaninate (0.43 mg, 1.1 mmol, 63%) LCMS (General 3 basic) RT: 1.77 min; area % (254 nm): 86%; m/z=404.2 [M+H]⁺.

Step 2. (2-(4-((tert-butoxycarbonyl)amino)phenyl)thiazole-4-carbonyl)-D-alanine was prepared following General experimental procedure 2. Methyl (2-(4-((tert-butoxycarbonyl)amino)phenyl)thiazole-4-carbonyl)-D-alaninate (0.43 g, 1.1 mmol) gave (2-(4-((tert-butoxycarbonyl)amino)phenyl)thiazole-4-carbonyl)-D-alanine (0.41 g, 1.1 mmol, quant.) as a yellow foam. LCMS (General 3 basic) RT: 1.16 min; area % (254 nm): 95%; m/z=392.2 [M+H]⁺.

Step 3. Methyl (2-(4-((tert-butoxycarbonyl)amino)phenyl)thiazole-4-carbonyl)-L-alanyl-L-serinate was prepared following General experimental procedure 3. (2-(4-((tert-butoxycarbonyl)amino)phenyl)thiazole-4-carbonyl)-D-alanine (0.41 g, 1.1 mmol) and H-Ser-OMe·HCl (0.25 mg, 1.2 eq., 1.38 mmol) gave methyl (2-(4-((tert-butoxycarbonyl)amino)phenyl)thiazole-4-carbonyl)-L-alanyl-L-serinate (0.24 mg, 0.50 mmol 47%) as a yellow foam. LCMS (General 3 basic) RT: 1.54 min; area % (254 nm): 85%; m/z=493.2 [M+H]⁺.

Step 4. Methyl (S)-2-(2-(2-(4-((tert-butoxycarbonyl)amino)phenyl)thiazole-4-carboxamido)propanamido)acrylate was prepared using the following procedure. To a solution of methyl (2-(4-((tert-butoxycarbonyl)amino)phenyl)thiazole-4-carbonyl)-L-alanylserinate (0.24 mg, 1 eq., 0.50 mmol) in MeCN (30 mL) was added triethylamine (0.14 mL, 2 eq., 1.0 mmol) and the resulting mixture was stirred for 6 days at room temperature. The mixture was concentrated and purified by automated FCC and automated reverse phase FCC to provide methyl (S)-2-(2-(2-(4-((tert-butoxycarbonyl)amino)phenyl)thiazole-4-carboxamido)propanamido)acrylate as white solid. LCMS (22010199D TFA LCMS-5 C8) RT: 1.453 min; area % (215 nm): 94.9%; m/z=475.2 [M+H]⁺. ¹H NMR (400 MHz, CDCl₃) δ 8.54 (s, 1H), 8.08 (s, 1H), 7.90-7.80 (m, 3H), 7.49-7.42 (m, 2H), 6.70 (s, 1H), 6.59 (s, 1H), 5.91 (d, J=1.4 Hz, 1H), 4.77 (p, J=7.1 Hz, 1H), 3.81 (s, 3H), 1.56 (d, J=7.0 Hz, 3H), 1.52 (s, 9H).

### Example 16: Compound 58

### Methyl (2-(2-(4-((tert-butoxycarbonyl)amino)phenyl)thiazole-4-carboxamido)acryloyl)glycinate

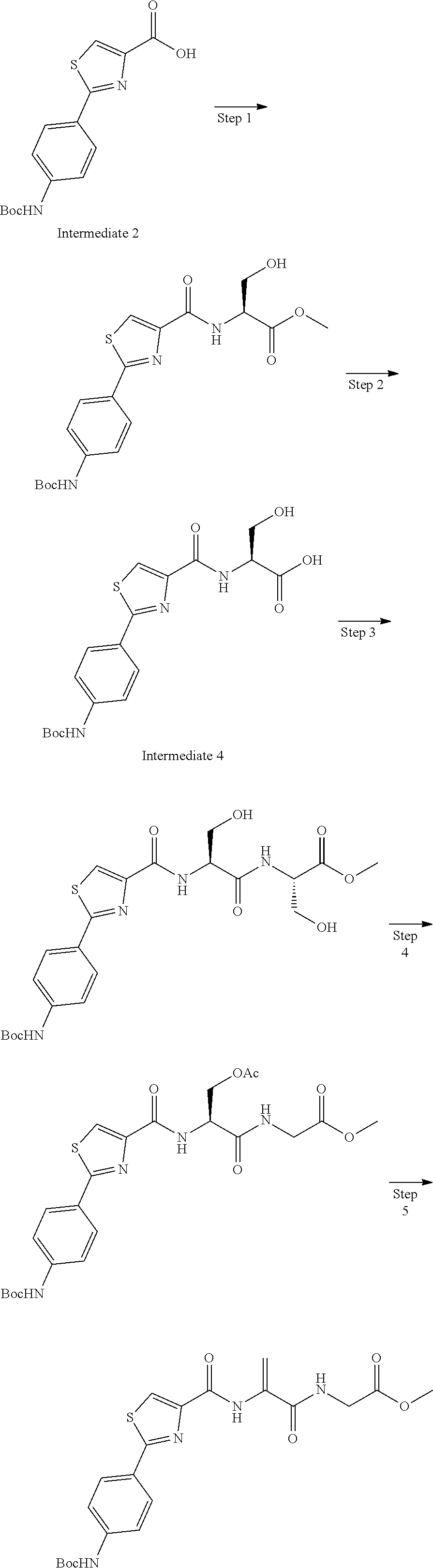 [SMILES: CC(C)(C)OC(=O)Nc1ccc(-c2nc(C(=O)O)cs2)cc1 ; COC(=O)[C@H](CO)NC(=O)c1csc(-c2ccc(NC(=O)OC(C)(C)C)cc2)n1 ; CC(C)(C)OC(=O)Nc1ccc(-c2nc(C(=O)N[C@@H](CO)C(=O)O)cs2)cc1 ; COC(=O)[C@H](CO)NC(=O)[C@H](CO)NC(=O)c1csc(-c2ccc(NC(=O)OC(C)(C)C)cc2)n1 ; COC(=O)CNC(=O)[C@H](COC(C)=O)NC(=O)c1csc(-c2ccc(NC(=O)OC(C)(C)C)cc2)n1 ; C=C(NC(=O)c1csc(-c2ccc(NC(=O)OC(C)(C)C)cc2)n1)C(=O)NCC(=O)OC]

Step 1. Methyl (2-(4-((tert-butoxycarbonyl)amino)phenyl)thiazole-4-carbonyl)serinate was prepared following General experimental procedure 3. 2-(4-((tert-butoxycarbonyl)amino)phenyl)thiazole-4-carboxylic acid (Intermediate 2) (1.0 g, 3.2 mmol) and L-Ser-OMe·HCl (0.59 g, 1.2 eq., 3.8 mmol) gave methyl (2-(4-((tert-butoxycarbonyl)amino)phenyl)thiazole-4-carbonyl)serinate (0.88 g, 2.1 mmol, 66%). LCMS (General 3 basic) RT: 1.54 min; area % (254 nm): 64%; m/z=420.2 [M−H]⁻.

Step 2. (2-(4-((tert-butoxycarbonyl)amino)phenyl)thiazole-4-carbonyl)serine (Intermediate 4) was prepared following General experimental procedure 2. Methyl (2-(4-((tert-butoxycarbonyl)amino)phenyl)thiazole-4-carbonyl)serinate (0.88 g, 2.1 mmol) gave (2-(4-((tert-butoxycarbonyl)amino)phenyl)thiazole-4-carbonyl)serine (Intermediate 4) (0.84 mg, 2.1 mmol, 99%) as a white solid.

Step 3. Methyl (2-(4-((tert-butoxycarbonyl)amino)phenyl)thiazole-4-carbonyl)serylglycinate was prepared following General experimental procedure 3. (2-(4-((tert-butoxycarbonyl)amino)phenyl)thiazole-4-carbonyl)serine (Intermediate 4) (0.42 g, 1.0 mmol) and H-Gly-OMe·HCl (0.18 g, 1.4 mmol) gave methyl (2-(4-((tert-butoxycarbonyl)amino)phenyl)thiazole-4-carbonyl)serylglycinate (0.35 g, 0.72 mmol, 70%) as a yellow solid. ¹H NMR (400 MHz, MeOD) δ 8.16 (s, 1H), 7.98-7.88 (m, 2H), 7.59-7.51 (m, 2H), 4.71 (t, J=4.9 Hz, 1H), 4.04-3.96 (m, 3H), 3.92 (dd, J=11.3, 4.8 Hz, 1H), 3.73 (s, 3H), 1.54 (s, 9H).

Step 4. Methyl O-acetyl-N-(2-(4-((tert-butoxycarbonyl)amino)phenyl)thiazole-4-carbonyl)serylglycinate was prepared following General experimental procedure 5. Methyl (2-(4-((tert-butoxycarbonyl)amino)phenyl)thiazole-4-carbonyl)serylglycinate (0.13 mg, 0.27 mmol) and acetic anhydride (26 μL, 1 eq., 0.27 mmol) gave methyl O-acetyl-N-(2-(4-((tert-butoxycarbonyl)amino)phenyl)thiazole-4-carbonyl)serylglycinate (153 mg, quant). LCMS (General 3 acidic) RT: 1.14 min; area % (254 nm): 92%; m/z=521.5 [M+H]⁺.

Step 5. Methyl (2-(2-(4-((tert-butoxycarbonyl)amino)phenyl)thiazole-4-carboxamido)acryloyl)glycinate was prepared using the following procedure. To a solution of methyl (2-(2-(4-((tert-butoxycarbonyl)amino)phenyl)thiazole-4-carboxamido)acryloyl)glycinate (51 mg, 99 μmol) in CH₂Cl₂ (1 mL) was added DBU (30 μL, 2 eq., 197 μmol) and the mixture was stirred at room temperature overnight. The reaction mixture was concentrated in vacuo and the crude material was purified by automated reverse phase FCC to provide methyl (2-(2-(4-((tert-butoxycarbonyl)amino)phenyl)thiazole-4-carboxamido)acryloyl)glycinate (12.5 mg, 27.1 μmol, 28%) as a white solid. LCMS (22010199D TFA LCMS-5 C8) RT: 1.403 min; area % (215 nm): 93.2%; m/z=461.2 [M+H]⁺. ¹H NMR (400 MHz, CDCl₃) δ 9.98 (s, 1H), 8.06 (s, 1H), 7.94-7.82 (m, 2H), 7.48-7.39 (m, 2H), 6.73 (s, 1H), 6.71-6.66 (m, 2H), 5.43 (t, J=1.7 Hz, 1H), 4.18 (d, J=5.0 Hz, 2H), 3.79 (s, 3H), 1.52 (s, 9H).

### Example 17: Compound 59

### Methyl (2-(2-(4-((tert-butoxycarbonyl)amino)phenyl)thiazole-4-carboxamido)acryloyl)-L-alaninate

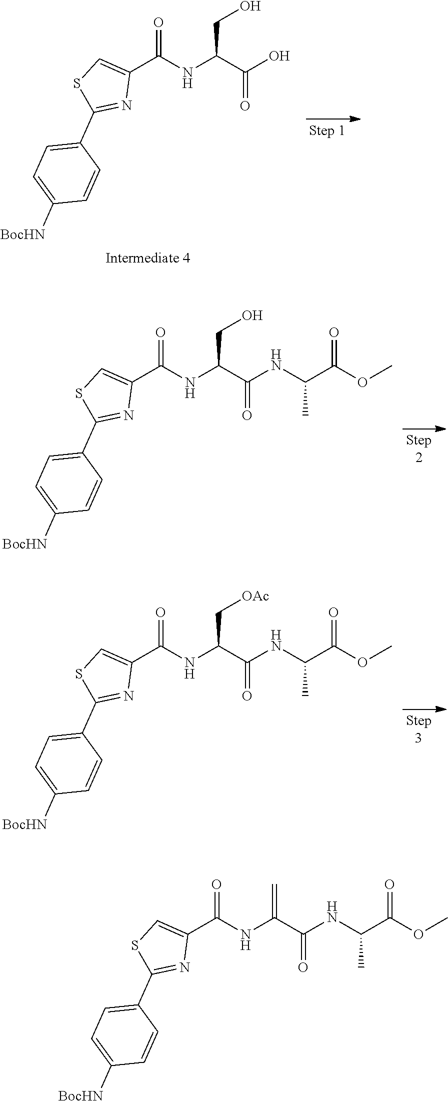 [SMILES: CC(C)(C)OC(=O)Nc1ccc(-c2nc(C(=O)N[C@@H](CO)C(=O)O)cs2)cc1 ; COC(=O)[C@H](C)NC(=O)[C@H](CO)NC(=O)c1csc(-c2ccc(NC(=O)OC(C)(C)C)cc2)n1 ; COC(=O)[C@H](C)NC(=O)[C@H](COC(C)=O)NC(=O)c1csc(-c2ccc(NC(=O)OC(C)(C)C)cc2)n1 ; C=C(NC(=O)c1csc(-c2ccc(NC(=O)OC(C)(C)C)cc2)n1)C(=O)N[C@@H](C)C(=O)OC]

Step 1. Methyl (2-(4-((tert-butoxycarbonyl)amino)phenyl)thiazole-4-carbonyl)seryl-L-alaninate was prepared following General experimental procedure 3. (2-(4-((tert-butoxycarbonyl)amino)phenyl)thiazole-4-carbonyl)serine (Intermediate 4) (0.42 g, 1.0 mmol) and H-Ala-OMe·HCl (0.19 g, 1.3 eq., 1.3 mmol) gave methyl (2-(4-((tert-butoxycarbonyl)amino)phenyl)thiazole-4-carbonyl)seryl-L-alaninate (0.24 g, 0.49 mmol, 48%) as a yellow foam. LCMS (General 3 basic) RT: 1.60 min; area % (254 nm): 81%; m/z=493.2 [M+H]⁺.

Step 2. Methyl O-acetyl-N-(2-(4-((tert-butoxycarbonyl)amino)phenyl)thiazole-4-carbonyl)-L-seryl-L-alaninate was prepared following General experimental procedure 5. Methyl (2-(4-((tert-butoxycarbonyl)amino)phenyl)thiazole-4-carbonyl)-L-seryl-L-alaninate (0.20 g, 0.41 mmol) and acetic anhydride (42 μL, 0.44 mmol) gave methyl O-acetyl-N-(2-(4-((tert-butoxycarbonyl)amino)phenyl)thiazole-4-carbonyl)-L-seryl-L-alaninate (0.23 g, 0.44 mmol, quant.) as a light yellow foam. LCMS (General 3 basic) RT: 1.59 min; area % (254 nm): 86%; m/z=535.2 [M+H]⁺.

Step 3. Methyl (2-(2-(4-((tert-butoxycarbonyl)amino)phenyl)thiazole-4-carboxamido)acryloyl)-L-alaninate was prepared using the following procedure. To a solution of methyl O-acetyl-N-(2-(4-((tert-butoxycarbonyl)amino)phenyl)thiazole-4-carbonyl)-L-seryl-L-alaninate (52 mg, 98 μmol) in CH₂Cl₂ (1 mL) was added DBU (30 μL, 0.20 mmol) and the resulting mixture was stirred for 16 hours at room temperature. The reaction mixture was concentrated in vacuo and the crude material was purified by automated reverse phase FCC to provide methyl (2-(2-(4-((tert-butoxycarbonyl)amino)phenyl)thiazole-4-carboxamido)acryloyl)-L-alaninate (10.8 mg, 22.8 μmol, 23%). LCMS (22010199D TFA LCMS-5 C8) RT: 1.472 min; area % (215 nm): 97.4%; m/z=475.2 [M+H]⁺. ¹H NMR (400 MHz, CDCl₃) δ 9.99 (s, 1H), 8.05 (s, 1H), 7.90 (d, J=8.4 Hz, 2H), 7.43 (d, J=8.3 Hz, 2H), 6.80 (d, J=7.2 Hz, 1H), 6.72 (s, 1H), 6.67 (d, J=1.8 Hz, 1H), 5.44-5.39 (m, 1H), 4.69 (p, J=7.2 Hz, 1H), 3.79 (s, 3H), 1.50 (m, 12H).

### Example 18: Compound 60

### N₃(CH₂CH₂O)₃CH₂C(O)-Cha-D-Arg-Cha-D-Arg-Cha-D-Arg-NH₂ HCl Salt

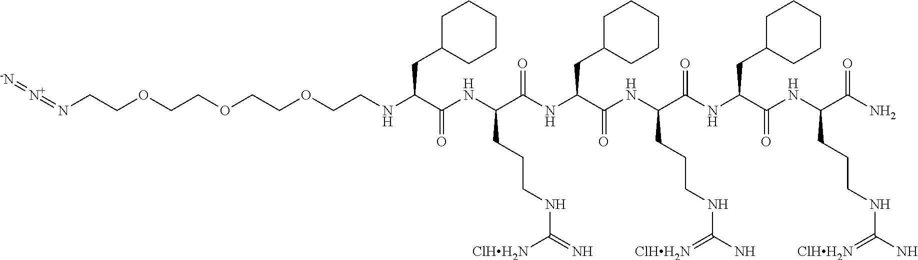

N₃(CH₂CH₂O)₃CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form.

N₃(CH₂CH₂O)₃CH₂C(O)-Cha-D-Arg-Cha-D-Arg-Cha-D-Arg-NH₂ HCl salt (53 mg, 42 μmol, 17%) was isolated as a white solid. LCMS (30833 LCMS-6): RT: 2.365 min; Area: 83.7% (215 nm), 99.8% (ELSD); m/z=1160.8 [M+H]⁺

### Example 19: Compound 61

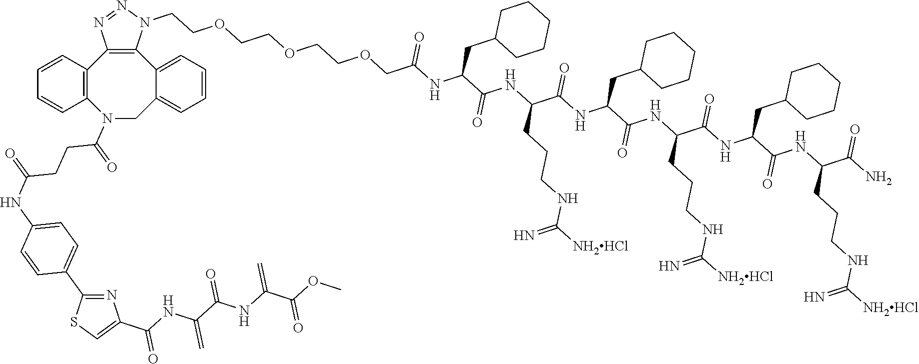

Compound 61 was prepared following General procedure for conjugation chemistry between azido-peptide and. Compound 49 (3.0 mg, 4.5 μmol) and Compound 60 (5.8 mg, 4.5 μmol) gave Compound 61 (4.5 mg, 2.3 μmol, 51%) as a white solid. LCMS (30833 LCMS-6): RT: 2.781 min; Area: 91.1% (215 nm), 99.4% (ELSD); m/z=1820.1 [M+H]⁺

### Example 20: Compound 62

### Methyl (E)-2-(2-(2-(4-((tert-butoxycarbonyl)amino)phenyl)thiazole-4-carboxamido)acrylamido)but-2-enoate

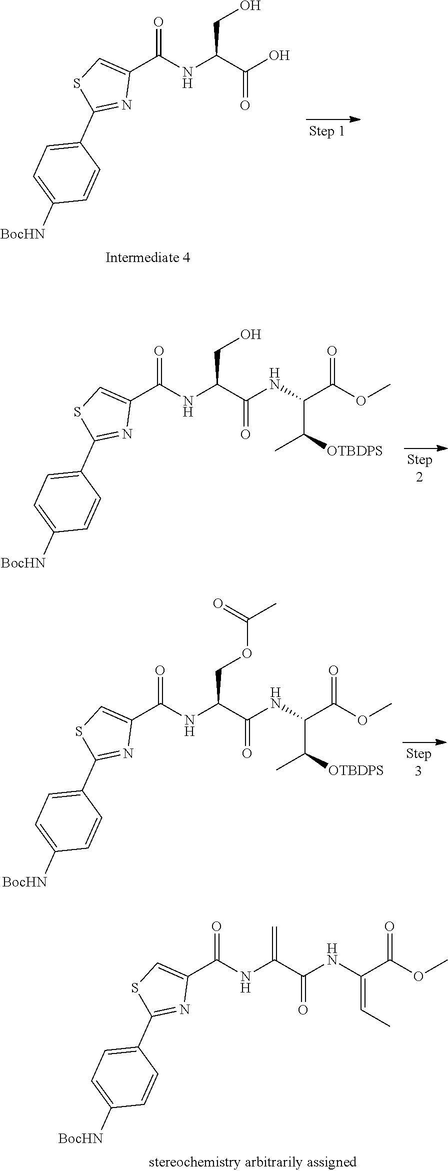

Step 1. Methyl N-((2-(4-((tert-butoxycarbonyl)amino)phenyl)thiazole-4-carbonyl)seryl)-O-(tert-butyldiphenylsilyl)-L-allothreoninate was prepared following General experimental procedure 4. (2-(4-((tert-butoxycarbonyl)amino)phenyl)thiazole-4-carbonyl)serine (Intermediate 4) (785 mg, 1.93 mmol) and methyl O-(tert-butyldiphenylsilyl)-L-allothreoninate (859 mg, 1.2 eq., 2.31 mmol) gave methyl N-((2-(4-((tert-butoxycarbonyl)amino)phenyl)thiazole-4-carbonyl)seryl)-O-(tert-butyldiphenylsilyl)-L-allothreoninate (1.54 g, 2.02 mmol, quant.) as a viscous oil. LCMS (General 3 acidic) RT: 1.81 min; area % (254 nm): 96%; m/z=761.3 [M+H]⁺.

Step 2. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate was prepared following General experimental procedure 5. Methyl N-((2-(4-((tert-butoxycarbonyl)amino)phenyl)thiazole-4-carbonyl)seryl)-O-(tert-butyldiphenylsilyl)-L-allothreoninate (1.6 g, 2.1 mmol) and acetic anhydride (0.22 mL, 1.1 eq., 2.3 mmol) gave methyl N—(O-acetyl-N-(2-(4-((tert-butoxycarbonyl)amino)phenyl)thiazole-4-carbonyl)seryl)-O-(tert-butyldiphenylsilyl)-L-allothreoninate (1.37 g, 1.71 mmol, 81%) as a yellow solid. LCMS (General 3 acidic) RT: 1.88 min; area % (254 nm): 75%; m/z=803.5 [M+H]⁺.

Step 3. Methyl (E)-2-(2-(2-(4-((tert-butoxycarbonyl)amino)phenyl)thiazole-4-carboxamido)acrylamido)but-2-enoate was prepared following General experimental procedure 7. Methyl N—(O-acetyl-N-(2-(4-((tert-butoxycarbonyl)amino)phenyl)thiazole-4-carbonyl)seryl)-O-(tert-butyldiphenylsilyl)-L-allothreoninate (306 mg, 0.38 mmol) gave methyl methyl (E)-2-(2-(2-(4-((tert-butoxycarbonyl)amino)phenyl)thiazole-4-carboxamido)acrylamido)but-2-enoate (4.6 mg, 9.5 μmol, 2.5%). LCMS (22010199C TFA LCMS-5 C3) RT: 1.470 min; area: 93.8% (215 nm); m/z 487.2 [M+H]⁺. ¹H NMR (400 MHz, CDCl₃) δ 9.98 (s, 1H), 8.17 (s, 1H), 8.06 (s, 1H), 7.95-7.88 (m, 2H), 7.44 (d, J=8.6 Hz, 2H), 7.33 (q, J=7.7 Hz, 2H), 6.70 (d, J=2.0 Hz, 1H), 6.59 (s, 1H), 5.42 (s, 1H), 3.88 (s, 3H), 2.15 (d, J=7.7 Hz, 3H), 1.52 (s, 9H).

### Example 21: Compound 63

### methyl (E)-2-(2-(2-(4-((tert-butoxycarbonyl)amino)phenyl)thiazole-4-carboxamido)acrylamido)but-2-enoate

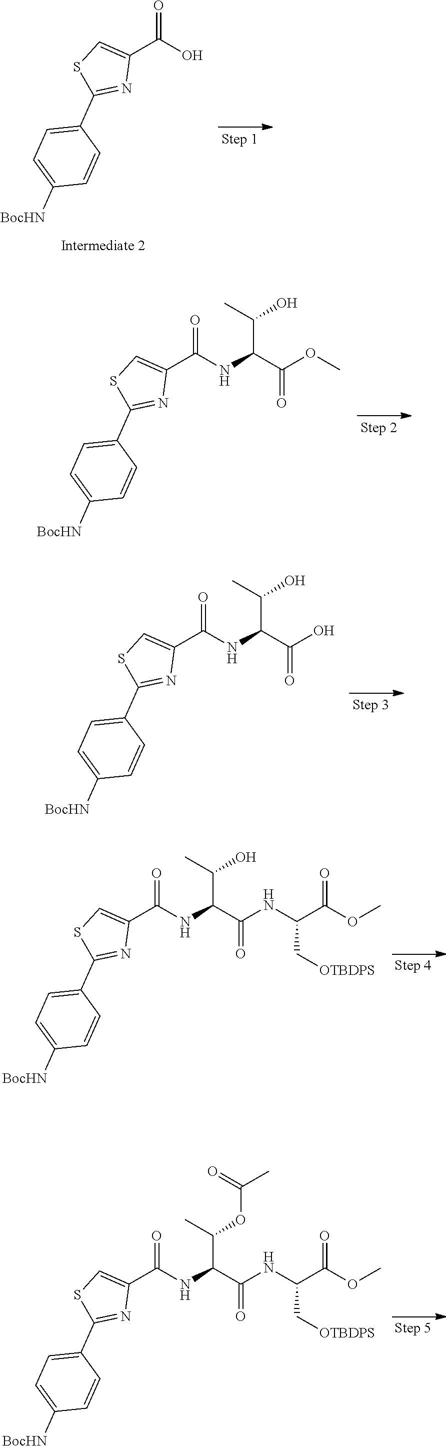

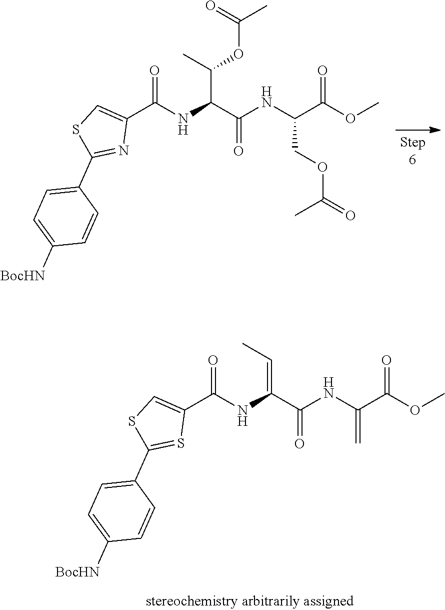

Step 1. Methyl (2-(4-((tert-butoxycarbonyl)amino)phenyl)thiazole-4-carbonyl)-L-allothreoninate was prepared following General experimental procedure 4. 2-(4-((tert-butoxycarbonyl)amino)phenyl)thiazole-4-carboxylic acid (Intermediate 2) (0.98 g, 3.1 mmol) and methyl L-allothreoninate hydrochloride (0.57 g, 1.1 eq., 3.4 mmol) gave methyl (2-(4-((tert-butoxycarbonyl)amino)phenyl)thiazole-4-carbonyl)-L-allothreoninate (1.6 g, 3.2 mmol, quant.) as a white solid.

Step 2. (2-(4-((tert-butoxycarbonyl)amino)phenyl)thiazole-4-carbonyl)-L-allothreonine was prepared following General experimental procedure 2. Methyl (2-(4-((tert-butoxycarbonyl)amino)phenyl)thiazole-4-carbonyl)-L-allothreoninate (1.6 g, 3.2 mmol) gave (2-(4-((tert-butoxycarbonyl)amino)phenyl)thiazole-4-carbonyl)-L-allothreonine (1.4 g, 3.2 mmol, quant.) as a white solid.

Step 3. Methyl N-((2-(4-((tert-butoxycarbonyl)amino)phenyl)thiazole-4-carbonyl)-L-allothreonyl)-O-(tert-butyldiphenylsilyl)-L-serinate was prepared following General experimental procedure 4. (2-(4-((tert-butoxycarbonyl)amino)phenyl)thiazole-4-carbonyl)-L-allothreonine (1.4 g, 3.2 mmol) and methyl O-(tert-butyldiphenylsilyl)-L-serinate hydrochloride (1.4 g, 1.1 eq., 3.5 mmol) gave methyl N-((2-(4-((tert-butoxycarbonyl)amino)phenyl)thiazole-4-carbonyl)-L-allothreonyl)-O-(tert-butyldiphenylsilyl)-L-serinate (2.3 g, 3.0 mmol, 93%)

Step 4. Methyl N—(O-acetyl-N-(2-(4-((tert-butoxycarbonyl)amino)phenyl)thiazole-4-carbonyl)-L-allothreonyl)-O-(tert-butyldiphenylsilyl)-L-serinate was prepared following General experimental procedure 5. Methyl N-((2-(4-((tert-butoxycarbonyl)amino)phenyl)thiazole-4-carbonyl)-L-allothreonyl)-O-(tert-butyldiphenylsilyl)-L-serinate (2.3 g, 3.0 mmol) gave methyl N—(O-acetyl-N-(2-(4-((tert-butoxycarbonyl)amino)phenyl)thiazole-4-carbonyl)-L-allothreonyl)-O-(tert-butyldiphenylsilyl)-L-serinate (2.2 g, 2.7 mmol, 90%).

Step 5. Methyl O-acetyl-N—(O-acetyl-N-(2-(4-((tert-butoxycarbonyl)amino)phenyl)thiazole-4-carbonyl)-L-allothreonyl)-L-serinate was prepared using the following procedure. To a solution of methyl N—(O-acetyl-N-(2-(4-((tert-butoxycarbonyl)amino)phenyl)thiazole-4-carbonyl)-L-allothreonyl)-O-(tert-butyldiphenylsilyl)-L-serinate (0.30 g, 0.37 mmol) in THF (6 mL) was added a solution of TBAF in THF (0.56 mL, 1M, 1.5 eq., 0.56 mmol) and the resulting mixture was stirred for 1 hour. The reaction mixture was concentrated in vacuo and the residue was dissolved in MeCN (6 mL). Triethylamine (0.13 mL, 2.5 eq., 0.93 mmol) and acetic anhydride (48 μL, 1.35 eq., 0.50 mmol) were added and the resulting mixture was stirred for 45 minutes at room temperature. Water was added and the mixture was concentrated in vacuo to remove MeCN. Then, the mixture was extracted with EtOAc. The organic layer was washed with brine, dried over Na₂SO₄, filtered and concentrated. The crude material was purified by automated FCC to provide a methyl O-acetyl-N—(O-acetyl-N-(2-(4-((tert-butoxycarbonyl)amino)phenyl)thiazole-4-carbonyl)-L-allothreonyl)-L-serinate (0.16 g, 0.27 mmol, 72%) as a white solid.

Step 6. Methyl (Z)-2-(2-(2-(4-((tert-butoxycarbonyl)amino)phenyl)thiazole-4-carboxamido)but-2-enamido)acrylate (E/Z stereochemistry arbitrarily assigned) was prepared following General experimental procedure 8. Methyl O-acetyl-N—(O-acetyl-N-(2-(4-((tert-butoxycarbonyl)amino)phenyl)thiazole-4-carbonyl)-L-allothreonyl)-L-serinate (0.15 g, 0.25 mmol) gave methyl (Z)-2-(2-(2-(4-((tert-butoxycarbonyl)amino)phenyl)thiazole-4-carboxamido)but-2-enamido)acrylate (30 mg, 62 μmol, 25%) as a white solid. %). LCMS (22010199C TFA LCMS-5 C3) RT: 1.387 min; Area: 99.6% (215 nm), 99.3% (306 nm); m/z 487.2 [M+H]⁺. ¹H NMR (400 MHz, CDCl₃) δ 8.73 (s, 1H), 8.48 (s, 1H), 8.13 (s, 1H), 7.95-7.88 (m, 2H), 7.48 (d, J=8.5 Hz, 2H), 6.73 (q, J=7.1 Hz, 1H), 6.69 (s, 1H), 6.63 (s, 1H), 5.94 (d, J=1.4 Hz, 1H), 3.81 (s, 3H), 1.89 (d, J=7.1 Hz, 3H), 1.54 (s, 9H).

### Example 22: Compound 67

### Methyl 2-(2-(2-(4-((tert-butoxycarbonyl)amino)phenyl)thiazole-5-carboxamido)acrylamido)acrylate

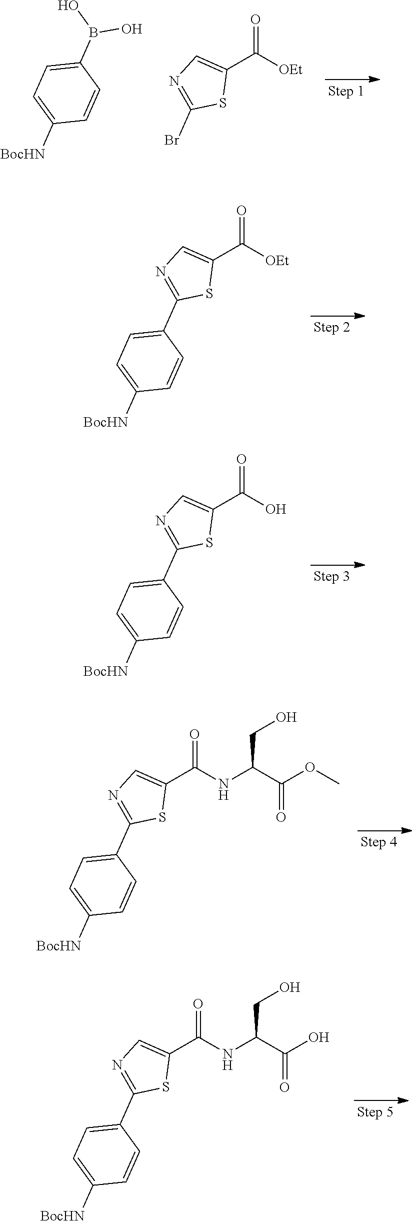 [SMILES: CC(C)(C)OC(=O)Nc1ccc(B(O)O)cc1 ; CCOC(=O)c1cnc(Br)s1 ; CCOC(=O)c1cnc(-c2ccc(NC(=O)OC(C)(C)C)cc2)s1 ; CC(C)(C)OC(=O)Nc1ccc(-c2ncc(C(=O)O)s2)cc1 ; COC(=O)[C@H](CO)NC(=O)c1cnc(-c2ccc(NC(=O)OC(C)(C)C)cc2)s1 ; CC(C)(C)OC(=O)Nc1ccc(-c2ncc(C(=O)N[C@@H](CO)C(=O)O)s2)cc1]

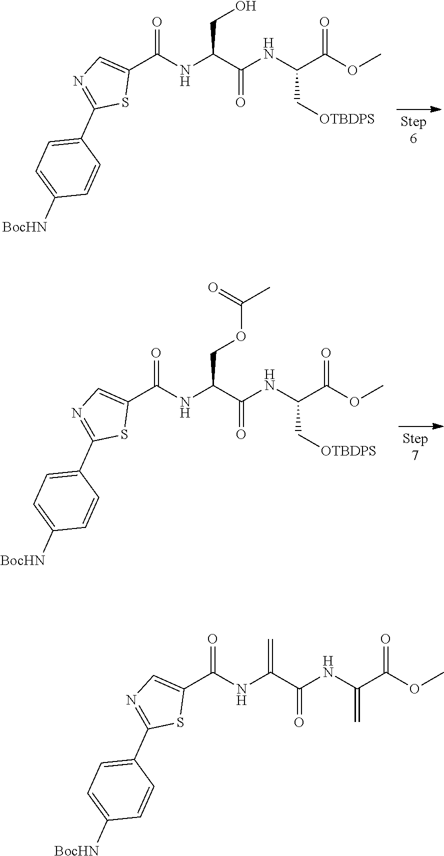

Step 1. ethyl 2-(4-((tert-butoxycarbonyl)amino)phenyl)thiazole-5-carboxylate was prepared following General experimental procedure 1. (4-((tert-butoxycarbonyl)amino)phenyl)boronic acid (2.0 g, 8.4 mmol) and ethyl 2-bromothiazole-5-carboxylate (2.0 g, 8.4 mmol) gave ethyl 2-(4-((tert-butoxycarbonyl)amino)phenyl)thiazole-5-carboxylate (1.26 g, 3.62 mmol, 43%) as a yellow solid. ¹H NMR (299 MHz, CDCl₃) δ 8.39 (s, 1H), 7.94 (d, J=8.8 Hz, 2H), 7.49 (d, J=8.6 Hz, 2H), 6.66 (s, 1H), 4.40 (q, J=7.1 Hz, 2H), 1.55 (d, J=1.0 Hz, 10H), 1.42 (t, J=7.1 Hz, 3H).

Step 2. 2-(4-((tert-butoxycarbonyl)amino)phenyl)thiazole-5-carboxylic acid was prepared following General experimental procedure 2. Ethyl 2-(4-((tert-butoxycarbonyl)amino)phenyl)thiazole-5-carboxylate (1.2 g, 3.4 mmol) gave 2-(4-((tert-butoxycarbonyl)amino)phenyl)thiazole-5-carboxylic acid (1.0 g, 3.1 mmol, 91%) as a white solid.

Step 3. methyl (2-(4-((tert-butoxycarbonyl)amino)phenyl)thiazole-5-carbonyl)-L-serinate was prepared following General experimental procedure 4. 2-(4-((tert-butoxycarbonyl)amino)phenyl)thiazole-5-carboxylic acid (1.6 g, 5.0 mmol) and H-Ser-OMe·HCl (0.93 g, 1.2 eq., 6.0 mmol) gave methyl (2-(4-((tert-butoxycarbonyl)amino)phenyl)thiazole-5-carbonyl)-L-serinate (1.4 g, 3.3 mmol, 67%) as an orange oil. LCMS (General 3 acidic) RT: 0.99 min; area % (254 nm): 88%; m/z=422.3 [M+H]⁺.

Step 4. (2-(4-((tert-butoxycarbonyl)amino)phenyl)thiazole-5-carbonyl)-L-serine was prepared following General experimental procedure 2. Methyl (2-(4-((tert-butoxycarbonyl)amino)phenyl)thiazole-5-carbonyl)-L-serinate (1.4 g, 3.3 mmol) gave (2-(4-((tert-butoxycarbonyl)amino)phenyl)thiazole-5-carbonyl)-L-serine (0.98 g, 2.4 mmol, 72%).

Step 5. Methyl N-((2-(4-((tert-butoxycarbonyl)amino)phenyl)thiazole-5-carbonyl)-L-seryl)-O-(tert-butyldiphenylsilyl)-L-serinate was prepared following General experimental procedure 4. (2-(4-((tert-butoxycarbonyl)amino)phenyl)thiazole-5-carbonyl)-L-serine (0.98 g, 2.4 mmol) and methyl O-(tert-butyldiphenylsilyl)-L-serinate hydrochloride (1.1 g, 1.2 eq. 2.9 mmol) gave crude methyl N-((2-(4-((tert-butoxycarbonyl)amino)phenyl)thiazole-5-carbonyl)-L-seryl)-O-(tert-butyldiphenylsilyl)-L-serinate (2.25 g) which was used as such in the next step

Step 6. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate was prepared following General experimental procedure 5. Crude methyl N-((2-(4-((tert-butoxycarbonyl)amino)phenyl)thiazole-5-carbonyl)-L-seryl)-O-(tert-butyldiphenylsilyl)-L-serinate (2.25 g) and acetic anhydride (0.34 mL, 3.6 mmol) gave methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.40 g, 0.51 mmol, 21% over 2 steps). LCMS (General 3 acidic) RT: 1.78 min; area % (214 nm): 100%; m/z=789.6 [M+H]⁺.

Step 7. Methyl 2-(2-(2-(4-((tert-butoxycarbonyl)amino)phenyl)thiazole-5-carboxamido)acrylamido)acrylate was prepared following General experimental procedure 7. Methyl N—(O-acetyl-N-(2-(4-((tert-butoxycarbonyl)amino)phenyl)thiazole-5-carbonyl)-L-seryl)-O-(tert-butyldiphenylsilyl)-L-serinate (0.19 g, 0.24 mmol) gave methyl 2-(2-(2-(4-((tert-butoxycarbonyl)amino)phenyl)thiazole-5-carboxamido)acrylamido)acrylate (7.8 mg, 16 μmol, 27%) as a white solid LC-MS (22010199A TFA LCMS-5 C1): RT: 1.353 min; Area 97.7% (215 nm), 96.7% (304 nm); m/z=473.2 [M+H]⁺. ¹H NMR (400 MHz, CDCl₃) δ (ppm) 8.66 (s, 1H), 8.57 (s, 1H), 8.24 (s, 1H), 7.92 (d, J=8.8 Hz, 2H), 7.48 (d, J=8.8 Hz, 2H), 6.72 (d, J=2.5 Hz, 1H), 6.67 (s, 1H), 6.63 (s, 1H), 6.04 (d, J=1.0 Hz, 1H), 5.49 (dd, J=2.3 Hz, J=1.3 Hz, 1H), 3.91 (s, 1H), 1.54 (s, 9H).

### Example 23: Compound 68

### Methyl 2-(2-(5-(4-((tert-butoxycarbonyl)amino)phenyl)thiophene-3-carboxamido)acrylamido)acrylate

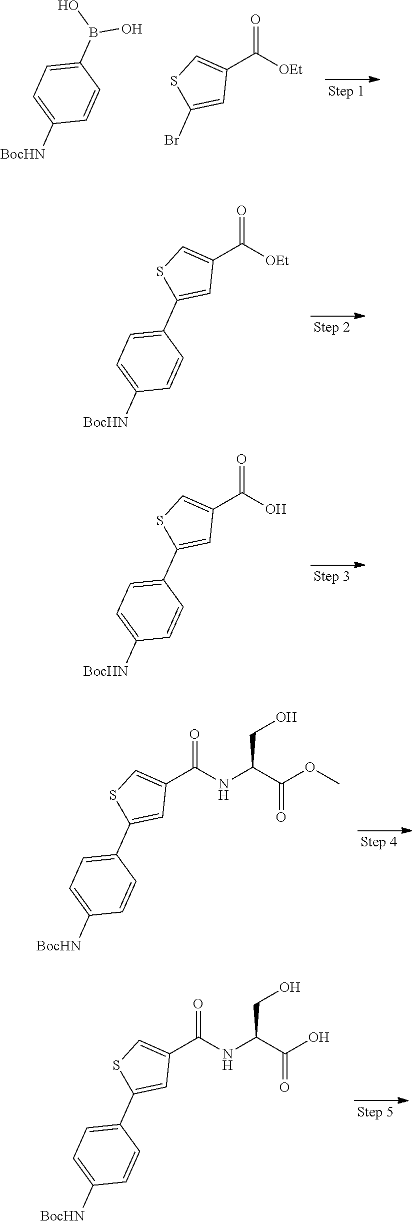 [SMILES: CC(C)(C)OC(=O)Nc1ccc(B(O)O)cc1 ; CCOC(=O)c1csc(Br)c1 ; CCOC(=O)c1csc(-c2ccc(NC(=O)OC(C)(C)C)cc2)c1 ; CC(C)(C)OC(=O)Nc1ccc(-c2cc(C(=O)O)cs2)cc1 ; COC(=O)[C@H](CO)NC(=O)c1csc(-c2ccc(NC(=O)OC(C)(C)C)cc2)c1 ; CC(C)(C)OC(=O)Nc1ccc(-c2cc(C(=O)N[C@@H](CO)C(=O)O)cs2)cc1]

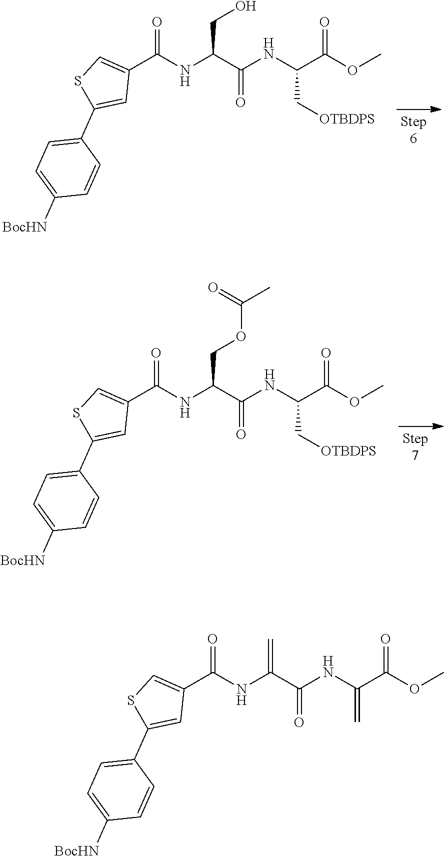

Step 1. Ethyl 5-(4-((tert-butoxycarbonyl)amino)phenyl)thiophene-3-carboxylate was prepared following General experimental procedure 1. Ethyl 5-bromothiophene-3-carboxylate (1.0 g, 4.2 mmol) and (4-((tert-butoxycarbonyl)amino)phenyl)boronic acid (1.0 g, 4.2 mmol) gave ethyl 5-(4-((tert-butoxycarbonyl)amino)phenyl)thiophene-3-carboxylate (1.1 g, 3.2 mmol, 76%) as white solid. ¹H NMR (299 MHz, CDCl₃) δ 8.00 (m, 1H), 7.65 (m, 1H), 7.55 (d, J=8.7 Hz, 2H), 7.41 (d, J=8.5 Hz, 2H), 7.28 (s, 4H), 6.54 (s, 1H), 4.37 (q, J=7.1 Hz, 2H), 1.55 (s, 9H), 1.40 (t, J=7.1 Hz, 3H).

Step 2. 5-(4-((tert-butoxycarbonyl)amino)phenyl)thiophene-3-carboxylic acid was prepared following General experimental procedure 2. Ethyl 5-(4-((tert-butoxycarbonyl)amino)phenyl)thiophene-3-carboxylate (1.1 g, 3.2 mmol) and 5-(4-((tert-butoxycarbonyl)amino)phenyl)thiophene-3-carboxylic acid (0.90 g, 2.8 mmol, 89%) as a white solid ¹H NMR (299 MHz, CD₃OD) δ 8.08 (m, 1H), 7.65 (m, 1H), 7.62-7.52 (m, 2H), 7.48 (d, J=8.7 Hz, 2H), 1.54 (s, 8H).

Step 3. Methyl (5-(4-((tert-butoxycarbonyl)amino)phenyl)thiophene-3-carbonyl)-L-serinate was prepared following General experimental procedure 3. 5-(4-((tert-butoxycarbonyl)amino)phenyl)thiophene-3-carboxylic acid (0.90 g, 2.8 mmol) and methyl L-serinate hydrochloride (0.44 g, 2.8 mmol) gave methyl (5-(4-((tert-butoxycarbonyl)amino)phenyl)thiophene-3-carbonyl)-L-serinate (1.0 g, 2.4 mmol, 84%) as a yellow oil. ¹H NMR (299 MHz, CDCl₃) δ 7.84 (m, 1H), 7.56-7.46 (m, 3H), 7.40 (d, J=8.5 Hz, 2H), 7.03 (d, J=7.2 Hz, 1H), 6.64 (s, 1H), 4.87 (dt, J=7.2, 3.6 Hz, 1H), 4.39 (q, J=7.1 Hz, 1H), 4.09 (t, J=4.0 Hz, 2H), 3.85 (s, 3H), 1.55 (s, 9H).

Step 4. (5-(4-((tert-butoxycarbonyl)amino)phenyl)thiophene-3-carbonyl)-L-serine was prepared following General experimental procedure 2. Methyl (5-(4-((tert-butoxycarbonyl)amino)phenyl)thiophene-3-carbonyl)-L-serinate (1.0 g, 0.24 mmol) gave (5-(4-((tert-butoxycarbonyl)amino)phenyl)thiophene-3-carbonyl)-L-serine (0.90 g, 2.2 mmol, 93%) as a yellow solid. ¹H NMR (299 MHz, CD₃OD) δ 9.05 (s, 1H), 8.25 (d, J=8.0 Hz, OH), 8.04 (d, J=1.4 Hz, 1H), 7.74 (d, J=1.4 Hz, 1H), 7.63-7.52 (m, 2H), 7.48 (d, J=8.6 Hz, 2H), 4.76-4.67 (m, 1H), 4.10-3.92 (m, 2H), 1.55 (s, 9H).

Step 5. Methyl N-((5-(4-((tert-butoxycarbonyl)amino)phenyl)thiophene-3-carbonyl)-L-seryl)-O-(tert-butyldiphenylsilyl)-L-serinate was prepared following General experimental procedure 4. (5-(4-((tert-butoxycarbonyl)amino)phenyl)thiophene-3-carbonyl)-L-serine (0.20 g, 0.49 mmol) and methyl O-(tert-butyldiphenylsilyl)-L-serinate hydrochloride (0.23 g, 1.2 eq., 0.59 mmol) gave crude methyl N-((5-(4-((tert-butoxycarbonyl)amino)phenyl)thiophene-3-carbonyl)-L-seryl)-O-(tert-butyldiphenylsilyl)-L-serinate (233 mg) which was used without purification in the next step

Step 6. Methyl N—(O-acetyl-N-(5-(4-((tert-butoxycarbonyl)amino)phenyl)thiophene-3-carbonyl)-L-seryl)-O-(tert-butyldiphenylsilyl)-L-serinate was prepared following General experimental procedure 5. Crude methyl N-((5-(4-((tert-butoxycarbonyl)amino)phenyl)thiophene-3-carbonyl)-L-seryl)-O-(tert-butyldiphenylsilyl)-L-serinate (233 mg) gave methyl N—(O-acetyl-N-(5-(4-((tert-butoxycarbonyl)amino)phenyl)thiophene-3-carbonyl)-L-seryl)-O-(tert-butyldiphenylsilyl)-L-serinate (0.10 g, 0.13 mmol, 26% over 2 steps)

Step 7. Methyl 2-(2-(5-(4-((tert-butoxycarbonyl)amino)phenyl)thiophene-3-carboxamido)acrylamido)acrylate was prepared following General experimental procedure 7. Methyl N—(O-acetyl-N-(5-(4-((tert-butoxycarbonyl)amino)phenyl)thiophene-3-carbonyl)-L-seryl)-O-(tert-butyldiphenylsilyl)-L-serinate (50 mg, 63 μmol) gave methyl 2-(2-(5-(4-((tert-butoxycarbonyl)amino)phenyl)thiophene-3-carboxamido)acrylamido)acrylate (3.9 mg, 8.3 μmol, 31%) as a white solid. LC-MS (22010199A TFA LCMS-5 C1): RT 1.483 min; Area 84.0% (215 nm), 88.7% (304 nm); m/z=472.0 [M+H]⁺

### Example 24: Compound 69

### Methyl 2-(2-(4′-((tert-butoxycarbonyl)amino)-[1,1′-biphenyl]-4-carboxamido)acrylamido)acrylate

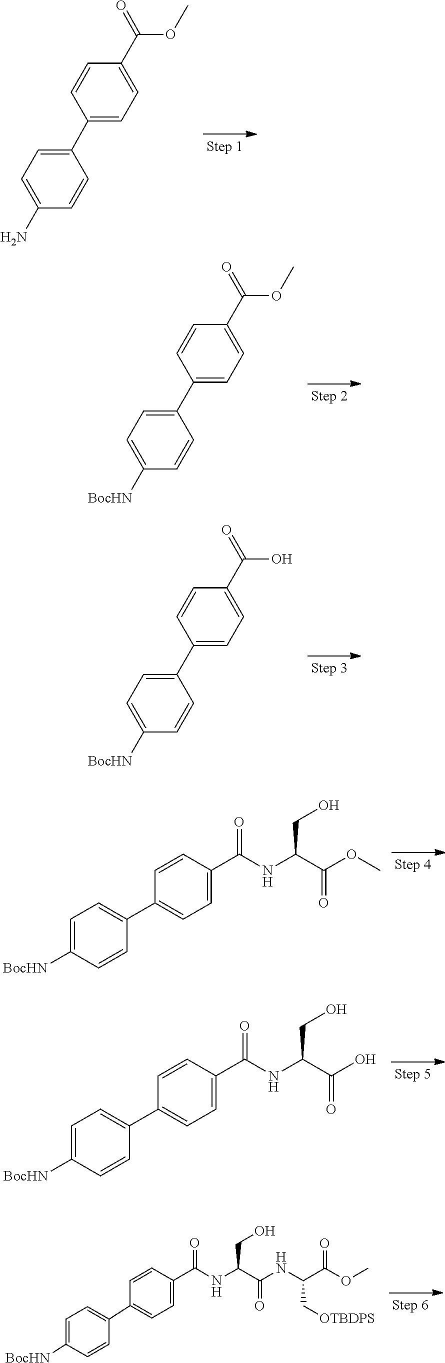

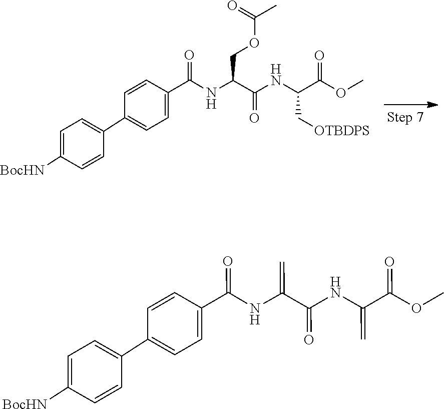

Step 1. Methyl 4′-((tert-butoxycarbonyl)amino)-[1,1′-biphenyl]-4-carboxylate was prepared using the following procedure. To a solution of methyl 4′-amino-[1,1′-biphenyl]-4-carboxylate (0.50 g, 2.2 mmol) in CH₂Cl₂ (20 mL) were added DMAP (0.54 g, 2 eq., 4.4 mmol) and Boc₂O (0.48 g, 1 eq., 2.2 mmol) and the resulting mixture was stirred for 16 hours at room temperature. The mixture was filtered and HCl (1M, aq. 50 mL) was added. The mixture was extracted with EtOAc and the organic layer was dried over Na₂SO₄, filtered and concentrated. The crude material was purified by FCC to provide methyl 4′-((tert-butoxycarbonyl)amino)-[1,1′-biphenyl]-4-carboxylate (0.30 g, 0.92 mmol, 42%). ¹H NMR (299 MHz, CDCl₃) δ 8.10 (m, 2H), 7.62 (m, 5H), 7.53-7.44 (m, 2H), 6.70 (s, 1H), 3.95 (s, 3H), 1.55 (s, 9H).

Step 2. 4′-((tert-butoxycarbonyl)amino)-[1,1′-biphenyl]-4-carboxylic acid was prepared following General experimental procedure 2. Methyl 4′-((tert-butoxycarbonyl)amino)-[1,1′-biphenyl]-4-carboxylate (0.52 g, 1.6 mmol) gave 4′-((tert-butoxycarbonyl)amino)-[1,1′-biphenyl]-4-carboxylic acid (0.34 g, 1.1 mmol, 66%)¹H NMR (299 MHz, CD₃OD) δ 8.13-8.00 (m, 2H), 7.77-7.58 (m, 4H), 7.53 (m, 2H), 1.55 (s, 9H).

Step 3. Methyl (4′-((tert-butoxycarbonyl)amino)-[1,1′-biphenyl]-4-carbonyl)-L-serinate was prepared following General experimental procedure 4. 4′-((tert-butoxycarbonyl)amino)-[1,1′-biphenyl]-4-carboxylic acid (90 mg, 0.29 mmol) and methyl L-serinate hydrochloride (90 mg, 2 eq., 0.57 mmol) gave methyl (4′-((tert-butoxycarbonyl)amino)-[1,1′-biphenyl]-4-carbonyl)-L-serinate (34 mg, 82 μmol, 29%). LCMS (General 3 acidic) RT: 1.07 min; area % (254 nm): 100%; m/z=415.1 [M+H]⁺.

Step 4. (4′-((tert-butoxycarbonyl)amino)-[1,1′-biphenyl]-4-carbonyl)-L-serine was prepared following General experimental procedure 2. Methyl (4′-((tert-butoxycarbonyl)amino)-[1,1′-biphenyl]-4-carbonyl)-L-serinate (34 mg, 82 μmol) gave crude (4′-((tert-butoxycarbonyl)amino)-[1,1′-biphenyl]-4-carbonyl)-L-serine (77 mg) which was used as such in the next step. LCMS (General 3 acidic) RT: 0.95 min; area % (254 nm): 82%; m/z=401.3 [M+H]⁺.

Step 5. Methyl N-((4′-((tert-butoxycarbonyl)amino)-[1,1′-biphenyl]-4-carbonyl)-L-seryl)-O-(tert-butyldiphenylsilyl)-L-serinate was prepared following General experimental procedure 4. (4′-((tert-butoxycarbonyl)amino)-[1,1′-biphenyl]-4-carbonyl)-L-serine (100 mg, 0.25 mmol) and methyl O-(tert-butyldiphenylsilyl)-L-serinate hydrochloride (118 mg, 0.30 mmol) gave methyl N-((4′-((tert-butoxycarbonyl)amino)-[1,1′-biphenyl]-4-carbonyl)-L-seryl)-O-(tert-butyldiphenylsilyl)-L-serinate (66 mg, 89 μmol, 36%). LCMS (General 3 acidic) RT: 1.72 min; area % (254 nm): 77%; m/z=740.6 [M+H]⁺.

Step 6. Methyl N—(O-acetyl-N-(4′-((tert-butoxycarbonyl)amino)-[1,1′-biphenyl]-4-carbonyl)-L-seryl)-O-(tert-butyldiphenylsilyl)-L-serinate was prepared following General experimental procedure 5. Methyl N-((4′-((tert-butoxycarbonyl)amino)-[1,1′-biphenyl]-4-carbonyl)-L-seryl)-O-(tert-butyldiphenylsilyl)-L-serinate (66 mg, 89 μmol) gave methyl N-(0-acetyl-N-(4′-((tert-butoxycarbonyl)amino)-[1,1′-biphenyl]-4-carbonyl)-L-seryl)-O-(tert-butyldiphenylsilyl)-L-serinate (44 mg, 56 μmol, 63%). LCMS (General 3 acidic) RT: 1.81 min; area % (254 nm): 100%; m/z=740.6 [M+H]⁺.

Step 7. Methyl 2-(2-(4′-((tert-butoxycarbonyl)amino)-[1,1′-biphenyl]-4-carboxamido)acrylamido)acrylate was prepared following General experimental procedure 7. Methyl N—(O-acetyl-N-(4′-((tert-butoxycarbonyl)amino)-[1,1′-biphenyl]-4-carbonyl)-L-seryl)-O-(tert-butyldiphenylsilyl)-L-serinate (42 mg, 54 μmol) gave methyl 2-(2-(4′-((tert-butoxycarbonyl)amino)-[1,1′-biphenyl]-4-carboxamido)acrylamido)acrylate (5.0 mg, 11 μmol, 20%). LC-MS (22010199A TFA LCMS-5 C1): rt 1.483 min; Area 78.4% (215 nm), 83.7% (304 nm); m/z=[M+H]⁺=466.2. ¹H NMR (CDCl₃) δ (ppm) 8.92 (s, 1H), 8.60 (s, 1H), 7.92 (d, J=8.3 Hz, 2H), 7.67 (d, J=8.1 Hz, 2H), 7.57 (d, J=8.4 Hz, 2H), 7.47 (d, J=8.4 Hz, 2H), 6.82 (m, 1H), 6.67 (s, 1H), 6.56 (s, 1H), 6.03 (s, 1H), 5.49 (s, 1H), 3.91 (s, 3H), 1.54 (s, 9H).

### Example 25: Compound 70

### Methyl 2-(2-(2-(4-((tert-butoxycarbonyl)amino)-3-fluorophenyl)thiazole-4-carboxamido)acrylamido)acrylate

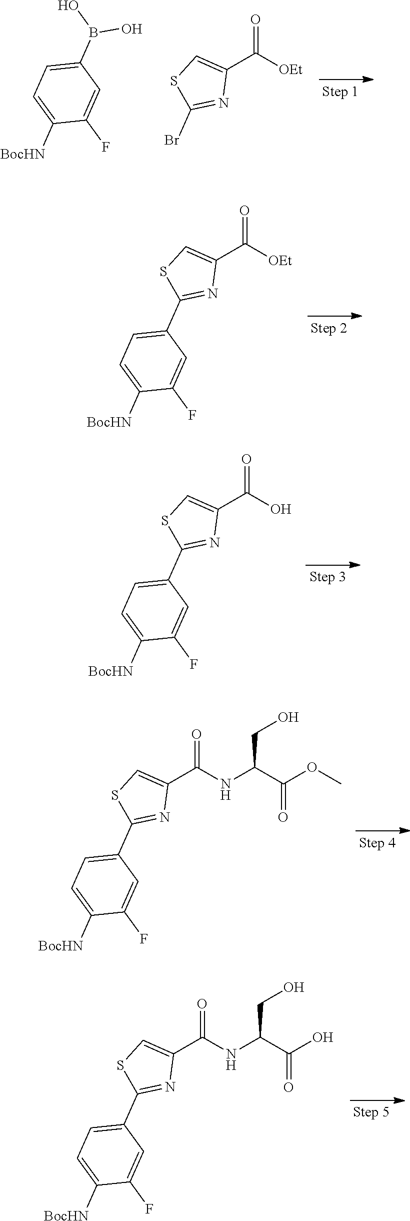 [SMILES: CC(C)(C)OC(=O)Nc1ccc(B(O)O)cc1F ; CCOC(=O)c1csc(Br)n1 ; CCOC(=O)c1csc(-c2ccc(NC(=O)OC(C)(C)C)c(F)c2)n1 ; CC(C)(C)OC(=O)Nc1ccc(-c2nc(C(=O)O)cs2)cc1F ; COC(=O)[C@H](CO)NC(=O)c1csc(-c2ccc(NC(=O)OC(C)(C)C)c(F)c2)n1 ; CC(C)(C)OC(=O)Nc1ccc(-c2nc(C(=O)N[C@@H](CO)C(=O)O)cs2)cc1F]

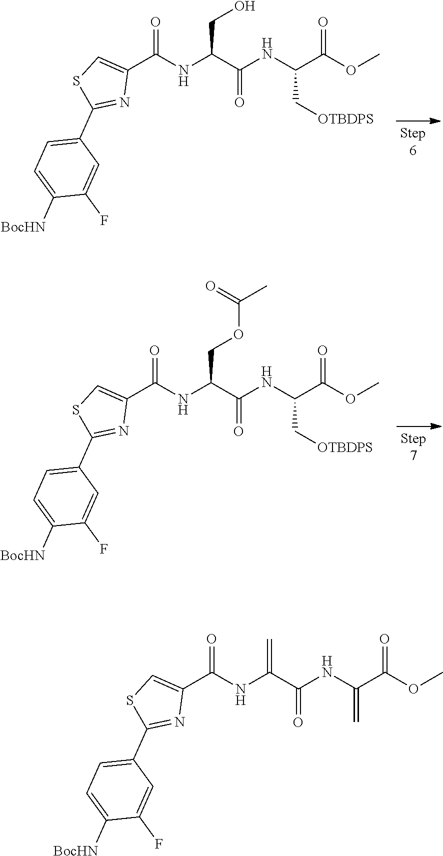

Step 1. Ethyl 2-(4-((tert-butoxycarbonyl)amino)-3-fluorophenyl)thiazole-4-carboxylate was prepared following General experimental procedure 1. Ethyl 2-bromothiazole-4-carboxylate (0.20 g, 0.85 mmol) and (4-((tert-butoxycarbonyl)amino)-3-fluorophenyl)boronic acid (0.20 g, 0.78 mmol) gave ethyl 2-(4-((tert-butoxycarbonyl)amino)-3-fluorophenyl)thiazole-4-carboxylate (0.15 g, 0.42 mmol, 53%) ¹H NMR (400 MHz, CDCl₃) δ 8.20 (t, J=8.2 Hz, 1H), 8.10 (s, 1H), 7.79 (dd, J=11.8, 2.0 Hz, 1H), 7.68 (d, J=8.6 Hz, 1H), 6.82 (s, 1H), 4.43 (q, J=7.1 Hz, 2H), 1.52 (s, 9H), 1.41 (t, J=7.1 Hz, 3H).

Step 2. 2-(4-((tert-butoxycarbonyl)amino)-3-fluorophenyl)thiazole-4-carboxylic acid was prepared following General experimental procedure 2. Ethyl 2-(4-((tert-butoxycarbonyl)amino)-3-fluorophenyl)thiazole-4-carboxylate (0.71 g, 1.9 mmol) gave 2-(4-((tert-butoxycarbonyl)amino)-3-fluorophenyl)thiazole-4-carboxylic acid (0.63 g, 1.9 mmol, 97%). ¹H NMR (400 MHz, CDCl₃) δ 8.25 (d, J=8.3 Hz, 1H), 8.21 (s, 1H), 7.74 (dd, J=11.7, 2.0 Hz, 1H), 7.70-7.63 (m, 1H), 6.86 (d, J=3.6 Hz, 1H), 1.53 (s, 9H).

Step 3. Methyl (2-(4-((tert-butoxycarbonyl)amino)-3-fluorophenyl)thiazole-4-carbonyl)serinate was prepared following General experimental procedure 4. 2-(4-((tert-butoxycarbonyl)amino)-3-fluorophenyl)thiazole-4-carboxylic acid (0.63 g, 1.9 mmol) and methyl L-serinate hydrochloride (0.32 g, 1.1 eq., 2.1 mmol) gave methyl (2-(4-((tert-butoxycarbonyl)amino)-3-fluorophenyl)thiazole-4-carbonyl)serinate (0.87 g, purity 90%, 1.8 mmol, 96%)

Step 4. (2-(4-((tert-butoxycarbonyl)amino)-3-fluorophenyl)thiazole-4-carbonyl)serine was prepared following General experimental procedure 2. Methyl (2-(4-((tert-butoxycarbonyl)amino)-3-fluorophenyl)thiazole-4-carbonyl)serinate (0.87 g, purity 90%, 1.8 mmol) gave (2-(4-((tert-butoxycarbonyl)amino)-3-fluorophenyl)thiazole-4-carbonyl)serine (0.85 g, purity: 90%, 1.8 mmol, quant.) as an off-white solid.

Step 5. Methyl N-((2-(4-((tert-butoxycarbonyl)amino)-3-fluorophenyl)thiazole-4-carbonyl)-L-seryl)-O-(tert-butyldiphenylsilyl)-L-serinaten was prepared following General experimental procedure 4. (2-(4-((tert-butoxycarbonyl)amino)-3-fluorophenyl)thiazole-4-carbonyl)serine (0.76 g, 1.8 mmol) and methyl O-(tert-butyldiphenylsilyl)-L-serinate hydrochloride (0.78 g, 1.1 eq., 2.0 mmol) gave methyl N-((2-(4-((tert-butoxycarbonyl)amino)-3-fluorophenyl)thiazole-4-carbonyl)-L-seryl)-O-(tert-butyldiphenylsilyl)-L-serinate (1.2 g, 1.5 mmol, 84%)

Step 6. Methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)-3-fluorophenyl)thiazole-4-carbonyl)-L-seryl)-O-(tert-butyldiphenylsilyl)-L-serinate (1.2 g, 1.5 mmol) gave methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate (1.1 g, 1.4 mmol, 93%). ¹H NMR (400 MHz, CDCl₃) δ 8.21 (t, J=8.2 Hz, 1H), 8.09 (d, J=7.7 Hz, 1H), 8.04 (s, 1H), 7.72 (dd, J=11.7, 2.0 Hz, 1H), 7.63 (d, J=8.4 Hz, 1H), 7.61-7.50 (m, 5H), 7.44-7.26 (m, 7H), 7.12 (d, J=8.1 Hz, 1H), 6.83 (d, J=3.5 Hz, 1H), 4.95-4.85 (m, 1H), 4.67 (dt, J=8.2, 2.9 Hz, 1H), 4.52 (dd, J=11.3, 5.6 Hz, 1H), 4.39 (dd, J=11.3, 6.2 Hz, 1H), 4.11 (dd, J=10.3, 2.7 Hz, 2H), 3.88 (dd, J=10.3, 3.0 Hz, 1H), 3.73 (s, 3H), 2.08 (s, 3H), 1.53 (s, 9H), 0.96 (s, 9H).

Step 7. Methyl 2-(2-(2-(4-((tert-butoxycarbonyl)amino)-3-fluorophenyl)thiazole-4-carboxamido)acrylamido)acrylate was prepared following General experimental procedure 7. Methyl N—(O-acetyl-N-(2-(4-((tert-butoxycarbonyl)amino)-3-fluorophenyl)thiazole-4-carbonyl)-L-seryl)-O-(tert-butyldiphenylsilyl)-L-serinate (0.30 g, 0.37 mmol) gave methyl 2-(2-(2-(4-((tert-butoxycarbonyl)amino)-3-fluorophenyl)thiazole-4-carboxamido)acrylamido)acrylate (30 mg, 59 μmol, 16%) as a white solid LCMS (22010199A TFA LCMS-5 C1): RT: 1.815 min; Area: 98.1% (215 nm), 96.8% (306 nm); m/z=491.2 [M+H]⁺. ¹H NMR (400 MHz, CDCl₃) δ 9.97 (s, 1H), 8.55 (s, 1H), 8.23 (t, J=8.3 Hz, 1H), 8.12 (s, 1H), 7.81 (dd, J=11.7, 2.0 Hz, 1H), 7.68 (dd, J=8.6, 1.3 Hz, 1H), 6.86 (m, 1H), 6.77 (d, J=2.2 Hz, 1H), 6.72 (s, 1H), 6.04 (d, J=1.4 Hz, 1H), 5.51 (t, J=1.9 Hz, 1H), 3.90 (s, 3H), 1.57-1.49 (m, 9H).

### Example 26: Compound 71

### Methyl 2-(2-(2-(3-((tert-butoxycarbonyl)amino)phenyl)thiazole-4-carboxamido)acrylamido)acrylate

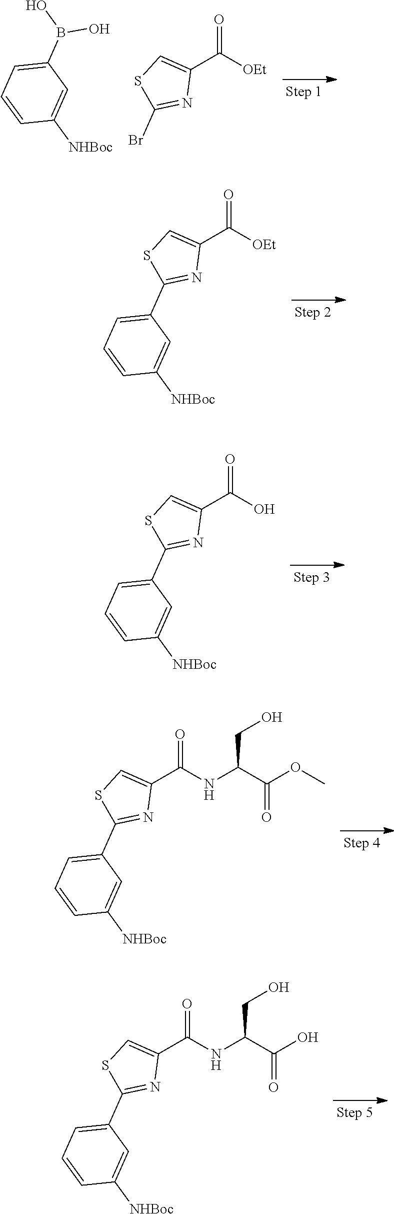 [SMILES: CC(C)(C)OC(=O)Nc1cccc(B(O)O)c1 ; CCOC(=O)c1csc(Br)n1 ; CCOC(=O)c1csc(-c2cccc(NC(=O)OC(C)(C)C)c2)n1 ; CC(C)(C)OC(=O)Nc1cccc(-c2nc(C(=O)O)cs2)c1 ; COC(=O)[C@H](CO)NC(=O)c1csc(-c2cccc(NC(=O)OC(C)(C)C)c2)n1 ; CC(C)(C)OC(=O)Nc1cccc(-c2nc(C(=O)N[C@@H](CO)C(=O)O)cs2)c1]

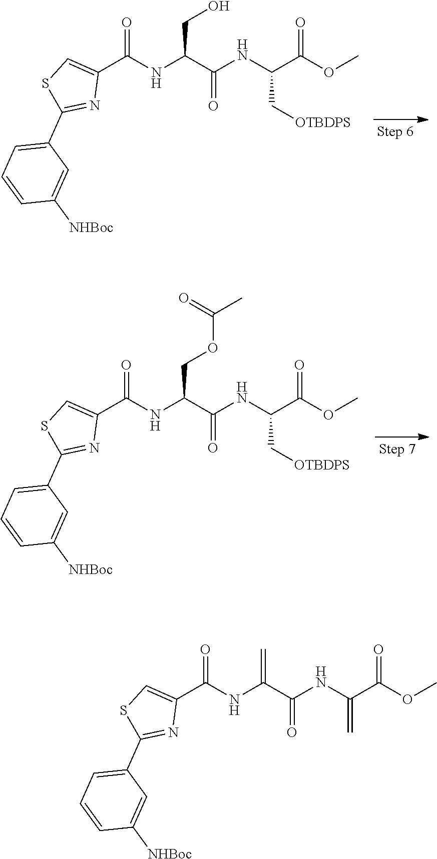

Step 1. Ethyl 2-(3-((tert-butoxycarbonyl)amino)phenyl)thiazole-4-carboxylate was prepared following General experimental procedure 1. Ethyl 2-bromothiazole-4-carboxylate (2.3 g, 1.05 eq., 9.7 mmol) and (3-((tert-butoxycarbonyl)amino)phenyl)boronic acid (2.2 g, 9.3 mmol) gave ethyl 2-(3-((tert-butoxycarbonyl)amino)phenyl)thiazole-4-carboxylate (1.4 g, 4.0 mmol, 43%) as a white solid. ¹H NM/R (400 MHz, CDCl₃) δ 8.13 (s, 1H), 7.94 (t, J=2.0 Hz, 1H), 7.64 (ddd, J=7.7, 1.7, 1.0 Hz, 1H), 7.55 (d, J=8.2 Hz, 1H), 7.35 (t, J=8.0 Hz, 1H), 6.56 (s, 1H), 4.43 (q, J=7.1 Hz, 2H), 1.51 (s, 9H), 1.41 (t, J=7.1 Hz, 3H).

Step 2. 2-(3-((tert-butoxycarbonyl)amino)phenyl)thiazole-4-carboxylic acid was prepared following General experimental procedure 2. Ethyl 2-(3-((tert-butoxycarbonyl)amino)phenyl)thiazole-4-carboxylate (1.4 g, 4.0 mmol) gave 2-(3-((tert-butoxycarbonyl)amino)phenyl)thiazole-4-carboxylic acid (1.3 g, 3.8 mmol, 950%) as a white solid. ¹H NMR (400 MHz, CDCl₃) δ 8.22 (s, 1H), 8.05 (s, 1H), 7.59 (d, J=7.6 Hz, 1H), 7.46 (d, J=8.2 Hz, 1H), 7.37 (t, J=7.9 Hz, 1H), 1.53 (s, 9H).

Step 3. Methyl (2-(3-((tert-butoxycarbonyl)amino)phenyl)thiazole-4-carbonyl)serinate was prepared following General experimental procedure 4. 2-(3-((tert-butoxycarbonyl)amino)phenyl)thiazole-4-carboxylic acid (1.3 g, 3.8 mmol) and methyl L-serinate hydrochloride (0.65 g, 1.1 eq., 4.2 mmol) gave methyl (2-(3-((tert-butoxycarbonyl)amino)phenyl)thiazole-4-carbonyl)serinate (1.5 g, 3.5 mmol, 93%). ¹H NMR (299 MHz, CDCl₃) δ 8.27 (d, J=7.6 Hz, 1H), 8.14 (d, J=1.5 Hz, 1H), 7.98 (d, J=1.9 Hz, 1H), 7.61 (m, 2H), 7.41 (t, J=8.0 Hz, 1H), 7.28 (s, 5H), 6.66 (s, 1H), 5.00-4.84 (m, 1H), 4.12 (m, 2H), 3.87 (s, 3H), 1.57 (s, 9H).

Step 4. (2-(3-((tert-butoxycarbonyl)amino)phenyl)thiazole-4-carbonyl)serine was prepared following General experimental procedure 2. Methyl (2-(3-((tert-butoxycarbonyl)amino)phenyl)thiazole-4-carbonyl)serinate (1.5 g, 3.5 mmol) gave crude (2-(3-((tert-butoxycarbonyl)amino)phenyl)thiazole-4-carbonyl)serine (1.5 g, 3.8 mmol, quant) which was used as such in the next step.

Step 5. Methyl N-((2-(3-((tert-butoxycarbonyl)amino)phenyl)thiazole-4-carbonyl)-L-seryl)-O-(tert-butyldiphenylsilyl)-L-serinate was prepared following General experimental procedure 4. (2-(3-((tert-butoxycarbonyl)amino)phenyl)thiazole-4-carbonyl)serine (0.80 g, 2.0 mmol) and methyl O-(tert-butyldiphenylsilyl)-L-serinate hydrochloride (0.75 g, 1.9 mmol) gave methyl N-((2-(3-((tert-butoxycarbonyl)amino)phenyl)thiazole-4-carbonyl)-L-seryl)-O-(tert-butyldiphenylsilyl)-L-serinate (1.2 g, 1.6 mmol, 79%).

Step 6. Methyl N—(O-acetyl-N-(2-(3-((tert-butoxycarbonyl)amino)phenyl)thiazole-4-carbonyl)-L-seryl)-O-(tert-butyldiphenylsilyl)-L-serinate was prepared following General experimental procedure 5. Methyl N-((2-(3-((tert-butoxycarbonyl)amino)phenyl)thiazole-4-carbonyl)-L-seryl)-O-(tert-butyldiphenylsilyl)-L-serinate (1.2 g, 1.6 mmol) gave methyl N—(O-acetyl-N-(2-(3-((tert-butoxycarbonyl)amino)phenyl)thiazole-4-carbonyl)-L-seryl)-O-(tert-butyldiphenylsilyl)-L-serinate (1.1 g, 1.4 mmol, 89%). ¹H NMR (400 MHz, CDCl₃) δ 8.11 (d, J=7.8 Hz, 1H), 8.07 (s, 1H), 7.90 (t, J=2.0 Hz, 1H), 7.62-7.50 (m, 6H), 7.44-7.26 (m, 8H), 7.13 (d, J=8.2 Hz, 1H), 6.57 (s, 1H), 4.92 (dt, J=7.9, 5.9 Hz, 1H), 4.67 (dt, J=8.1, 2.8 Hz, 1H), 4.51 (dd, J=11.3, 5.7 Hz, 1H), 4.39 (dd, J=11.3, 6.2 Hz, 1H), 4.11 (dd, J=10.3, 2.7 Hz, 1H), 3.87 (dd, J=10.3, 3.0 Hz, 1H), 3.73 (s, 3H), 2.08 (s, 3H), 1.52 (s, 9H), 0.96 (s, 9H).

Step 7. Methyl 2-(2-(2-(3-((tert-butoxycarbonyl)amino)phenyl)thiazole-4-carboxamido)acrylamido)acrylate was prepared following General experimental procedure 7. Methyl N—(O-acetyl-N-(2-(3-((tert-butoxycarbonyl)amino)phenyl)thiazole-4-carbonyl)-L-seryl)-O-(tert-butyldiphenylsilyl)-L-serinate (0.30 g, 0.38 mmol) gave methyl 2-(2-(2-(3-((tert-butoxycarbonyl)amino)phenyl)thiazole-4-carboxamido)acrylamido)acrylate (51 mg, 0.11 mmol, 57%) as a white solid. LCMS (22010199A TFA LCMS-5 C1): RT: 1.704 min; Area: 98.9% (215 nm), 98.6% (306 nm); m/z 473.2 [M+H]⁺. ¹H NMR (400 MHz, CDCl₃) δ 10.00 (s, 1H), 8.55 (s, 1H), 8.15 (s, 1H), 7.93 (t, J=2.0 Hz, 1H), 7.67 (d, J=7.8 Hz, 1H), 7.62 (d, J=8.3 Hz, 1H), 7.40 (t, J=8.0 Hz, 1H), 6.78 (d, J=2.2 Hz, 1H), 6.71 (s, 1H), 6.66 (s, 1H), 6.03 (d, J=1.3 Hz, 1H), 5.51 (t, J=2.0 Hz, 1H), 3.91 (s, 3H), 1.55 (s, 9H).

### Example 27: Compound 72

### Methyl 2-(2-(2-(2-((tert-butoxycarbonyl)amino)pyrimidin-5-yl)thiazole-4-carboxamido)acrylamido)acrylate

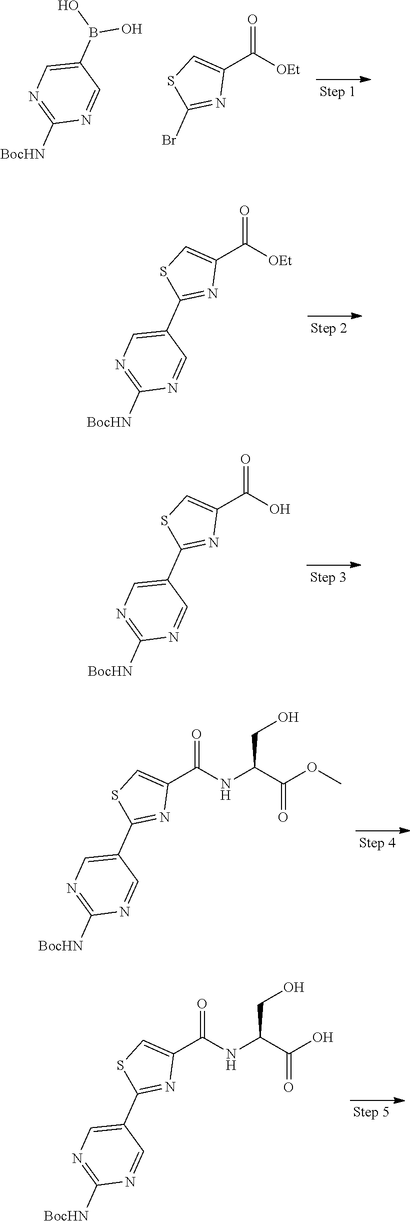 [SMILES: CC(C)(C)OC(=O)Nc1ncc(B(O)O)cn1 ; CCOC(=O)c1csc(Br)n1 ; CCOC(=O)c1csc(-c2cnc(NC(=O)OC(C)(C)C)nc2)n1 ; CC(C)(C)OC(=O)Nc1ncc(-c2nc(C(=O)O)cs2)cn1 ; COC(=O)[C@H](CO)NC(=O)c1csc(-c2cnc(NC(=O)OC(C)(C)C)nc2)n1 ; CC(C)(C)OC(=O)Nc1ncc(-c2nc(C(=O)N[C@@H](CO)C(=O)O)cs2)cn1]

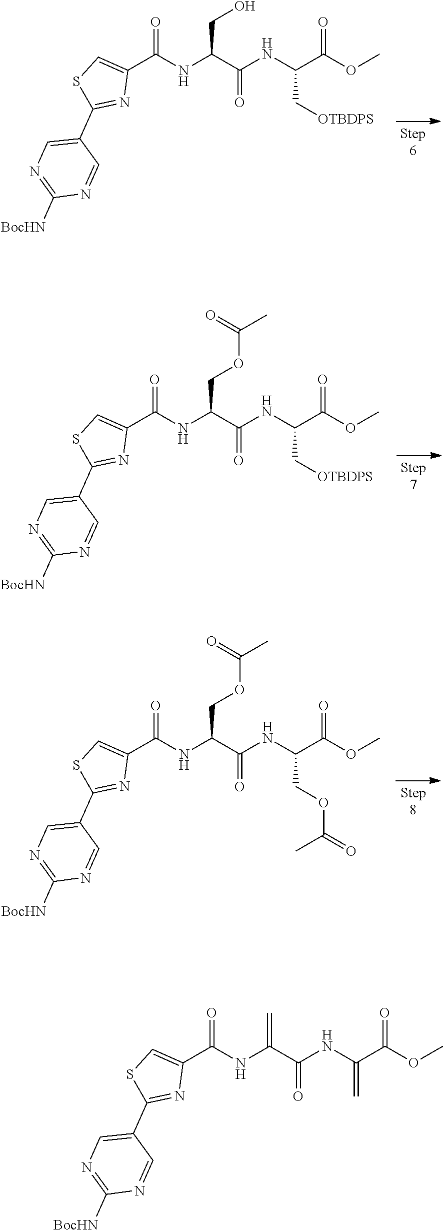

Step 1. Ethyl 2-(2-((tert-butoxycarbonyl)amino)pyrimidin-5-yl)thiazole-4-carboxylate was prepared following General experimental procedure 1. (2-((tert-butoxycarbonyl)amino)pyrimidin-5-yl)boronic acid (1.2 g, 5.2 mmol) and ethyl 2-bromothiazole-4-carboxylate (1.4 g, 1.1 eq. 5.8 mmol) gave ethyl 2-(2-((tert-butoxycarbonyl)amino)pyrimidin-5-yl)thiazole-4-carboxylate (1.1 g, 3.1 mmol, 61%) ¹H NMR (400 MHz, CDCl₃) δ 9.15 (s, 2H), 8.62 (s, 1H), 8.18 (s, 1H), 4.43 (q, J=7.1 Hz, 2H), 1.63 (s, 1H), 1.56 (s, 9H), 1.41 (t, J=7.1 Hz, 3H).

Step 2. 2-(2-((tert-butoxycarbonyl)amino)pyrimidin-5-yl)thiazole-4-carboxylic acid was prepared following General experimental procedure 2. Ethyl 2-(2-((tert-butoxycarbonyl)amino)pyrimidin-5-yl)thiazole-4-carboxylate (2.1 g, 5.9 mmol) gave crude 2-(2-((tert-butoxycarbonyl)amino)pyrimidin-5-yl)thiazole-4-carboxylic acid (2.0 g) which was used without purification in the next step.

Step 3. Methyl (2-(2-((tert-butoxycarbonyl)amino)pyrimidin-5-yl)thiazole-4-carbonyl)serinate was prepared following General experimental procedure 4. 2-(2-((tert-butoxycarbonyl)amino)pyrimidin-5-yl)thiazole-4-carboxylic acid (0.64 g, 2.0 mmol) and methyl L-serinate hydrochloride (0.34 g, 1.1 eq. 2.2 mmol) gave methyl (2-(2-((tert-butoxycarbonyl)amino)pyrimidin-5-yl)thiazole-4-carbonyl)serinate (0.93 g, 2.0 mmol, quant.).

Step 4. (2-(2-((tert-butoxycarbonyl)amino)pyrimidin-5-yl)thiazole-4-carbonyl)serine was prepared following General experimental procedure 2. Methyl (2-(2-((tert-butoxycarbonyl)amino)pyrimidin-5-yl)thiazole-4-carbonyl)serinate (0.84 g, 2.0 mmol) gave (2-(2-((tert-butoxycarbonyl)amino)pyrimidin-5-yl)thiazole-4-carbonyl)serine (0.55 g, 1.3 mmol, 67%). ¹H NMR (400 MHz, DMSO) δ 10.48 (s, 1H), 9.19 (s, 1H), 8.41 (s, 1H), 8.37 (d, J=8.1 Hz, 1H), 4.49 (dt, J=8.3, 4.2 Hz, 1H), 3.84 (m, 2H), 1.48 (s, 9H).

Step 5. Methyl N-((2-(2-((tert-butoxycarbonyl)amino)pyrimidin-5-yl)thiazole-4-carbonyl)-L-seryl)-O-(tert-butyldiphenylsilyl)-L-serinate was prepared following General experimental procedure 4. (2-(2-((tert-butoxycarbonyl)amino)pyrimidin-5-yl)thiazole-4-carbonyl)serine (0.54 g, 1.3 mmol) and methyl O-(tert-butyldiphenylsilyl)-L-serinate hydrochloride (0.57 g, 1.1 eq., 1.4 mmol) gave methyl N-((2-(2-((tert-butoxycarbonyl)amino)pyrimidin-5-yl)thiazole-4-carbonyl)-L-seryl)-O-(tert-butyldiphenylsilyl)-L-serinate (0.42 g, 0.56 mmol, 43%) as an off-white solid. LCMS (General 3 acidic) RT: 1.53 min; area % (254 nm): 76%; m/z=749.7 [M+H]⁺.

Step 6. Methyl N—(O-acetyl-N-(2-(2-((tert-butoxycarbonyl)amino)pyrimidin-5-yl)thiazole-4-carbonyl)-L-seryl)-O-(tert-butyldiphenylsilyl)-L-serinate was prepared following General experimental procedure 5. Methyl N-((2-(2-((tert-butoxycarbonyl)amino)pyrimidin-5-yl)thiazole-4-carbonyl)-L-seryl)-O-(tert-butyldiphenylsilyl)-L-serinate (0.40 g, 0.53 mmol) gave methyl N—(O-acetyl-N-(2-(2-((tert-butoxycarbonyl)amino)pyrimidin-5-yl)thiazole-4-carbonyl)-L-seryl)-O-(tert-butyldiphenylsilyl)-L-serinate (0.24 g, 0.30 mmol, 57%). LCMS (General 3 acidic) RT: 1.63 min; area % (254 nm): 96%; m/z=691.5 [M-Boc+H]⁺.

Step 7. Methyl O-acetyl-N—(O-acetyl-N-(2-(2-((tert-butoxycarbonyl)amino)pyrimidin-5-yl)thiazole-4-carbonyl)-L-seryl)-L-serinate was prepared using the following procedure. To a solution of methyl N—(O-acetyl-N-(2-(2-((tert-butoxycarbonyl)amino)pyrimidin-5-yl)thiazole-4-carbonyl)-L-seryl)-O-(tert-butyldiphenylsilyl)-L-serinate (0.24 g, 0.30 mmol) in THF (5 mL) was added a solution of TBAF in THF (1M, 0.45 mL, 1.5 eq., 0.45 mmol) and the resulting mixture was stirred for 1 hour. The mixture was concentrated and the residue taken up in MeCN (5 mL). Triethylamine (0.10 mL, 2.4 eq., 0.72 mmol) and acetic anhydride (38 μL, 1.3 eq., 0.40 mmol) were added and the resulting mixture was stirred at room temperature for 45 minutes. Water was added and the mixture was extracted with EtOAc. The organic layer was washed with brine, dried over Na₂SO₄, filtered, and concentrated. The crude material was purified by automated FCC to provide methyl O-acetyl-N—(O-acetyl-N-(2-(2-((tert-butoxycarbonyl)amino)pyrimidin-5-yl)thiazole-4-carbonyl)-L-seryl)-L-serinate (87 mg, 0.15 mmol, 48%).

Step 8. Methyl 2-(2-(2-(2-((tert-butoxycarbonyl)amino)pyrimidin-5-yl)thiazole-4-carboxamido)acrylamido)acrylate was prepared using the following procedure. To a solution of methyl O-acetyl-N—(O-acetyl-N-(2-(2-((tert-butoxycarbonyl)amino)pyrimidin-5-yl)thiazole-4-carbonyl)-L-seryl)-L-serinate (86 mg, 0.14 mmol) in CH₂Cl₂ (1.5 mL) and THF (1.5 mL) was added at 0° C. DBU (87 μL, 4 eq., 0.58 mmol) and the mixture was stirred at 0° C. for 2 hours. Water was added and the aqueous phase was adjusted to pH 4 by addition of 1M HCl. The mixture was extracted with CH₂Cl₂ and the organic layer was washed brine, dried over Na₂SO₄, filtered and concentrated. The crude material was purified by automated reverse phase FCC to provide methyl 2-(2-(2-(2-((tert-butoxycarbonyl)amino)pyrimidin-5-yl)thiazole-4-carboxamido)acrylamido)acrylate (27 mg, 57 μmol, 39%). LCMS (22010199A TFA LCMS-5 C1): RT: 1.460 min; Area: 96.8% (215 nm), 96.2% (306 nm); m/z 473.0 [M−H]⁻. H NMR (400 MHz, CDCl₃) δ 10.01 (s, 1H), 9.17 (s, 2H), 8.54 (s, 1H), 8.20 (s, 1H), 7.96 (s, 1H), 6.77 (d, J 2.3 Hz, 1H), 6.70 (s, 1H), 6.03 (d, J=1.3 Hz, 1H), 5.51 (t, J=1.9 Hz, 1H), 3.90 (s, 3H), 1.57 (s, 9H).

### Example 28: Compound 75

### Methyl 2-(2-(4-(4-((tert-butoxycarbonyl)amino)phenyl)thiazole-2-carboxamido)acrylamido)acrylate

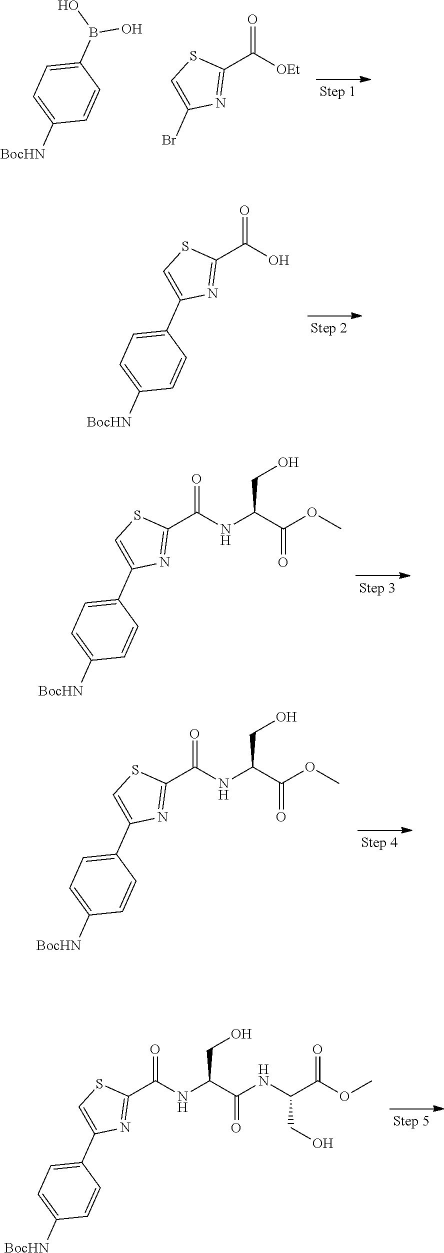 [SMILES: CC(C)(C)OC(=O)Nc1ccc(B(O)O)cc1 ; CCOC(=O)c1nc(Br)cs1 ; CC(C)(C)OC(=O)Nc1ccc(-c2csc(C(=O)O)n2)cc1 ; COC(=O)[C@H](CO)NC(=O)c1nc(-c2ccc(NC(=O)OC(C)(C)C)cc2)cs1 ; COC(=O)[C@H](CO)NC(=O)c1nc(-c2ccc(NC(=O)OC(C)(C)C)cc2)cs1 ; COC(=O)[C@H](CO)NC(=O)[C@H](CO)NC(=O)c1nc(-c2ccc(NC(=O)OC(C)(C)C)cc2)cs1]

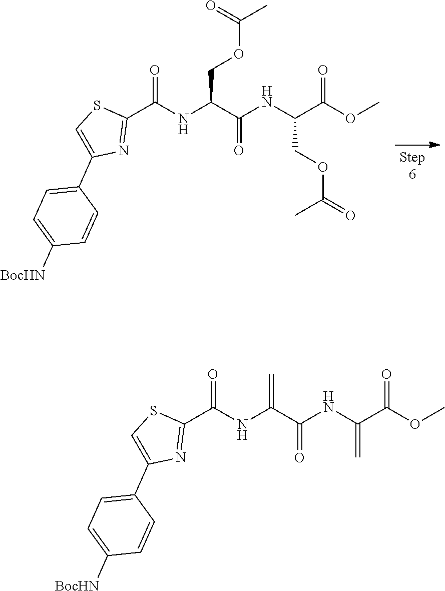 [SMILES: COC(=O)[C@H](COC(C)=O)NC(=O)[C@H](COC(C)=O)NC(=O)c1nc(-c2ccc(NC(=O)OC(C)(C)C)cc2)cs1 ; C=C(NC(=O)c1nc(-c2ccc(NC(=O)OC(C)(C)C)cc2)cs1)C(=O)NC(=C)C(=O)OC]

Step 1. 4-(4-((tert-butoxycarbonyl)amino)phenyl)thiazole-2-carboxylic acid was prepared using the following procedure. To a solution of ethyl 4-bromothiazole-2-carboxylate (1.0 g, 4.2 mmol) and (4-((tert-butoxycarbonyl)amino)phenyl)boronic acid in 1,2-Dimethoxyethane (25 mL) were added sodium carbonate (aq. 2 M, 10 eq. 42 mmol) and palladium tetrakis (0.24 g, 0.05 eq., 0.21 mmol) and the resulting mixture was stirred at 80° C. for 16 hours. The mixture was cooled to room temperature, diluted with ethyl acetate and washed with water. The aqueous phase was acidified with 1M HCl solution and extracted with ethyl acetate. The organic layer was dried over Na₂SO₄, filtered, and concentrated under reduced pressure to afford 4-(4-((tert-butoxycarbonyl)amino)phenyl)thiazole-2-carboxylic acid (1.1 g, purity 43%, 1.4 mmol, 35%) as a mixture with the decarboxylated product. The mixture was used as such in the next step.

Step 2. Methyl (4-(4-((tert-butoxycarbonyl)amino)phenyl)thiazole-2-carbonyl)-L-serinate was prepared following General experimental procedure 4. Crude 4-(4-((tert-butoxycarbonyl)amino)phenyl)thiazole-2-carboxylic acid (1.1 g, purity 43%, 1.4 mmol) and methyl L-serinate hydrochloride (0.26 g, 1.2 eq. 1.7 mmol) gave methyl (4-(4-((tert-butoxycarbonyl)amino)phenyl)thiazole-2-carbonyl)-L-serinate (0.61 g 1.45 mmol, quant.).

Step 3. (4-(4-((tert-butoxycarbonyl)amino)phenyl)thiazole-2-carbonyl)-L-serine was prepared following General experimental procedure 2. Methyl (4-(4-((tert-butoxycarbonyl)amino)phenyl)thiazole-2-carbonyl)-L-serinate (0.60 g, 1.4 mmol) gave (4-(4-((tert-butoxycarbonyl)amino)phenyl)thiazole-2-carbonyl)-L-serine (0.59 g, 1.4 mmol, quant).

Step 4. Methyl (4-(4-((tert-butoxycarbonyl)amino)phenyl)thiazole-2-carbonyl)-L-seryl-L-serinate was prepared following General experimental procedure 4. (4-(4-((tert-butoxycarbonyl)amino)phenyl)thiazole-2-carbonyl)-L-serine (0.58 g, 1.4 mmol) and methyl L-serinate hydrochloride (0.27 g, 1.2 eq., 1.7 mmol) gave methyl (4-(4-((tert-butoxycarbonyl)amino)phenyl)thiazole-2-carbonyl)-L-seryl-L-serinate (0.52 g, 1.0 mmol, 72%). LCMS (General 3 acidic) RT: 0.94 min; area % (254 nm): 91%; m/z=509.4 [M+H]⁺.

Step 5. Methyl O-acetyl-N—(O-acetyl-N-(4-(4-((tert-butoxycarbonyl)amino)phenyl)thiazole-2-carbonyl)-L-seryl)-L-serinate was prepared following General experimental procedure 5. Methyl (4-(4-((tert-butoxycarbonyl)amino)phenyl)thiazole-2-carbonyl)-L-seryl-L-serinate (0.52 g, 1.0 mmol) and acetic anhydride (0.25 mL, 4 eq., 2.7 mmol) gave methyl O-acetyl-N—(O-acetyl-N-(4-(4-((tert-butoxycarbonyl)amino)phenyl)thiazole-2-carbonyl)-L-seryl)-L-serinate (0.33 g, 0.56 mmol, 55%)

Step 6. Methyl 2-(2-(4-(4-((tert-butoxycarbonyl)amino)phenyl)thiazole-2-carboxamido)acrylamido)acrylate was prepared following General experimental procedure 8. Methyl O-acetyl-N—(O-acetyl-N-(4-(4-((tert-butoxycarbonyl)amino)phenyl)thiazole-2-carbonyl)-L-seryl)-L-serinate (0.35 g, 0.59 mmol) gave methyl 2-(2-(4-(4-((tert-butoxycarbonyl)amino)phenyl)thiazole-2-carboxamido)acrylamido)acrylate (85 mg, 0.18 mmol, 30%) as a white solid. LC-MS (22010199A TFA LCMS-5 C1): RT: 1.731 min; Area 98.6% (215 nm), 97.8% (304 nm); m/z=473.2 [M+H]⁺. ¹H NMR (400 MHz, CDCl₃) δ (ppm) 9.92 (s, 1H), 8.55 (s, 1H), 7.89 (d, J=8.7 Hz, 2H), 7.67 (s, 1H), 7.45 (d, J=8.6 Hz, 2H), 6.76 (d, J=2.4 Hz, 1H), 6.71 (s, 1H), 6.59 (s, 1H), 6.04 (d, J=1.0 Hz, 1H), 5.54 (dd, J=2.3 Hz, J=1.6 Hz, 1H), 3.90 (s, 3H), 1.54 (s, 9H).

### Example 29: Compound 76

### Methyl 2-(2-(2-(4-((tert-butoxycarbonyl)amino)-2-chlorophenyl)thiazole-4-carboxamido)acrylamido)acrylate

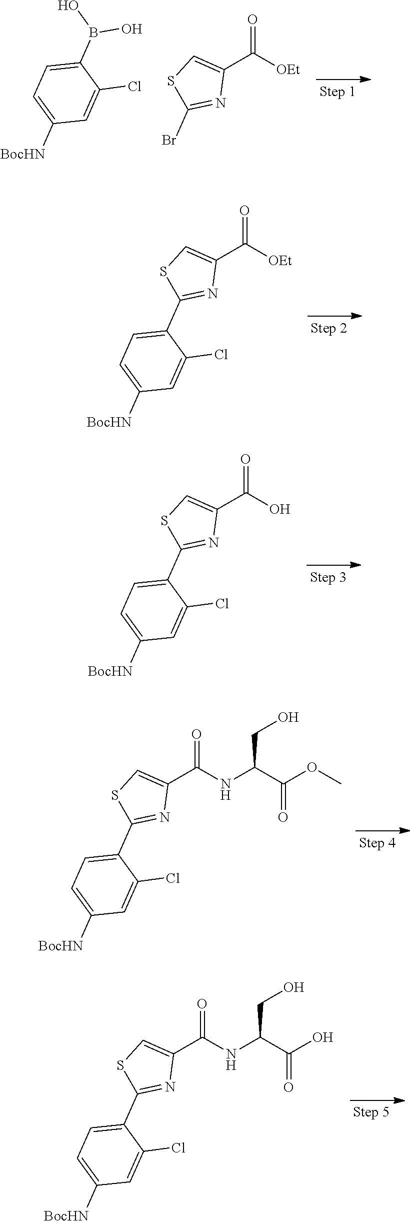

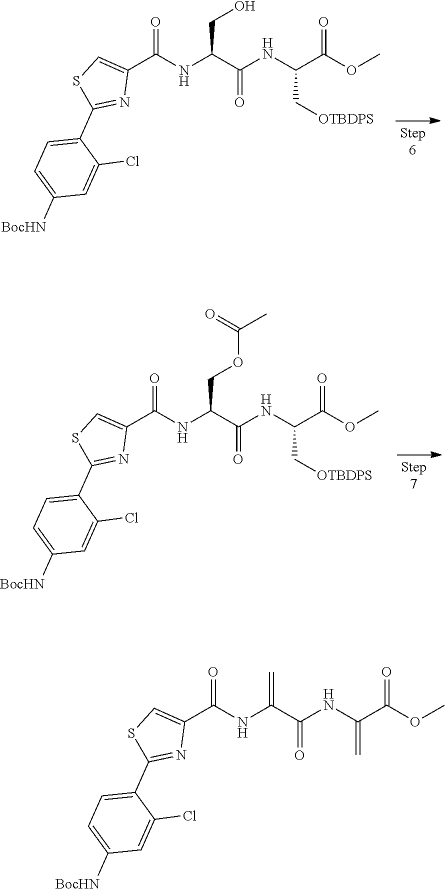

Step 1. 2-(4-((tert-butoxycarbonyl)amino)-2-chlorophenyl)thiazole-4-carboxylate was prepared following General experimental procedure 1. Ethyl 2-bromothiazole-4-carboxylate (0.47 g, 2.0 mmol) and tert-butyl (3-chloro-4-(4,4,5,5-tetramethyl-1,3,2-dioxaborolan-2-yl)phenyl)carbamate (0.70 g, 2.0 mmol) gave ethyl 2-(4-((tert-butoxycarbonyl)amino)-2-chlorophenyl)thiazole-4-carboxylate (0.38 g, 1.0 mmol, 51%). LCMS (General 3 acidic) RT: 1.58 min; area (254 nm): 96%; m/z=383.2 [M+H]⁺.

Step 2. 2-(4-((tert-butoxycarbonyl)amino)-2-chlorophenyl)thiazole-4-carboxylic acid was prepared following General experimental procedure 2. Ethyl 2-(4-((tert-butoxycarbonyl)amino)-2-chlorophenyl)thiazole-4-carboxylate (0.38 g, 0.99 mmol) gave 2-(4-((tert-butoxycarbonyl)amino)-2-chlorophenyl)thiazole-4-carboxylic acid (0.30 g, 0.84 mmol, 85%)¹H NMR (299 MHz, CDCl₃) δ 8.36 (s, 1H), 8.25 (d, J=8.9 Hz, 1H), 7.82 (s, 1H), 7.31-7.22 (m, 2H), 6.78 (s, 1H), 1.56 (s, 9H).

Step 3. Methyl (2-(4-((tert-butoxycarbonyl)amino)-2-chlorophenyl)thiazole-4-carbonyl)-L-serinate was prepared following General experimental procedure 4. 2-(4-((tert-butoxycarbonyl)amino)-2-chlorophenyl)thiazole-4-carboxylic acid (0.44 g, 1.2 mmol) and methyl L-serinate hydrochloride (0.23 g, 1.2 eq., 1.5 mmol) gave methyl (2-(4-((tert-butoxycarbonyl)amino)-2-chlorophenyl)thiazole-4-carbonyl)-L-serinate (0.42 g, 0.92 mmol, 75%) %). LCMS (General 3 acidic) RT: 1.23 min; area % (254 nm): 93%; m/z=456.3 [M+H]⁺.

Step 4. (2-(4-((tert-butoxycarbonyl)amino)-2-chlorophenyl)thiazole-4-carbonyl)-L-serine was prepared following. General experimental procedure 2. Methyl (2-(4-((tert-butoxycarbonyl)amino)-2-chlorophenyl)thiazole-4-carbonyl)-L-serinate (0.42 g, 0.92 mmol) gave (2-(4-((tert-butoxycarbonyl)amino)-2-chlorophenyl)thiazole-4-carbonyl)-L-serine (0.33 g, 0.75 mmol, 81%) as an orange solid.

Step 5. Methyl N-((2-(4-((tert-butoxycarbonyl)amino)-2-chlorophenyl)thiazole-4-carbonyl)-L-seryl)-O-(tert-butyldiphenylsilyl)-L-serinate was prepared following General experimental procedure 4. (2-(4-((tert-butoxycarbonyl)amino)-2-chlorophenyl)thiazole-4-carbonyl)-L-serine (0.33 g, 0.75 mmol) and methyl O-(tert-butyldiphenylsilyl)-L-serinate hydrochloride (0.35 g, 1.2 eq., 0.90 mmol) gave methyl N-((2-(4-((tert-butoxycarbonyl)amino)-2-chlorophenyl)thiazole-4-carbonyl)-L-seryl)-O-(tert-butyldiphenylsilyl)-L-serinate (0.37 g, 0.48 mmol, 64%).

Step 6. Methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate was prepared following General experimental procedure 5. Methyl N-((2-(4-((tert-butoxycarbonyl)amino)-2-chlorophenyl)thiazole-4-carbonyl)-L-seryl)-O-(tert-butyldiphenylsilyl)-L-serinate (0.37 g, 0.48 mmol) and acetic anhydride (68 μL, 1.5 eq. 0.72 mmol) gave methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate (0.22 g, 0.27 mmol, 57%). ¹H NMR (299 MHz, CDCl₃) δ 8.28-8.12 (m, 3H), 7.85 (s, 1H), 7.57 (m, 4H), 7.46-7.30 (m, 6H), 7.19 (m, 2H), 6.66 (s, 1H), 4.95 (q, J=6.3 Hz, 1H), 4.70 (d, J=8.1 Hz, 1H), 4.56 (dd, J=11.3, 5.7 Hz, 1H), 4.43 (dd, J=11.3, 6.2 Hz, 1H), 4.14 (d, J=8.1 Hz, 1H), 3.96-3.86 (m, 1H), 3.77 (d, J=1.2 Hz, 3H), 2.11 (s, 3H), 1.56 (s, 9H), 1.00 (s, 9H).

Step 7. Methyl 2-(2-(2-(4-((tert-butoxycarbonyl)amino)-2-chlorophenyl)thiazole-4-carboxamido)acrylamido)acrylate was prepared following General experimental procedure 7. Methyl N—(O-acetyl-N-(2-(4-((tert-butoxycarbonyl)amino)-2-chlorophenyl)thiazole-4-carbonyl)-L-seryl)-O-(tert-butyldiphenylsilyl)-L-serinate (0.22 g, 0.27 mmol) gave methyl 2-(2-(2-(4-((tert-butoxycarbonyl)amino)-2-chlorophenyl)thiazole-4-carboxamido)acrylamido)acrylate (15 mg, 30 μmol, 29%) as a white solid. LCMS (22010199A TFA LCMS-5 C1): rt 1.880 min; Area 96.5% (215 nm), 95.6% (304 nm); m/z=507.2 [M+H]⁺. ¹H NMR (400 MHz, CDCl₃) δ (ppm) 10.02 (s, 1H), 8.55 (s, 1H), 8.31 (d, J=8.7 Hz, 1H), 8.23 (s, 1H), 7.84 (d, J=1.8 Hz, 1H), 7.21 (dd, J=8.7 Hz, J=2.3 Hz, 1H), 6.78 (d, J=2.2 Hz, 1H), 6.70 (s, 1H), 6.66 (s, 1H), 6.02 (d, J=1.1 Hz, 1H), 5.48 (t, J=1.8 Hz, 1H), 3.90 (s, 3H), 1.54 (s, 9H).

### Example 30: Compound 77

### Methyl 2-(2-(4′-((tert-butoxycarbonyl)amino)-[1,1′-biphenyl]-3-carboxamido)acrylamido)acrylate

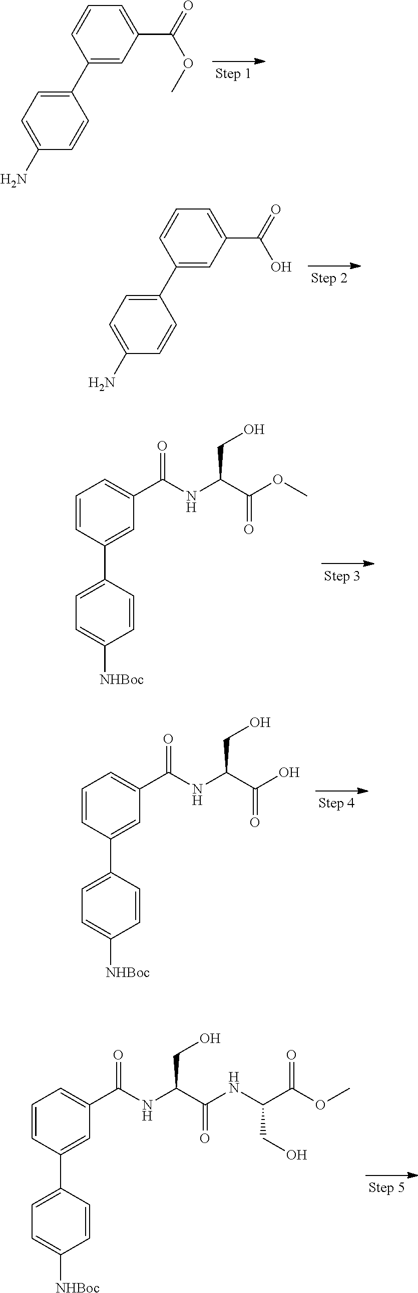 [SMILES: COC(=O)c1cccc(-c2ccc(N)cc2)c1 ; Nc1ccc(-c2cccc(C(=O)O)c2)cc1 ; COC(=O)[C@H](CO)NC(=O)c1cccc(-c2ccc(NC(=O)OC(C)(C)C)cc2)c1 ; CC(C)(C)OC(=O)Nc1ccc(-c2cccc(C(=O)N[C@@H](CO)C(=O)O)c2)cc1 ; COC(=O)[C@H](CO)NC(=O)[C@H](CO)NC(=O)c1cccc(-c2ccc(NC(=O)OC(C)(C)C)cc2)c1]

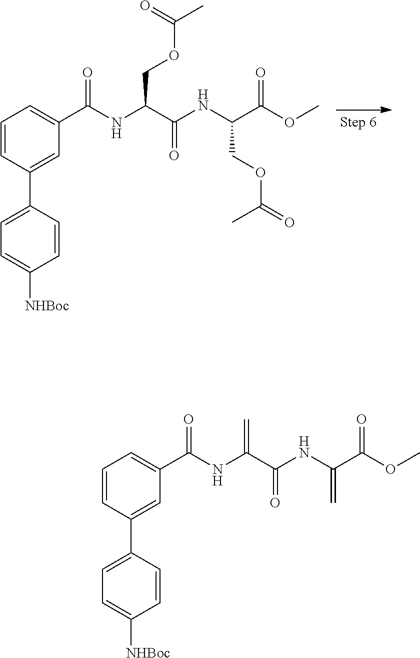 [SMILES: COC(=O)[C@H](COC(C)=O)NC(=O)[C@H](COC(C)=O)NC(=O)c1cccc(-c2ccc(NC(=O)OC(C)(C)C)cc2)c1 ; C=C(NC(=O)c1cccc(-c2ccc(NC(=O)OC(C)(C)C)cc2)c1)C(=O)NC(=C)C(=O)OC]

Step 1. 4′-((tert-butoxycarbonyl)amino)-[1,1′-biphenyl]-3-carboxylic acid was prepared using the following procedure. To a solution of methyl 4′-amino-[1,1′-biphenyl]-3-carboxylate (0.50 g, 2.2 mmol) in CH₂Cl₂ (20 mL) was added DMAP (0.54 g, 2 eq., 4.4 mmol). Then Boc₂O (0.72 g, 1.5 eq., 3.3 mmol) was added portionwise and the mixture was stirred over 16 hours at room temperature. The mixture was washed with water and brine, dried over Na₂SO₄ and concentrated in vacuo. The crude material was dissolved in THF (20 mL) and a solution of NaOH (aq., 1M, 7 eq. 15.4 mL, 15 mmol) was added and the resulting mixture was stirred for 64 hours. The mixture was quenched with 1M HCl, extracted with EtOAc. The organic phase was dried over Na₂SO₄, filtered, and concentrated in vacuo to provide 4′-((tert-butoxycarbonyl)amino)-[1,1′-biphenyl]-3-carboxylic acid (0.47 g, 1.5 mmol, 68%) as a yellow solid.

Step 2. Methyl (4′-((tert-butoxycarbonyl)amino)-[1,1′-biphenyl]-3-carbonyl)-L-serinate was prepared following General experimental procedure 4. 4′-((tert-butoxycarbonyl)amino)-[1,1′-biphenyl]-3-carboxylic acid (0.45 g, 1.4 mmol) and methyl L-serinate hydrochloride (0.27 g, 1.2 eq. 1.7 mmol) gave crude methyl (4′-((tert-butoxycarbonyl)amino)-[1,1′-biphenyl]-3-carbonyl)-L-serinate (0.60 g) which was used as such in the next step.

Step 3. (4′-((tert-butoxycarbonyl)amino)-[1,1′-biphenyl]-3-carbonyl)-L-serine was prepared following General experimental procedure 2. Methyl (4′-((tert-butoxycarbonyl)amino)-[1,1′-biphenyl]-3-carbonyl)-L-serinate (0.58 g, 1.4 mmol) gave (4′-((tert-butoxycarbonyl)amino)-[1,1′-biphenyl]-3-carbonyl)-L-serine (0.37 g, 0.92 mmol, 66% over 2 steps) as a yellow oil. LCMS (General 3 acidic) RT: 0.97 min; area % (254 nm): 90%; m/z=401.3 [M+H]⁺.

Step 4. Methyl (4′-((tert-butoxycarbonyl)amino)-[1,1′-biphenyl]-3-carbonyl)-L-seryl-L-serinate was prepared following General experimental procedure 4. (4′-((tert-butoxycarbonyl)amino)-[1,1′-biphenyl]-3-carbonyl)-L-serine (0.20 g, 0.50 mmol) and methyl L-serinate hydrochloride (0.93 mg, 1.2 eq. 0.60 mmol) gave crude methyl (4′-((tert-butoxycarbonyl)amino)-[1,1′-biphenyl]-3-carbonyl)-L-seryl-L-serinate (0.16 g) which was used as such in the next step. LCMS (General 3 acidic) RT: 0.95 min; area % (254 nm): 84%; m/z=502.4 [M+H]⁺.

Step 5. Methyl O-acetyl-N—(O-acetyl-N-(4′-((tert-butoxycarbonyl)amino)-[1,1′-biphenyl]-3-carbonyl)-L-seryl)-L-serinate was prepared following General experimental procedure 5. Crude methyl (4′-((tert-butoxycarbonyl)amino)-[1,1′-biphenyl]-3-carbonyl)-L-seryl-L-serinate (0.16 g) and acetic anhydride (0.12 mL, 2.6 eq. 1.3 mmol) gave methyl O-acetyl-N-(0-acetyl-N-(4′-((tert-butoxycarbonyl)amino)-[1,1′-biphenyl]-3-carbonyl)-L-seryl)-L-serinate (130 mg, 0.22 mmol, 44%) and mono-eliminated product methyl O-acetyl-N-(2-(4′-((tert-butoxycarbonyl)amino)-[1,1′-biphenyl]-3-carboxamido)acryloyl)-L-serinate (0.10 g, 0.19 mmol, 38%)

Step 6. Methyl 2-(2-(4′-((tert-butoxycarbonyl)amino)-[1,1′-biphenyl]-3-carboxamido)acrylamido)acrylate was prepared following General experimental procedure 8. A mixture of methyl O-acetyl-N—(O-acetyl-N-(4′-((tert-butoxycarbonyl)amino)-[1,1′-biphenyl]-3-carbonyl)-L-seryl)-L-serinate (130 mg, 0.22 mmol) and methyl O-acetyl-N-(2-(4′-((tert-butoxycarbonyl)amino)-[1,1′-biphenyl]-3-carboxamido)acryloyl)-L-serinate (0.10 g, 0.19 mmol) as prepared in step 5 gave methyl 2-(2-(4′-((tert-butoxycarbonyl)amino)-[1,1′-biphenyl]-3-carboxamido)acrylamido)acrylate (6.6 mg, 14 μmol) as a white solid. LCMS (22010199A TFA LCMS-5 C1): RT: 1.468 min; Area 98.0% (215 nm), 97.2% (304 nm); m/z=466.2 [M+H]⁺. ¹H NMR (400 MHz, CDCl₃) δ 8.95 (s, 1H), 8.61 (s, 1H), 8.08 (t, J=1.9 Hz, 1H), 7.78 (ddt, J=15.4, 7.9, 1.3 Hz, 2H), 7.62-7.45 (m, 6H), 6.85 (d, J=2.3 Hz, 1H), 6.68 (s, 1H), 6.60 (s, 1H), 6.05 (d, J=1.4 Hz, 1H), 5.53 (t, J=1.8 Hz, 1H), 3.93 (s, 3H), 1.56 (s, 9H).

### Example 31: Compound 78

### methyl 2-(2-(2-(3-((tert-butoxycarbonyl)amino)bicyclo[1.1.1]pentan-1-yl)thiazole-4-carboxamido)acrylamido)acrylate

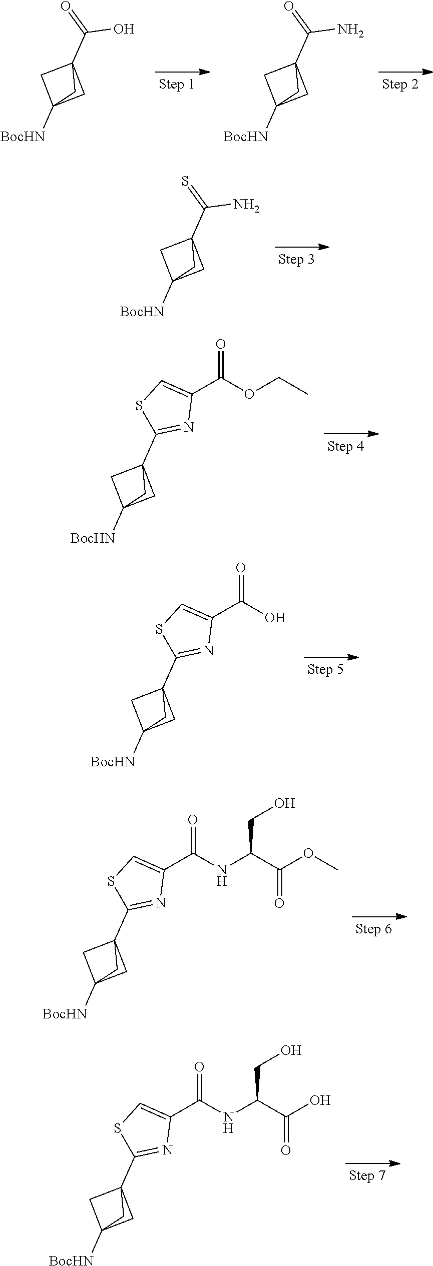

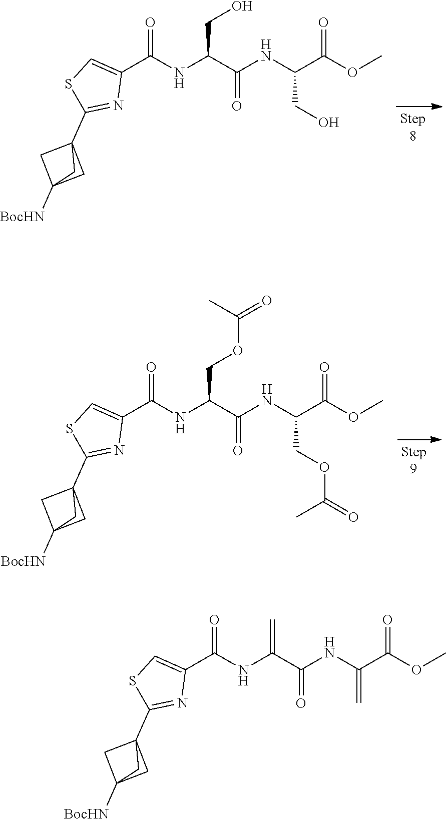 [SMILES: COC(=O)[C@H](CO)NC(=O)[C@H](CO)NC(=O)c1csc(C23CC(NC(=O)OC(C)(C)C)(C2)C3)n1 ; COC(=O)[C@H](COC(C)=O)NC(=O)[C@H](COC(C)=O)NC(=O)c1csc(C23CC(NC(=O)OC(C)(C)C)(C2)C3)n1 ; C=C(NC(=O)c1csc(C23CC(NC(=O)OC(C)(C)C)(C2)C3)n1)C(=O)NC(=C)C(=O)OC]

Step 1. tert-butyl (3-carbamoylbicyclo[1.1.1]pentan-1-yl)carbamate was prepared following General experimental procedure 9. 3-((tert-butoxycarbonyl)amino)bicyclo[1.1.1]pentane-1-carboxylic acid (2.0 g, 8.8 mmol) gave tert-butyl (3-carbamoylbicyclo[1.1.1]pentan-1-yl)carbamate (1.8 g, 8.1 mmol, 92%) as a white solid.

Step 2. tert-butyl (3-carbamothioylbicyclo[1.1.1]pentan-1-yl)carbamate was prepared following General experimental procedure 10. tert-butyl (3-carbamoylbicyclo[1.1.1]pentan-1-yl)carbamate (1.8 g, 8.1 mmol) gave tert-butyl (3-carbamothioylbicyclo[1.1.1]pentan-1-yl)carbamate (1.5 g, 6.4 mmol, 78%) LCMS (General 3 acidic) RT: 0.79 min; area % (254 nm): 100%

Step 3. Ethyl 2-(3-((tert-butoxycarbonyl)amino)bicyclo[1.1.1]pentan-1-yl)thiazole-4-carboxylate was prepared following General experimental procedure 11. tert-butyl (3-carbamothioylbicyclo[1.1.1]pentan-1-yl)carbamate (1.5 g, 6.1 mmol) gave ethyl 2-(3-((tert-butoxycarbonyl)amino)bicyclo[1.1.1]pentan-1-yl)thiazole-4-carboxylate (1.8 g, 5.4 mmol, 88%). ¹H NMR (400 MHz, CDCl₃) δ 8.06 (s, 1H), 5.00 (s, 1H), 4.41 (q, J=7.1 Hz, 2H), 2.49 (s, 6H), 1.46 (s, 9H), 1.39 (t, J=7.1 Hz, 3H).

Step 4. 2-(3-((tert-butoxycarbonyl)amino)bicyclo[1.1.1]pentan-1-yl)thiazole-4-carboxylic acid was prepared following General experimental procedure 2. Ethyl 2-(3-((tert-butoxycarbonyl)amino)bicyclo[1.1.1]pentan-1-yl)thiazole-4-carboxylate (1.8 g, 5.4 mmol) gave 2-(3-((tert-butoxycarbonyl)amino)bicyclo[1.1.1]pentan-1-yl)thiazole-4-carboxylic acid (1.7 g, 5.4 mmol, quant.)

Step 5. Methyl (2-(3-((tert-butoxycarbonyl)amino)bicyclo[1.1.1]pentan-1-yl)thiazole-4-carbonyl)-L-serinate was prepared following General experimental procedure 4. 2-(3-((tert-butoxycarbonyl)amino)bicyclo[1.1.1]pentan-1-yl)thiazole-4-carboxylic acid (1.7 g, 5.4 mmol) and methyl L-serinate hydrochloride (0.92 g, 1.1 eq., 5.9 mmol) gave methyl (2-(3-((tert-butoxycarbonyl)amino)bicyclo[1.1.1]pentan-1-yl)thiazole-4-carbonyl)-L-serinate (1.6 g, 90% purity, 3.6 mmol, 67%) as a white solid. ¹H NMR (400 MHz, CDCl₃) δ 8.07 (d, J=7.7 Hz, 1H), 8.00 (s, 1H), 4.83 (m, 1H), 4.13-3.98 (m, 2H), 3.81 (s, 3H), 2.44 (s, 6H), 1.45 (s, 9H).

Step 6. (2-(3-((tert-butoxycarbonyl)amino)bicyclo[1.1.1]pentan-1-yl)thiazole-4-carbonyl)-L-serine was prepared following General experimental procedure 2. Methyl (2-(3-((tert-butoxycarbonyl)amino)bicyclo[1.1.1]pentan-1-yl)thiazole-4-carbonyl)-L-serinate (1.6 g, 90% purity, 3.6 mmol) gave (2-(3-((tert-butoxycarbonyl)amino)bicyclo[1.1.1]pentan-1-yl)thiazole-4-carbonyl)-L-serine (1.5 g, 85% purity, 3.2 mmol, 89%)

Step 7. Methyl (2-(3-((tert-butoxycarbonyl)amino)bicyclo[1.1.1]pentan-1-yl)thiazole-4-carbonyl)-L-seryl-L-serinate was prepared following General experimental procedure 4. (2-(3-((tert-butoxycarbonyl)amino)bicyclo[1.1.1]pentan-1-yl)thiazole-4-carbonyl)-L-serine (1.5 g, 3.2 mmol) and methyl L-serinate hydrochloride (0.70 g, 1.4 eq., 4.5 mmol) gave methyl (2-(3-((tert-butoxycarbonyl)amino)bicyclo[1.1.1]pentan-1-yl)thiazole-4-carbonyl)-L-seryl-L-serinate (1.6 g, 3.2 mmol, 98%)

Step 8. Methyl O-acetyl-N—(O-acetyl-N-(2-(3-((tert-butoxycarbonyl)amino)bicyclo[1.1.1]pentan-1-yl)thiazole-4-carbonyl)-L-seryl)-L-serinate was prepared following General experimental procedure 5. Methyl (2-(3-((tert-butoxycarbonyl)amino)bicyclo[1.1.1]pentan-1-yl)thiazole-4-carbonyl)-L-seryl-L-serinate (1.6 g, 3.2 mmol) gave methyl O-acetyl-N—(O-acetyl-N-(2-(3-((tert-butoxycarbonyl)amino)bicyclo[1.1.1]pentan-1-yl)thiazole-4-carbonyl)-L-seryl)-L-serinate (1.2 g, 2.1 mmol, 68%) as a white solid.

Step 9. Methyl 2-(2-(2-(3-((tert-butoxycarbonyl)amino)bicyclo[1.1.1]pentan-1-yl)thiazole-4-carboxamido)acrylamido)acrylate was prepared following General experimental procedure 8. Methyl O-acetyl-N—(O-acetyl-N-(2-(3-((tert-butoxycarbonyl)amino)bicyclo[1.1.1]pentan-1-yl)thiazole-4-carbonyl)-L-seryl)-L-serinate (0.17 g, 0.32 mmol) gave methyl 2-(2-(2-(3-((tert-butoxycarbonyl)amino)bicyclo[1.1.1]pentan-1-yl)thiazole-4-carboxamido)acrylamido)acrylate (85 mg, 0.18 mmol, 58%) as a white solid. LCMS (22010199A TFA LCMS-5 C1): rt 1.319 min; Area 99.0% (215 nm), 98.7% (304 nm); m/z=463.2[M+H]⁺. ¹H NMR (400 MHz, CDCl₃) δ 9.81 (s, 1H), 8.51 (s, 1H), 8.05 (s, 1H), 6.73 (d, J=2.1 Hz, 1H), 6.69 (s, 1H), 6.01 (d, J=1.3 Hz, 1H), 5.47 (t, J=1.9 Hz, 1H), 5.02 (s, 1H), 3.90 (s, 3H), 2.48 (s, 6H), 1.47 (s, 9H).

### Example 32: Compound 79

### methyl 2-(2-(2-(4-((tert-butoxycarbonyl)amino)cyclohexyl)thiazole-4-carboxamido)acrylamido)acrylate

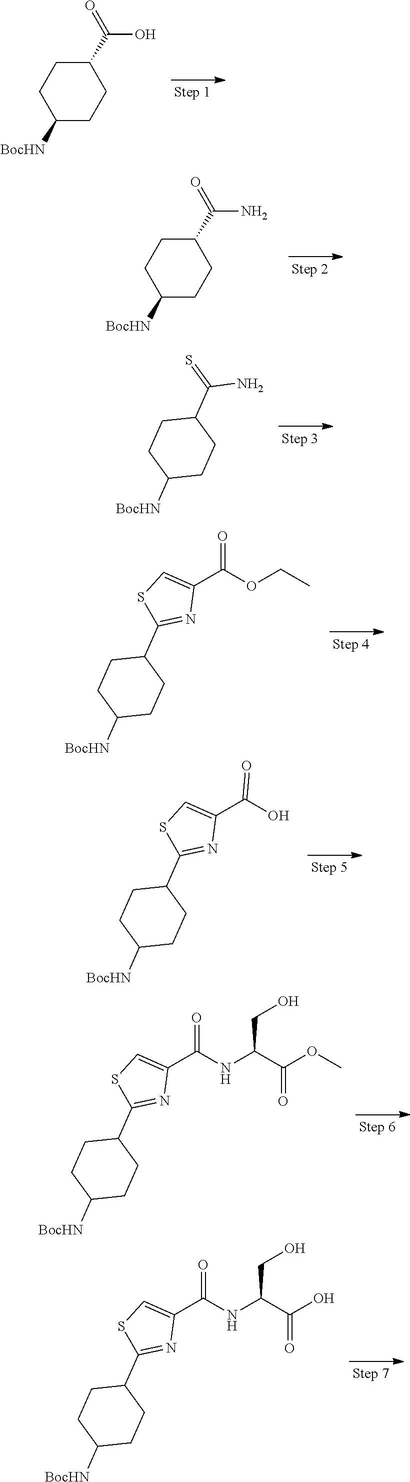

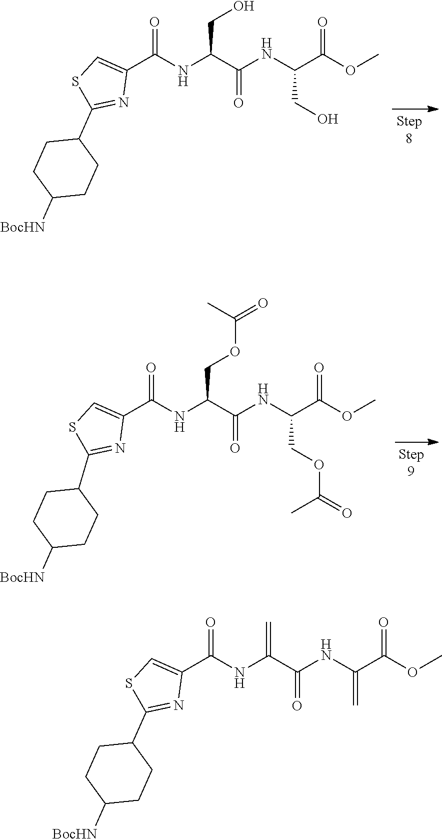 [SMILES: COC(=O)[C@H](CO)NC(=O)[C@H](CO)NC(=O)c1csc(C2CCC(NC(=O)OC(C)(C)C)CC2)n1 ; COC(=O)[C@H](COC(C)=O)NC(=O)[C@H](COC(C)=O)NC(=O)c1csc(C2CCC(NC(=O)OC(C)(C)C)CC2)n1 ; C=C(NC(=O)c1csc(C2CCC(NC(=O)OC(C)(C)C)CC2)n1)C(=O)NC(=C)C(=O)OC]

Step 1. tert-butyl (trans-4-carbamoylcyclohexyl)carbamate was prepared following General experimental procedure 9. Trans-4-((tert-butoxycarbonyl)amino)cyclohexane-1-carboxylic acid (2.0 g, 8.2 mmol) gave tert-butyl (trans-4-carbamoylcyclohexyl)carbamate (1.9 g, 7.7 mmol, 94%) as a white solid.

Step 2. tert-butyl (4-carbamothioylcyclohexyl)carbamate was prepared following General experimental procedure 10. tert-butyl (trans-4-carbamoylcyclohexyl)carbamate (1.9 g, 7.7 mmol) gave tert-butyl (4-carbamothioylcyclohexyl)carbamate (1.2 g, 4.7 mmol, 61%, trans cis 97:3) as an off-white solid.

Step 3. Ethyl 2-(4-((tert-butoxycarbonyl)amino)cyclohexyl)thiazole-4-carboxylate was prepared following General experimental procedure 11. tert-butyl (4-carbamothioylcyclohexyl)carbamate (1.2 g, 4.7 mmol) gave ethyl 2-(4-((tert-butoxycarbonyl)amino)cyclohexyl)thiazole-4-carboxylate (1.1 g, 3.0 mmol, 64%, trans cis 9:1).

Step 4. 2-(4-((tert-butoxycarbonyl)amino)cyclohexyl)thiazole-4-carboxylic acid was prepared following General experimental procedure 2. Ethyl 2-(4-((tert-butoxycarbonyl)amino)cyclohexyl)thiazole-4-carboxylate (1.1 g, 3.0 mmol) gave 2-(4-((tert-butoxycarbonyl)amino)cyclohexyl)thiazole-4-carboxylic acid (0.97 g, 3.0 mmol, 99%, trans cis 9:1)

Step 5. Methyl (2-(4-((tert-butoxycarbonyl)amino)cyclohexyl)thiazole-4-carbonyl)-L-serinate was prepared following General experimental procedure 4. 2-(4-((tert-butoxycarbonyl)amino)cyclohexyl)thiazole-4-carboxylic acid (0.97 g, 3.0 mmol) and methyl L-serinate hydrochloride (0.51 g, 1.1 eq., 3.3 mmol) gave methyl (2-(4-((tert-butoxycarbonyl)amino)cyclohexyl)thiazole-4-carbonyl)-L-serinate (1.1 g, 89% purity, 2.2 mmol, 75%)

Step 6. (2-(4-((tert-butoxycarbonyl)amino)cyclohexyl)thiazole-4-carbonyl)-L-serine was prepared following General experimental procedure 2. Methyl (2-(4-((tert-butoxycarbonyl)amino)cyclohexyl)thiazole-4-carbonyl)-L-serinate (1.1 g, 89% purity, 2.2 mmol) gave (2-(4-((tert-butoxycarbonyl)amino)cyclohexyl)thiazole-4-carbonyl)-L-serine (1.0 g, 88% purity, 2.1 mmol, 97%).

Step 7. Methyl (2-(4-((tert-butoxycarbonyl)amino)cyclohexyl)thiazole-4-carbonyl)-L-seryl-L-serinate was prepared following General experimental procedure 4. (2-(4-((tert-butoxycarbonyl)amino)cyclohexyl)thiazole-4-carbonyl)-L-serine (1.0 g, 88% purity, 2.1 mmol) and methyl L-serinate hydrochloride (0.36 g, 1.1 eq., 2.3 mmol) gave methyl (2-(4-((tert-butoxycarbonyl)amino)cyclohexyl)thiazole-4-carbonyl)-L-seryl-L-serinate (0.55 g, 90% purity, 0.96 mmol, 45%).

Step 8. Methyl O-acetyl-N—(O-acetyl-N-(2-(4-((tert-butoxycarbonyl)amino)cyclohexyl)thiazole-4-carbonyl)-L-seryl)-L-serinate was prepared following General experimental procedure 5. Methyl (2-(4-((tert-butoxycarbonyl)amino)cyclohexyl)thiazole-4-carbonyl)-L-seryl-L-serinate (0.55 g, 90% purity, 0.96 mmol) gave methyl O-acetyl-N—(O-acetyl-N-(2-(4-((tert-butoxycarbonyl)amino)cyclohexyl)thiazole-4-carbonyl)-L-seryl)-L-serinate (0.46 g, 0.77 mmol, 80%, trans cis 87:13)

Step 9. Methyl 2-(2-(2-(4-((tert-butoxycarbonyl)amino)cyclohexyl)thiazole-4-carboxamido)acrylamido)acrylate was prepared following General experimental procedure 8. Methyl O-acetyl-N—(O-acetyl-N-(2-(4-((tert-butoxycarbonyl)amino)cyclohexyl)thiazole-4-carbonyl)-L-seryl)-L-serinate (0.20 g, 0.33 mmol) gave two batches of methyl 2-(2-(2-(4-((tert-butoxycarbonyl)amino)cyclohexyl)thiazole-4-carboxamido)acrylamido)acrylate 79-1 and 79-2

79-1 (81 mg, 0.17 mmol, 51%, trans/cis 89:11) LCMS (22010199A TFA LCMS-5 C1): RT: 1.492 min; Area: 86.2% (215 nm), 88.4% (306 nm); m/z 479.2 (M−H+); RT: 1.520 min; Area: 10.80% (215 nm), 11.61% (306 nm); m/z=479.2 [M+H]^{+ 1}H-NMR (major isomer) (400 MHz, CDCl₃) δ 9.90 (s, 1H), 8.53 (s, 1H), 8.03 (s, 1H), 6.74 (d, J=2.2 Hz, 1H), 6.70 (s, 1H), 6.01 (d, J=1.3 Hz, 1H), 5.46 (t, J=1.9 Hz, 1H), 4.43 (s, 1H), 3.90 (s, 3H), 3.51 (s, 1H), 2.97 (tt, J=12.1, 3.6 Hz, 1H), 2.25 (d, J=13.4 Hz, 2H), 2.17 (d, J=12.7 Hz, 2H), 1.66 (qd, J=13.1, 3.2 Hz, 2H), 1.46 (s, 9H), 1.27 (qd, J=12.7, 3.4 Hz, 2H).

79-2 14 mg, 29 mol, 8.7%, trans/cis 63:37. LCMS-5 (22010199A TFA LCMS-5 C1): RT: 1.493 min; Area: 55.3% (215 nm), 54.5% (306 nm); m/z 479.2 (M−H+); RT: 1.526 min; Area: 42.9% (215 nm), 43.6% (306 nm); m/z=379.2 [M-Boc+H]⁺. ¹H NMR (400 MHz, CDCl₃) δ 9.99 (s, OH), 9.90 (s, 1H), 8.54 (m, 1H), 8.03 (m, 1H), 6.74 (d, J=2.0 Hz, 1H), 6.70 (d, J=3.7 Hz, 1H), 6.01 (s, 1H), 5.47 (m, 1H), 4.79 (s, OH), 4.43 (s, OH), 3.90 (s, 3H), 3.78 (s, OH), 3.50 (s, OH), 3.16 (s, OH), 3.02-2.91 (m, 1H), 2.25 (m, 1H), 2.17 (m, 1H), 2.01 (s, 1H), 1.97-1.74 (m, 2H), 1.74-1.59 (m, 2H), 1.45 (m, 9H), 1.37-1.20 (m, 2H).

### Example 33: Compound 80

### Methyl 2-(2-(2-(4-((tert-butoxycarbonyl)amino)bicyclo[2.2.2]octan-1-yl)thiazole-4-carboxamido)acrylamido)acrylate

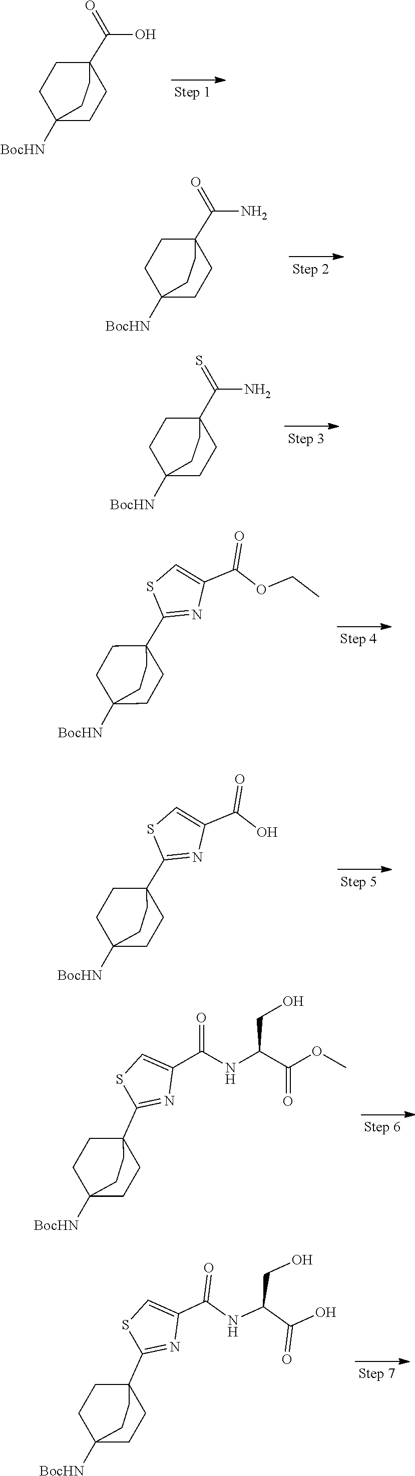

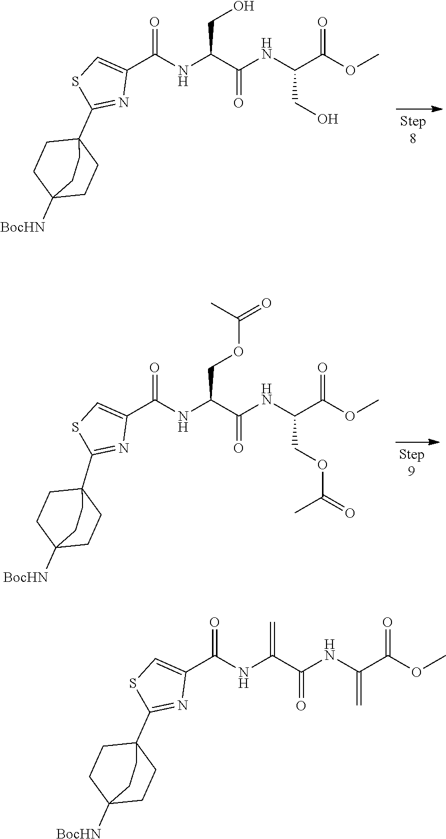 [SMILES: COC(=O)[C@H](CO)NC(=O)[C@H](CO)NC(=O)c1csc(C23CCC(NC(=O)OC(C)(C)C)(CC2)CC3)n1 ; COC(=O)[C@H](COC(C)=O)NC(=O)[C@H](COC(C)=O)NC(=O)c1csc(C23CCC(NC(=O)OC(C)(C)C)(CC2)CC3)n1 ; C=C(NC(=O)c1csc(C23CCC(NC(=O)OC(C)(C)C)(CC2)CC3)n1)C(=O)NC(=C)C(=O)OC]

Step 1. tert-butyl (4-carbamoylbicyclo[2.2.2]octan-1-yl)carbamate was prepared following General experimental procedure 9. 4-((tert-butoxycarbonyl)amino)bicyclo[2.2.2]octane-1-carboxylic acid (1.0 g, 3.7 mmol) gave tert-butyl (4-carbamoylbicyclo[2.2.2]octan-1-yl)carbamate (1.2 g, 82% purity, 3.5 mmol, 95%)

Step 2. tert-butyl (4-carbamothioylbicyclo[2.2.2]octan-1-yl)carbamate was prepared following General experimental procedure 10. tert-butyl (4-carbamoylbicyclo[2.2.2]octan-1-yl)carbamate (1.2 g, 82% purity, 3.5 mmol) gave tert-butyl (4-carbamothioylbicyclo[2.2.2]octan-1-yl)carbamate (0.48 g, 1.7 mmol, 47%) as a white solid.

Step 3. Ethyl 2-(4-((tert-butoxycarbonyl)amino)bicyclo[2.2.2]octan-1-yl)thiazole-4-carboxylate was prepared following General experimental procedure 11. tert-butyl (4-carbamothioylbicyclo[2.2.2]octan-1-yl)carbamate (0.48 g, 1.7 mmol) gave ethyl 2-(4-((tert-butoxycarbonyl)amino)bicyclo[2.2.2]octan-1-yl)thiazole-4-carboxylate (0.31 g, 0.82 mmol, 49%) as a white solid. ¹H NMR (400 MHz, CDCl₃) δ 8.00 (s, 1H), 4.37 (q, J=7.1 Hz, 2H), 2.12-1.99 (m, 6H), 1.99-1.91 (m, 6H), 1.41 (s, 9H), 1.36 (t, J=7.1 Hz, 3H).

Step 4. 2-(4-((tert-butoxycarbonyl)amino)bicyclo[2.2.2]octan-1-yl)thiazole-4-carboxylic acid was prepared following General experimental procedure 2. Ethyl 2-(4-((tert-butoxycarbonyl)amino)bicyclo[2.2.2]octan-1-yl)thiazole-4-carboxylate (0.31 g, 0.82 mmol) gave 2-(4-((tert-butoxycarbonyl)amino)bicyclo[2.2.2]octan-1-yl)thiazole-4-carboxylic acid (0.32 g, 90% purity, 0.82 mmol, quant.)

Step 5. methyl (2-(4-((tert-butoxycarbonyl)amino)bicyclo[2.2.2]octan-1-yl)thiazole-4-carbonyl)-L-serinate was prepared following General experimental procedure 4. 2-(4-((tert-butoxycarbonyl)amino)bicyclo[2.2.2]octan-1-yl)thiazole-4-carboxylic acid (0.32 g, 90% purity, 0.82 mmol) and methyl L-serinate hydrochloride (0.14 g, 1.1 eq., 0.90 mmol) gave methyl (2-(4-((tert-butoxycarbonyl)amino)bicyclo[2.2.2]octan-1-yl)thiazole-4-carbonyl)-L-serinate (0.33, 88% purity, 0.64 mmol, 78%)

Step 6. (2-(4-((tert-butoxycarbonyl)amino)bicyclo[2.2.2]octan-1-yl)thiazole-4-carbonyl)-L-serine was prepared following General experimental procedure 2. Methyl (2-(4-((tert-butoxycarbonyl)amino)bicyclo[2.2.2]octan-1-yl)thiazole-4-carbonyl)-L-serinate (0.33 g, 0.64 mmol) gave (2-(4-((tert-butoxycarbonyl)amino)bicyclo[2.2.2]octan-1-yl)thiazole-4-carbonyl)-L-serine (0.30 g, 0.66 mmol, quant).

Step 7. Methyl (2-(4-((tert-butoxycarbonyl)amino)bicyclo[2.2.2]octan-1-yl)thiazole-4-carbonyl)-L-seryl-D-serinate was prepared following General experimental procedure 4. (2-(4-((tert-butoxycarbonyl)amino)bicyclo[2.2.2]octan-1-yl)thiazole-4-carbonyl)-L-serine (0.30 g, 0.66 mmol) and methyl L-serinate hydrochloride (0.11 g, 1.1 eq., 0.73 mmol) gave methyl (2-(4-((tert-butoxycarbonyl)amino)bicyclo[2.2.2]octan-1-yl)thiazole-4-carbonyl)-L-seryl-D-serinate (0.30 g, 0.55 mmol, 83%). ¹H NMR (400 MHz, CDCl₃) δ 8.25 (d, J=7.4 Hz, 1H), 7.96 (s, 1H), 7.67 (d, J=7.9 Hz, 1H), 4.69 (m, 2H), 4.36 (s, 1H), 4.17-4.08 (m, 1H), 4.08-3.92 (m, 2H), 3.88-3.81 (m, 1H), 3.79 (s, 3H), 3.77-3.70 (m, 1H), 3.61 (m, 1H), 2.07-1.92 (m, 12H), 1.42 (s, 9H).

Step 8. Methyl O-acetyl-N—(O-acetyl-N-(2-(4-((tert-butoxycarbonyl)amino)bicyclo[2.2.2]octan-1-yl)thiazole-4-carbonyl)-L-seryl)-L-serinate was prepared following General experimental procedure 5. Methyl (2-(4-((tert-butoxycarbonyl)amino)bicyclo[2.2.2]octan-1-yl)thiazole-4-carbonyl)-L-seryl-L-serinate (0.30 g, 0.55 mmol) and acetic anhydride (0.13 mL, 2.5 eq., 1.4 mmol) gave methyl O-acetyl-N-(0-acetyl-N-(2-(4-((tert-butoxycarbonyl)amino)bicyclo[2.2.2]octan-1-yl)thiazole-4-carbonyl)-L-seryl)-L-serinate (0.26 g, 0.41 mmol, 76%).

Step 9. Methyl 2-(2-(2-(4-((tert-butoxycarbonyl)amino)bicyclo[2.2.2]octan-1-yl)thiazole-4-carboxamido)acrylamido)acrylate was prepared following General experimental procedure 8. Methyl O-acetyl-N-(0-acetyl-N-(2-(4-((tert-butoxycarbonyl)amino)bicyclo[2.2.2]octan-1-yl)thiazole-4-carbonyl)-L-seryl)-L-serinate (0.15 g, 0.23 mmol) gave methyl 2-(2-(2-(4-((tert-butoxycarbonyl)amino)bicyclo[2.2.2]octan-1-yl)thiazole-4-carboxamido)acrylamido)acrylate (65 mg, 0.13 mmol, 55%) as a white solid. LCMS (22010199A TFA LCMS-5 C1): RT: 1.783 min; Area: 99.9% (215 nm), 99.6% (306 nm); m/z=505.2 [M+H]⁺

### General Procedure for Solid Phase Peptide Synthesis

Fmoc Rink amide AM resin (0.70 mmol/g) was swelled by washing with CH₂Cl₂ (2×1 min, 10 mL/gram resin). The resin was washed with DMF (3×1 min, 10 mL/gram resin) and treated with 20% piperidine in DMF (10 mL/gram resin) for 30 minutes. The resin was washed with DMF (3×1 min, 10 mL/gram resin). The resin was treated with a solution of Fmoc-AA(PG)-OH (3 eq.), HATU (3 eq.) and DiPEA (3 eq.) for 2-3 hours or 16 hours. The resin was washed with DMF (3×1 min, 10 mL/gram resin). The cycle of wash-deprotection-wash-coupling was repeated for everyone amino acid coupling. After completion of the coupling cycles the resin was treated with TFA:TIPS:water (95:2.5:2.5, 10 mL/gram resin) for 2 hours. The resin was removed by filtration and the filtrate was precipitated with MTBE:Heptane (1:1 v/v). After centrifugation (5 min, 3000 rpm), the supernatant was discarded and the residue was resuspended in MTBE:heptane (1:1 v/v) and centrifuged again (5 min, 3000 rpm). The supernatant was discarded and the pellet was taken up in water/MeCN 1:1. The solution was partially concentrated and lyophilized. The crude peptide was purified by automated reverse phase FCC.

### Example 34: Compound 81

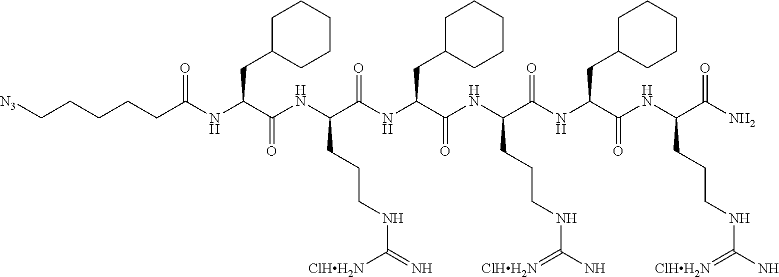

N₃(CH₂)₅C(O)-Cha-D-Arg-Cha-D-Arg-Cha-D-Arg-NH₂ was prepared following General procedure for solid phase peptide synthesis on 2.5 mmol scale. Purification using HCl buffers provided the desired HCl salt form. N₃(CH₂)₅C(O)-Cha-D-Arg-Cha-D-Arg-Cha-D-Arg-NH₂HCl salt (0.58 g, 0.50 mmol, 20%) was isolated as a white solid. LCMS (30833 LCMS-6): RT: 2.403 min; Area: 78.0% (215 nm), 99.6% (ELSD); m/z=1084.7 [M+H]⁺

### Example 35: Compound 82

### N₃(CH₂CH₂O)₆CH₂CH₂C(O)-Cha-D-Arg-Cha-D-Arg-Cha-D-Arg-NH₂ HCl Salt

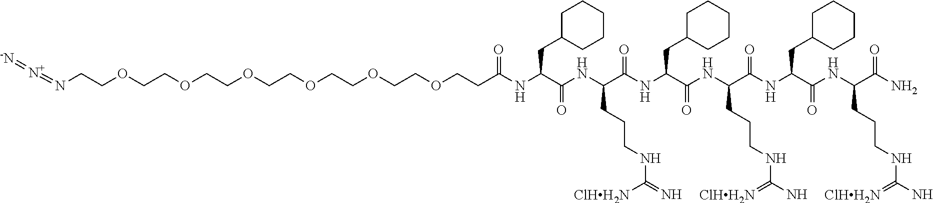

N₃(CH₂CH₂O)₆CH₂CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form. N₃(CH₂CH₂O)₆CH₂CH₂C(O)-Cha-D-Arg-Cha-D-Arg-Cha-D-Arg-NH₂ HCl salt (39 mg, 28 μmol, 11%) was isolated as a white solid. LCMS (30833 LCMS-6): RT: 2.365 min; Area: 97.8% (215 nm), 99.6% (ELSD); m/z=1306.8 [M+H]⁺

### Example 36: Compound 83

### N₃(CH₂CH₂O)₉CH₂CH₂C(O)-Cha-D-Arg-Cha-D-Arg-Cha-D-Arg-NH₂ HCl Salt

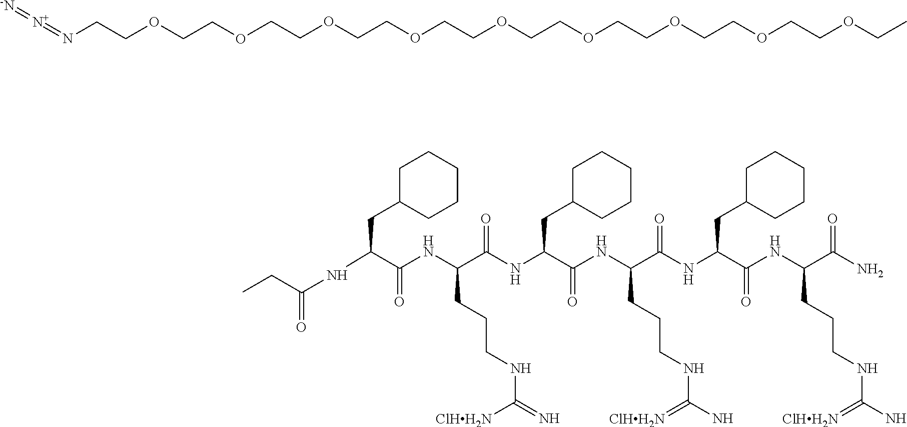

N₃(CH₂CH₂O)₉CH₂CH₂C(O)-Cha-D-Arg-Cha-D-Arg-Cha-D-Arg-NH₂ HCl salt was prepared following General procedure for solid phase peptide synthesis on 0.25 mmol scale. Purification using HCl buffers provided the desired HCl salt form. N₃(CH₂CH₂O)₉CH₂CH₂C(O)-Cha-D-Arg-Cha-D-Arg-Cha-D-Arg-NH₂ HCl salt (34 mg, 22 μmol, 8.8%) was isolated as a white solid. LCMS (30833 LCMS-6): RT: 2.415 min; Area: 98.4% (215 nm), 99.8% (ELSD); m/z=1438.9 [M+H]⁺

General procedure for conjugation chemistry between azido-peptide and Compound 49 To a solution of Compound 49 in DMF/water (1:1, 2 mL) was added the azido-peptide and the resulting mixture was stirred for 3 hours. The mixture was directly purified using automated reverse phase FCC.

### Example 37: Compound 84

### Compound 49 Coupled to Compound 82

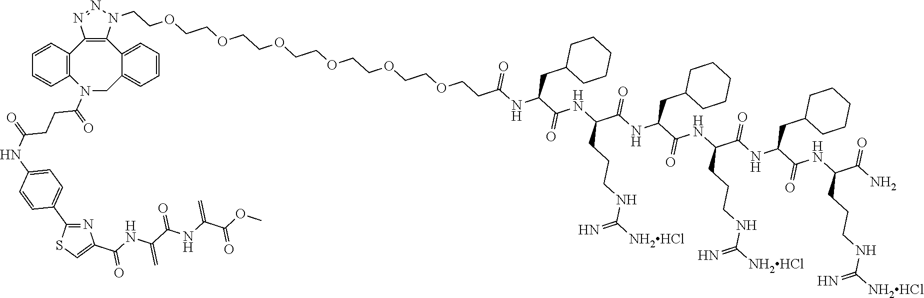

Compound 84 was prepared following General procedure for conjugation chemistry between azido-peptide and 49. Compound 49 (3.0 mg, 4.5 μmol) and Compound 82 (6.4 mg, 4.5 μmol) gave Compound 84 (4.3 mg, 2.1 μmol, 46%) as a white solid. LCMS (30833 LCMS-6): RT: 2.791 min; Area: 81.9% (215 nm), 98.1% (ELSD); m/z=1966.2 [M+H]⁺

### Example 38: Compound 85

### Compound 49 Coupled to Compound 83

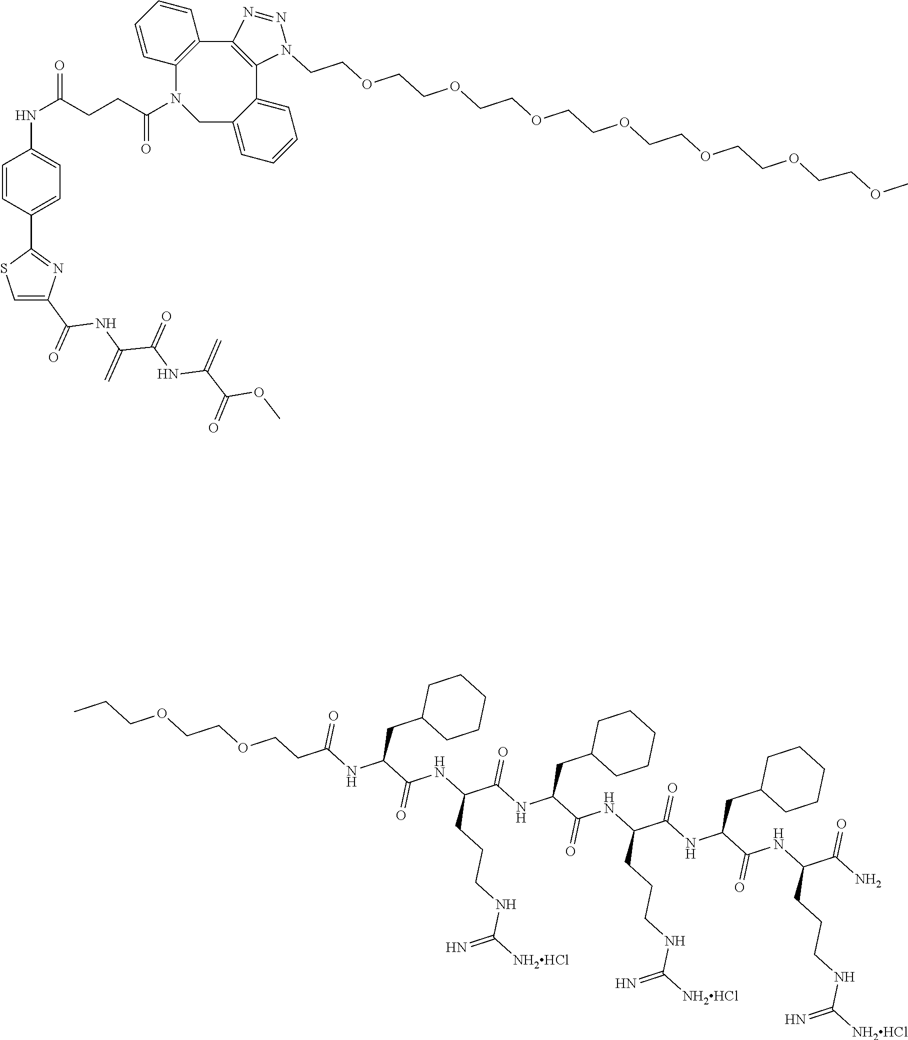

Compound 85 was prepared following General procedure for conjugation chemistry between azido-peptide and Compound 49 (3.0 mg, 4.5 μmol) and Compound 83 (7.0 mg, 4.5 μmol) gave Compound 85 (4.3 mg, 2.0 μmol, 43%) as a white solid. LCMS (30833 LCMS-6): RT: 2.802 min; Area: 59.7% (215 nm), 85.5% (ELSD); m/z=1049.7 [M+2H]²⁺

### Example 39: Prophetic Assays

To test the mechanism of binding and to explore the binding target, cell treatments and western blotting will be carried out as in Cunniff et al. 2015. Covalent crosslinking of enzymes is detectable by protein western blotting using specific antibodies.

Briefly, human tumor cell lines (HMESO cell line derived from a patient with malignant mesothelioma) will be cultured in appropriate medium and treated with varying concentrations of test compounds for 24 hours (0.1 μM-100 μM). After 24 hours of exposure to test compounds, cellular lysates will be generated in standard lysis buffer (RIPA Buffer). Protein abundance will be quantified, and equal protein concentrations will be separated by SDS-Polyacrylamide Gel Electrophoresis (SDS-PAGE). Protein western blotting will be conducted using antibodies specific to proteins, such as PRX1, PRX2, PRX3 and PRX4. Covalent crosslinking modifications are detectable by the presence of an ˜46 kD antibody reactive species on the protein western blot.

Cell death assays will be conducted as in Nelson et al. 2021. Briefly, human tumor cell lines (HMESO cell line derived from a patient with malignant mesothelioma) will be cultured in 96-well plates and incubated with test compounds for 48 hours. The amount of residual cell material will be stained with crystal violet and total cell counts will conducted to determine % cell viability.

### Example 40: Cell Viability Study

Cell Lines were plated in 96-well plates (Corning, Kennebunk, ME, USA) at a density of 2500 cells per well. The following day, cells were treated with test compounds diluted in complete media followed by incubation for 48 h. Post-incubation cells were washed with PBS (Corning Cellgro, Manassas, VA, USA), fixed with 3.0% formaldehyde (Fisher BioReagents, Fair Lawn, NJ, USA) in PBS, and stained for 30 min with 0.1% crystal violet (Acros Organics, Fair Lawn, NJ, USA) in water. Crystal violet stain was removed, and plates were washed with H₂O and allowed to dry. To quantify cell viability, plates were imaged using the Lionheart Plate reader (BioTek Instruments, Winooski, VT, USA) and/or analyzed by absorbance at 540 nm (crystal violet dye dissolved in 100% methanol) using the Synergy HTX plate reader (BioTek Instruments, Winooski, VT, USA). To determine the effective cytotoxic concentration (IC₅₀) of test compounds, the data were plotted using a 4-parameter non-linear regression model using GraphPad Prism7 software (GraphPad Software, San Diego, CA, USA).

Cell viability assay data in HMESO mesothelioma cells treated with compounds (1)-HCl or (5) HCl are summarized in the below Table 2. IC₅₀ (Concentration of drug required to kill 50% of cells) is shown in μM concentration

TABLE 2

| | TS | (1)-HCl | (5)-HCl | DMSO |
|---|---|---|---|---|
| IC₅₀ | 1.013 | 747.8 | 1.930* | Unstable |

*95% confidence interval (CI) of 1.699-2.187

### Example 41: Crosslinking Study

Malignant Mesothelioma (MM) cells (H-MESO cell line) were plated into 6-well plates in complete tissue culture media. Cells were allowed to adhere for 24 hours before being treated with indicated concentrations of thiostrepton (TS), (1) or (5) (DMSO Stocks) for 24 hours. Cell lysates were generated using standard RIPA buffer, protein concentrations were determined using a Bradford Assay and 20 μg of total protein per sample were separated by reducing SDS-Polyacrylamide Gel Electrophoresis. Proteins were transferred to a PVDF membrane, blocked with 5% Bovine Serum Albumin (BSA) for 1 hour and incubated with PRX3 primary antibody overnight at 4° C. in 1× Tris Buffered Saline with Tween (TBST). Membranes were washed 3× with 1×TBST and incubated with horseradish peroxidase conjugated (HRP) secondary antibody for 1 hour at room temperature. Membranes were washed 3× in 1×TBST and HRP signal was developed using Enhanced Chemiluminescence and visualized on a GE digital imager.

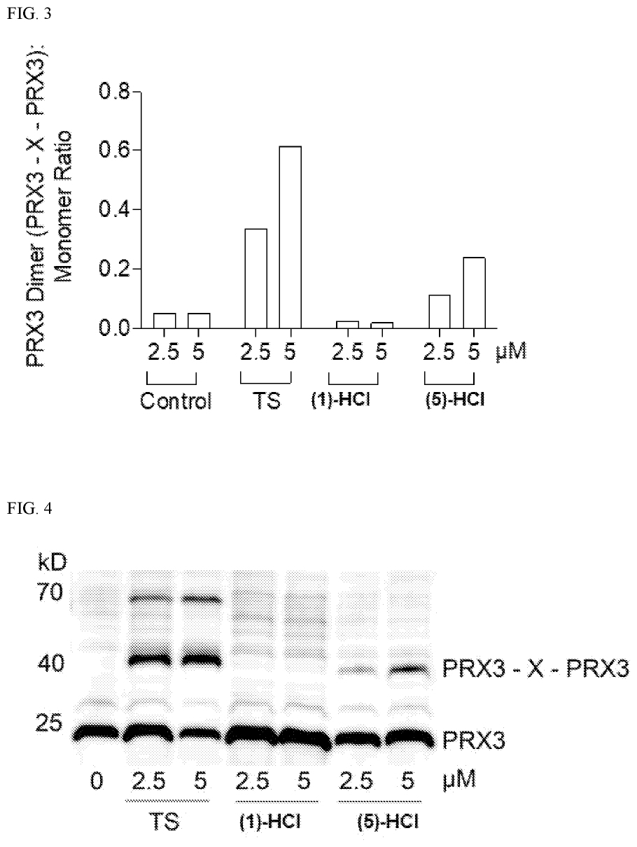

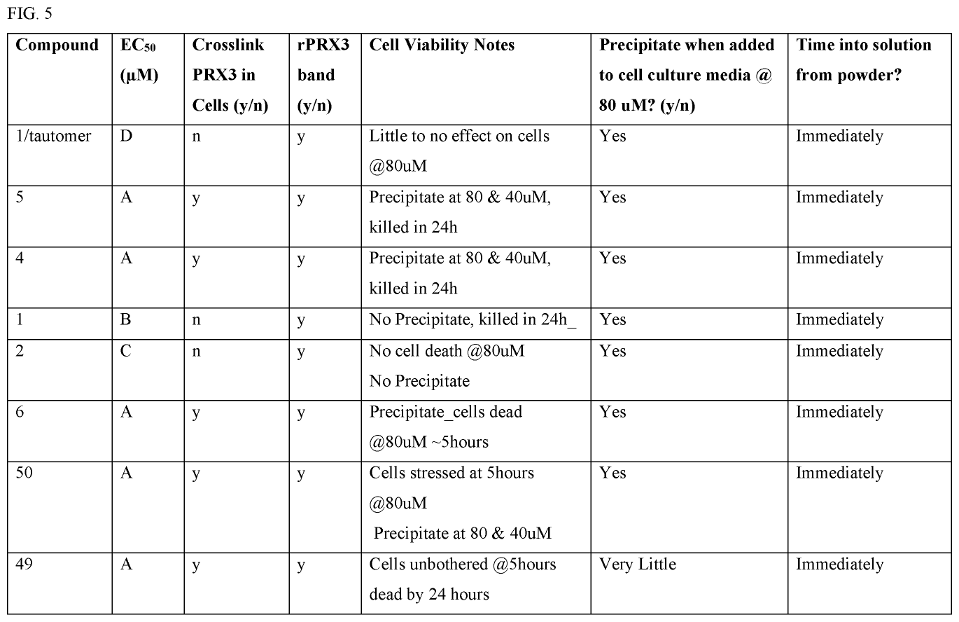

Treatment of cells with 2.5 or 5 μM of TS resulted in covalent PRX3 crosslinking (PRX3-X-PRX3). Similar, yet less robust, results were observed for (5)-treated cells (FIG. 3). No PRX3-X-PRX3 crosslinking was observed in cells treated with (1) (also FIG. 3).

### Example 42: Cytotoxic Activity of Test Compounds in Malignant Mesothelioma Cell Lines

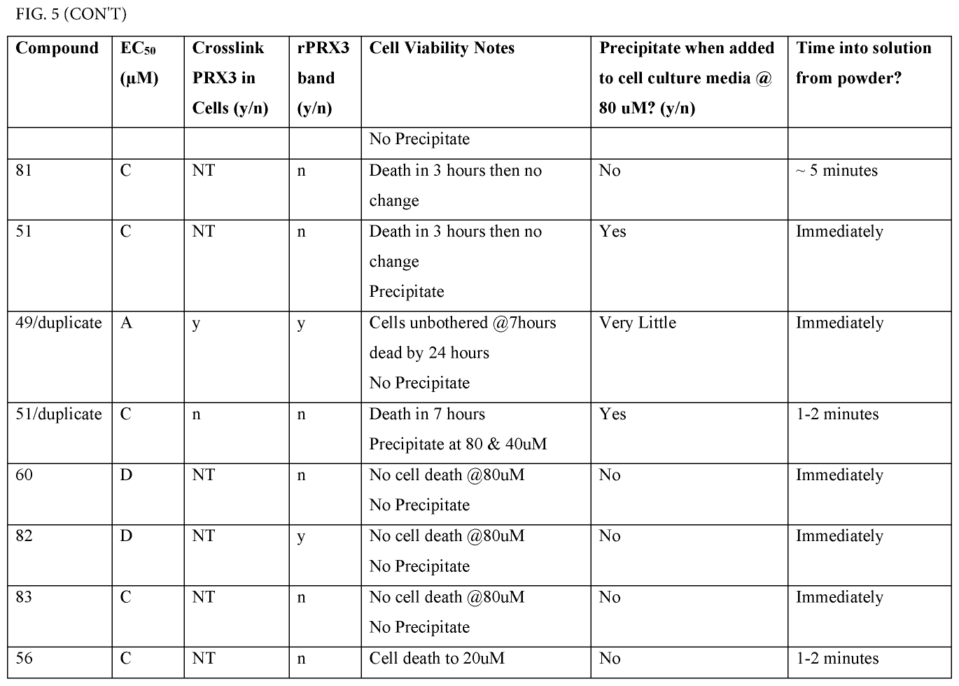

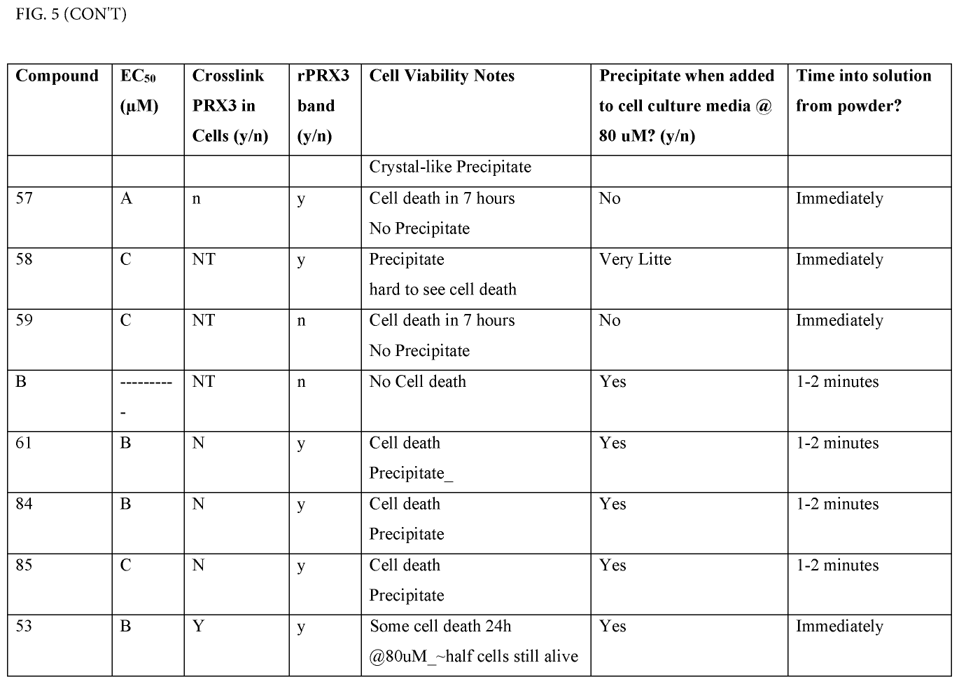

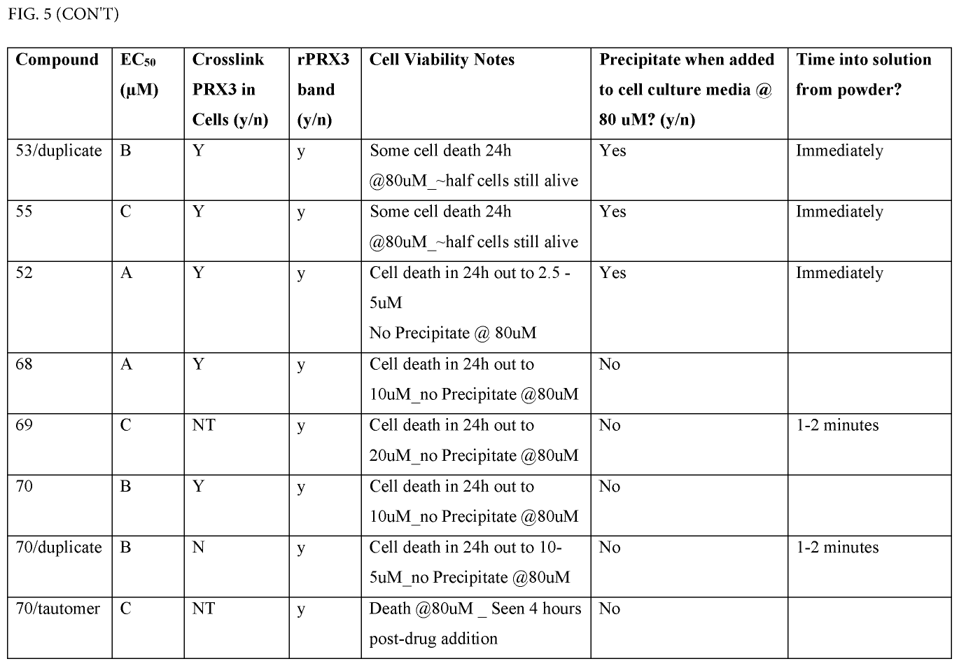

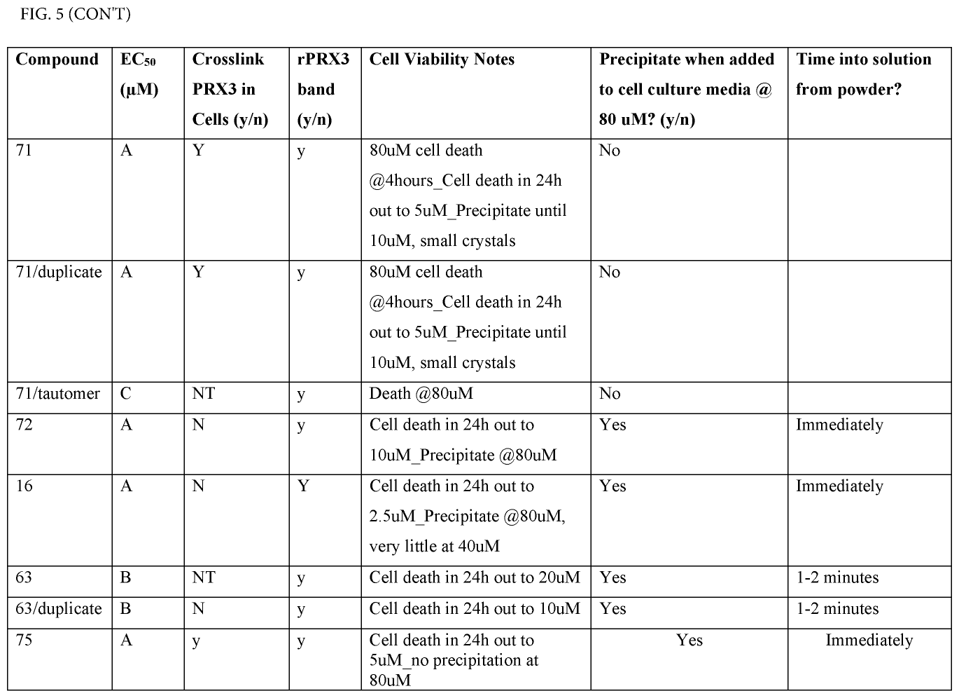

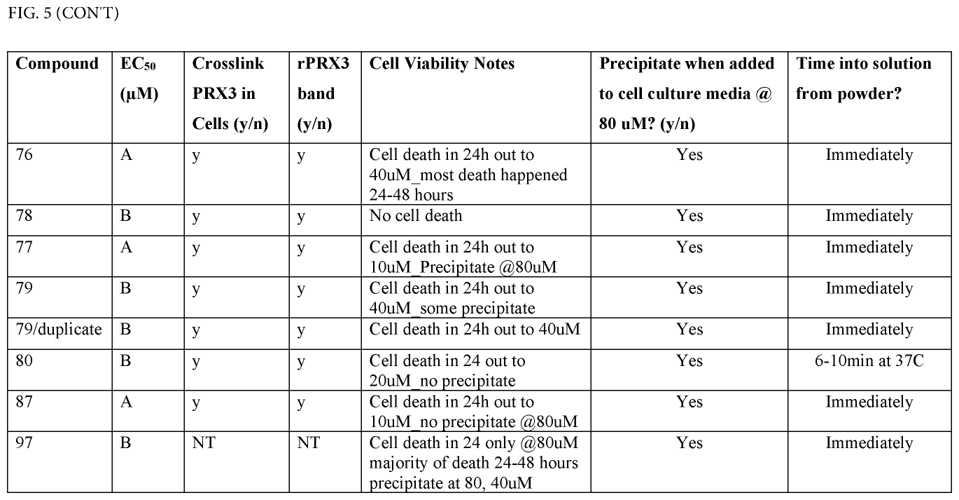

Malignant mesothelioma (HMESO cell line) cells were plated in 96-well plates (Corning, Kennebunk, ME, USA) at a density of 2500 cells per well. The following day, cells were treated with test compounds diluted in complete media followed by incubation for 48 h (in technical duplicates). Post-incubation cells were washed with PBS (Corning Cellgro, Manassas, VA, USA), fixed with 3.0% formaldehyde (Fisher BioReagents, Fair Lawn, NJ, USA) in PBS, and stained for 30 min with 0.1% crystal violet (Acros Organics, Fair Lawn, NJ, USA) in water. Crystal violet stain was removed, and plates were washed with H₂O and allowed to dry. To quantify cell viability, plates were imaged using the Lionheart Plate reader (BioTek Instruments, Winooski, VT, USA) and/or analyzed by absorbance at 540 nm (crystal violet dye dissolved in 100% methanol) using the Synergy HTX plate reader (BioTek Instruments, Winooski, VT, USA). To determine the effective cytotoxic concentration (EC₅₀) of test compounds the data were plotted using a 4-parameter non-linear regression model using GraphPad Prism7 software (GraphPad Software, San Diego, CA, USA). Results are tabulated in FIG. 5.

### Example 43: Covalent Crosslinking of Recombinant Peroxiredoxin 3 (rPRX3) by Test Compounds

Master Mix reagents in Table 3 were combined for a 1× reaction in an Eppendorf tube on ice. The reaction was scaled by the number of test compounds being tested. 16 μL of master mix were added to a new Eppendorf tube containing 1 μL of test compound (10 mM stock diluted in DMSO) and mixed by gentle flicking and quick centrifugation at 1,000 RPM. Reactions were incubated at 37° C. for 18 hours. Reactions were removed from incubation and quenched by addition of 2 μL of Laemmli buffer containing 0.2 M dithiothreitol (DTT) and 10% sodium dodecyl sulfate. Samples were boiled at 98° C. for 5 minutes. Samples were separated by polyacrylamide gel electrophoresis, transferred to a PVDF membrane, and subjected to protein western blotting using an anti-PRX3 antibody (AbFrontier, LF-PA0255). Membranes were incubated with ECL Reagent (ThermoScientific, Rockford, IL, USA) and visualized using a GE Amersham Imager chemiluminescent detection system. Unmodified rPRX3 was visualized as a single band at ˜23 kDa and rPRX3 covalently modified by test compounds runs as a ˜45 kDa band. This is a qualitative assay evaluating the presence or absence of the ˜45 kDA band. Qualitative results are tabulated in FIG. 5.

TABLE 3
Master Mix

| Reagent | uL |
|---|---|
| 20 mM Tris pH 8 | 11 |
| rPRX3 (1 mg/ml) | 2 |
| 0.5M TCEP | 3 |

### Example 44: Covalent Crosslinking of Peroxiredoxin 3 (PRX3) in Malignant Mesothelioma Cells by Test Compounds

Human malignant mesothelioma (HMESO cell line) cells were plated in 6 well plates at a density of 200,000 cells per well. After 24 hours, cells were treated with test compounds diluted in DMSO and cell culture media. Cell lysates were harvested at 24 hours post treatment using RIPA buffer (50 mM Tris-HCl, 150 mM NaCl, 1 mM EDTA, 1% NP-40, 0.25% Sodium deoxycholate, 0.1% sodium dodecyl sulfate, in deionized (DI) water) for reducing samples to be analyzed by reducing SDS-PAGE. Protein concentrations were determined via Bradford Assay (ThermoScientific, Rockford, IL, USA). Lysates (15 μg protein/well) were resolved by SDS-PAGE under reducing conditions on 4-12% gradient Bis-Tris Midi gel (Invitrogen, Carlsbad, CA, USA) at constant 200 V for 50 m. The gel was transferred to a PVDF membrane at constant 1A for 50 min, blocked with 5% BSA diluted in 1× Tris-buffered saline with 1% Tween-20 (TBS-T) for a minimum of 1 hour, and incubated with anti-PRX3 antibody in 5% BSA TBS-T at 4° C. overnight. The membrane was washed with 1×TBS-T for 1 hour, incubated with appropriate secondary antibody 1 hour, and washed again with 1×TBS-T for 1 hour. Membranes were incubated with ECL Reagent (ThermoScientific, Rockford, IL, USA) and visualized using a GE Amersham Imager chemiluminescent detection system. Qualitative results are tabulated in FIG. 5.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A compound having the structure of Formula (IA): 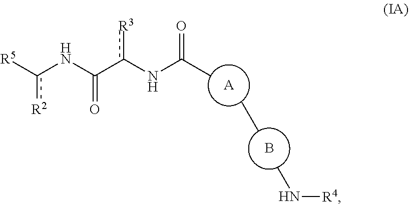
or a pharmaceutically acceptable salt thereof, wherein:
R¹ is —NH₂, —NH(CH₃), —O—CH₃, or —NH—CH₂—C(O)—NH₂;
R² is —H, —CH₃, ═CH₂, or ═CH(alkyl);
R³ is —H, —CH₃, ═CH₂, or ═CH(alkyl);
R⁵ is —C(O)—R¹ or —CN;
Ring A is aryl, heteroaryl, cycloalkyl, or heterocyclyl;
Ring B is absent or present and, when present, is aryl, heteroaryl, cycloalkyl, or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety;
each [symbol] is independently a single bond or a double bond; and
any hydrogen atom is optionally replaced with a deuterium.

2. The compound of claim 1 wherein R⁵ is —C(O)—R¹; and R¹ is —OCH₃.

3. The compound of claim 1, wherein R⁵ is —C(O)—R¹; and R¹ is —NH₂.

4. The compound of any one of claims 1-3, wherein, when Ring A is polycyclic, then Ring B is absent.

5. The compound of any one of claims 1-4 having the structure of Formula (IA-1): 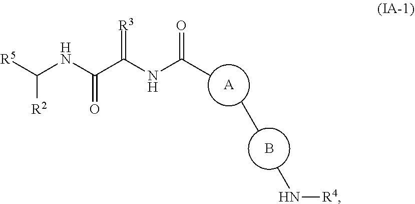
wherein
R² is —H or —CH₃; and
R³ is ═CH₂ or ═CH(alkyl).

6. The compound of claim 5, wherein R² is —H and R³ is ═CH₂, or R² is —CH₃ and R³ is ═CH₂.

7. The compound of any one of claims 1-4 having the structure of Formula (IA-2): 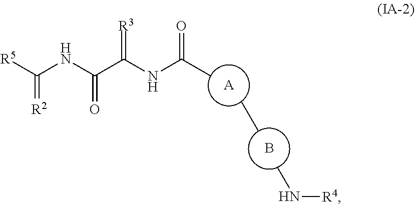
wherein
R² is ═CH₂ or ═CH(alkyl); and
R³ is —H or —CH₃.

8. The compound of claim 7, wherein R² is ═CH₂ and R³ is —H, or R² is ═CH₂ and R³ is —CH₃.

9. The compound of any one of claims 1-4 having the structure of Formula (IA-3): 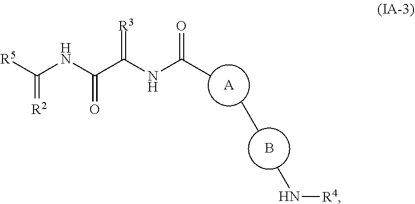
wherein
R² is ═CH₂ or ═CH(alkyl); and
R³ is ═CH₂ or ═CH(alkyl).

10. The compound of claim 9, wherein R² is ═CH₂ and R³ is ═CH₂, R² is ═CH₂ and R³ is ═CH(CH₃), or R² is ═CH(CH₃) and R³ is ═CH₂.

11. The compound of claim 1 having the structure of Formula (IB): 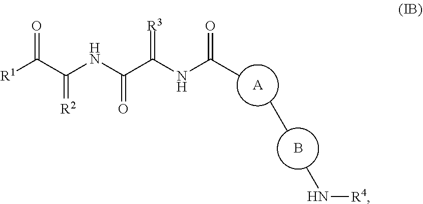
or a pharmaceutically acceptable salt thereof, wherein:
R¹ is —NH₂ or —O—CH₃;
R² is CH₂ or CH(alkyl);
R³ is CH₂ or CH(alkyl);
Ring A is heteroaryl, cycloalkyl, or heterocyclyl;
Ring B is aryl, heteroaryl, cycloalkyl, or heterocyclyl;
R⁴ is hydrogen, a protecting group, —C(O)—CH₃, -L′, or -L-Y;
L′, when present, is a reactive linker moiety;
L, when present, is a linker moiety;
Y, when present, is a mitochondrial targeting moiety; and
any hydrogen atom is optionally replaced with a deuterium.

12. The compound of claim 11, wherein R² and R³ are different.

13. The compound of claim 11, wherein R² and R³ are the same.

14. The compound of any one of claims 11-13, wherein R² is CH(Me) or —CH₂.

15. The compound of any one of claims 11-13, wherein R³ is CH(Me) or —CH₂.

16. The compound of claim 13, wherein R² and R³ are each CH₂.

17. The compound of any one of claims 1-16, wherein Ring A is a 5-membered ring or a 5-membered ring fused to a second ring.

18. The compound of any one of claims 1-16, wherein Ring A is a 5-membered heteroaryl.

19. The compound of claim 18, wherein Ring A is thiazolyl, thiophenyl, oxazolyl, or imidazolyl.

20. The compound of claim 19, wherein Ring A is thiazolyl, thiophenyl, or oxazolyl.

21. The compound of claim 20, wherein Ring A is thiazolyl.

22. The compound of claim 18, wherein Ring A is: 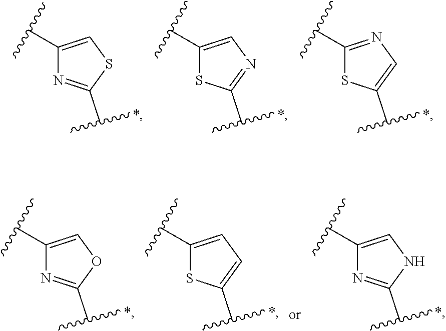 [SMILES: *c1csc(*)n1 ; *c1cnc(*)s1 ; *c1cnc(*)s1 ; *c1coc(*)n1 ; *c1ccc(*)s1 ; *c1c[nH]c(*)n1]
wherein * denotes a bond to Ring B.

23. The compound of claim 18, wherein Ring A is: 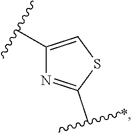 [SMILES: *c1csc(*)n1]
wherein * denotes a bond to Ring B.

24. The compound of any one of claims 1-16, wherein Ring A is a 5-membered cycloalkyl or heterocyclyl.

25. The compound of claim 24, wherein Ring A is cyclopentyl or tetrahydrofuranyl.

26. The compound of claim 25, wherein Ring A is 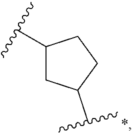 [SMILES: *C1CCC(*)C1]
wherein * denotes a bond to Ring B.

27. The compound of any one of claims 1-16, wherein Ring A is a bridged bicyclic cycloalkyl or heterocyclyl.

28. The compound of claim 27, wherein Ring A is bicyclo[2.1.1]hexyl or oxabicyclo[2.1.1]hexyl.

29. The compound of claim 28, wherein Ring A is 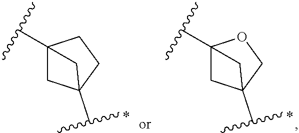 [SMILES: *C12CCC(*)(C1)C2 ; *C12COC(*)(C1)C2]
wherein * denotes a bond to Ring B.

30. The compound of any one of claims 1-16, wherein Ring A is phenyl.

31. The compound of claim 30, wherein Ring A is 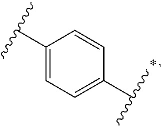 [SMILES: *c1ccc(*)cc1]
wherein * denotes a bond to Ring B.

32. The compound of any one of claims 1-16, wherein Ring A is a polycyclic aryl, heteroaryl, cycloalkyl, or heterocyclyl.

33. The compound of claim 32, wherein Ring A is a bicyclic heteroaryl.

34. The compound of claim 33, wherein Ring A is: 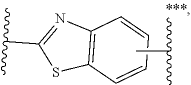
wherein *** denotes a —NH—R⁴.

35. The compound of any one of claims 1-31, wherein Ring B is a 6-membered ring.

36. The compound of claim 35, wherein Ring B is phenyl.

37. The compound of claim 36, wherein Ring B is unsubstituted phenyl.

38. The compound of claim 36, wherein Ring B is a halogen-substituted phenyl.

39. The compound of claim 37 or 38, wherein Ring B is: 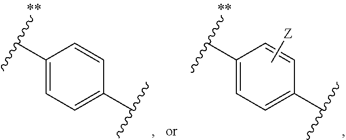
or wherein Z is halo and ** denotes a bond to Ring A.

40. The compound of claim 35, wherein Ring B is a 6-membered heteroaryl.

41. The compound of claim 40, wherein Ring B is pyridinyl or pyrazinyl.

42. The compound of claim 41, wherein Ring B is: 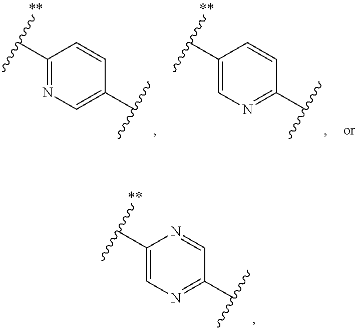 [SMILES: *c1ccc(*)nc1 ; *c1ccc(*)nc1 ; *c1cnc(*)cn1]
wherein ** denotes a bond to Ring A.

43. The compound of any one of claims 1-31, wherein Ring B is a bridged bicyclic cycloalkyl.

44. The compound of claim 43, wherein Ring A is bicyclo[2.2.2]octanyl or bicyclo[1.1.1]pentanyl.

45. The compound of claim 44, wherein Ring B is: 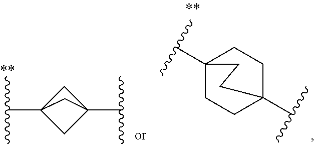
wherein Z is halo and ** denotes a bond to Ring A.

46. The compound of one of claims 1-45, wherein R⁴ is hydrogen, a protecting group, or —C(O)—CH₃.

47. The compound of claim 1 selected from: 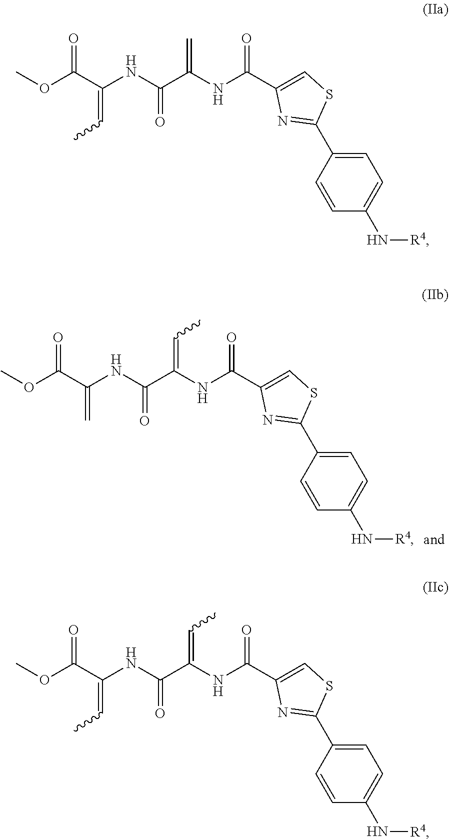
wherein R⁴ is hydrogen, a protecting group, or —C(O)—CH₃; and
wherein a bond drawn as “ [symbol] ” denotes either possible stereochemistry of the attached alkene, E or Z.

48. The compound of claim 1 selected from: 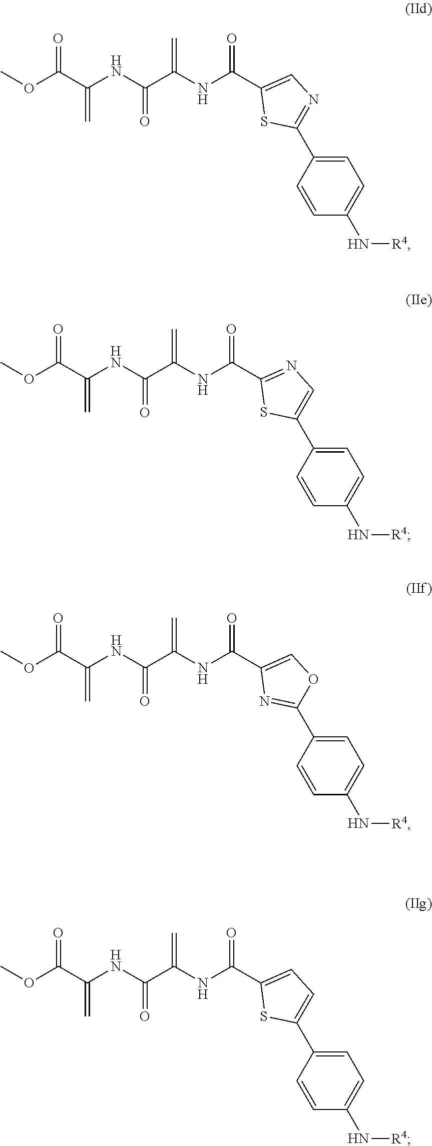 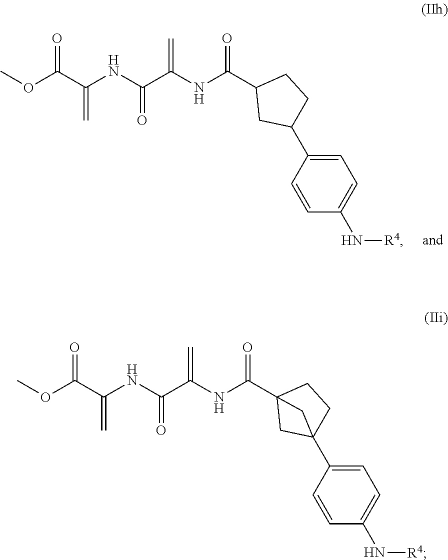
wherein R⁴ is hydrogen, a protecting group, or —C(O)—CH₃.

49. The compound of claim 1, selected from:

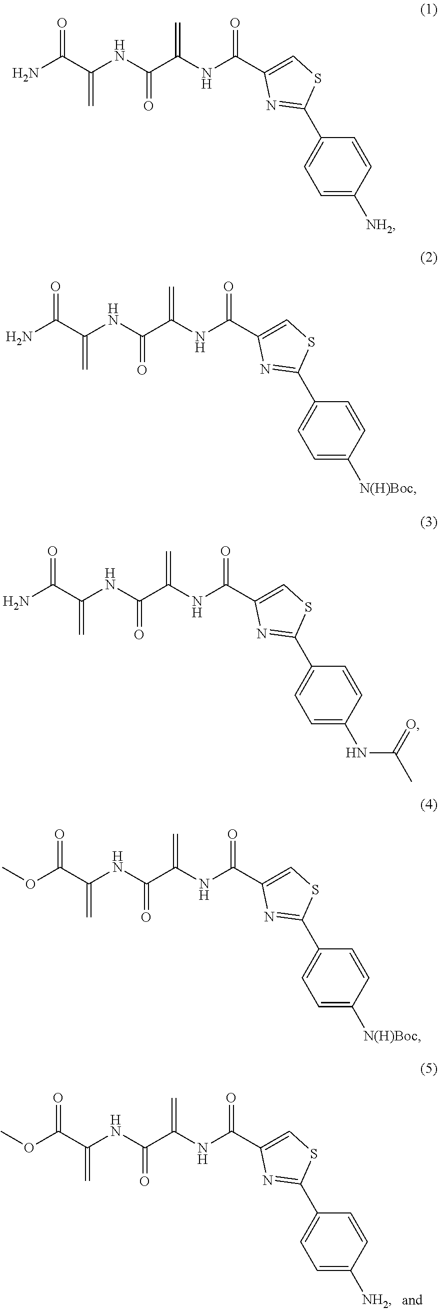

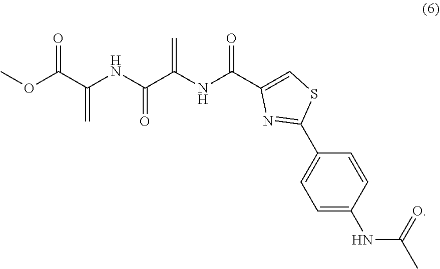 [SMILES: C=C(NC(=O)c1csc(-c2ccc(NC(C)=O)cc2)n1)C(=O)NC(=C)C(=O)OC]

50. The compound of claim 1, selected from: 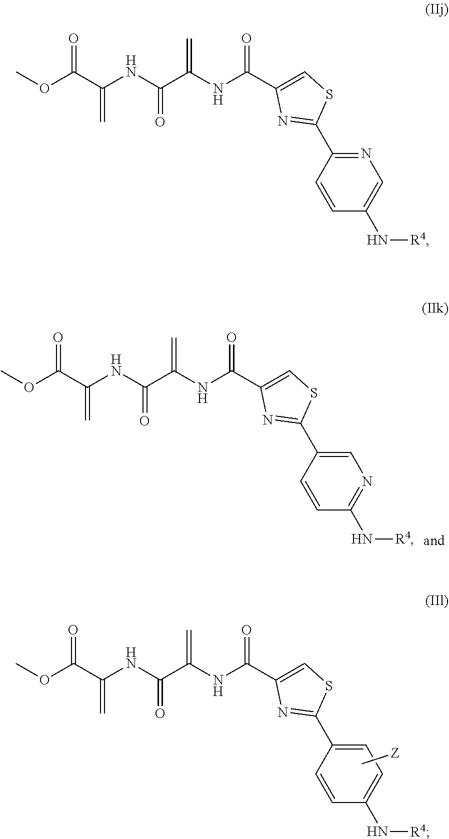
wherein R⁴ is hydrogen, a protecting group, or —C(O)—CH₃;
and wherein Z is selected from fluorine, chlorine, bromine, and iodine.

51. The compound of claim 1, selected from: 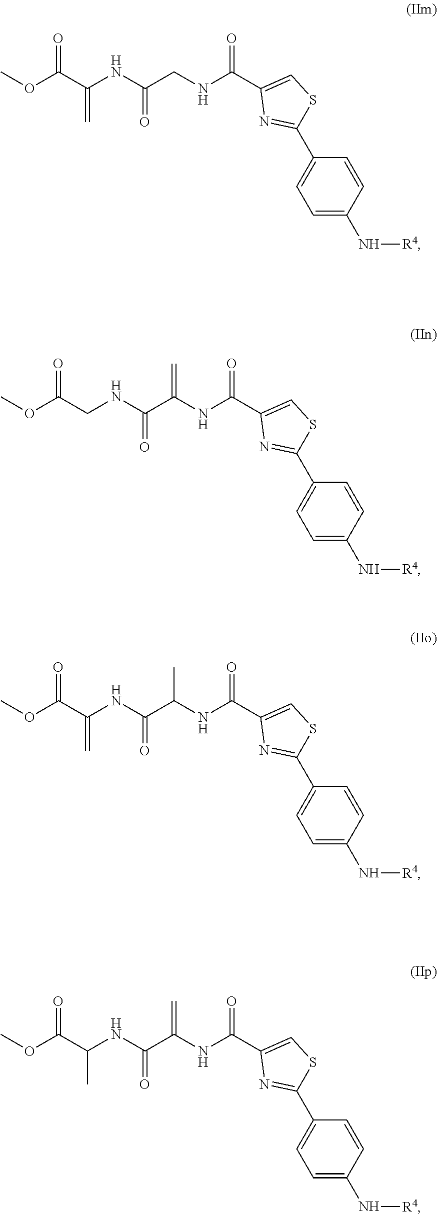 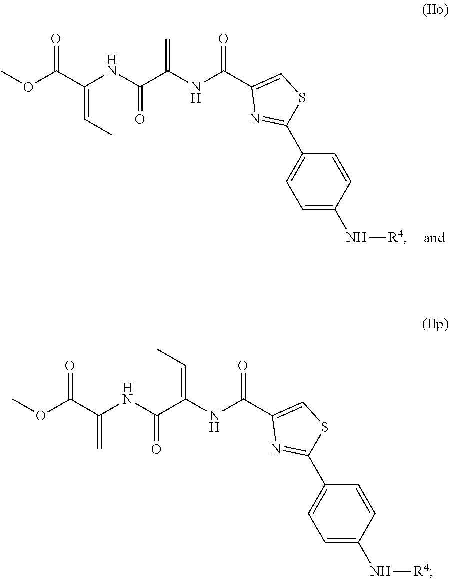
wherein R⁴ is hydrogen, a protecting group, or —C(O)—CH₃.

52. The compound of claim 51, wherein the protecting group is Boc.

53. The compound of any one of claims 1-45, wherein R⁴ is -L′.

54. The compound of claim 53, wherein:
L′ is —C(O)—X—C(O)OH or —C(O)—X—C(O)NH₂;
X is —(CH₂)ₙ—; and
n is 2, 3, 4 or 5.

55. The compound of claim 54, selected from: 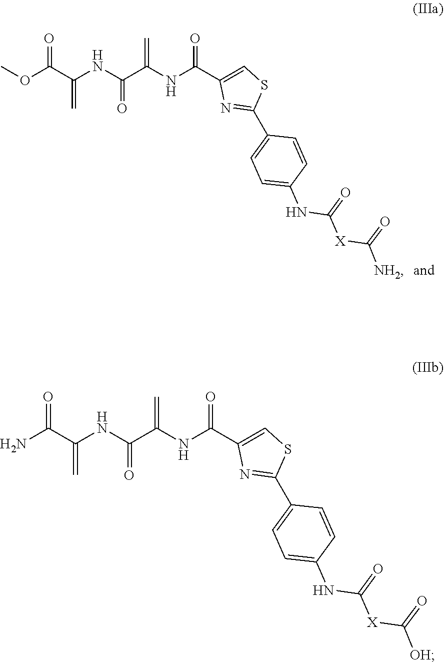
wherein X is —(CH₂)ₙ—; and
n is 2, 3, 4 or 5.

56. The compound of claim 54, selected from: 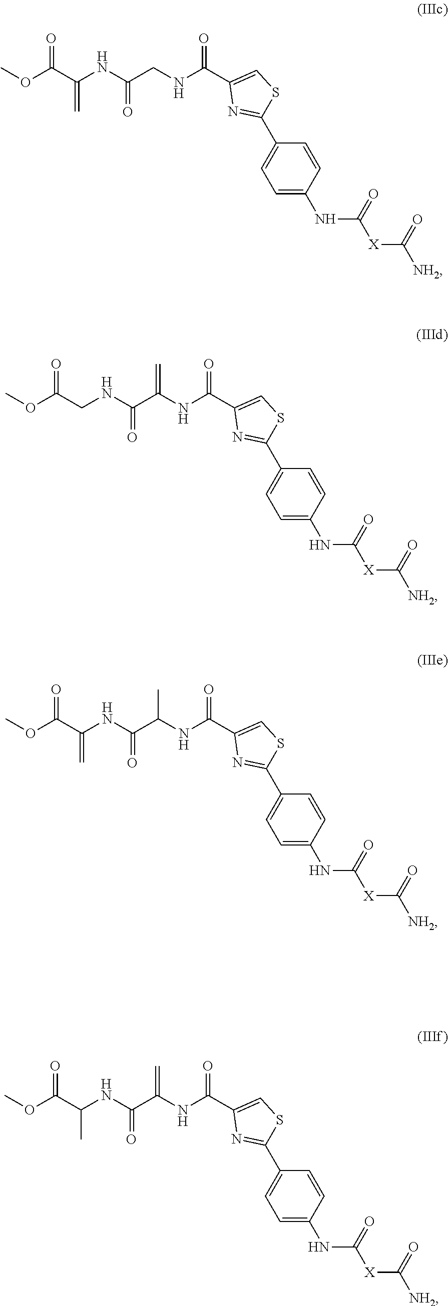 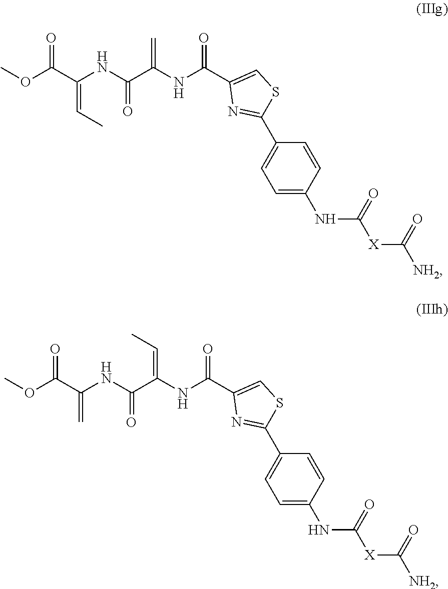 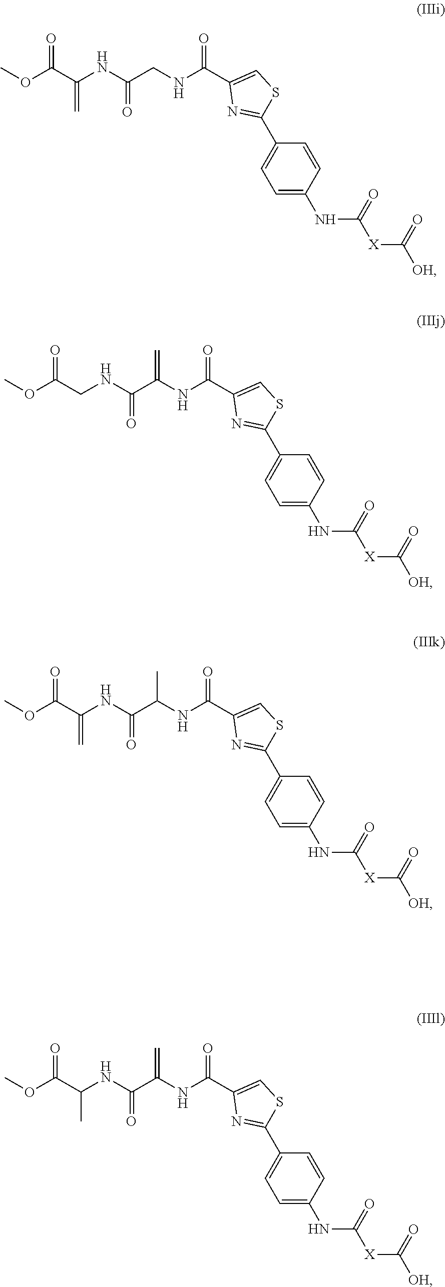 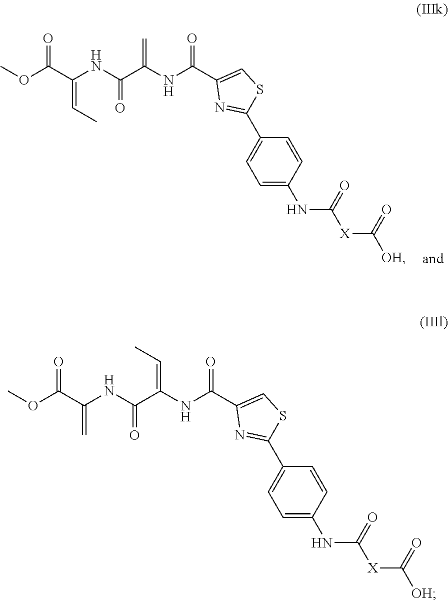
wherein X is —(CH₂)ₙ—; and
n is 2, 3, 4 or 5.

57. The compound of any one of claims 1-45, wherein R⁴ is -L-Y.

58. The compound of claim 57, wherein L is a cleavable linker.

59. The compound of claim 57, wherein L is a non-cleavable linker.

60. The compound of any one of claims 57-59, wherein L has a chain length of about 2 to about 30 atoms.

61. The compound of claim 60, wherein L has a chain length of about 5 to about 20 atoms.

62. The compound of claim 57, wherein:
L is —C(O)—X—C(O)—;
X is —(CH₂)ₙ—; and
n is 2, 3, 4 or 5.

63. The compound of claim 57, wherein:
L is —C(O)—X—C(O)—;
X is —(CH₂CH₂—O—)ₘ—(CH₂CH₂)—; and
m is 2, 3, 4, 5, or 6.

64. The compound of claim 1 selected from: 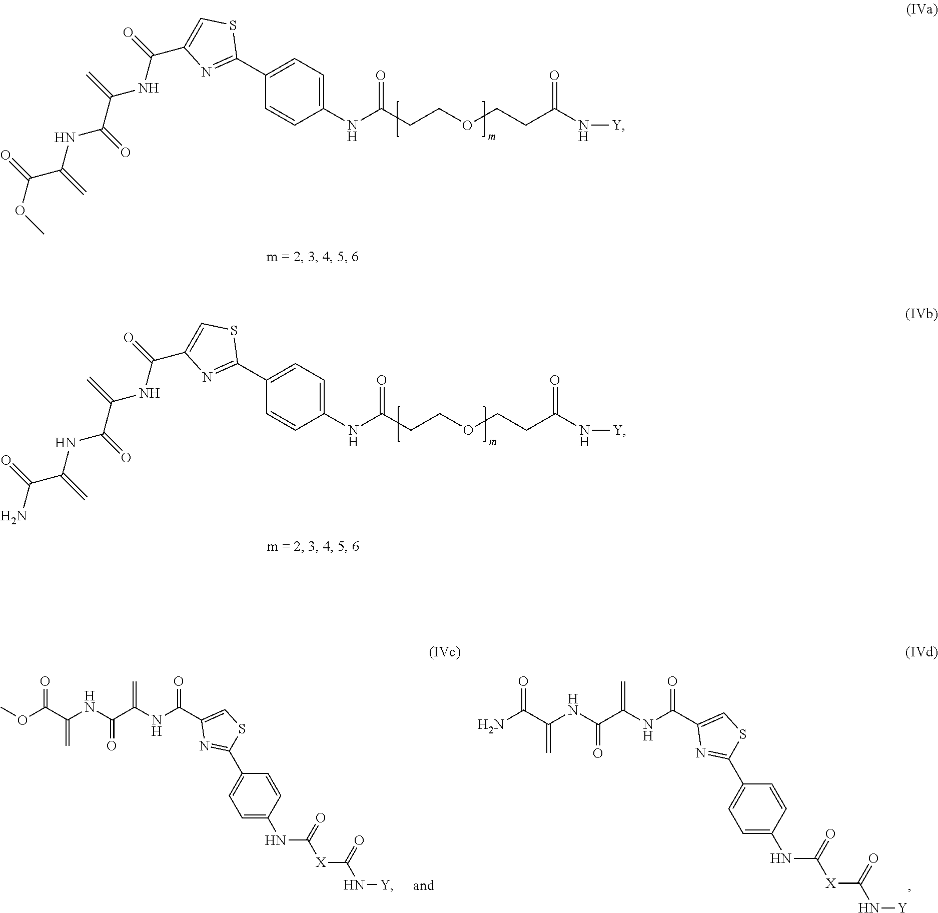
wherein X is —(CH₂)ₙ—; and
n is 2, 3, 4 or 5.

65. The compound of claim 1 selected from: 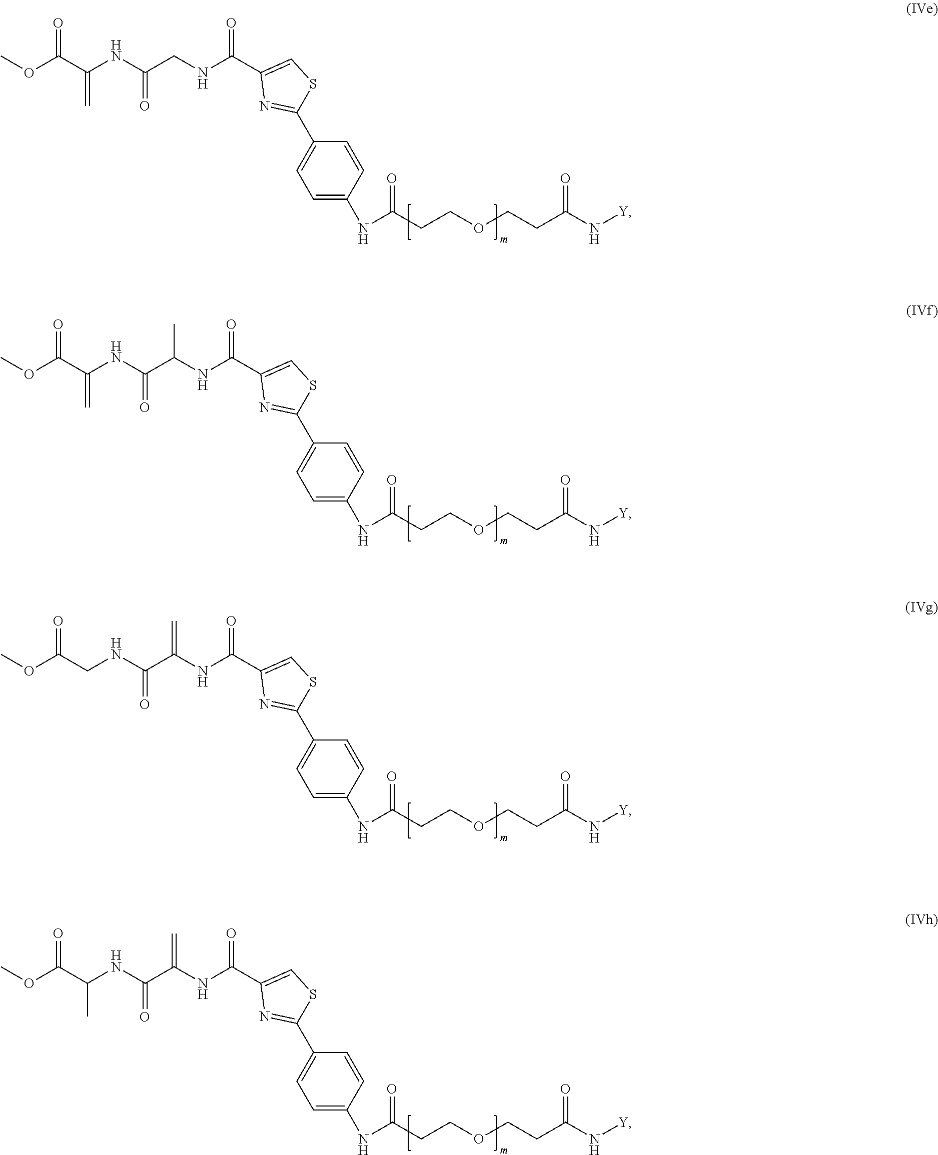 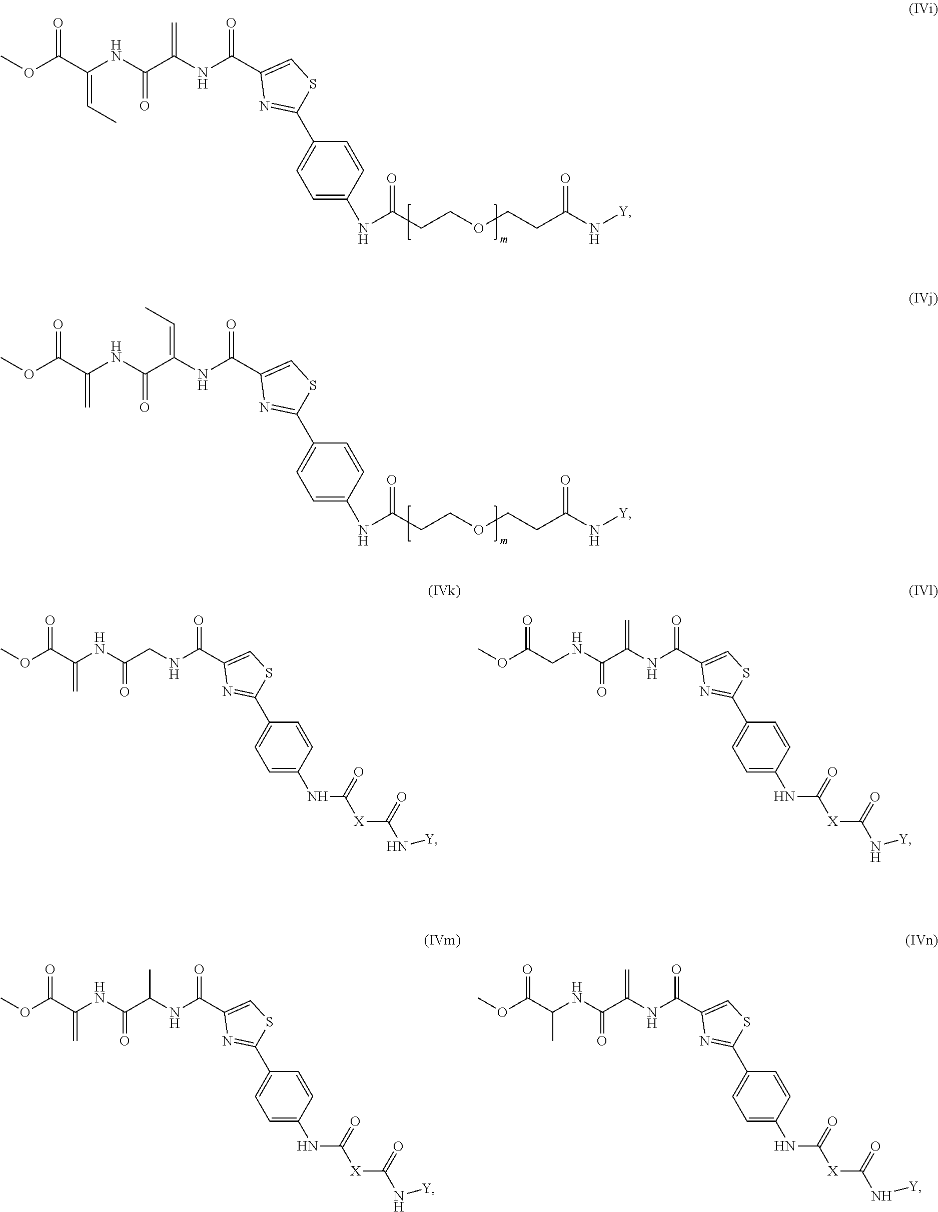 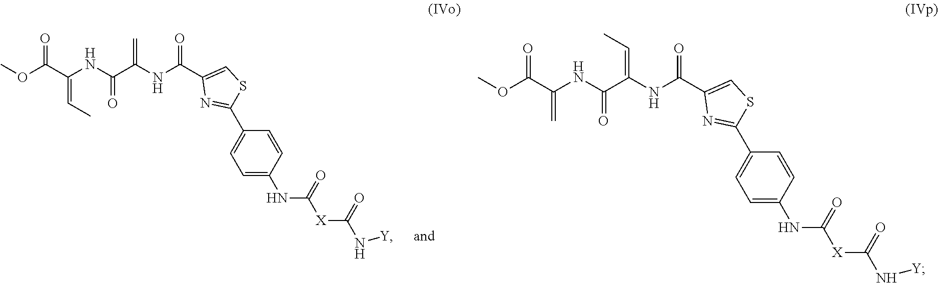
wherein X is —(CH₂)ₙ—;
m is 2, 3, 4, 5, or 6; and
n is 2, 3, 4 or 5.

66. The compound of claim 53, wherein L′ comprises an alkynyl or azido.

67. The compound of claim 66, wherein
R⁴ is —C(O)—X′—C≡CH or —C(O)—X′—N₃;
X′ is —(CH₂)ₙ—; and
n is 2, 3, 4 or 5.

68. The compound of claim 57, wherein L comprises a heteroayl.

69. The compound of claim 68, wherein L comprises a triazolyl.

70. The compound of claim 69, wherein
R⁴ is 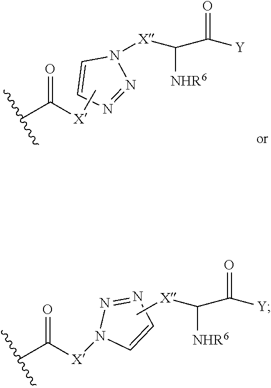
R⁶ is —H or —C(O)CH₃;
X′ is —(CH₂)ₙ—;
X″ is —(CH₂)ₒ—;
n is 2, 3, 4 or 5; and
o is 2, 3, 4 or 5.

71. The compound of claim 70, wherein
R⁴ is 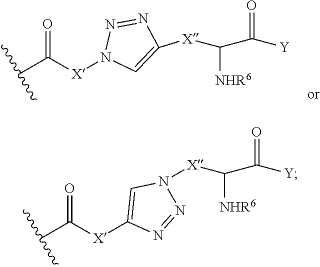
R⁶ is —H or —C(O)CH₃;
X′ is —(CH₂)ₙ—;
X″ is —(CH₂)ₒ—;
n is 2, 3, 4 or 5; and
o is 2, 3, 4 or 5.

72. The compound of claim 69, wherein
R⁴ is 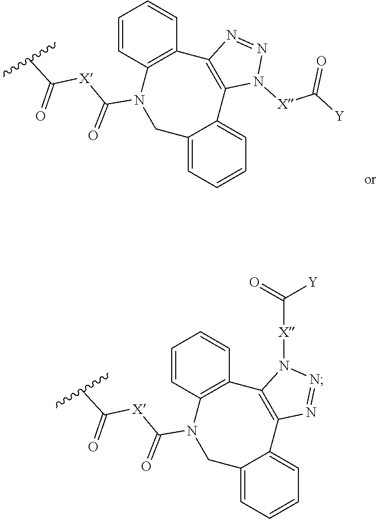
X′ is —(CH₂)ₙ—;
X″ is —(CH₂)ₒ—;
n is 2, 3, 4 or 5; and
o is 2, 3, 4 or 5.

73. The compound of any one of claims 57-72, wherein:
Y is a berberin cation, rhodamine cation, an indolium cation, a pyridinium cation, a tetraguanidinium cation, cyanine derivatives, a guanidinium cation, a biguanidinium cation, a triphenylphosphonium cation, a triethylammonium cation, a triphenylamine, a tetraphenylethene moiety, arylphosphonium cation, an SS peptide, a mitochondrial penetrating peptide (MPP), a mitochondrial targeting sequence (MTS) peptide, a hemigramicidin S-linked nitroxide, a Dequalinium (DQA) cation, a delocalized lipophilic cation, F16 ((E)-4-(1H-indol-3-ylvinyl)-N-methylpyridinium iodide), (L-cyclohexyl alanine-D-arginine)3, a mitochondrial-targeted nanocarrier, a DDDK peptide, glycyrrhetinic acid, α-tocopheryl succinate (α-TOS), a graphene oxide nano carrier, PEG-proapoptotic peptide (KLAKLAK)2, a Dmt-D-Arg-Phe-Lys-NH₂ peptide, pyruvaldehyde, N-Nonyl acridine orange, quinoline, styryl fluorophores, or 15d-PGJ2.

74. The compound of claim 73, wherein Y is a mitochondrial penetrating peptide (MPP).

75. The compound of claim 73, wherein Y has the structural formula (V):

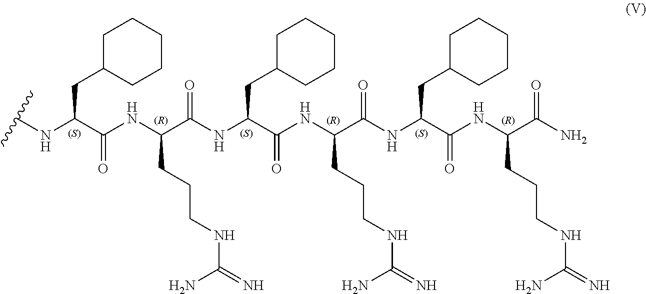

76. The compound of claim 1, selected from: 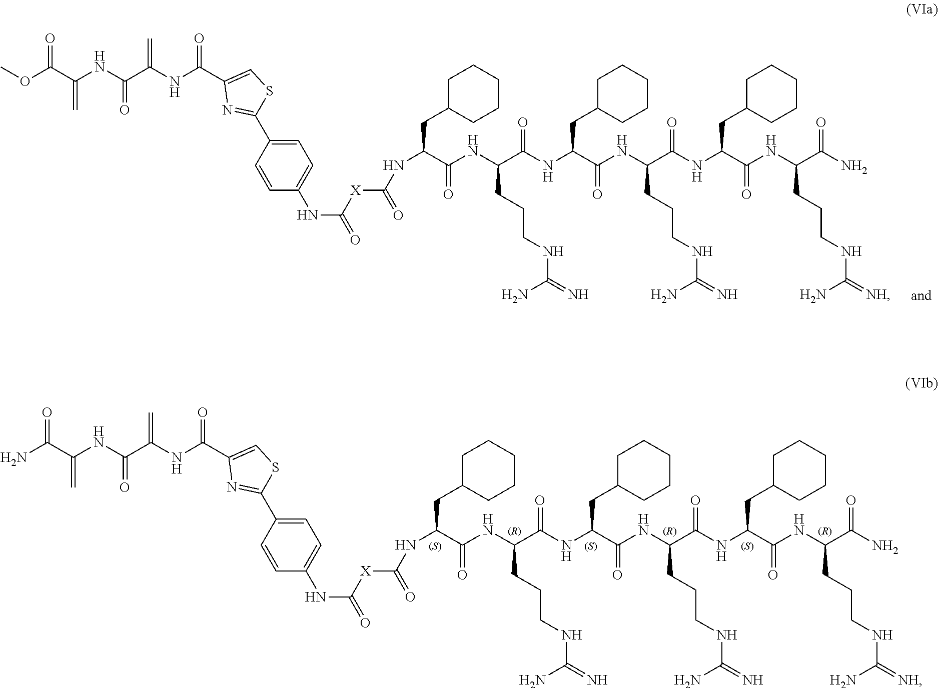
or a pharmaceutically acceptable salt thereof;
wherein X is [—(CH₂)ₙ-] or [—(CH₂CH₂—O—)ₘ—(CH₂CH₂)—];
n is 3, 4 or 5; and
m is 2, 3, 4, 5, or 6.

77. A pharmaceutically acceptable composition comprising a compound of any one of claims 1-76; and a pharmaceutically acceptable carrier.

78. The composition of claim 77, formulated for oral or parenteral delivery.

79. A composition comprising a compound of any of the compounds of claims 1-76, wherein the compound is contained within a nanoparticle, liposome or micelle, wherein the nanoparticle, liposome, or micelle is conjugated to a mitochondrial targeting moiety.

80. A composition comprising a compound of Formula (IA), wherein R⁴ is hydrogen, a protecting group, or —C(O)—CH₃,
wherein the compound is contained within a nanoparticle, liposome or micelle, wherein the nanoparticle, liposome, or micelle is conjugated to a mitochondrial targeting moiety.

81. A composition comprising a compound of Formula (IB), wherein R⁴ is hydrogen, a protecting group, or —C(O)—CH₃,
wherein the compound is contained within a nanoparticle, liposome or micelle, wherein the nanoparticle, liposome, or micelle is conjugated to a mitochondrial targeting moiety.

82. The composition of any one of claims 79-81, wherein the nanoparticle, liposome or micelle is selected from poly(ethylene glycol), poly(ε-caprolactone), polysaccharides, poly[(2-hydroxypropyl)-methacrylic acid], poly(lactic-co-glycolic acid), and any combinations of the foregoing.

83. A method of treating a cancer (e.g., solid tumor or hematological cancer) comprising administering to a subject in need thereof a therapeutically effective amount of a compound of any one of claims 1-76, or a composition of any one of claims 77-82.

84. The method of claim 83, wherein the cancer (solid tumor or hematological) is selected from lung, breast, prostate, melanoma, esophageal, leukemia, cervical, liver, colon, gastric, colorectal, glioblastoma, head and neck, pancreatic, mesothelioma, and ovarian.

85. The method of claim 84, wherein the cancer is selected from mesothelioma, lung, ovarian, and breast.
